(19) United States
(12) Patent Application Publication
(10) Pub. No.: US 2004/0220387 A1
(43) Pub. Date: Nov. 4, 2004

(54) Methods

(75) Inventors: Angela Veronica Flannery, Cheshire (GB); Maria Christina Martina Orr, Cheshire (GB)

Correspondence Address: ROPES & GRAY LLP, ONE INTERNATIONAL PLACE, BOSTON, MA, 02110-2624, US

(73) Assignee: AstraZeneca UK Limited, London (GB)

(21) Appl. No.: 10/779,271

(22) Filed: Feb. 13, 2004

Related U.S. Application Data
(63) Continuation-in-part of application No. 09/463,844, filed on Feb. 1, 2000, now abandoned.
Application No. 09/463,844 is a 371 of international application No. PCT/GB98/02259, filed on Jul. 28, 1998.
(60) Provisional application No. 60/535,986, filed on Jan. 12, 2004.

(30) Foreign Application Priority Data
Aug. 1, 1997 (GB) 9716162.4

(51) Int. Cl. G01N 33/53; C07H 21/04; C07K 14/705

(52) U.S. Cl. 530/350; 435/69.1; 435/320.1; 435/325; 435/7.1; 536/23.5

(57) ABSTRACT

The invention relates to a method for the detection of bipolar affective disorder or a predisposition thereto in a human. The invention also relates to methods and materials for analysing allelic variation in the ZGGBP-1 gene, and to the use of ZGGBP-1 polymorphism in the diagnosis and treatment of diseases in which modulation of ZGGBP-1 activity could be of therapeutic benefit, particularly disease states associated with neurological affective disorders such as bipolar affective disorder.

### RELATED APPLICATIONS

This application is a continuation-in-part of U.S. application Ser. No. 09/463,844, filed on Feb. 1, 2000, which is a national stage filing under 35 U.S.C. 371 of International Application PCT/GB98/00259, filed on Jul. 28, 1998, which claims priority from United Kingdom Application No. 9716162.4, filed Aug. 1, 1997. This application further claims priority to U.S. Provisional Application Ser. No. 60/535,986, filed on Jan. 12, 2004. All of the above specifications are incorporated by reference herein. International Application PCT/GB98/00259 was published under PCT Article 21(2) in English.

This invention relates to polymorphisms in the human ZGGBP-1 gene. The invention also relates to methods and materials for analysing allelic variation in the ZGGBP-1 gene, and to the use of ZGGBP-1 polymorphism in the diagnosis and treatment of diseases in which modulation of ZGGBP-1 activity could be of therapeutic benefit, particularly disease states associated with affective neurological disorders such as bipolar affective disorder (BPAD).

Affective neurological disorders comprise a broad and heterogeneous category of psychiatric illness with a prevalence of up to 20% in the population. The most severe of these disorders is bipolar type I, which affects approximately 1% of the population and this rate is fairly consistent across countries. The disease affects young adults, with a mean age of onset of 22 years. Treatment depends upon the phase of the disease and pharmacological agents include lithium carbonate, carbamazepine or valproic acid or tricyclic antidepressants. Monoamine oxidase inhibitors and selective serotonin re-uptake inhibitors are now also being used. The success rate of individual drugs is variable (normally approximately 50% although this can rise to 70-80% overall) and some patients are treated with a combination of agents, although most have some unwanted side-effects. At present, the precise diagnosis of individual affective disorders is difficult and new, gene-based, diagnostic methods are desirable.

Family, twin and adoption studies have suggested the importance of genetic predisposition to bipolar affective disorder (BPAD). On this basis, several groups have undertaken genetic linkage analysis in families with a high incidence of the disorder to find a causal gene. Many of the studies show conflicting data suggesting that a single gene is unlikely to be the cause. Rather, multiple interacting genetic traits may be involved.

A study carried out by Stine et al. (Am. J. Hum. Genet. (1995) 57(6), 1384-1394) identified two regions on chromosome 18 showing strong linkage to the disease including a region located on 18q21. Based on this data, the human ZGGBP-1 gene was first identified in PCT patent application WO99/06539 and is localised to human chromosome 18q21. ZGGBP-1 shows appreciable sequence homology to the Nedd-4 gene on chromosome 15. Nedd-4 is the human homologue of the mouse nedd-4 gene which is known to be differentially expressed during neural development and to be involved in signal transduction. Human Nedd-4 has been shown (Schild et al. EMBO J. (1996) 15, 2381-2387; Straub et al. EMBO J. (1996) 15, 2371-2380) to be a negative regulator of a sodium channel which is deleted in Liddle's syndrome (a hereditary form of hypertension).

Nedd-4 was originally isolated as a partial cDNA clone from a mouse brain library (Kumar et al. Biochem. Biophys. Res. Commun. (1992) 185, 1155-1161) as one of a set of genes which were differentially expressed during development (neural precursor cells expressed developmentally down-regulated). The derived amino acid sequence contains three copies of the WW domain (Andre & Springael, Biochem. Biophys. Res. Commun. (1994) 205, 1201-1205; Bork & Sudol, Trends Biochem Sci (1994) 19, 531-533; Hofinann & Boucher, FEBS Letts. (1995) 358, 153-157), a Ca lipid binding (CaLB/C2) domain (Brose et al. Proc. Natl. Acad. Sci. USA. (1995) 92, 2563-2567) and a Hect (homologous to the E6-AP carboxyl terminus) domain, which has homology to a ubiquitin ligase (E3) enzyme (Huibregtse et al. Proc. Natl. Acad. Sci. USA (1995) 92, 2563-2567). The human homologue of Nedd-4 was isolated as a randomly cloned EST (KIAA0093) from immature myeloblast mRNA (Nomura et al. DNA Res. (1994) 2, 3743) and shown by sequence comparison to have 86% identity at the amino acid level to the mouse sequence. The human sequence, however, has a fourth copy of the WW domain.

The WW domain is a 40 amino acid sequence found in several unrelated proteins. The two highly conserved tryptophans give it its name. The function of the domain is thought to be involved in protein-protein interactions. Despite their functional diversity, the proteins listed all appear to be involved in cell signalling or regulation. It has been shown that the WW domains of Nedd-4 interact with the proline-rich PY motifs in the epithelial sodium channel in the kidney (Schild et al. EMBO J. (1996) 15, 2381-2387). Mutational deletion of the PY motifs in the epithelial sodium channel in Liddle's syndrome, an inherited disease causing systemic hypertension characterised by hyperactivity of the sodium channel, has been shown to abrogate binding of Nedd-4 (Straub et al. EMBO J. (1996) 15, 2371-2380). It is therefore likely that Nedd-4 has a negative regulatory role when bound to the channel.

The Hect domain is an E3 ubiquitin-protein ligase domain and enzymes with this domain catalyse polyubiquitination, which is involved in several cellular processes including proteolytic degradation, processing protein trafficking etc.

The CaLB/C2 domain is thought to be involved in calcium-dependent phospholipid binding, although some proteins containing this domain do not bind calcium and other putative functions for the C2 domain such as binding to inositol-1,3,4,5-tetraphosphate have been suggested. Examples of proteins containing this domain are Protein Kinase C (PKC) isoenzymes and synaptogamins.

In PCT patent application WO02/12439, ZGGBP-1 shows homology to a DNA encoding a gene differentially expressed in diseased kidney tissue.

In PCT patent application WO97/12962, ZGGBP-1 shows 99% homology over 60% of its length with a gene annotated as Human protein ubiquitin ligase pub3. This gene is involved in cdc25 phosphatase and p53 ubiquitination of cell growth and proliferation.

In PCT patent application WO97/37223, ZGGBP-1 shows 99% homology over 36% of the length with the novel human gene designated WWP4. The gene was identified and isolated from a cDNA expression library generated from LNCap prostate cancer cell line

Drugs which change the level of a ZGGBP-1 mediated response or change the biological activity of ZGGBP-1 are expected to be useful in the treatment of all conditions in which ZGGBP-1 plays a pathophysiological role. Such drugs are particularly useful in the treatment of conditions associated with neurological affective disorders such as bipolar affective disorder (BPAD).

DNA polymorphisms are variations in DNA sequence between one individual and another. DNA polymorphisms may lead to variations in amino acid sequence and consequently to altered protein structure and functional activity. Polymorphisms may also affect mRNA synthesis, maturation, transportation and stability. Polymorphisms which do not result in amino acid changes (silent polymorphisms) or which do not alter any known consensus sequences may nevertheless have a biological effect, for example by altering mRNA folding or stability.

Knowledge of polymorphisms may be used to help identify patients most suited to therapy with particular pharmaceutical agents (this is often termed “pharmacogenetics”). Pharmacogenetics can also be used in pharmaceutical research to assist the drug selection process. Polymorphisms may be used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40438, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

A cDNA encoding ZGGBP-1 is set out as SEQ ID NO:2, with the first nucleotide of the ZGGBP-1 coding region accorded position 336. A peptide sequence of ZGGBP-1 is set out as SEQ ID NO: 3.

The genomic DNA sequence of the human ZGGBP-1 gene has been determined. Part of the genomic DNA sequence of the human ZGGBP-1 gene is set out as SEQ ID NO: 1, and shows the first nucleotide of exon 2 accorded position 1. The positions of exons 2-29 as defined in SEQ ID NO: 1 are set out in the table below.

| | Start Position of | End Position of |
|---|---|---|
| Region | SEQ ID NO: 1 | SEQ ID NO: 1 |
| Exon 2 | 1 | 76 |
| Exon 3 | 119850 | 119918 |
| Exon 4 | 199489 | 199569 |
| Exon 5 | 202960 | 203000 |
| Exon 6 | 206065 | 206115 |
| Exon 7 | 270043 | 270093 |
| Exon 8 | 276486 | 276545 |
| Exon 9 | 277291 | 277395 |
| Exon 10 | 279057 | 279224 |
| Exon 11 | 283057 | 283188 |
| Exon 12 | 284799 | 284974 |
| Exon 13 | 287879 | 287953 |
| Exon 14 | 295099 | 295229 |
| Exon 15 | 295739 | 295858 |
| Exon 16 | 296967 | 297164 |
| Exon 17 | 303598 | 303675 |
| Exon 18 | 305052 | 305105 |
| Exon 19 | 311255 | 311313 |
| Exon 20 | 318329 | 318394 |
| Exon 21 | 320060 | 320290 |
| Exon 22 | 321808 | 321927 |
| Exon 23 | 324457 | 324528 |
| Exon 24 | 327235 | 327330 |
| Exon 25 | 337307 | 337380 |
| Exon 26 | 339588 | 339647 |
| Exon 27 | 341500 | 341559 |
| Exon 28 | 343146 | 343252 |
| Exon 29 | 344707 | 344805 |

All positions herein of polymorphisms in the intronic regions of the human ZGGBP-1 gene are defined with reference to SEQ ID NO:1 unless stated otherwise or apparent from the context. The present invention is based on the discovery of seven polymorphisms in the intronic sequence of the human ZGGBP-1 gene.

According to one aspect of the invention there is provided a diagnostic method for the detection of bipolar affective disorder or a predisposition thereto in a human which method comprises determining the sequence of the human at any one of the following positions: positions 119726, 221374, 270159, 294977, 298758, 324317 and 343331 of SEQ ID NO: 1, wherein the base at position 119726 is A, C or G; the base at position 221374 is A, C or G; the base at position 270159 is A, C or G; the base at position 294977 is T, C or G; the base at position 298758 is A, C or G; the base at position 324317 is A, C or G; and the base at position 343331 is A, C or T.

In one embodiment of the invention, preferably the base at position 119726 is C; the base at position 221374 is C; the base at position 270159 is C; the base at position 294977 is G; the base at position 298758 is C; the base at position 324317 is C; and the base at position 343331 is A.

According to a further aspect of the present invention there is provided a method for the detection of a polymorphism in the human ZGGBP-1 gene, which method comprises determining the sequence of the human at any one of the following positions: positions 119726, 221374, 270159, 294977, 298758, 324317 and 343331 of SEQ ID NO: 1.

The term “human” includes both a human having or suspected of having a ZGGBP-I mediated response and an asymptomatic human who may be tested for predisposition or susceptibility to such a response.

The term “detection of a polymorphism” refers to determination of the genetic status of an individual at a polymorphic position (in which the individual may be homozygous or heterozygous at each position).

The term “ZGGBP-1 mediated response” means any disease in which changing the level of a ZGGBP-1 mediated response or changing the biological activity of ZGGBP-1 would be of therapeutic benefit.

The term “polymorphism” includes single nucleotide substitution, nucleotide insertion and nucleotide deletion, which in the case of insertion and deletion includes insertion or deletion of one or more nucleotides at a position of a gene and variable numbers of a repeated DNA sequence.

In one embodiment of the invention preferably the polymorphism is further defined as:

polymorphism at position 119726 is presence of T and/or C;

polymorphism at position 221374 is presence of T and/or C;

polymorphism at position 270159 is presence of T and/or C;

polymorphism at position 294977 is presence of A and/or G;

polymorphism at position 298758 is presence of T and/or C;

polymorphism at position 324317 is presence of T and/or C; and

polymorphism at position 343331 is presence of G and/or A.

Preferably, two or more polymorphisms are detected.

Preferred methods for detection of nucleic acid polymorphism are amplification refractory mutation system (ARMS) and restriction fragment length polymorphism (RFLP).

Allelic variation at each position in the ZGGBP-1 gene, including preferred variation is described herein.

The status of the individual may be determined by reference to allelic variation at any one, two, three, four, five, six or all seven positions optionally in combination with any other polymorphism in the gene that is (or becomes) known.

The test sample of nucleic acid is conveniently present in a sample of blood, sputum, skin, or other body fluid or tissue obtained from an individual. It will be appreciated that the test sample may equally comprise a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures that may be used to detect the presence or absence of variant nucleotides at one or more polymorphic positions of the invention. In general, the detection of allelic variation requires a mutation discrimination technique, optionally an amplification reaction and optionally a signal generation system. A number of mutation detection techniques, some based on PCR, may be used in combination with a number of signal generation systems, a selection of these are highlighted below. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| CMC | Chemical mismatch cleavage |
| COPS | Competitive oligonucleotide priming system |
| DGGE | Denaturing gradient gel electrophoresis |
| dHPLC | Denaturing High Performance Liquid Chromatography |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SSCP | Single-strand conformation polymorphism analysis |
| SSR | Self sustained replication |
| TGGE | Temperature gradient gel electrophoresis |
| UTR | Untranslated region |
| WAVE | See - www.transgenomic.com |

Mutation Detection Techniques

General: DNA sequencing, Sequencing by hybridisation

Scanning: PTT*, SSCP, DGGE, TGGE, dHPLC (using WAVE), Cleavase, Heteroduplex analysis, CMC, Enzymatic mismatch cleavage * Note: not useful for detection of promoter polymorphisms.

Hybridisation Based: Solid phase hybridisation: Dot blots, MASDA, Reverse dot blots, Oligonucleotide arrays (DNA Chips).

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York).

Extension Based: ARMS™, ALEX™ —European Patent No. EP 332435 B I (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay

Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Ultra Violet absorption (WAVE) Colorimetric, Hybridisation protection assay, Mass spectrometry.

Further Amplification Methods

SSR, NASBA, LCR, SDA, b-DNA

Protein Variation Detection Methods

Immunoassay

Immunohistology

Peptide sequencing

Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom. Histological techniques are described in Theory and Practice of Histological Techniques by J D Bancroft & A Stevens, 4^{th} Edition, Churchill Livingstone, 1996. Protein sequencing is described in Laboratory Techniques in Biochemistry and Molecular Biology, Volume 9, Sequencing of Proteins and Peptides, G Allen, 2^{nd} revised edition, Elsevier, 1989.

Preferred mutation detection techniques include DNA Sequencing, dHPLC & WAVE, ARMS™, ALEX™, COPS, Taqman, Molecular Beacons, RFLP, restriction site based PCR and FRET techniques, polyacrylamide gel electrophoresis and capillary electrophoresis.

Particularly preferred methods include ARMS™ and RFLP based methods. ARMS™ is an especially preferred method.

In a further aspect, the diagnostic methods of the invention are used to assess the pharmacogenetics of a drug in the treatment of ZGGBP-1-mediated diseases, particularly disease states associated with neurological affective disorders such as BPAD.

Assays, for example reporter-based assays, may be devised to detect whether one or more of the above polymorphisms affect transcription levels and/or message stability.

Individuals who carry particular allelic variants of the ZGGBP-1 gene may exhibit differences in their ability to regulate protein biosynthesis under different physiological conditions and may display altered abilities to react to different diseases. In addition, differences in protein regulation and/or the protein's properties arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition of an individual to diseases mediated by ZGGBP-1. This may be particularly relevant in the regulation of metabolism and in diseases that are mediated by ZGGBP-1, for example neurological affective disorders such as BPAD. The present invention may be used to recognise individuals who are particularly at risk from developing these conditions.

In particular, a positive association has been observed between the presence of the polymorphism at position 221374 of ZGGBP-1 of SEQ ID NO:1 and a BPAD cohort (see Example 2). The results indicate that this polymorphism in the ZGGBP-1 gene of an individual may be used to detect the presence of or likely future development of BPAD. Therefore in a preferred aspect of the invention we provide the use of the polymorphism at position 221374 in the ZGGBP-1 gene of SEQ ID NO:1 to detect the presence of BPAD or a predisposition thereto.

In a further aspect, the diagnostic methods of the invention are used in the development of new drug therapies, which selectively target one or more allelic variants of the ZGGBP-1 gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the invention there is provided a human ZGGBP-1 gene or its complementary strand comprising a allelic variant polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 contiguous bases of the human ZGGBP-1 gene and comprising an allelic variant selected from any of the following:

| Region | Variant | Position of SEQ ID NO: 1 |
|---|---|---|
| Intron 2 | C | 119726 |
| Intron 6 | C | 221374 |
| Intron 6 | C | 270159 |
| Intron 13 | G | 294977 |
| Intron 16 | C | 298758 |
| Intron 22 | C | 324317 |
| Intron 28 | A | 343331 |

According to another aspect of the present invention there is provided a human ZGGBP-1 gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The scope of the invention does not extend to any nucleic acid as it is found in nature. A nucleic acid of the invention is preferably in isolated form, for example through being at least partially purified from any substance with which it occurs naturally.

Novel sequence disclosed herein, may be used in another embodiment of the invention to regulate expression of the gene in cells by the use of antisense constructs. To enable methods of down-regulating expression of the gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides, which are complementary to and hybridisable with any portion of novel gene mRNA disclosed herein, are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from previously known polynucleotides are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

The invention further provides nucleotide primers which can detect the polymorphisms of the invention.

According to another aspect of the present invention there is provided an allele specific primer capable of detecting a ZGGBP-1 gene polymorphism, at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a ZGGBP-1 gene polymorphism at one or more of the positions defined herein.

The allele specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a ZGGBP-1 gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s), nucleotides, and polymerase(s) such as thermostable polymerases, for example taq polymerase.

In another aspect of the invention, a polymorphism of this invention may be used as a genetic marker in a linkage study. This particularly applies to polymorphisms of relatively high frequency.

Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each polymorphic position and n is the number of polymorphic positions. One approach to identifying mutations or polymorphisms, which are correlated with clinical response, is to carry out an association study using all the haplotypes that can be identified in the population of interest. The frequency of each haplotype is limited by the frequency of its “rarest” allele, so that polymorphisms with low frequency alleles are particularly useful as markers of low frequency haplotypes. As particular mutations or polymorphisms associated with certain clinical features, such as adverse or abnormal events, are likely to be of low frequency within the population, low frequency polymorphisms may be particularly useful in identifying these mutations (for examples see: De Stefano V et al., Ann Hum Genet (1998) 62:481-90; and Keightley A M et al., Blood (1999) 93:4277-83.

In another aspect of the invention we provide a method for the diagnosis of ZGGBP-1-mediated disease, which method comprises:

i) obtaining sample nucleic acid from an individual,

ii) detecting the presence or absence of a variant nucleotide at any one of the following positions: positions 119726, 221374, 270159, 294977, 298758, 324317 and 343331 in the ZGGBP-1 gene as defined by the positions in SEQ ID NO:1;

iii) determining the status of the individual by reference to polymorphism in the ZGGBP-1 gene.

According to another aspect of the present invention there is provided a method of treating a human in need of treatment with a ZGGBP-1 drug in which the method comprises:

i) detection of a polymorphism in ZGGBP-1 in a human, which detection comprises determining the sequence of the nucleic acid at one or more of: positions 119726, 221374, 270159, 294977, 298758, 324317 and 343331 of SEQ ID NO:1, and determining the status of the human by reference to polymorphism in ZGGBP-1; and

ii) administering an effective amount of the drug.

The term “ZGGBP-1 drug” means any drug which changes the level of a ZGGBP-1 mediated response or changes the biological activity of ZGGBP-1. For example the drug may be an agonist or an antagonist of a natural ligand for ZGGBP-1.

Preferably determination of the status of the human is clinically useful. Examples of clinical usefulness include deciding which drug or drugs to administer and/or establishing the effective amount of the drug or drugs.

Drugs which change the activity of ZGGBP-1 may be of value in a number of disease conditions, including disease states associated with neurological affective disorders such as BPAD.

According to another aspect of the present invention there is provided use of a ZGGBP-1 drug in the preparation of a medicament for treating a ZGGBP-1-mediated disease in a human determined as having a polymorphism defined herein.

Preferably the ZGGBP-1-mediated disease is a neurological affective disorder. Preferably the neurological affective disorder is BPAD.

According to another aspect of the present invention there is provided a pharmaceutical pack comprising a ZGGBP-1 drug and instructions for administration of the drug to humans tested for a polymorphism therein, preferably at one or more of the positions defined herein.

A ZGGBP-1 protein may in particular be used to screen for compounds which regulate the activity of ZGGBP-1 and the invention extends to such a screen and to the use of compounds obtainable therefrom to regulate the activity of the protein ill vivo.

Thus according to a further aspect of the invention we provide a method for identifying a compound capable of modulating the action of a ZGGBP1 protein which method comprises subjecting one or more test compounds to a screen comprising:

(a) a protein comprising the amino acid sequence of SEQ ID NO: 3 or a homologue or fragment thereof,

or

(b) a polynucleotide comprising the nucleic acid sequence of SEQ ID NO: 2 or a homologue or fragment thereof,

or

(c) a host cell expressing a ZGGBP1 polypeptide or a homologue or fragment thereof, and measuring an effect of the test compound on ZGGBP1 activity.

The screen according to the invention may be operated using conventional procedures, for example by bringing the test compound or compounds to be screened and an appropriate substrate into contact with the protein or a cell capable of producing it and determining affinity for the protein in acccordance with conventional procedures.

Any compound identified in this way may be used in the treatment of humans and/or other animals of one or more of the above mentioned diseases. The invention thus extends to a compound selected through its ability to regulate the activity of the protein in vivo as primarily determined in a screening assay utlising the protein containing an amino acid sequence shown in SEQ ID NO: 3 or a homologue or fragment thereof, or a gene coding therefor for use in the treatment of a disease in which the over- or under-activity or unregulated activity of the protein is implicated.

The ZGGBP1 gene of the invention may also be used as the basis for diagnosis, for example to determine expression levels in a human subject, by for example direct DNA sequence comparison or DNA/RNA hybridisation assays. Diagnostic assays may involve the use of nucleic acid amplification technology such as the PCR and in particular the Amplification Refractory Mutation System (ARMS) as claimed in our European Patent No. 0 332 435. Such assays may be used to determine allelic variants of the gene, for example insertions, deletions and/or mutations such as one or more point mutations. Such variants may be heterozygous or homozygous.

Thus according to a further aspect of the invention we provide a diagnostic assay for the detection of ZGGBP1, which assay comprises measuring the presence or absence of a protein comprising the amino acid sequence of SEQ ID NO: 3 or a polynucleotide comprising the nucleic acid sequence of SEQ ID NO: 2.

According to another aspect of the present invention there is provided a computer readable medium comprising at least one novel polynucleotide sequence of the invention stored on the medium. The computer readable medium may be used, for example, in homology searching, mapping, haplotyping, genotyping or pharmacogenetic analysis or any other bioinformatic analysis. The reader is referred to Bioinformatics, A practical guide to the analysis of genes and proteins, Edited by A D Baxevanis & B F F Ouellette, John Wiley & Sons, 1998. Any computer readable medium may be used, for example, compact disk, tape, floppy disk, hard drive or computer chips.

The polynucleotide sequences of the invention, or parts thereof, particularly those relating to and identifying the polymorphisms identified herein represent a valuable information source, for example, to characterise individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases, using state of the art searching tools such as “GCC”. Thus, the polynucleotide sequences of the invention are particularly useful as components in databases useful for sequence identity and other search analyses. As used herein, storage of the sequence information in a computer readable medium and use in sequence databases in relation to polynucleotide or polynucleotide sequence of the invention covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data.

The invention provides a computer readable medium having stored thereon one or more polynucleotide sequences of the invention. For example, a computer readable medium is provided comprising and having stored thereon a member selected from the group consisting of: a polynucleotide comprising the sequence of a polynucleotide of the invention, a polynucleotide consisting of a polynucleotide of the invention, a polynucleotide which comprises part of a polynucleotide of the invention, which part includes at least one of the polymorphisms of the invention, a set of polynucleotide sequences wherein the set includes at least one polynucleotide sequence of the invention, a data set comprising or consisting of a polynucleotide sequence of the invention or a part thereof comprising at least one of the polymorphisms identified herein.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e. screen for the presence of a polymorphism.

The invention will now be illustrated but not limited by reference to the following Examples. General molecular biology procedures can be followed from the methods described in “Molecular Cloning—A Laboratory Manual” Second Edition, Sambrook, Fritsch and Maniatis (Cold Spring Harbor Laboratory, 1989) or “Current Protocols in Molecular Biology”, Volumes1-3, Edited by F M Asubel, R Brent & R E Kingston, published by John Wiley, 1998.

### EXAMPLES

### Example 1

Identification of the Genomic Structure of ZGGBP-1

The ZGGBP1 cDNA sequence was used to search EMBL and EMBLNew nucleotide databases, using BLAST2 (NBCI). Clones were assembled using SeqMan (Lasergene99) and the intron:exon structure of the gene determined via alignment of the cDNA to the genomic sequence.

Identification of ZGGBP-1 Polymorphisms

Seven polymorphisms were identified in the ZGGBP-1 genomic DNA sequence.

| | Position | Position on | | | Resultant | |
|---|---|---|---|---|---|---|
| Polymorphism | of SEQ ID | Genomic | Reference | Minor | codon | Frequency of |
| ID | NO: 1 | Sequence | allele | allele | change | minor allele |
| BP24 | 221374 | Intron 6 | T | C | N/A | 27% |
| BP42 | 298758 | Intron 16 | T | C | N/A | 32% |
| BP48 | 119726 | Intron 2 | T | C | N/A | 48% |
| BP59 | 270159 | Intron 6 | T | C | N/A | 45% |
| BP64 | 343331 | Intron 28 | G | A | N/A | 6% |
| BP65 | 324317 | Intron 22 | T | C | N/A | 15% |
| BP69 | 294977 | Intron 13 | A | G | N/A | 49% |

ZGGBP-1 polymorphisms were identified using two approaches: 1) screening of exons and 2) random screening of a genomic DNA clone. Details of which method was used for identification of the polymorphisms is shown below.

| | | Method of |
|---|---|---|
| Polymorphism | Position of | polymorphism |
| ID | SEQ ID NO: 1 | identification |
| BP24 | 221374 | Random Screening |
| BP42 | 298758 | Exon with flank |
| | | Screening |
| BP48 | 119726 | Exon with flank |
| | | Screening |
| BP59 | 270159 | Exon with flank |
| | | Screening |
| BP64 | 343331 | Exon with flank |
| | | Screening |
| BP65 | 324317 | Exon with flank |
| | | Screening |
| BP69 | 294977 | Exon with flank |
| | | Screening |

1) Screening of Exons

Based on the genomic structure, primers were designed to enable PCR amplification of each exon including some flanking intronic sequence. Sequencing was undertaken using a MegaBACE Dye Terminator protocol in a panel of 29 unrelated Caucasians. The sequence data was analysed using Consed and polymorphisms were identified using PolyPhred.

2) Random Screening of a Genomic Clone

The genomic clone AC011331 was identified as containing the majority of the ZGGBP-1 gene. Primers were designed to random 300 bp regions, amplified in a panel of 29 unrelated Caucasians and sequenced using a MegaBACE Dye Terminator protocol. The sequence data was analysed using Consed and polymorphisms were identified using PolyPhred

ZGGBP-1 Polymorphism Genotyping

Two methods were used for ZGGBP-1 polymorphism genotyping: 1) SNaPshot and 2) TaqMan. The method selected for analysis of each polymorphism is listed below:

| Polymorphism | Position of | Genotyping method |
|---|---|---|
| ID | SEQ ID NO: 1 | used |
| BP24 | 221374 | SNaPshot |
| BP42 | 298758 | SNaPshot |
| BP48 | 119726 | TaqMan |
| BP59 | 270159 | TaqMan |
| BP64 | 343331 | TaqMan |
| BP65 | 324317 | TaqMan |
| BP69 | 294977 | TaqMan |

1) SNaPshot

Initial PCR amplification of the region containing the polymorphism was carried out using the following primer pairs:

| | Position of | Forward amplification | Reverse amplification |
|---|---|---|---|
| SNP ID | SEQ ID NO: 1 | primer | primer |
| BP24 | 221374 | SEQ ID NO: 4 | SEQ ID NO: 5 |
| BP42 | 298758 | SEQ ID NO: 6 | SEQ ID NO: 7 |

PCR conditions were as follows:

| | |
|---|---|
| 19 μl | Abgene 2 mM Reddy load |
| 1 μl | 5 uM primer pair mix |
| 2 μl | Genomic DNA (5 ng/ul) |

PCR cycling conditions:

94° C. 1 min (94° C. 30 sec, 58° C. 30 sec, 72° C. 2 min)*34; 72° C. 10 min PCR products were treated with 2 U Shrimp Alkaline Phosphatase(SAP) and 2 U Exonuclease I(ExoI) at 37° C. for 60 min. The reactions were then incubated at 72° C. for 10 min to inactivate the enzymes.

The following extension primers were designed which bind immediately adjacent to the polymorphism (in the forward direction):

| Polymorphism | Position of | Extension |
|---|---|---|
| ID | SEQ ID NO: 1 | primer |
| BP24 | 221374 | SEQ ID NO: 8 |
| BP42 | 298758 | SEQ ID NO: 9 |

The primer extension reaction was carried out using the following reaction conditions:

| | |
|---|---|
| 1.1 × Seq buffer | 4.0 μl |
| SNaPshot reaction mix | 0.5 μl |
| Extension Primer (1.5 □M) | 0.5 μl |
| Purified PCR product (SAPExo treated) | 0.5 μl |
| (96° C. 10 s, 50° C. 10 s, 60° C. 30 s) * 25 | |

When completed 0.5 U SAP was added to the extension reaction mix and incubated for 60 min at 37° C. The enzyme was inactivated at 72° C. for 10 min. Samples were then analysed on an ABI 3700.

2) TaqMan

Initial PCR amplification of the region containing the polymorphism was carried out using the following TaqMan amplification primer pairs:

| | | Forward | Reverse |
|---|---|---|---|
| | Position of | amplification | amplification |
| Polymorphism ID | SEQ ID NO: 1 | primer | primer |
| BP48 | 119726 | SEQ ID NO: 10 | SEQ ID NO: 11 |
| BP59 | 270159 | SEQ ID NO: 12 | SEQ ID NO: 13 |
| BP64 | 343331 | SEQ ID NO: 14 | SEQ ID NO: 15 |
| BP65 | 324317 | SEQ ID NO: 16 | SEQ ID NO: 17 |
| BP69 | 294977 | SEQ ID NO: 18 | SEQ ID NO: 19 |

Allele specific probes (TaqMan allelic discrimination primers) for each polymorphism are listed below:

| Polymorphism | Position of | Allele 1 Primer | Allele 2 Primer |
|---|---|---|---|
| ID | SEQ ID NO: 1 | (Vic Tag) | (6FAM Tag) |
| BP48 | 119726 | SEQ ID NO: 20 | SEQ ID NO: 21 |
| BP59 | 270159 | SEQ ID NO: 22 | SEQ ID NO: 23 |
| BP64 | 343331 | SEQ ID NO: 24 | SEQ ID NO: 25 |
| BP65 | 324317 | SEQ ID NO: 26 | SEQ ID NO: 27 |
| BP69 | 294977 | SEQ ID NO: 28 | SEQ ID NO: 29 |

The TaqMan reaction was carried out as a single PCR step as follows and results were read using the Automation Queue software:

| | |
|---|---|
| 2.5 ul | ABI Universal Mastermix (2×) |
| 0.031 ul | Primer/Probe mix |
| 2.47 ul | H₂O |

92° C. 10 min (92° C. 15 sec, 57° C. 15 sec, 57° C. 45 sec) * 45

Statistical Analysis

The genotyping data was analysed using the Chi Square test for case/control association.

### Example 2

Association of ZGGBP-1 Polymorphisms in a Collection of Psychiatric Samples with BPAD

Based on the biology of the ZGGBP-1 gene and its location within a genomic region implicated by linkage analysis, the role of ZGGBP-1 in causing BPAD was pursued. To determine its role in the disease, an association study was undertaken using ZGGBP-1 polymorphisms and to genotype a sample population. A collection of psychiatric samples was collected through an SRF funded collaboration with Prof. D. Blackwood (MRC Human Genetics Unit, Edinburgh). This sample set consisted of 192 SZ (schizophrenia) cases, 288 BPAD cases, 96 RUP (recurrent unipolar depression) cases and 288 controls samples.

All 7 SNPs where genotyped in the complete cohort and the data analysed based on the complete cohort (SZ, BPAD and RUP) as well as stratified for the BPAD cohort only. Results are given below for both populations:

| Polymorphism | Position of SEQ ID | P-Value (Full | P-Value (BPAD |
|---|---|---|---|
| ID | NO: 1 | Cohort) | only) |
| BP24 | 221374 | 0.0039 | 0.0003 |
| BP42 | 298758 | 0.8393 | 0.4248 |
| BP48 | 119726 | 0.4902 | 0.5949 |
| BP59 | 270159 | 0.5182 | 0.8050 |
| BP64 | 343331 | 0.4197 | 0.3901 |
| BP65 | 324317 | 0.2578 | 0.5190 |
| BP69 | 294977 | 0.9560 | 0.8130 |

Association of Polymorphism at Position 221374 of SEQ ID NO: 1 with BPAD

A weak positive association was observed between the presence of the polymorphism at position 221374 of SEQ ID NO: 1 with the complete cohort. When the data was stratified based on disease a stronger association was detected specifically with the BPAD cohort. This finding was replicated in a second cohort of BPAD cases (p=0.007). When the data from both cohorts was combined a statistically significant association with BPAD was noted (p=0.0003) indicating that ZGGBP-1 is a gene involved in BPAD:

| | Position of | | |
|---|---|---|---|
| | SEQ ID | | |
| Polymorphism ID | NO: 1 | Cohort | P-value |
| 24 | 221374 | 200 BP/200 SZ/200 | P = 0.053 |
| | | UNI/200 controls | |
| 24 | 221374 | 200 BP/200 controls | P = 0.013 |
| | | (stratified to BP cohort) | |
| 24 | 221374 | 100 BP/100 controls | P = 0.007 |
| 24 | 221374 | Combined cohorts | P = 0.0003 |
| | | (300BP/300 controls) | |

1. A polynucleotide comprising a nucleic acid sequence which encodes the polypeptide of SEQ ID NQ 2, or a homolog or fragment thereof.

2. A polynucleotide as claimed in claim 1, which comprises the cDNA sequence of SEQ ID NO:1.

3. Polymorphic variants of the polynucleotide as claims in claim 2, selected from:
i) T at position 3554 is replaced by C,
ii) C at position 4828 is replaced by G,
iii) T within an intronic region associated with ZGGBP1 is replaced by C, and
iv) C is inserted at position 4032.

4. A polynucleotide which comprises an animal homolog of the nucleic acid claimed in claim 1.

5. A polynucleotide as claimed in claim 4 which comprises the cDNA sequence of SEQ ID NO:3, or a homolog or fragment thereof.

6. A polynucleotide which is capable of specifically hybridizing to eight or more contiguous nucleotides comprised in SEQ ID NO:1 or SEQ ID NO:3 or comprised in the complementary strands thereof.

7. A polynucleotide which comprises a ZGGBP1 gene fragment.

8. A vector comprising a polynucleotide of any of claims 1-7.

9. A host cell transformed with a vector of claim 8.

10. A polypeptide comprising the amino acid sequence of SEQ ID NO:2 or a homolog or fragment thereof.

11. A polypeptide comprising the amino acid sequence of SEQ ID No:4 or a homolog or fragment thereof.

12. A fusion protein in which a polypeptide of claim 10 or claim 11 is fused with glutathione-S-transferase.

13. A method for producing cells which express a polypeptide of claim 10 or claim 11 or a fusion protein of claim 12, comprising:
a) culturing a host cell of claim 9 under conditions suitable for the expression of the polypeptide.
b) recovering the polypeptide form the host cell culture.

14. A method for identifying a compound capable of modulating the activity of a ZGGBP1 protein, which method comprises subjecting one or more test compounds to a screen comprising:
a) a protein as claimed in any of claims 10-12 or a homologue or fragment thereof, or
b) a polypeptide as claimed in claims any of claims 1-7 or a homologue or fragment thereof,
c) a host-cell expressing a polypeptide of a ZGGBP1 molecule, and measuring an effect of the test compound on ZGGBP1 activity.

15. A compound that modulates the activity of a human ZGGBP1 identified by the method of claim 14.

16. A pharmaceutical composition comprising a compound that modulates the activity of a protein identified by the method of claim 14.

17. A diagnostic assay for the detection of ZGGBP1, which assay comprises measuring the presence of absence of a polynucleotide as claimed in any of claims 1-7.

18. An antisense molecule comprising a complement of the polynucleotide in claim 1 or a biologically effective fragment thereof.

19. Use of a polynucleotide as claimed in claim 1 in gene therapy.

20. An antibody specific for a protein of any of claims 10-12 or a fragment thereof.

21. A set of amplification primers for selective amplification of a ZGGBP1: gene sequence.


SEQUENCE LISTING (29 sequences)

SEQ ID NO: 1 - DNA, 344805 bp, Homo sapiens
gacaggctcgcgttatggctccgctttctgcggctctcctaccctggcatggtgtgtgtgtgtgcctgtgtgctacgtaagtgtggaatgaaaggtattttggggtgggaagggtgtttagggtatgaatttttttcttagcggtgctaggcaaagcttgatgtatttatttgatcaaagaatgatcacagtgcaaataatgtgtgatttgtgtgtttattttgcctcgctgtgcttttgtaaaataatttcccgttttgtgtaactacctggggataggtgacagtagcattgtttcattgacccggggggcttctcctctccctcctggcttctctctttgaggagctttcccttgcatcctctacttctccatttaattagtggcagtagagggcaggccctgagttgcagagacgcagacattcctaaacaaaggggctgcttgggttgacgtattgtggggggtgggggtgaggacagctgcatttcatcctaggttcttttaaaaaatgttttaataacatgattatttgtaaaaactcccccgaaaaaacgattttaagagtgttttgggaagtgcagctggataaatgaaacacgttaatgtcactgttgtttgtgctttatttacattggcagtgagtgtcagaatctacctagagcacatcttgccacttttgtccacaggagtcactactaatcatcataactgatgtctagtgtgaaagtgaagcttggaatgtaaacagagtccgagtgtgtgttggcatgtgactggattcactcctgcatgttgctagaagggaatttggggctgtataggaaatagcaagaggtgatgaagacaccttttattatctatttattgccatgtcgctggtagtatttagttccacacacattgtatttcacaagatgagtctcagccgtcttcctttttcaactttctcctaccagtgaaaaaataagccagggcagttctgatgacttcacatttcattggtatttgtttttcatgggcagtttcatgctgtcacgttgcccttcagagaatcagggaatgccttcgtagaatattaacgacaaattgttgaatttgcacagcttgttttgtctccctgaattagtatgtgtaagaaaaaccaatggaagattttagcaggctctgaaagtagaatgcccgctgaagcaaacggcagggttgttcttccaaggcgtattaaatatgggtacacttagtttgcagacttggtgtgctgcagaatatcacatatgaagatattttgaccgctatttagaagagtgacctcataaatcttttagcaaagtagacacacatacctaccacatttcttaaaagtagattcgtattttgatttgagattgttgaaaagcatggtatgtttggagtgcacttagttatgtcccatctcataaagactgttgagctctctgcactgctgatgatgaagcattgcatgaacccagaaaacggggatgccaaagcacagggcttaggtgctgttccttgcgtcatcagcagaggtctgtccactttgtagaaggtaaggggttgagatgtgggctttgcttgtgtgaatgtatgtgctgtttaattttttctgtgtgtttcgggcggaggtttctctggtgagttataattcatatttaccctgatttaagtcatgtcactttaggtagccagattcaaaaccatcagatgaaggacttttggtgttagagggattagcagtttaacaaaacttctgattcatggcccaagatcagatttggtttggaatcagaacactttggactagtttctgcatctggcttgagtccttgtggatttcttgttttatttctttatttttggcaatctgtagtgcattttggcatagtttacttatgaatgttaccgctaactttctgaaatcttttattccattttagaaaggtatcatttattgaaaagatttttctactgaacaaaatactgggattgaatggcagcttcttatcacattagatatgacattaagtaatttcgttagtagttaaaatgaagcaaatggcatagaaatgaaggccaggctgacataggataaatggcaccttctctccttatcctgtacaaacacggatagttggctacttttgaatgcctcttctataccaatggtaaaaatccggcttaattggggccacattctagcctagtcacaatcagtggcttgcattatgtcctttgagatgtggcagatctgcttataggtgccagtcaccacggctccctgctggcttgggggtctttctactcatttacatataattatctacttcttaatcaaccattaaggttaacttttggcatggatcctgtatgcttttcagtttaagatgctgtcacttttaagatgcgccatgagagcaaagtgctaagccttgtgcacatactatgtcatttagttctcatgagaactgtgtgaggtaggctgtgatttacagatgaggaatgaaagctcaaagaggttacgtgacctgcataaggacacaaaacaagttaagttatggagccagctattttgaatagaaaggcctagtttttaacatgctgaatatcctccccttctgacaaatgccttgaaaggtcttactatctagattatttaagataatgtaagagaaataaacaccactggttgcaaactgatggctgctgggcccgatttgaccaggaaccctgcttaatgtggtctctagagtgttttaaagcttggaaatttcatgaaaatggtaaaaatctggcttaattggggccacattctaacctagttacagtcagtggctggcattatgtcctttgagatgtggcagctagttgtgctatggttgcagtcaccactactttctgttgtcttaggggtctttctacttgtttacatataattatccacttcataacaaacctttaaggctaacctttggcgtggatgccatatttctttcactttagtgtgctgtcacttttaagatatgccattatttcatgtaccacgaagaaaaaattgctgtcaagttatgacatactgtccatcttatgaagcatccttatttcagagaagttgaaatatgagaaaaaggatattttagaattgatgaattacagtgactagaaacctgggaaaactccatgccatcagtttcaatatccccaatcattcagtacttttaagtcaacgagtagtcagatttctgcttttctgtgtttgaggaaagtgctagaaaaagagaagtgctcttaaataggcattagcacagaaggaactgggggcctagatcatagccaactggatggccgtctgtaagtgattaagaatagttgttagtttttaaaaagtggcctgtgtgtattttcaagagattacttgggaaaccctatgtgccatctttgcagctgtgcaaaacggtgaggacaagattgctttcaaaaattgtggtttagattttgcttggatctcaaggtgaatgctgacaggccgttttctgaagagagagaaggcttgcccagtcatgccaagtccaagtgcctgtgtcgggcctagatgctggatggtagactgtatacccaggacatataggtaatgtttgagaaacaaatgttaaaaggacctagttttcatggtgacatttaaaatcagtttttaaaattcatgaatataacatgttgttttaaaatttgattatagggaccgggtgcagtgatcactcctgtaatcctagcattttgggaggccgaggcaggagaatcacttgagttcaggagtttgaggccaggctgggcaacgtggtgaaaccccgtctctacaaaaaatagaaaagttagccaaacatggcggcacgtgcctgtagtcccagctacttgggaggctgaggtggaaggatcacttaaacctggaggcagaggttgcagtgagccaagatcatgccactgcactctagcctgggcgacagagtgagaccctgtctccaaaaaaaaaaaaaaaaaaaaaaagactaatgaatagaatttattttcttcaatgatgtagtgtctgtggaatttgtagacattgacatttgaattttcattatatttagttttattaaatcagaatgttatgattgtttatttcattattgaacatcagtttccttaatttaaggaataaaaataattttgctcaggccagacatagtggctcacatctgtaatcccagcacgttggaggccaaggctggtggatcacttgaggtcaggagtttgagaccagcccagccaacatggtgaaaccctgtctctacaaaataaataaataaataaatgaaaaaaaaatcagctgggtgtggtggtggacacctgtaatcccagctactctggaggctgaggcaggagaatagcttgaaccctgaaggcagagattgcagtgagccgagatcgcaccactgcactccagactgggtgacagagtgagactctgtcacaataataacaggctggggcagtggctcatgcccgtaatcccagcactttgggaggccgaggctggaggatcacctgaggtcaggagtttgagaccagcctggccaacatggtgaaactccgtctttactaaaaatacaaaaattagctgggcttggtggcaggcaactgtaatcccagttactcgggaggctgaggcaggagaattgcttgaacctgggaggcagaggttgcagtgagacaagttcgggccattgtgctccagcctgggcaacaagagcaaaactaaaaataaaaaataaaaaaagaatagtaatagttttgcttaaagggtgaggacagtgacaaatggtgatcctcagatgactgtagtttctttaccattacagaatgcaattcaaatgaaaaccattgcatggtactcaggcatttcttcaatgcagtgtgtgcccacatcaccaggaagcttacttgggaaagtttgttttgggattctggagtacctcaatatatctggtatattttcccaagaaaagtgaataatcttggggggccgaggtggacagattgcttgagcccaggagtttgacaccagcctgggcaatgtggtgaaaccccacctctacaaaaaatacaaaaacttagttgcgtgttgtggcacgcacctgtggtcctggctgctggggaggctgaggtgggaggatggcttgagcctgggaggttgaggctgcagtgagccatgatcatgccactgcactccagcctgggtgacagagcaagacactgtctcagaaaacaaaggcaaacaaacaaagatcaccccaaagaagaactcccaacatattaaaagtcattgagttttgtacccttcaaatggatgaattgtgttgtgtatgaattacatctgaataaaactgtttaaaaatattaagaatgattgagtgtgacattattgcagcagtacattggagtttctgctaaattacttttgcattcatttcaaaaggaagcaactatgcccattgctaatttaataacctgtttgcttgtgcggtgtttaaaatggcatcatgaaaacatggcaatgtgtgatagattaagagccaattaaattttttattttgagttacaagatctgcattaaacagattttattgttgcccttgaatttccagtcttagatgagtagcagcagtgaaaaaaagagatcctaacttaagagttttcaagctttttgatgttgcaggtatttgaataagtgggtctgtcttactgaaatatttgttaattagcgataaagcaaactatatgttgatcttttggtagtccatgctaaggaggattagaataatatataaataatataatggtaataagtatcagggcaggaggattaaagaagggtggaagaagtgtgacaaaatgttactttctgtttttacattgtttctcttccaggtcacaactgtatttcaaatgtatttcattttcagactataggtggcaatgtttgtgctgttcactgtttaatgttactgttggctcgggtgttgcttgttgctggaatatggaaatgatagcataatttagagttgagaataaaaaaaactagatttagaaaactagaaactagatttagatttagtagagactttctaaatagtcctagttctgctgtgtggacccttctgtaactttctgttactctgagaaggagctgataattatcacaactactggtgtttgattagcagagacattcttgattcctgatgcaagaccgtggctgaagttgtagcagggcgtttggtggttggcgatgtaaagatctgggcctcttgtttcatgaataatagaatctcactatgattgtaatattttgttttgaactggagtgctgattattgtaatccctttaggtgagaaaagccttattatataagtgtatctggggtaactagcttaaatttgacactccagtcaactttttgtccctgacttcagtcctgcattgattcaaaacgcaccaagttaaatgtcaaggctctcagattcccttgctgtcatcaaattcagtgtttggtgacttcttggcaggcagacatctaccaagagattttgtattctttttatacctcctattcaccaaaagaaaagggccattcaaaagtgagtttttctttttttttttttttaagtttaaaaagctatgatgtggccacccatggtggctcatgcctataatcctagcactttgggaggccagggcaggcagatcacttcaggccaggagttcaagaccagcctgaccaacatggcaaaaccccgtttctactaaaaatacaaaaattagctgggcgtcgtggcgtgcccctgtaatcccagctacttgggaggctgaagcaggagaatcgcttgaacctgggagatggaggttgtggtgagctgagattgcaactctgcactccaccctgggtgacagagtgagactttgtctcaaaaaaaagctgtgatgtgaaacctgttctgtgaaggtggaacttggaaaggcttgtaaggatggtggtcccatgaagaggatgaacaaaccaagggaactgtcctcttgctggtttgggtgatgtcccatcacctgtcactttgtttgtttgggctgcagtggaccaatggtttcccaggcacactgaagtgacatgtagatggattgcttgagtggctccggcatgtttccatctcctgtctaccaggcagagaccgtgttccctgttttgaggggatccagaagtgcagtgacaagggcctgatttgcatctccagctcctggtcttgcctgtgttaacaattttttttttttctttccagatggagtctcactctgtcgcccaggctggagtgcagtggtgcaatctcggctcactgcaagctctgcctcccgggttcacgccattcgcctgcctcagcctcccaagtagctgggactacaggtgcccaccaccacgcccggctaatttttttgtatttttagtagagacggggtttcaccatgttaggcaggatggtctcgaactcctgaccttgtgatccacctgcctcggcctcccaaagtgctgggattacaggcatgagccaccgtgcccggccaacaatttttacattctaattaatttccagtttccaacctgactcaaggtttagctcttcgtctcttgggtgcagttttggatcatgtcttataatgtatatttaaaagtgtgttgtaaagctggaattcccattgtgggtaaatgctgagattgttgtaatactgagaagtgcacttgggaaccatggccaacactgccctagctgaccggaggttgcactgagttgtcccctttgaacatctgttatgaatgaatgttggaaaatgcaaagccaatcatttcttttacctgacctatgtgatgttaagttttagttttatgttttggcaagtgagtgttggatgtcactaatgtgaggaggccggtgagggcagactttgggtcagaggtaatgtaggagatgaagaccctttctattaatttttcttgtttaattgaagatgttcgtgttgaatgggttttatttacatgtataagttctttttttcttaatgctggaagttactttggtttttcagaaaatgcaagtaaccctaatctatttagagaattactttgcttagtaagcactcagtactatttagtaatggggaaaaaatgtgtttgtgtcccacagcgtgacaggctccacaaacttacttacgaatctcctaactaccctgagaacctgaagaatgggactttgtggagaatcttctgctttctttgctagtctctatttttccttctcagttattaagtgagttgtctttgggtgtcctcatcatgctgacttcatcttgcccagtttgttttgggtagaggaaggagtgcaaggtttaaaggaaaagcaaagcctgtacaacatggtgagaccccatgcctgtggtcccagctacttgagaggcccaggtggaaagataatttgagcaccggggttcaggaatgcgctgagtaatgattgcaccaccgcactcgagtctgggcaacagaatgagacctcatttcaaaagaaaaagaaaaagcaagactgaatcatttcactttgtattctcctccttgctctcattccttttgtgcttcatgcattgtgtgtgacaattggatgcagccgtaattattgtcagtaacttggtttttaagcttcagactgtatttcctcctcacttgttaacaccagaagataaatgaatgtggctcttggattgaactacatctacagggagctgaacttcccatgttgacgttgaattctagagtatgcttatgctgattcttgagaaattggtatgtgccgtgcctttggaattgataccttaattttcagtgaatattattggtattattattaaagagaacactttaagccaggagtaagagcttttcctccttttgagaagtgaggtacagaagagtatactgcagctgtgggagtgatgggtggggtggggcagtgttgaagaatatttaataccacgaagatgtcaccagtataatattaaatagaaaaagtaggttataagacagtggatccagtagaatcctattgtggttaagtgtttatatgtagagaaaatgactaggggctaggcacagtggctcacgcctgtaatctgaaccactttgggagatagaggtgggaggatcacttgaacccaggagttcaagaccagcctgggcaacatagtgagactctgtctctataaaacaagaaaaaagaaaaactaactagggatatattccagcatgttagtggaggttatctttgagcaaaggggattataggtaattttttattgtcttgtgttttgcttacatgtgttttctatatttctacaataaacctttttttcttaatataaaagtaagtttaaaagtgagtttaaaaatccctgaaaggcatgtacttcaagcttcaagtgaaagttatagtacttgttcagcacaaatactgatatagtgtttgttcagtggaacacttatttagggtctgccttgtgttttgtgtctaaggattaccaggtcattttcatattaggagctcaccactacgttgggagttggagagatagagcaaggactattttaaggaaaaaatgactggtaaattgtatttctttttccccacttgcagtttgcaaaatagatgtttcatttctctccactgtggacacttaaagtttgaatttccatccttctgtgcactccagttgcgtgaaagtaatttacattgctagtagtggtttgggttctctgaaattttttgtgtgtgctatggaaattgtctcctgtgatcagactcaaatccatttctccttattattgttttcttaggttttgagttagagaccttttcatgaccgtataaggtgttgaataggtttagatatcaaagtgatagctatgcatagctatactcacctgtgagcggtcagctcttcagtgtttctttcaactgacttgtaatatccagccactgttgggtgtgacctaaagtgtatctcttcccaaacagaagggaggagttgacaaacaccatgttacttttaatcagagacttgtccttgtcccccttcccccagccttttaataattctgaattcatgttgcagtcgatatgtgtttaaagtgttggtaggtttcttgtactcacaccatcctcctccactcctctcacccccaaaaagtgacagctgacagttgatggcatcttagaattgtgaggcttgagttggtctgtctcaattcgtgttttatggtggcccataaattgtgaaagataattgatagttgtcatgggaactagttatgttgttcagctatgatgtctcaattaggaaatgttcacctagacagtagggaaatgggccagctcagggatgtcttcaggaaccaggttcattgttttttctgctttcccttctacagcctggctctttccagggcttcttggtcctaggctggctgccactgacagagctatgcctttttttatttttttactttttttcaagacagtctcactctgtagcccaagcaagagtgcagtgacaccatcttggttcactgcaacctccacctcctgggtttaagcgattctcctgccccatcctcccgagtagctgggactacaggcatgtaccaccacacccagctaatttttgtatttttaatggagatggggtttcaccatgttggccaggctggtcttgagctcctgacctcaagtgatccacctgcctcggcctaccaaagtgctgggattacaggtgtgagccaccatgcccagccagagctatgccctcttttttttttttttttgtttgtttgtttataactgtgtacttgtaaaggaaagagaacggcctcctgtgccttaaatcttctgttgtgtctcagggcccacagaggctgagatctgttcatcctgcaaccagtcaccgtggcaaggaggagatagaattacctttttttcttaccttttgagccaggggtaaagtcaggcacggtgaccctgcattccacaccttggataatccatccaccttcagaaatgcccctggcttcgtgaaatgcacatgctccctttctccagggaggtgacccgaatatgcagctaagtattgcaaggatttctgggtaatattacacctgaaaggaggttttttttttttttaattatctggtgatttatgactaaataggctatgtctctcaaaacattgaacctgcaatgggcaagaaagaacaggatgactacaatgaaccttctatttggaaaaggggaaaataagacactcacagtggggtagctggctgaacaggaacagctgggagcgtttgctttggtcagccggtctggcagcttgcctctgctgaagtccgtccagggtgagaatctcgcttggtcattgtcctctgcagctgcacctggatggggtaggcactggcaaagctccttttcttgggtgctgcttggcattagtctggggcttcaggattggttgtcttggggttgaatagccacaggcttgttccagccttactgagaatgtctttggcaaaactgctcccaggaaaactcaggctctgagttttgtgtctagtcaattccatgggctggtaatggcagccagtgatctcacctagatgaggtctgtggttctgggaattcctgggtcttaacagtaagtcccacagccctgctaatccccaccacatctgccttgaggcaattttaaaatagcctagaatgaagaggcacacagttttgggtctaacagacaacaggggcagtttaacccactctgcttttgctctctggtgtatcaagaatgagcaacttccccacactgggattgtgggactctgagtacccacttcccttcactcagccattccaatttcttaaagcaatttttaaaaaatggtggtaaggccggccgcggtggctcacgcctgtaatcccagcactttgggaggctgaggcaggtggatcacgaggtcaggaggtagagaccatcctggccaatatggtgaaaccccgtctctactaaaaatacaaaaattagctgggtgtggtggcacgcacctgtaatcccagcctctcgggaggctgaggcaggagaatcgcttgaacctgagaggcggagattgcagtgagccaagatggtgccactgcactctagcctggcgacagagcgagactccatctcaaaaaggtggtaaaatacacatgacaaaatggactctcttagccacattaaagtgtatggtttggtggcattacatgcatgcacactgttggcagccatcacccctgtctagccatggaactctttcatcttgcaaaactggaaatgccttactcgtgaaccagtacctccccattcccagccattccagtttcttgtagcgcatcttctctttgatacaaaatctttgatagggttttgattgcaggcaacagcaattgaatctgatgaattcaacagagaaagaattaactgaaggaatttggggtagcactggagtacttgtcacactggagagacaggcctggacaggggtcagggaccaatgaagcctgggcatccagaatcacagctgttagtgtgttttattatttaagagtgagctcagaggtcactgttcaaagctgggcactctgtctccaactatccctcttcacatcccagactcaatagcacctttctgcatagttacttctactgaatacttcactgacagcaatgaaggattgttggtttatgtgtctgtctcgtatagattaggcacctcttcagagccacagccacatcccaatgatttatctccagtcccaagcaccgaatcagaccccttgaccgtttactccattcaagtgatgatctcaggttagataatcatgaggtcagttggtcaaagcaaaaacatctttttaaatgtctttttgaattctaatttgtttatgcaatatagattactcctcctattttcttcttctttcttttttttccttaagagatgggatctcagtatgttggccaggctggcctctaactcctgggctcagcggtttctcccaccttggtttcctaaagttcagggatgacacgtgtgagccaccgtgctcagcccctcctcctattttctagtctgtgtcattactgagaattttactctttattctctttcctcaaattctaagtgaaaagacctctcctctctaggccccaagaaaggcaaatgtgttccacggaagaaggttgcagacccatgtttgattgctttataccatatttcccccccagaggctcttaaaagggcaaagaaggtgttgaaactgccactgtcaataccagagcatctgcttgaaaccaatgtttcttttgtgactccaggatccatgctgcctggctaagaagggcagtgtcctccttgcccaggtgttaaaccacaacagacctagaaaactgagatgtcactcctcccctggagtgacagcagtgttaggaagtttggaatctgccaacccacctcatcctgtccatgaagttgtcacttggctaggtgttgatgtaaatgtgctacagaaacaaactcacctcttgtgggacatggtgactctggttagtaacaatgtattcttcaaaattgccaagacagtagattttaaatgttcttcgtatgcaaacttggtaagtatgtggggtaatgcataggttaactagcttgaattaaccattccgcagtgtgtatgtatttcgaaataacatgtacatgacaaacagatacaattttcatcaattaaaataaacatttagaaaaataacataaatcctcacaatagccctttgaggtaggtgctgtcattttataggtggggaaactgaggttcaaggaggttaaataacttgcccagggtcacagaactacttagtaagtggctgagccaggacttgaacaagcagtccggttgtagacacccggagtggaaccatcacgctgtggtgtgtccgggagtgcttcaagcaggggttggttgggggtggggttggggtggggaaggcacagctgaggcctgagtggccagactgtaaaagccaaaggaagaagtggctggatgtgaacagctttgtggacctcatcaaggagctgggactatattgtttgaggcaccttgagaaacctgttgagatctgggcttttcttattagtaagtatgttgcccagactggtctcgaacccctggcctcaggtaatcctcccatctcagccttctgagtcgctgggattacaggtgcaagtcgttgggcccggcaatctgggcttttctaaagctcaccatgcagctttggggagattggatttgagttggacaaccctaggcagttgcagagctgtccaggcagaaatggtgatgacggtgaaaatgagccaggggataaggcaggaggtatttaggaggcagattccattggatttggtcactgattgattaggtacaaaaaaagggaggtgtctagaataatccctaggtttcttcagaggactaggtagatgggaggaggggaagaagcctatctctattaaatacttagtacagtggctctaaactggggcctgtcttgcacccttccttctcccctaggggacatttggcaatgcctggagacttttttttgagacggagttttgctcttgttgcgcaggctggagtgcaatggcgtgatctcagctcactacaacctccgcctcctgggttcaagcgattctcctgcctcagcctcacgagtagctggggttacaggtgcctgccaccacacccagctaattgttgtattttttagtagaaatggggtttcaccatgttggtcaggctggtctcaaactcctgacctcaagtgatgtaccggtctcggcctcccaaagtgctgggattacaggcgtgagccactgtgcccggccgcctggagacatttttgattgtcacagcttaggagatgcctctgacatctagaaggtggaggccagagatgcagcagaacatcttacacggcacagtacagctcccacaacgatcatctcgccccaaatgctgccgaggttgagaaaccctgctgtagtgtagtatcagttttacgtggttatctaattggatccgtgagccagtcatgttagatcataaatatgatttttcctgtttttcagataaaaaactgaggcatagacaggatgggtcacttggccaaagctctgcagctagtaagtgttagagccagaatttgaacgcaagtcttccagatgtcagaatctactccttctgcttaccctgctgcttcatatctcacagtgatgccaaggaagatgcacattggactgtgggttgttttttttttttttctttctgtcatggtggctccagactagaggaggcaggaaggaaggtggttatttatagtcagctgtgcataggatcgatttttttcaatcctgaaggacttgcagcaggtagttcaaagaaacctcctttagacgttgactttcccttgcagttgtaaatatgggaacatttaccatcttttgctttaaaatatactgttataattaaggtaaaatgtaaaacttataacttaaaaactttactccctgtgcatatgaactatggcaagcttctcaaattaattactatccctttgggataaattaaaatattttgatttgctagaggtaaggagcccttttgagaattagtttgttttagttgagaatgattagagggctagaatgatatatagctctaaataggaaggtttctgattcttttggctactctccacttttactggttattttccagttctgggaagattctgcatgtatgtagcagcctcccttggctatccatggtattagagctctagggacagagttctcctagttgaactcttcataatatctcattattggtattttcaaagttatatgtgtattttaagatagtctttggatgatgttccaaagaaggctgctttgttgagattgagatagttgttttgcagttgtgatttctctttgtaaaaagaagtctttgtgtcggtcccaggtcttattttttgcttgatcacccagtacgtgggtgcacttgggctgttgggaccatctggtgagtgtgaggtctctgtacttcacctgtggagtctgttgtcctgaaaggtgccggctcaggtggtggtgctttgaggggagcttgtttgctgcaccttccagtctgctagaaaatagatgcttagaggggcatttcggggtggaatctgatcatggagtttggtgagcaaacagggcctcaagttttatgttgtgagaaataaatactgagtttatgttttaattatggcacagattaatacaaaatctggtgaaaaatggagaaaaggagagcttaaaacttagctgtttgttgctgtcatctaaatgtagttggtagccagcctagatgggaccacagcatggcactaaagcgcggctgctgcttttcagatcccatgttcatattcgcatgtgtggagcatgaaagtaccaatagaaaattttgacaaagaaattgaagggttgggccagggacggtggctcacgcctataatcccagcactttgagaggctgaggcaggcagatcacgaggtcaggaaattgagaccatcttggctaacacggtgaaaccccgtctctgctaaaaatataaaaaattagccgggccttgtgacaggcgcctgtagtcccagctactcgggagtctgaggcaggaaaatggcatgaacccgggaggcagagcttgcagttagccgagattgtgccactgcactccagcctgggcgacagagcgaaactccatctcaaaaaaaaaaaaaaaaagaaatcgaagggtcgataatttcatttatttgttcttttttttttttttttttttttttgagacagtatctcactctgttgcccaggctggagtgcagtggcgttatcacggttcactgtagccttgacctccgaagtccacacgatcctcctaccacagcctcccaagcagctgggactacaggcactcgccaccatgcctggctaattttttatttttatttttattttttgtagagacaagttcgtgctgtgttgcccaggctggtctcgaactcctgggctcaggcagtcatcctgcctcagcttcccaaattgctggatttattgtcatgagccaccacacctggccagggtcagtaagttaaaagataccttctaaactcagcccaatattgaagagtaaacataaaaatacatgttttgtctattatactattactgttactcaatgcttaactaattgtgcttatgtttttttctttgccaaaatttctcttcaaggagcaagtttttctaatattgcttttcttgggaagaacaattagtcacgagatagtacagcttttttttttttttgagacagattctcactctgtcatccaggctggagtgcagtggtgcgatctcggctcactgtaacctccacctgccgggttcaagtgattctcctgcctcagcctcccgagtagctgggactacaggcactcgccaccatgcccggctaatttttgtattttttgtagagacggggtttcaccatattgtctcgaactcctgaccttgtgatccacccgcctcggcctcccaaagtgctgggattacaggcgtgagccactgtgcccagccgtagtgcagtatttttaagcttcggtaaggtagaacccctttagagaaaaaacatcagtgagaagccatgttgccttatttttactataatgtttctttttttttttttttttttttttttttttttttgagacggagtcttgttctgttgcccaggctggagtgcagtggcgtgatctcggctcactgcaagctccgccttccgggttcatgccattctcctgcctcagcctctcaagtagctgggactacaggcacccgccaccacgcccggctaactttttatatttttagtagagacggggtttcaccgtgttagccaggatggtcttgatctcctgacttcgtgatctgcccgtctcggcttcccaaagtgctgggattacaggcgtgagccaccgcgcctgacccaatgtttcttaaaatacatccaaacataaaaagaacaaggatgtttatagctggtatatcaccatagccatcactataacaccgtaacaaacatgcaaatttgttacaaatctaattctcaaatgaataccattcaaattaatagcatgattttaatgtgacaggtgatgctattttattaaaactctatcatctgtgctcaggaagtcatggctgctagggtaagttttcctttcctaaccaattatgtgccagttggtggtaacataacaccgttttcctttgtttcctttattcaaacttctcttgtactatgtactagagcatgaaaacgtctttgacctacagttatgttagctggagacacagcagaactactgttgtcatcttgatcctgaatacaagggccgattgaagccccagaaatcatggggaagtactcgtttacataaagtggtcatgtagataatctaaaatatgcccgttagtgtattaagatcataactgcctaaagcttcctgtgccaggagtggtcttagctctcaaagaagtcatagtctggccaggcgtgatgactcatccctctcatctcagcactttgggaggcgaaaggattgcgtgaggccaggagtttgagaccagcccgggcaacatagcttgatcccgtttcttaaaaaatggaagtgatatccaggtggggaaaccttcctgctgtagtggagcctcttgagatggcacacgtattttgctcacaggtgctatcaatatatagctctggttcttcagaatataaataactatcacctgaccccaataccttatctatattgagattgttcattagatccaaaacatcttgtgcatttcttgctttttagtcatgcacagaccttgtctttactttcaaagacagtttggtactgaaaattgctctggtaaattaacacaatttatttaatccctgctctttccaatctttagcaacgtttttttcattgtcaccatcagatgaccttgctgagcttccaagtggcaattgcttttgttgtattgtttaaaggcaaacaaaaatcaattattattggctctagagtgcctggcgaggtggagctcagttctttgtgcattgcgcactatgctgctggtgtctcagctgccatgcttatggttgcattactttcctgtctagctttggtattcttgcctttaaaaatttctttttccatttgcttttcttagccagtgatgacagtaaaataaggaacgacctatttattgttttaagagatacgctggatggtgcccaggcagatgagcattgccattcaaggtggcttcttggtagactgcatttactcttaatcgggtgcctgtgggtcagtgcatttcttggtcatccttactctgtcaaagctgccttgttagccaggttactagccatactagtctcattaccttacgataacacctcattcaaatggccttaacgaagttgaacacttatcttagtcactatggttggctttgaaccttttctagaagttaagtttattccctgggaaaaaacgtgtttgccaccacacagagtgctgggtgtactgggaacccggcttcaaaggcaactactccagtagggaaggttgaggagggtattgaatgatagctcactgcttaaataagtgtttagagtcccaaggtatatgtaatggcatatgaaatgcctagtgtttaaagaatattcagaaataatcagtcatactctttaatattcatatctcatagagttagcatcaggaaggaaacttggtagattaaataaaatagcagaaattactgctggatttttgtacagtagaaatgtatgcacatttttctcccctagtagtcacttaagttgcatatccatttcctgttacatctcatgacatgttttgggtaaaccgtttttggaagcgtccatttgtgtagaagttaggtggttctgagtttaactttgtacacatttctaggcacaaggaaattaaataactggctgagtgagagtgatctcgttgaatgctgaggcttgtttgggaattgggtttattttggtgaaatgtaaatttagatccactctgtattagtttttttttttttttttttttttaacggagtctctctctttcacccaggctgaagtgcagtggcatgatctcagcttactgccaccatgccaggctaacttttgtatttttagtagagacggggtttcaaccatgttggccaggctggtcttgaacgcctgacctcgtgttccactcaccttggcctcccaaactgctaggattacaagcatgagccatcgcgcccggccaagtccgtttttacactcctataacgacatacatgagactgcgtaatttataaagaaaagaggtttaattggctcacgattctgcaggttgtacaggcttctgcttctggggaggcctcaggaaacttacagtcataagggaaggtgaagaggaagcaagcatgtcttacatggaagaatcaggaggaagaggggtggggtgccacatatttttaaataaccaagcacagactcactatcacgagagcaacactgaggggcaaaatccactcccatcatctcatctccttgtaccaggcccctcctacaacactggggattgcaattccacatgagatttgagttgggacacaaatctaaaccatataaccctcatacacatgtattgtaattcagaatttgtcattttaaaaaaaaatttttaagattagcttatcaatttaaatgtttccagccctgaactctcaaagtcaactttaatgagttttgatttttgccgagaaagatcaacagcagaagaaagagaagtataacaaaacttagctccacaggagcaaactggagaattggagtactggtttatgattttggtcagttaagcagaatgcagtcctagtttaaacaagactggataaaagtccaaattcaaataccattcttacaagtgtttgaatttaattttgtcagtgtacatacatatatctagtaccatagtgatctcaggctttgaagaatgaaaagcaaaagaaatgatttagagtcctggtccaaccactcagctatctgatatccttttgctattttcgtttaaggggttatcagggaatgagctaatttgacattttctgcttaaagcaatggttggggctgggttggggtggagagagctgctctgaggaggtggtgtggggggcctgcgaggacacacttgggttacctggtacatctgcagttcccatggaattgggtctcaaaactgagaaatggaagtgtgagctaggaagaggagtagcttaatggggactttgccacacttttagtgctcctgtagctgcacacatgttgttctttgtaatgtatgatttgtgtcccagtggaacacttgaggagtgggagctggagaaaggaatgttgtacagtgttgtgcagatgtgggtgagatgaggtgatctgggaagctggcagtcttagttggcccaggaatgccaggacctgagatgacagctgctggtggcagcggggaatgcaggtgtatctgtgtcgaggttgggaattggactttgaagggctgggacttccagaaggtaaaatgggattgcactgtttgggagaaggggtacagatttttttgtttgcttttgagataaggtctccctctgtcacccaggctggagtgcagctgtgtgatcatagctcattgcagcctcaacctcctaggctcaagcaatcctccttcctcagcctcctgagtagctgggactacagacatgtgccactacacccagtgaatttttttttcttaaagatgaggtattgctatgttgcccagactgatcttgaactcctagaattctttttttttttttttttgttttgttttgagacggagtctcgctctgttgcccaggctggagtgcagtggcatgatcttggctcattgcaaactccgcctcccaggttcaccccattctcctgcctcagcctcctgagcagctgagactacaggcgcctgccaccacgcctgactaattttttgtatttttagtagagacggggtttcaccatgttagccaggatggtcttgatctcctgaccttgtgatccacctgcctcggcctcccaaagtgctgggattacaggcatgagccaccgctcacagccgaactcctagaattcttaaacattaaggacagaaacatctctgttcttaatcatgatttgcttttagtctcagcagatggaaaaatgatttacttgcaaagtgagagagctagatgaggaaggaaagaatatgattttgtaccactcagagaaaaatgtgagcatttctctactgtcttgggttggttattttccgttgattcaaaattgctctgcaggagacataggagaggcttgagagatgtcagtgggtggccatggctcagccttgatcctgagtgggtgggaatactctgttaatggggcaaagtggaccctccaggtccttcatgctccttttggaaagaagcgttcctggccagacacagtggcttacgcctgtaatcccagcactttgggaggctgaggcaggcagatcatttgaggtcaggagttcaagatcagcctggccaacatggcaaaaccccgtctctactaaaaatataaaaattagctgggtgtgatgatgtgcacctgtagtcccagctacttcagagctgaggcacaagaatcgcttgaacctgggaggtggaggttgcagtgagccaagatcgtacccctgtgctccagcctgtgtgatagagtgagaatctgtctccaaaaaaaaaaaaaaaaaaaaaaataggaaagaaaatatatgttcccctgtggttccctgttataccctcttctcttgaatagtttatcagaagggacaaccagctcctcatgcccttcctcagcaagtgtcaacagtgtgggagtgcagttgataaagcttgattttcacaactctgcaagtagctgagctccaccagtgttagaactgaaattgaggttatctcaccaggaaccaactttcagatgttgaagccagtagaaatttagtaatgtgggaaactgataactaaaagtattccaggttctaattattttgtcagctcagttttaggaagcagtcctagggattcccctggaagaaagtttggctggatagagggcagtgattgtgaattttctagatcttttgtggactgcttctgttgttaaaaaagcagcagtctctcagcacacgaatgggaagtgttaatgctgtaactgagtaacactgatactgagtttaaaataatggtaaatatcccactgaaatttcacccccagtacagattagggccttggaatgctggatagtggattaaggacatctgaaaatctacttcataaaagcagtgagaacattggcaaaacctattaatttcaagtttttcagaaccatggaaattagttaaagatttctagcaatctgaggaatgattgttcaagaaaacactgggatctcagtaggaacagtgaaccttgtgttttaacctgctgcaatcccattcccggcccctgaccacctccttggtggccttgaaaaccagcagcctcacagccatggcagctgtgaaactcagaagtctgttaaaccactggaagggacagaacagaactggagctcttgaaggaccatcctcaaagaaatgtcacaattcgaccatttggcagcttgcttgaaagggcctttctgagggctttgtctttgtttcacccaacacaggtcagctcacattgtgcaaacagctctacctgctgagcatttgttgaaaacaatcggtggtagttgtttaatcacagctgtctgaggtggtgttgctaattgaggccaagtggctgaccaaaaaattttttatttttattttatttatttatttatttatttttttgagacagggtctctctctgtcacctagcctggagtgcagtggtgtaatcgtgactcactgcagcctcaacctcctgaatttaagtgattggtcctcccacctcagcttcccaagtaggtgggaacacagatatgtgccaccatgcccagctaatcttttaattttttttgtagagatggagtctccctatgttacccaggctcaaacagtttttttaaaatggagaagtaaatgttccttgacgacttcaaaaagctccaacatattcctgggaatctagaaggctctgagcattctgagaaggctctaccttctctgctgtggcgatcttgaggctttatgcaagcctcaaggaagactaaggtagaattataaaccattcagcagagtgttgaaggcatgccccaatacccacacccacacgccctctcacctacccacccacccacccttagtaaaggctggaaaatttattaattcaaggcatgtaaggaaatctctgtgtagttactagcgaaccactaaactaagagtcttcagtggccatacatggcaaacaaaaaacaaaacaaacagactttatagaattagttcctgaaaatcactaatcagatagcaacaaaacaaaccctgggatgggggaagaatttgatttttagaattgacacattgtattagataaaatttctagtctttgacaaaaattagaagctatctcaagaaacaggaagatatgtcccatatacaggttaaaaaaacacacacacacacacacctcggtttctgcctagtttctaggatagaaactgtccttgatgaagcgcagatgttgggcatacttaaaacttaagtcactatgaaagttatgttcaaataactataggaaagcatgtctaaagaatcacatgaaaatatgcttgtgaggttgaagccaggaggattgcttgagcccaggagtttgaggctgcagtgagccatggtcatgatcaggccactgtactccagtctgggtgacagagcaagactctgcctcttaaaaaaaatattgggtgaccaaacagagaatgggaataaagaagtagagattataaaaaaaagaaccaatagaaattctggagtcataaactctagtaactgaaatgaagaattcactggaggggcttagcaggtatgaactggcagaagaatcagtgaactggaagacaggtcatttgagatagttcagtctgaacagcaaaaataaaagaatagaaaaatgaacaaagccttagagacctgtgtaacatcatcaaccatacccataatgggagtcccagaagagggaagatgtaaagggaaaaagaaggtatactgtgaatagttgtgtgtttgttctcatgccgctaataaagacatacctgggactgagtaatttataaaggaaagaggtttaattgacttacagttccacatggctggggaggcctcacaatcatggcagaaggcgaatgaggtgcaaagtcatgtcttacatggcaccaggcaagaataggagttgtgccggggaactcccatttataaaaccatcagatctcatgagacttattcagtaccatgagaacagtatgggagaaacttcccttatgattcagttatctccaccttatcctgcccttgacatgtggggattacaattcaaggtgagatttgggtggggacacagagccaaaccatatcaagttgtatgccaacaaattaggtaacctaaatggaatttatacaaattcatgtagatgcaaatggccataactgactaaagaatttgaaaatctgaataggccaataacaggtaaagagattgaaatagcaatcaggaaaccttccaccaaaaaatactgggtcacatggtttactgggtaaattcactgggtgaattctaccgagtattattataaactctttcaaaattttataattaacctgataccaaactaaagacatcacaagaaaactatggaacaatatcccttataaatacagaggccaaaatcctcagtgcaatgctagctttcctcccattctcactacttgtattctgtcataaaaaaaggattgtacaccgtgacaaagtggaatttatcccaggaacaaaaacttggttcaccataccaaaaccactcaatgtaatctgtcatgttaatagaataaaagacaaaaaccacatgatcatctcagtaaatgcaggaaaatcatttgataatattcagtactttttcttgatgaaaactatcaactagtaatagaaggaaaattctacaatttgataaagactgtctatgaaaaactcacagttaacatcatacttcatggtgagaaattgcatgctttctcctaagattatgagtgagacaaggatagtcattctcaccacttctatgtagcattgtactggagattctatctaggacaattagtaaaaataaaatagtaatataaataaaagcattgagattgtaatggagattggaatagaactataactagaaaagtatctgtagttacagttgacatgatcttgtatatagaaaatcctaaggcgcgcgcgcgcgcgcacacacacacacacacacacacacacacacacaaatcttgaactaataattgagtttacaagcttgtacatacaagaacattgaagcatccagaaatgaaattaataaaacaattccatgatacagtagtatcaaaaagaataaaatacttgggaataagtttaataagtttatgatttgtacacaaactataaaacattcttgaaggaaagtaaagaagacctaaaaaacaaaagaaaggacattccatgttcacggattagaagacttacattgttaatacggccaatactccccagattgagctgcagatggagtgcaatacctacaaaatcccagctggcttttttttttctcagaaattaacaagtttattttaaaaatcacatggaaatgtaagggacccagaataaccaaaacaaccttgaaaaagaacaaagctggaagactcacacttctcaatttcaaagctttgctacaaatctacagttatcaagacagtgtggtactactgtgtggacagccatagagatccatggaattgaattgagagtccagaaatagacccttaactttacagtcagttgtgtttttgacaggggtgttaggacatgaatggataaagaagatgtggtatatccatacaatgggatattattcagccattaaaccactaagctcagataaatgcatggatgaaccttgaaaacgtatggtaggtgaaagaagtcagacacaaaaggccacacgtttatttgaaaattctggaataagcaaattcagagacaagaaatagatgagtgttaatatgtgctggaggctggtgggaggggagaatagggtgtgatggtagtgggtatagagtttcctttgggatgatgaaattttctgtgattagctaatggtgatagatgcatagttttggagacacattaaaatcaatgaattatacatttgagaggggcagactttatggtatatgaaatctcaataaaacgtatttaaaaacaatatacagttaagagcgcatccagatacacctaaggtattagtaaagcggtgcattaagctgaaatatctggcttgtgcaaacttggggcatttgtaccttatgtagaattccaaaatctgcttaggattttccctgtccactggactgccactggcagggtctgcttccctgttcctgggttgagggagcagcattggagcatccctcaattcctgaatgaggaagttagttaaaagggcaatgtgagctgtagggccacactcatctaacaggtgagcttaagtttccattgatggcagacctatcagatgtttcctcccattctcactacttgtattctgtcattttgctcttatcctcccaaaatcatgctaagattttttttttttttttaagacagagtctcactctgtcccaggttggagtgcagtgatgccatctcggctcactgcaacctctgcctcccgggttcaagcaattctcctgcctcagcttcctgagtagctgggattacagttgcgtgccaccatgcctggctaattttttgtattttttttttttttttttttaatagagttggggtttcaccatgtgagccaggatagtctccatctcctgaccttgtgatccgctcacctcggcctcccaaagtgctgggattacaggcgtgaggcagtgtgtctggccactaagaatttttaaaataagtgacacaatgaagaacaaggccatagagatgatcttgggtaatttgagggtatcaaaaagtatctgcctgatggatggcttgagacactaaaaatgagtaaatgattacaaagtaaggaatgataatttgttttgaagaataaaacagattcttgtctctggtggttgagggatacgactgatgctgtatattaagacaagaagatgaagcccaaatggcagggaaattgaagagaagatacaggggtatctaggcacagggttggctgtgaaagtttcaatatgtaaaagatgataggctgaaaggaagaggatggtggaaaatacaaaagaggcattgataattcaggagtgtggagtgtgagagaagcttctccataggtgaagagttggatggacagtggttgttaggtcaaaataacaataataataacctagcacatatgtcagatatctgtttgggttcatcttacgcaccattctcttgggagggagcatgctttgaacagatccaatttatgatggagttttaggaccagaaagcgtcgtagtagcagagacttctcgtcctagagagcaaaggggaagtcattttttagttagttagtttgtgttttagacagtcttgctctgtcgccttcgcctcctggatgttcaaacagttctcccgcctcagcctcctgagtagctgggattacaggcgcacaccaccatgcccgactactttttctacttttttagtaaagatggggtttctccatgttgtccaggctggtctcgaactcctgaccttgtgatccaccctcctcagccccacaaagtactgggattacaggcgtgagccaccgcatccagccttagtttgtttctgagatagagttttgctcgtttcatcacccaagctggagtgcagtggtgtgatcatagctcactgcatcctcaaacttgtggggtcaagggatcccctcacttcagcatcccaaaatgctgggattacaggtgtgagccactgcatctagctgggaagtacttttaagagtatttttttcaggctgggtgtggcgcctcatgattgtaatccaagaggtttggaaggctgaggcgggagggtcacttgagaaaaggagttcgagaccagcctgggctaacatagcaagacccccatccctacaaaattttttttttaaattagctggtggtggtggtgtgcactggtagtcccagctactcgggagtgaggcaggaagattgcttgaatccaggaaattgaggctgcagtgaaccatgattgcaccacggcactccagcgtgagtgacagggcgagacccctccagatgatagctggagatcatgttattttcttactgattttcccccggtttagcaatacaaagtgatcgttaggttggcacaaagcacgatggaaaataaccattgttggcatcaagagaaatctcacaccacttgataaatatagtgaactagattactaaagagtgatttccctggattgcttgaaccagggctcagtgaaaatgttgctgtttgctttttttgtggtcaggattttttgattaaatgagaagcctacaaatatgcaagctagaaggtgttgtattttgggattaagccagactgtagcaggatgttagtaatttgagttgcataaactaatctggggaggcagagtgagtagctgatgtcacatgaagagcaaacccttgattccattcgtaatacataatagaacaccttcgtttctaaaactgggtgcagtaaagcataaaaaaacctttagtagctgatgaaagattctgccccaggcttgagtctattaaccagaaaaactgaaacaaaattaggcaaggtggtaagttttgctttcttttagtggaatattaggatatacagcaaatggtagcaggaccacgttgggaaaagggccaaaagactttttaaaaatcctgtttgaggtttggatccattttctttcatttttggacatacctttgagtagtgggtaaactgagagctcagtgaggtgagcctgtgtctttcagcgggcgggcgggctggagttggaactcctgtttcagtggttttcagatcctgctgatctccagaatggctccagttcccccacccctaatgctttattatgaaaaatttcaagcctgcagaaaaggtaaagaatgtacagtgagcacccatataccaaccacatggattctgcaatgaacattttaatatacctgcttttttttttttttttttgagatggagtcttgctgtgtcacccaggctggagtgcagtggtatgatctcagctcactgcaaccactgcctcctggattcaagcaattcttctgcctcagccttctgagtagctgggactacaggtgcgcgccactatacccggctaattttttgtatttttagtagagatggggttttgctaacaggccagtctggtcttgaactcctgacctcaggtaatctgcccgcctcagcctcccaaagtgctgggattacaggcgtgagccaccatacccagcctcataacggctttttttttttttttttttttttttttttttgagacagggtcccactttgtcactcagtttggagtgcagtggtgtgatctctgcagccttgacctcctggactcaagtgatcctctggcctcagcttcctgagtagctggagctacaggcatgtactaccatgcccagctaatttttgtattttttatagagataggcttttgccatgttgcccaggctggcctggaattcctgagttcaagcaatctacccacctcggcctcccaaagtactggaatctctttttttggaatacgtttcaaagcatgtgatagacatcggtacagtacagtcctaaacactgagtgtgcacgtcgttaactacagtttaatatttgttttgtttctggatcttttccagggatttttttcaagattaaattccaagcgccatgatagtttctgagttagaaggatggggcaggcccaggaatcagtactttgaaaagctcccaagtgcttcttgtgatgcatcgggtttgggatcttctgccccagggagcgcttcccgattacttcatagctaatcctcacctaaaatgcagaggtgggcagttatctttaacctgaccatagagctccatttttggttttcttggaatcttttgtttaaaacagtggttctcaaccaggggcagttttgtgcctcgagagacattggacaatgtctgggatggtttggttatcacaccagggggcatctagtgatgagagatcagggatgctgccgagcatcctgtaatgcaccggacagccccacaggatggggagttatctggaccaaaatgccaatagtgctgaggctgagaaacgtatttttttttttttttttgcctaaaagaaaattgcttgctccagatgactccagacaaaccaaaataaaaataaaaagtaaaaactaaaatgacaacatgaaatgaatttttaagagccaaatgagacctttagagacaatctcatcactgcctacctggtttccgtcaaatacagatttatgctccagaaagtacctaatttattaaaataagctgatttctaaaagcttgttagatggtcagtaatctcaaactttgagtgaatttacttgaaaaggtaatgttgctggtggcctttagtttcttttaatatatcttaaatatgatatcaaagaaaaacagaacatgatgctttgggatgaatgtaatgaatgaaaagcaggattctttggcaggtaactgtgtcccaggccctgtcctgcagggtggcagggatgtgctgagctgggtgaggggggaccgctgagggcttctctcagtagttgctctgtgtagaattttctgagtgtcttgggagaatgggggcatttttcctgaaggtctcacttgactccacagtgaatggctttacctgtctcttgagtaagatgcatttctagtaatgctctgactacttagtccacgtttgactagtttttaggaaaacaatatgtacaatggaacagaaagcttacagaaataaagggcagttcttttcttgaggtgaaagtgatgtggggagccagtaggtgacaggatttgctctggcccctccgagggaggaggaagatgaaggtgaaacgtggtattggtcagtccgtttcctgcttggttgatttcagggacctgatgagacaatatggtggtgaaaacagactgctggattgcaatgggcggaactgggcttagtctctggataggtgtcagttcatttcacacctgtcattgagggaaaataatacctgtatggctgttggaagattaaatgagactatgaacgggagctgtgtggataaaatgtaccaggcactcccagtgccagaaggtgttcttgttagttctgggtgtggcgatgtgcgaagacacccgtgtctgcggggtgtgtgtgagctgcagataaccagcaggtcctgctgatgaggcaaccttcttgggagtcttggctcagttccggggtttaattacgatgtatccttgggcaagtggcttaatgtctctgattgtaatttccttcattttttcatatctaaaatgaaagaggtgcttggatattcctaagggttcatctttaggagtctgtacttctggtgtggagctttctggaaaaaggcttctgggaatctgaaatggcagtagtgatgggcgtggtaacagcagtgttcatacgcttcctcagtaattttatgtaacaaacactcacagtgtacttactgcccctgagatcttaaaaactctcactttcaactttcacgacaccctataacttaggtaactgctattatccctggtatacagatggggaaactgagcctcagagaggttggtgatttgccccaagtcatagctgggaagtggcagacctcgatgggatgcaggcaccctgccctctgggctccgatcacttggccacctccccgcatgcaccacatgggctgtggagttggaggcatttgtgggcttcaagaatgagagttggtggggagtatgtgtgaggaaccggaggggtcatttccctgggactgggattgtggaggttttgattttgcttttccctttttaaactgggtgaggtatgagcatgggaactctgtacggaatgtgtcattggagagggacagcagggacaatgaaggtagtccccagagaagtatgagaaactggcattcagtgctcatgcagaattctctttgttttaataataatacaaataataacattaatattatttttagaggcagagcctcactctgttgcccaggctggggggcagtggcacgatcatggctcactgtagccttgaactcctgggctcgagccatcctctcacctccctcagcctcctgagtagttgccatgcgtaacaccgtgccttgctaagttttaaattttttgtagagaccgggtctcactgtcttgcccaggctggtctcgaactcctggcctcaagcagtcctccaccacagcctcccagagtgctgggattaaaggtgtgagccactgcaccctgcctaggattctctgtagatggggagcccacctgggtgagatgccagacacctggctccggcatatgcacagcagtttcacacgagcgtctcttactcaggtttcagagttctggtaaaagatttaggtttgacatccaccctttgaggtttttggttctcacatgtttgataacctgaagcttgtgggtttgatatgggcagtaaagtgggattatttctgttagagatccttattccctagagactttctggtctggaaaggtgagaggctatatattcattagctggaaccaccatgtggatcatttgttggaagaactgttaggaagaagacagatattgctatagagagcagggccagatgagcgagaaggcaaatgtacttcattcctgtacctgcaggcttattagggacagatgccctcaggcggggatgtgtagcttcacttgactgctctaaataaggagcgagagggtggtgtgtgtgttaaccatcaagattaagcaagcaagaaaaagacaaaagaaagccatcatttagtaactggtggagtgagttggagcctgggagctgccccaggacaactgttatttcccttgatcttcctcaaaccctaccaggaagtgagggcaacagttccctaaagcagccccatcaagctcttaagaaagcagtttcactgggagtggggagaggtagaattgggaggtggtagagggctggggggtatgaaatgtgagggccggagtcctcagcatcggaactccagaaataaaaagttgtctgggtctttgaaagcctcctatatgataatgttacagtaacagtgttctctgctactttcaatgaataaaaaacctagggtttctcagctacagtgtgcattaagagacatttgtggtctagttttggcacttcatttctatttgggaacatgcattctgagttttagacaactagcttccaaatgaaattttggaacaccaccctttttttttttttttttttttgagatggagtcacgctctgtcgtctgggctggagtgcagtggcgcgatctcggctcactgcaagctctgcctcctgggttcacgccatgctcctgcctcagcctcccgtgtagctgggactacaggtgcctaccaccgcgcccggctaattttttatatttttagtagagacggggtttcaccgtgttagccaggaaggtctcgatctcctgacctcctgatccacccgccttggcctcccaaagtgctacgattacaggtgtgagccaccgcacccagctggaacaccacccattttttaaagcagagaatttattgttatttgcacaaaaggacgtttgaacactttaggaaatctggatgctggaattaactcggtcaaaacaaagcaaaacacacagaatctcgtatcttttcctgccctgatgctgaactgcagccttgatttatgttttgtttttccacttttgtttctgaacctatgtggtacaggctgaacccttgtccctgcttcggagcacttaccttaattattatagaaaaaaattaagagttggctgactctgtgctgaactttctggtttttttccaggcagtttctccataacaaatgcgttttgcatagaattgaaattgcctatttacctcaatgacaccatcctaatagttggtggtaactacctaatctaataatctctcattgtgtgtgatagggctatttattgtattttagagtgaaataatgcattttgtttgcattttaaaaaggaatggtacaggaaatgattgtaatccaggactaaatatacagtttgcagtctttttattctaattgctttctgatgaatatatatatatatatatatatatttttttttttttttttttcttgagatggagtcttgctcttgtcgcccagactggagtgcaatggcatgatctcagctcacttcaacccctgcctcccgggttcaagcaattctcctgcctcagcctcccaagtagctgggattacaggagcctgccacaacgcccggctaatttttgtatttttagtagagacggggtttcaccatgttggccaggctgggctcgaacttctgacctcgtgatccacccgcctctgcctcccaaagtgctgggattacaggccttctaataaatattattttacacatcgagatagtactgtttatgcattttgtatgctgcctcttaaatctataagcattttcttttcttaaaaaaaagctttgggctgggtgcattggctcatgcctgtaatcccagcactttggggggctgaggcaggtggatcacctgcggtcaggagttcaagaccagcccggccaacatggtgaaaccccgtctctgctaagaacacaaaaattagtcgggcatggtggtgcatacatgtagtcccatctacttgagcgtctgaggcaggagaattgcttgaacctgggaagcggaggttgcagtgagtggaggttgcagtgagctgagatcacaccactgcactctggcctgggtgacaagagcaagactccatctcaaaaaaaaaaaaaaaaagctttgataaatattgtacatgattgcaaagcgtcccatcgtgtggatgttcacttagttatttgatacatatttttgtgttcttgggctgtttctaatatttctgtattacaaataaggtgatgggaagcagtttcattcatcaatcattctttttaaatgtcccttcctatcagtttctgttaatcagtcaaaagcgtatacattcttagcaggaacttaaattcttagtaggaatcttaacaggttaagatttctaagggtagagagggtacctgttttgttaaccggtggcttaataccatttaataccagtaaaggctcaataaatgtttattgactgaatgaacagaaaagatcagtgatcatgaagaatggcccagttagtggtatatatacgagaagtgctgcagtggttcacagtaacatgataataactaattgtgtaatgatttggcacgaggtactgatctaaatgatttatacattgttgcatgggattttggaaggggagggagtatacctctaaattagttcatgctatgggatggaagtaagactttaaatttaggtgggctcaaaaaacagaaacaaaccaggggccgggtgcggtggctcacgcctgtaatcccagcactttgggagtctgaagcgggtggatcacgaggtcaggagatcgagaccatcctggctaacacagtgaaaccctgtctctactaaaaatacaaaaaaattagccgggcgtggtggcgggtgcctgtagttccagctactcaggaggctgaggcaggagaatggcatgaactcgggaggcagagcttgcagtcagcagaggttgcaccactgcactccagcctgggcgacagagcgagactccatctcaaaaaacaaaaaaaacaaaacaacaacaacagaaaaaaatcaaatgttcgtcagctgatgagtggataaacaaaatgtggtatatccatacaatgaatgctatgctatgctgccataaaatggagtgaaacattcatacatgttgcaatatggatgaacctgccactcaagtagctggtactacaggcatgttccaccatgcctggctaatttttgtattcttagtagagacagggtttcaccatgttggccggcctggtcttgaactcctgacctcaggtgatccacctgcctcagcccctcaaagtgctgggattacaggcgtgagccagtgcgcccggcccaaagcttttttttaagaaaagaaaatgcttatagatttaagaggcagcatacaaaatgcataaacctggaaaacattatgggaactgaacgaatccaggcacaaaagaccacatattgtgtgattccacatattccacatattgtgtgattatatggaatgtttggaacaggcaaatccacagagacggaaagtagattagtggtggcatagggctggaagaggaaggaaacaaggactaactgctaatgggtacaggattttttttcctcaagtgatgaaatattcttagattatgaggatgcttgtacgactctgtggatatactcaaaacgatttaattgtatactctaaatgggtaaattgtgtataaattatgtctaaataaaaccatgaaaaattggatgaggtttagcattgtagaaggaaagatgggggtggtggtgtgttagtttttgagggctgccatagcaaaacaccacacactgggtggcttaaacagtaaccatttattttctcacagttctggaggctagaagtccaaaatcaagatgtcagcaggtttggttttccctgaagcctctctccttggcttgcagatggccacctggccaccttctcactatatcttgtggggtttttgtgtgtgtgtgtgtgtgtgtgtgtgtgtgtgtgtgtgtttgagacagggtcttcctctgtcacctaggctggaatgcagtggtatgatcatggctcattgcagcctcaacttcctgggctcaagagatcctcccaccccagcctccagagtagctgggactacaggtgtgtgccgcaatgcccagctaagtttcgtaattttggtagagactgggtcttcctatgttgcccagcctggtcttgaattcctgggctcaagtgatcctcccacctcagccccccaacatgctaggattgcaggtgtgagccactgcgcccagcctggtcttgtctttgggcacaaatatccgctagtgtctcattgcgtgtccacatttcctcatcagctctactggattagagcccagcgtaatggcctcattttaacttaatcacttctttaaagtcccagtctccaaatacagtcccattctgaggtagtgaaggttaaggctccaatgtgaattctgggggaacatggttcaacccaatgagaacaatggttatctgttgggagtgaggactgatatgagtggagatgcatcggtaaagatgcgcggaccacagatgtgacgcagcaggtgtgcacagaggtatgggatggggtcagtgtggagggccgtgtgccaggcagcagtgtggacttggtcctctacccagcgtaggggggtcattctgggggctgaaccgagggtgacactgtaaaaccatgcttcaggaaaattcatctctgcgccctggaggatggactgaggagggaagaggctgagggaaatgagtgtgcaagataatgaggcgatcccaagggaagtaaaaggattggaggccttgcaaaagcaaagcgaacccaagcttcagaacggaggcatctgagatgatgtcacagttttgaaccttatttatagaagaaagagggtgaaatggggaattaggtaaatgaaaatataggtcccattgttggtaagatacatggagtgtcattgattcagtatttagtgtgcttttccttattttaaacagctttcatagctcacttccaaactgtatttttgcaaccaactctttttctctctctattaaaaggagaatcttttattttacactaattaacagctgattctcctaagagagttttcaggaacctaaacctcagagttagatggagacaagtgtctttgttaatcatttatgtgaggtcagattgtacaatttgtaacagattttacagctggttcctttattaagggatgggtaggattcctactaattattattttgagttttactttgtttttaaccattgaaacttgggggatgtactctttggtttgggtagattttgcttatagatattgaagtttgagttgttgaatttcatacatattttattttgacttttaatcctttaaaacatttgactctgactccattgagctctgtagaacatctgtcatccagatcttttgccagaatatagagccttgattggacaccttgccagctaatttaaagcttcccaagatgcagaatactgtgtaaaatgtcctacctgttgtgtcagaagggagaaaataagacgagattttcatatttgcttgtagatccataagaaacttgggaagaattcctaagaaactagcagtagtggttccctgcagggtgggggctctggatgtgggtgtggcaggggatgggagagactggcaggttgaggagcagggtgagagggatgtttttatgatgtacattctcatatatttattttgaactatttgaatgtattactcatgtaggaagacaaacctttactttccaaaaatattagggaaaaactttcaagtctctcatttttagcctttttgttgttctgatatttccacacaggacagacggtaacatgcccgatacattttaagtacaaacaaggctgtaagttgccttacttgactttcttcactattttctattcaaaaaagtatttttgaatccaagggaattagaggaataatcttgaaatgattatgttttgggggaaaagtatattattaatatgatagttatctaatgttgtgtaacaaattgcctccaaatttagcatatgaaaacaataagcattcattgtgtcacacattcattctttcttttcagtgaaagttaacctgtttccatctgaatagatttctcactctgtatcctgccagtaatcattttgtactgtttctgtgcctttcaatccaagagtccaacctgatgaggttctgctggtgtaattcttttaaaagcattgtggatgtcctgtgaatctcactggagttcactttttaaacattagacttctgtggagactgctgaaggacaacacccttacttcttagggcctttggtttgccagaattgttctctgagccattgccttggattttttcctgacttcttaacaaaggattttataggtgggaccttcaacactctggaaattgccttagctacattcagccatctccttaggtacattttccatctcccacattgtagcaggcaatggcgttgctaaactttccatcaggaaataacaaagatctccttttccagtttccagtaacattttcctctctacaccatcatccacagtctcctgaaaggccctccaggttctagcttttccagggctcttcaggggttttcgaactctctttttacagtctttccagttcctgcccattgccacgttccaaagtcactcacacattttaggaatttttctttttttctttttagcaagcataccaattccagttaccagaatctgttagtttttgttatgtgttgccgcagtacagattgcctcaaagcttagtggctgaaaacaaggaacattactggccaggcacagtggctcatgcctgtaatcccagcactttgagaggccgaggcgggcggatcacctgaggttgggagtttgagaccatcctgaccaacatggagaaaccccatctctactaaaaatacaaaattagctgggtgtggtggtgcatgcctgtaatcccagctactcgggaggctgaggcaggagaatctcttgaacctgggaggcggaggttacggtgagctgagatcgcaccattgcactccagcctggacagcaagagtgaaactccatctcaaaacaaacaaaccaaacaaaaatgaacatttactgccatacacacttcctgggggtcagggatcccagagtgtctccgctgtgtcagtatgtctcagggtttcctgaggctgcagtcattatgcccgctagggcttgggtgggactgaagcacctgcttccaagttgcctcactcacatacctggcaagttggtgccgttgctggcaggaagcctgcttttcttgccaggtggacttctctgaggggtgcgtgaatgccttcactacagggcagctgacttccctcaggacacatgattcaggagaccaggggaaagctgcagtgtcattttatcacataatcccagaagtaacactctgtcatttctgcaatatcctgtgggttacacgtcagcattattcattgtgagaggtgactacatgagggtgagtccaggaggcaagaattattgggggctatcatggaggtttgataacataattggtaagaaattcagatacacctcagacttgattgtggcataccagtgtgttgtaaaacttgtgagaatggcttgccttagaaagacaagtagtccatccagtgtcttattcttatgttaagttgggattgctattttgttacccttgggcctctatatacaaagcctctcatagtaatagatctaattgggttgaagtttagtattatgtatatgccctctgcttttattagtagattttactttcgtgaaaggaacatataaacttcattattgcatttttgtataaatgcagctggggacatggttttattacccaatgtccattttgtaaatatttagagaactttttttttgcatcataaatcagtataaatagaagcttggtttccattgttgccctggctatgaggaagctgagagtgaaatttgtcgttcaagtgtggcagttatgttactgactggcgtatttatattctcctttatttgatattttgctatgagatgttccagtttacttgtgggaaaaaaagccaggtgttcttgaactcaacaaatatttattgaattccacttgctggccagacagcattccatgtgccccagatatggcactggacagatagataaggtccccactcgcctacagcttccaacctggtcgtaagaaaataccttagcttttgctccataatgtggaagaattggcctcagagaaaacctaattcatatgtaccacgtttctacccattcaagttttatttcataatttttttttttttttttttttttttgagatggagtcttgctctgtcacccaggctggagtacagtggcacaatctcagttcagtgcaacctttgcctcctgggttcaagcgattctcctgcctcagcctcccgagtaggtgggattacaggtgcatgccaccatgcctgactaatcttttgtatttttagtagagatgggctttcaccatattggccagcctggtcttgaactcctgacctcaggtgatccacctgcctcagacttccaaagagctgggattacaggcgtgaaccacgatgcctggccgtatttcataattctatctttccatagaaagaattggctggggcacgtataactaatgtgagttactgatttcagaggaagcttttggaagtggttgataggcactgaatgggatggggtggtgagtggtttaaaataacacttgaacctttggattcaaatggttttaaatgcatctcagaaaagtgaaaggtgaatgggagagctacatccattgagagcaagggttcatcagctagggctgtggcttaaactaagcagatattctttcctggcagttctacaggccagaagcctaagatcaaggcgtcatgagggttggtttctctgaggctcccagacagtcatcttctccctgtgtcttcacggggtctttattctgtgtctgtgtccaaacttccttttccttttttttttttttttgagacagagtcttactctgtttcccaggctggagtgcagtggcactatctcagctcactgcaacctccatctcccaagctcaagtgactctcctgcctcagcctcctgagtagctgggattacaggtatgtgccaccacgcccggctaatttttgtaattttagtagagacggggtttcaccatgttggtcaggctggtctcgaactcctgacctcgtgatctgctcacctcgtcctcccaaagtgctgggattaccggcataagccaccatgcccagcccaaacttccttttcttagagggtgggctttatctgtggttcttagttagcctcaccttcattcttccttttggtgttacggtcttgcttactgctttgggaatggatttgttgaaactacatgtgactagtttagggacccagtaggaggttttactataagtgtaaattaacatgtgcctttgggggaccttaaaagtttttgtcattgggccatgagatctgagtgcagctggtagaacctccttgttttctttggtatgagaactcctgggcttttgttggttccttccgttttctccggtgtattaccatttcacatcacagtgatattatccagataacacccatgatttgtttttaacggagtttcactctgttgcccaggctggaatgcagcggtgcgatcttggctcactgcaaccttcacctcctggttcaagcgattctcctgcctcagcctcccaaatagctgggactgcaggtgtgcaccaccacgcctggctaatttttgtatttttagtagaggcggggattcgccatgttggccagactggacttgaattcctgacctcaattgatccacccaccttggcctcccaaagtgctgggattacaggcacgagccaatgcacctggccacacctgtgatttattttatttcatttgagatttcagactttgtcgcttgttctttctgtagaagttttttcatagaagtatgttgtttcaaaagcaaccttcaaatagtagacttagcttggatgagaatctaaaagtctttgagtccaacaaccaagcaggtcccctgtccaggaagtaccttatagctgatgtttgagtatccctgcagatgggaactcactacctcctgaagcaccttacttctacccttggaatagtaacagagattaaaatgtccttccttaatttgaggtgaaatctgccttcttgtccattttacacacactaaccttccttccaccatgcaagctgagagagagccaggttgagcctgtgtttccacaacaactcttctcagacctcctgctgccacctaccatgcacttgctcttcactggctaggttccagttgctctgtacctttcctaaagtgtggtgaccagaatttggcacagtgagggaaggttccagagccctttagtgttatcgggggcacagagcttgggctttggagtcgctcagatcttgagttgatatgcacttctttgtccttgagcaaatgattgaaccttcttggactctcagttcctcattaataaaatgaagatgaaacaagatgagatggactgtgcacatttcctgatgggggctggtatgtgctggtgcccagtgcaaagtggctgctggcagaatggtgagtcaccagtacctgctcccttcactgagtgcttcctgtgtgcctggcaccattgctcagggctttacatggacttactcatttcattttcactgcagcctagagcgtaagtaagtatttgctttatccccatcttatagatgaggcatttgattctcaaaaaggtttcatagcttgcccatgttctagcagctagtaaaaggagaagccagagttttcatttaggtgggagtcctcaacctgctttcttaactgctgtgatctgcactgtggcatgagagtttgtcttaatgtaaatgcacatgtaatagcattattcacattgcctgagactgtggaagctcctttggcaaccagatgccacaggggactcatttcatgcttttagaagacttgagccactacatgccctgggaactgtgttgcttccccactggccatcactcttgcttttgtaagggatgtcacattgagccattaagtgtcatctctggaactcatgactctgttattccacatattaggttttctttgtagctttgtgtcaactccaagccaggtaaataaatcttggaagaaccaagttacagatgtgagtaaaagaggggatggcagggggcagaagtcagggcaggccctgtgacttgccaccagcatcctcccttcgggggaggtcctttcatctgggtgctagctggaggaattggcaacgtcggagataaaggtgtagatgcaccagctgaacgtcttgctctgtttcccattttacgtataagatgggtgatggcatgctgcattcagatccccatgggtgtatttgggatttggggtgggggattgtgtgtagaaggcaggaaagattgcccagcaactctcatttgaattgagggtggaagcggttctcgggcacagtatgccaaggcccagggcgtccaagggacagagcagagatcacagatgctgaacgggtggggaaggagcactaggcagggcatttctgatgagactttgatttgggaggtacgaaagttgtatgaggaatcttgataaactgcttagagtgaatggagatgagtaggaatcaccaccattcacatactggctctgcaagccactcagcacagctctgcacctgctcttatggaggagaagacaagcagaagagaatgttctgatctagaagagttcgtgtaagagcatgaaggcaacagcacttgagatgtggtgcacatgagcaaaatttcagtttatgtggaatttaagattaacagattaaaaggtkaaaattctctccctggtaccsayaaaaaaaaaaaaaaatcwcatgtactccctctctctcttttttttttgtttttttttttggagatggagtctcgctctgtcccccaggctggagtgcagtggcatgatctcggctcactgcaagctccgcctcccaagttcacgccattctcctgcctcagccttccgagtagctgggactacaggtgcccgccaccacacctggctaatttttttttgtatttttagtagggacggagtattccctctcttgactgatgtttcagcagtatgtggacagattcttttttaaggtaaatgaccaggggcttgtaatatgagtgtcacttaggcaacaggacatttcaatgaaatggtgatggaaataggttctttttgtagattgtgagctcttgggagcatggattttgctaaatatactgatttttaaatttttttgacatctagcatggtgcatttcacataggagtttttcaagaagtactcatttcacaggcatcttgaatgatagatgtttggtaagaactgcgcaagacagtgtactaatcactcattctcattcagcctctcaagctatcatggttggttagtaataacatttcaaaattatatttgtcccttaggctagcagagaatactgtttaacaggtttttcttgaaaagtaggagtagcgagctagagttgtctctaggtggggtaaataaaacatctcccaccccacccaaattgaagctgaataccttcaattcactactgtgcgcatattcatgtttcatgctgagcagttcacttgagactttctcacggaaagaagtgcgatacagttggtggtagtcttatatttgctacgtgctcatgtgttgtctgatgatagaatgcagtggtcaaatattgcacaatttacgccagaggaggagaattcccatcaactctccaaccatcataaatactttattccacacaataaggaaattgaacttttcagaagaatgatatagaacatctaaaataggttaaacttggaggaattatactgtccattgtaaaattggagccactcttgaaaaattgctgcatgccccactgaatgctagagcaagacatgcagtcccagctccaggggcttccagagtgattgaggcagggctgtgggctgctttcctgaagcctgactcctttctttcctcattacactgccacagatggtggcagatacagcatggattggcataagtcactctctactacctgcagaataaaggcccaatttcctagtgcagcttgcaaagtccttctcatctgccaccttcctgcctttgtgatttctcctcctgggactcctgcacccccttttttttttttaatttttcttttcttttttttgagatagggtctccatctgttgcccaggctggagtgtggtggcgcgatcatagcttattgcaggcttgacctcctgggctcatatgatcctcttgccacagcctcccagatagctgggaccacaggcacatgccaccccgcctggctaattttttgtatttttttatagagacgggatttctccatgttgcccaggctggtctcgagctcccgggctcaagcgatctgcccgcctcagcctcctacagtgttgggattacagacgtgagccactgcacccagccactcctgcacccttaccttttcgccgccctggccggctggacgactgcacccatttcacccggcattcttgtgcctgactgtgctctggccatttcctgtgtctggaatacccccattctcccatgctcagccccgtggactgtgtttattcgtaatcacacctcagagcccttccccttctccatggactccacccctgctgtgtagatggccccaacatcattttgctgagtggagtgccactgcaaggcatggcatggaggagcctgactgcataggtggcggcaccttgagggccttacagacctggcagttgaaggtatgggctagaagacagatgattccaaattacggctaagaaataggtaggctgtaaggcttgattgccgtgaatgcctctagaaagagcacttaaaaaaaaaaaaaaaaaagaagggatttccctgttcatggcctatgagtttaaaaacaagcaccccatgtcattagccctttgctcaccacagagttgctgacacattagacgatctagatatgtgtctgagattttggatgcctattgattgtgtattcacttagactttggtgcttttctagatgtttcacaagttaagatgttgttaagaggttctcattgttttattcctatgttctttccttccactcccaactcactccgatgtcactcatctgggttcttctgctcctgcctgggctgcttagtatttattgagctacacactgtgcttggcacccagggccatggaggcaggcaaagaggggggacatttattcagcagcttcttactgagggtatgggtgccaggtgctcgctctgggaaggtgcaaatgaaatggatgatggctgagcctgaagaattcccagggtgaacagatggggggaagccagttcgggcagagggagtggcaaaagctgctgcttaaagtcgttgctcccaccgcgtggggagtaattggatctgtctgcagcactgaggcctggacaggaagatgggactggaatgcagaggggccttgcagggctcagtcggggccaaagggcagagaggcagggcacagagagaagctgtggccgccttggttggtcccatggaatcctcgcccattggggccatgggcattgatcgacactgtggagcttgaaatctcaccaggtggaaacatctctgccagccctgtaatcatttccatagtttcttctggttcttttcatgaacattttccaatatttttatttgagcattaataaaaggtttgcctatatacttaaatttctgccagaaagctttgacttccgtgatctagagcaagcttgtgcaaccacaagcaggtcacatgcagcccaagatggccttgaatgtgacccaacacaaattcgtaagcattcttaaaacattatgagccttttgtgtgatttttttttttttaaactcatcagctgtcattggtgttagtgtattttatgtgtggcccaagacaattcttcttcttccagtgtggcccagggaagccaaaagattggacacccctgatttagagttttaggacttaggttttgggaatgccaaatgtttacatggaaaggctcatccattaggatgagcatggttttcctcatgaactccttggtagaacctaatgttagtgtgtggctcttttcctttattattttcttctgtacagggtactttataaccaatagctgtttctgaaaatatgggaaaactatttttgaatattgaatcaattacattttcagggtagtttgataaaaggcattctgtggtcatctttatctaaaacgaatcctcctggccttagtgtgattattttattttatttattttattttattttattttattttattttattttattttattttattttattttattttattttatttatttgacagagtcttgctctgtcacccaggctggagcgcagtggcgcagtcttggctcgctgcaacctgcacctcccagattcaagcaattctcctgcctcagcctcccgagtagctgggattacaggcatgcaccacaatgcctggctaatttttgtatttttagtagagatggggttttgccatgttggccaggctgatcttgaccttagatgatccacccaccttagcctcccaaagtgctgggattacaggcatgagccaccccacctggccttagtgtgattttgtattttatgttgtgagttttgtttttaaaaaagaacatcagtaagttcagaaaccaactccatgcttatttgctgtaatgaatttttcccttacattggaaaaacacagttccatctgtcctgaaggcccataagtcacagaatgcggtgtgttggaatgtgaaatcctttcagccttgcagaatctttatcattgtgctaaattgccatgtggaagaatggcaggtggcagttagaacttagggctttctatttattctctttctattttgcattagtcagttaaggttctgatcttcagaactttgtatgaaagtcagtgcttcatacaatttcacatcaaactgtggcattgcaagaggcaaaccatttggaattcttccttaaattacaaaaagaaaaaaaaaagtgaatgcacgaagattgttttgttcgcaagacttatgacacagccctccagctctgtggcttttcattctgatgggaaggaaatgagaccattgtaaatgactgttcagaatattagtgccagctccatgccaagtgctgttaagaacatggagagagcattcgagtgtttgcctctgaatttttgcccaggccatagttcgcagttggccggcaggctgcaagggccattccaagtgggagaagcattccatccctgggacaagtaaacagaacctaaaacaaacacatgtttagttgaagtgatacagcaccaggcaaagtgttctacttttaatgagtgaaggacatcagaaacaaaggatcagagccccccacgttgcattctcaactaattatagcactaaggcaggaatccttggccctttatctggagtgagtcagagagtatatcatgccttatctaaagtctaaagttaattgagcccaaggcccttctgaatagacacacactgtaatcagatatacagatggagtgggaagaggtaaatggtttcagatgccagcctggtgtagttgcatgccggaggggacttaaaggaaacagtgagaagtattagaaaactgtagttgaccaggtatggtggcttatgcttataatcccagcactttgggagactcaggctggaggatcgcttgaacccaggagttcaagaccagcctggacaacatagctagaccccatctctacaaaaaataaaaaattagctggatgtggtagtgaacctatagtcccagcaacttgggaggcggatgtgggaggatcgcttgagcctgggaggtcaaggctacagtgagccatgatcacgccattgcactgcagcctgcgcgacagagtgagattctgtctcaaaatagcaacaaaaacaaaaaccaaaagtatagttgcagagaatggctttccctgcttagttttatccagatgactgttagaatagtggtgaagaatctgacaagtggcagaatgcagggccatgggaagggggagagttcatgcctaaggtcagctgagcagaggagacttggagaactgagaggggatccctctcctgtgtttgttgggagggacagagcctgttgcccagcctgcaggtgtgggagggaccctgcaagaagtccctttggtcaccctcttcaggccccacattgcctgatgaggttcagactcctaggcctatgaagggtaataaggcccgtctgttcctgcctttacttttcttctatcagtttctaagcactctcagcctccgggcctttgatcgtggagcgccatgatacagtggcgttgtcctgagtggttagaggtgtgaattctcgaggccgactgcctgggtttgaatccttgcttggccatcttccagctgtgtgaccactctatgcctcagtttcctcatccgtatgataggaatagtggcagtacctatttcatggtgttattgagctgtacagttgctagctgctgccatcatcattgtcatttcctcatttcttttagacccaaactctcacactttccggaattatttctggagccccccagaaggaattggtctcttttattctagattccctaagaggcacagagggatactagggtgagaagatagtggtggctcacagtcatcactctgtacttactagttttgaaaatgtatcaaattgctgacgtctctgagcaccagtttcccaatgtgttaaagaaggatggtgatacccaacccatgtcacagggtttcgtcagtaaatgctaaaccatgtggtaagcagatgttctcagtgagcaccaggtcactattattatgttacttgtcatgacgagttttacttgccaccttgtattatagttgtgatgactatcttttttctagctccacattgggacacatgaaattatagagttaaatgaactcatttgtagatacttgaaaattgtaataatctttgaatatctgtacctgtttgtgtccagattcttctggttattttgacttccttataccccagtgtccttttttttttggagacaagtttttttctcttgcacaggctgaagtgcagtggtgccgtcgtagctcactgcaaccctgaagtcctgggctcaagcaaatcccagtgccttataagcaggcttcctcaaggcctgttgtcttactcatctgtattttccttatagcatctaggtcagtgccttgcttataatgggcatttggcaaatgtttgttgaatgcatttatcattacttagtaataaatactaaactattttttaatgggaaaataggcaggtttgggttctcatgcacatgacccacatttgtcgaatgtctgctgtgtaccagccagcagctttcttcctttagtgctatttcctgttagagtgggatagtggtatttcagtccacctgatgaccatttaggccaatcacatgttaatgggaattgaatgcatgcattaaacacaacaccagcgggtgctgtccacattaaaaccaggaggtctgcttcattggttcttgactatattgaaagagttggaaatctgacctgggcatttgaggtagactctaaggggtgtaagacggccctttgttttagagacttaggattggtgtgagtgactggcagggactgctttagtgcccagacctttagtagcacagacacaatataggaaaactcttcaaggaaaaccagccccaaataagagagtaggtaatgttctgcttctcttgctttatattctgcttgctttacagaataatctgagatagctaataatcaaagtgcaggtcccccaaaatgaaagtgatcatcatagaaccagaaaagaagggataaaatactgatttcaaggactcctgaagccaatggcagctctgagtttcctggcagccaagacaaagacagaagcatgctgggatgatgcatcctcgggctcggtaaaggaagcacaggacctgcctgctgctgtgggctcagggaagtgagtactattttccctgtctggagtggggaggagcacttgtctgtggtagggtgaggaggaccacttccttgggctatcagtcattaaggccccttaacatcatggccgattctgaaattacctctgaaagttgagatttatgttagactgtagctctcaagccagttcccttagaggccagagtaatgacagtgtaatttacttgccacctgcttgagagagacaattggtggttaagggactttcccaaggtgataaattgttttagcaaacagatccagaggacagctcctttcccctggtctatgtatgccttttgaacgccaagaccttatcctaaactcaagtttcacgaataggattaaaataccctttggggacctttcttacattgagaacagaggctgggccacagcttggctaaagcattccagatctctcttctgagcaagagattttgacttgttcataaaccaggctctctctcatagtgggcaaggccttaaaaacaaacaaacctgcaaaacctgaggaaacatcttcctcctacgtacttagctgatgtaggcattaatggaaatggatattggagtgggaggctccttttagggggcagaaaagaaagaatgtttttatttttaattaaaaaaatattttcttttgagacagggtcttgctctgccatccaggctggagtgcagtggtgcgaatatggctcactgcagccttgacctccttgctcaagtgaccctctagccttggcctcccgagtagctgggatgacaggcgcacaccaccatgtctggctaatttttgtattttttgtagagatgaggtctcactggcttgcccaggctggtttcaaggtcctgggctcaagggatcctccctcctttgccttccaaagtgctgggattataggtgtgagccaccgtgcctggatgaaaaagaaataacttgattatgggttatggaagtttagcaatggcttcaaaaaatgtcagcagggaatggatgtctttttctatcagatgaaatttcagtagtcatctgatacagccaaccaggagaaggaaaactactaagtagaaagacttagataagtggagtcgaacatctctgggcttaaaaggagcatcactcctggcatattcagcgtggcccagctgtgtttctctgagggcgatgtcctcctcagggtctgtgaatggctgaactcagaacaaggcttttgcaatctgcgccttctgaatacagtttttgttctgttggtttaatttgcatttggtgtctggagtgcctggtcatggaaagagcccgttctttgttttgggtaatgagggctgaggtttccaataagtatttctcggttccgtccgtgtcgagtgtcatttgagtagcaagtgactgttgctcattgcactgggcctgcgagcctccatttgcacattagatttttgcataccctgcaattagtaaattacagaatgacatcatgaaatagaaaactgtattcccagatggaaatgctttccgtattctatgcatttttattcaaaggcagatgcatttgtaaacctctttggatggttgttctgcctttgtcagttacttcatagcaaatgtcaactgtaaaccaagagagagaatgagccaggatggagaacttttttttgtttaattgtgtcatgttgggtgctttttggaaagtgggtgaattattcatgatgacatgaatgggggtattattttaaattagtatagaagcaaatggttcacaacccatttattaagaccctaatgttgatgtggctaccaagaagctttaccagcaagggccagtgtgttgccttccttggttggttagtttttctacatccacttaactcctagcctaattttatcttttttttcctctgtttttctttccctgaatgtttatttaagctctgataaatcattttgaattaaggtcgggtatgtatttcctttcttctcatcttcttccccctccatgactgcctgcatttgaatacatttccattgtgtgagaggcagtaatggtacagaggggtggggggtggtgactgcttggtgagctgtggctcacatgtctccagagacacagggcctaggaagactgattaaaaatcaacttttaactactataacggctcactttcctgaaacttgttatgctgtaaccccacctattgaagacataatcagaaccagaaagtgaacaaaattgaccctggaaatatgtggcaaaaattctgtgcaataagtatgataacacttttctttcagtgaattctctctcagggcccactcacagaaccaattttgacatcaaattttcttccttttaaaaatacagttaacccccacccccatctgcaggttctccctctgcagattcaaccaactgcagattgaagatattcagaaatttagaaaaatgaaaaataacaatataacaaaaagtaatgcaaataacgcagtatcacatctatttatgtagcacttgtgttgtattaggtattatatgtaatctagagatgatttaaagtatatgagagggtgtgtgtaggttatatgcaaatcctataccattttatgtcagagacttgaggatctgtgaattttagtatcctcaggtgtcctggaactactccaccatcaatgctgagggacaactgtatataatataaaataaataaaatatgtactgtaaaattgtaatcttgaaagtttgatcctgtggcagctgctattttcaggcagctcagcctttgtttgcatgctcaaaaccagcttgtgatttggtttctaagatcacggctggcttattatctgtgctggcttatcatgacagtggctcggtgttatggacacagtgagcatttggtgtcgatactcccagaatgaactgagtagaatggacaggagactgacctttgtctatttgtctatgtagcagtaatgccactaacttgttgcagtgctctttatcctcaaactatttgctatatatagtaatgcttaaattacatatattatatatattacatatacatattatatatgtggatatattatatattataattatatataatatatataattatatattatatagatggttctgactttgcacagttctgatatgcatgagttgcaattaccatgatttaaataataccagttccccaacaactctagtcaaatctcagttacccccgtgtgttaattgagtaattgcatgaagtacacaagcttgctgctagtgcttcagtatacaactcaccatgtaaataacagatgcatgtcagaatccgtggccaatcacagcacttctttcaaagcccatgggtgattggtcagtgcacatgttccttagttcatgcacagacagcagagtgtgtagttctgtggccttcttggctattggggataaaccagtgtgacattttacaaaaataccaaaaatcgagagggaattggtgaacgaagataaaagtgcagcaaagaagctgaaagtgataatgctagaagtgaaaccaaaacctacatggaattatagaagaaatagctgactgtgggaatattggcagtacagctgttggagagactgcctgcgcagcccgaggaggagaatgcaggtttaccacataaacaaggaaagtggttgtgaagaaaagaataaagctgtcctagaggacatgaccttggcaaaacaaacaaatttcacattaaaggatctctcagaaccatttcccagcatagaaaatgcaaaggatgaaatgtatgcttattcaaacttacggctcctagcagcactgttcacaatagtggaaaggtagaaacagcccagttatccatcagtgggtgaatagattaacaaatggatgaatagattaaaaaacagattgtgatatatccacgcactggaatattgatttaaaaagggacgaagtactgatacctgctacaatgcggatgaacctgaaagataccatgctaagtgaaagaagccagatacaaaggtcacatatgataaggttctatttatatgaacgatcttggtaaatctgtagagaaagaacacagatttgtggtttggtttctaagatcacagcaagcttatctctaagatcggggctggggcgcggtagggaaatgaggaacaactgcttaatggatgtggagattccttctggggcgacagaaaatgttttgcaaccacgcggacgtggtggttgtatgacattgtgaatgtactaaatgcgcttaattgtttactttaaaatggtgatgttatatgaatttcacctcgattttaaaaacgtgacatttcaccaagtcatagaaaagatgcttcgcttgtataagttatgcgacaaaaagataagcacttttaaatatttcttgataagttttttgtttccagggttttaaatggcaacgtgctgaatatttcactatttttcacttccttgtacatttataactgacagtaatagagtttttaatgctttgacaaaatttttaagaagtcacagaacaattgcattttttccttttcatcactaacattgttttgcaccgtttcagcttctatggacattcttatggtttcatactgagtactgcctgtgcacatgtatttcactacacccccgccccccaaccaacccctccaccccccaaacacatcccatacccattacagcacgttgttgcagggaggattctgagatgcgtcttctacaatagaaaaagatgtgctgggcttgctgtgaaaaagcagaggaagttagaggtgggatattagtggtgaagttggtagaacctcagctgcttctagggaagagggaaggtaaagtcagcaggtattagggaccaggggaagcctgtaacttataaggtatggtaactatcagagcagtttgtgggattggctagcagctgtgtggccagtagctggaagaaccagtggtctggacatgtcctagttttgagaaatctgtcggcaaggagagcgctaggactgaaggtatgatgcctgaagcccagaaacgcataatgggctgtgacaatgctgtttcacttgagctcatcggatgctttcgaggattcaatccatatctccctgttctccctgcattttgacacaggccgtgcttgcatgacagttgggttagtgtattcccctctccaccagagacttgggagggtaggggagtgtgccttagtcattctcgagactcctgtgcctggcagtgcctagcaacggcttaataaatacttgctaatgaacctaattattatgacattaagattttaatctaaatcttctaaatgactatgggaatctgtttatttttgcttaggatcattggtaatgttttctggccaaagctgcttttttgtctgagctgtgtgtttatttttcattcctgtctcttatttatattgtgaaaatgattaagaaaaataggtggtatggctcatggtctgatatcacgagtcaatgtgttgttttgaagccaactccacacataatggttcaagccctctgactacatcttagttttactccagattttctccatcaatgttgcttgcatttttcagtggtactttttggaaggaaagaatacgaaagccagcccgcaaaagatcactgggtgggggtcatagtctgtgatggtgactgaattatctggtaaagcatatttttgccgttttcagggtcaggcttcagcataatgagattccagaaattgttatgaaaaaagaaaccttaggttacccagcagctaaatgattactgcatcagctagcagctccggaatggccatttggccctgtagaaagatcattcttgggcaggctgttgatgtggagagtgagaggagcagctgttctttcacctgaggccccatctgggtagcagatggtccccagctaggcacaagccctgtgtagggacatccgtgctgcccaggatcctgggacactgcccaaatcaactgtcaggctgggagcctgtgtccagaactgtggatggcattcactttgggttgtggaagttggtttgtacctactcttaagtcttgccccttcaactccagctcaggctttttaaccctagtagaagctttcttatctgtaggtatttaagataagtttgaacctactggtctgtgggaaggtaccgaaaaagccaaaagctgcagctcctggggagcttacaggctcccccttgttgagagtctcccgagtgctaggtactgtgggtttaagggagaccctgcagtggtcaaaaggctgcctggtgaaggggggccggttcccatacttctcagatgtgcctttgggcaagccacttgcctcctggtgcttctcttttgtcatctgttaaaggggttagtgaaggacccttctcatagggttgttatgaagattaaatgatttaaagtatgcagagtgctaagaacagtgcctggcagatgattaaaaaaattgttacagaaatacagtacaataacatttttggtcacatttaatcctcaaagcaatcatgcagaaagggcaattgtcatacctattttgcatgagagactcaaccactcgcccaaagtcacagtgctagaaaggacttgagccaggatctgaacctgtcccctgaaatcttcacctgctttgcttcttgacacggcaggagaatgatgaggaagtgggttagtctgctagggctgctgtaacaactgcagatggggtggccaaaacctcagacatttatttatttttttaatataatcttggatgttggaagtcgaagatcaaagtgttgacaggcatgggttcttctgagacatccctccttggcttgcaattggtcaccttcttgcttcttcacatggtcctccttctgtgtgtgactgtgtccaaatttcctttctgtaaggacacagccatattggattaggcccaccctattgacctcatcttaacttatttactcctttaaaaaccctgactccttatacagtcacactccgaggtactggggattaggatttcaatgtatgaattttggaggtgagaggacacaattcagccaataccagtaaatgattagtaatcaaacacagggattgaatacgcagatattaaggattggaatgaagccagaatttggaaggattagaactgatggaggcaggtgcttggtctgggtgatttttgaaaatgattttcaggccaggtgaggtggctgattcctgtaatcccagcacttttggaggccgaggctggcagatcacttgaggccaggagtttgagaccagtctgggtaacatggcaaaaccctgtctctacaaaaattacaaaatatcagccagaagtggtggcttgtgcctgtaggcccagctcctctggaggctgaggtgggaggatcactggagcctgggaagtcaagtctgcagtgagcaaagatctgtgcctctgcaccccaagctggacaacagagcaagaccctgtctccagaaaaaaaaaaaaaggctttcaacagagtcagtcgtggacaaagaggaaaggacttttttcctcccctgcagctttaacctcccaggctcatatgatcctcccattccggcctcccaagtaattgggactacaggtacgcatgccactatgcccagctaattttttttttgtatttttagtaaagatggggttttgccatcttgcttctgctggtcgtgaactcctgggctcaagcaatcctcctgccttagcctcccaaagtactgggattacaggcatgagccactgcacccagctggaaaggactttttgtgcggtgagaggaggatcctcagaatacggagatctggggtccgcagacccattgtgggatgcacattgatttagttagatcttgagtgaagagatactgtgttagctgactgcgtgataaacgttagcctgagaaattagtttgttattttgataggcatttgagtcatttattgaactaaaagctttggggtgcctcatgtactaggcacgaaatcacagtacagttcttatcaggggagagatcatttgggtctcagtttcctaaaagtagcttttaactccaaaattgttttaatctatgtttgtagaggaacatcatgaaaatattttgaaattaaacttcttaagtataccttaattttatactgttattattttatttcatttttatgtttattttttgagacaagtttcactgtgtcgcccaggctggagtgcagtggtgcgatctcagctcactgcaacctccgcctcctgggttcaagtgattctcgtgtctcagcctcccgagtagctgggattttaggcatccgccaccacgcccagctaatttttgtatttttagtagagacagggtttcatcatgttggccaggctggtctcaaactcctgagctcaggtgatccacccacctcggccccccaaagtgttgcgattacaggcatgagccaccgtgcccagcctactgttattattttaattagtaaattttggtataatagagactggattttgacttaaggctatctgggtttaaatcctggctctgtcacttaatagctttgtcagatataccatatgacataaataggcagagtttccatcttatcacctgtgaaaagtggataactgtacacatcacattggattgccacatgcagtaatggatgtggaggttctgtccagcattgtgtctttcccatgataggcacttgtgctaatgctatgcaagaaggtagaaagttgaaagtggatagctaaaagtggaggcaaattaagggacttcttaaaatatttttatttaatagaaaacaatatatagcaagaaaatttggatcaattctttcatgcattcaacaattgaagtctgagaaccagccgtgattaggtgctccaaattagaataaggcaaggctcctgccctcattgagcttccaggcaggcgggaagcccaggagggcgtgataagtgctacaattcaggtgtgttcaggttgtcatgcaggtattgaagagggctacttttggatttatgcccacagaagagtttcacagtttctgaatttttgccccaggatgggagcaggcacttgcattaacatttgaattaatcacccagagaagagtggaaaagttatttcaggcaatggtaacagcatgtgcagaagtctagagggatgggagaggccacatggcatggttggaaaatagcagctcaggaatccatttgctgtgggggtggtaagtggaggactagatttgtgaaaggggacagatcacacaggctttgggaacaactaggaggttttttttttgtttttttttttttttttgagacggagtctcactccatcacccaggctggagtgcagtggcatgatctcggctcactcaacctctgcctcctgggtacaagcaaccctcctgcctcagcctcccaagtagctgggattacaggtgccccccaccacacctggctaattttgtatttttagtagagacggggtttcgccatgttggccaggctggtctctaactcctgacctcgtgacccacctgccttggcctcccaaagtgctaggattacaggcatgagccaccaaacccggctgactaggagatttttaagcagggaagcaatttgatgatatttacagttttaggaagaccaaactgacagcagcttggaggctgatttggaaaggaggcagagtggaaggcagggttgttgaggggggatgttttagaaagattttgtgatgattctgctgagactgaggttctgaacttgggctgccctagggctgccctacagcagcccaggaggacagattgcagagctttgagatagttggcatcaagatgggtgtgtctggggtgggcatgactgggtttctagcttgggtgtccaagtggataaatagcctttcatgtggaggctgagggcatgggcaggttgagtactgtccttcttccccaaaggtagctggccagccatgggagtcaatccacatgggatggaaggatggagaatgccagctcctctttaaagacttggccctgaagaatagagcttgcaattttgtatctagggtagtcattaagatccgttcacacaggtttggatctttaatctccagaatagagggtgggatgctccctgctccattgagcccaggtcatcatgtgacaacctgtgactaattaggcagtgagctgatgagattcgtgtggctcttgagctaagcttttgaagccagggtgagactctctggccctcattatctcagctgtggtgcttggcatgtcccagaaggtagccgtgctgtggacctcagtccccgagggagcgctgtgccaccgccacccacgatggacctgccctgagagagaagcctttgttttaggctctttaagatgacatggggattttggtggtgtttttctagcctaatctagcccttcctgactcaagtttttgtgtaagagaccaaaccttgctcattttcttttccttagctacaaatcagttttgaacttaccctttttcagtatatgcctgtgcatgtgtatgcctttgtgcgcaggcagacaaatatagctaggaccaaaagcatttctgctgttaccattatccttttgtccaaggtatttttcaatgaactaaatgtcagaaacacacacgctttcttaaaaaagcagctttattgagatatgattcacataccttacaattcacccatttacagtgtataattcaattacttttattatattcaacgttgtgcaaccatcattatgatgttaatttttttgtttgttcctcttgttgagacagagtctcactgtcgcccaggctggagtggagtggcacaatctcggcttactgcaacctctgcctcctgcgttcaagcaattctcctggctcagcctcccaggtagctaggactacaagctcatgccactacacccagctagtttttgtatttttagtagagacagggtttcatcatattggtcaggctggtcttgaactcctgacctcaggtgatccacctgccttggcctcccaaagtgctgggattacaggtgtgagccactgtgcctggctgatgttaatatttttattacccataaaaggaagcctgtacgatataaccatcaccccccagttctcccacacatccctctccccatccctaggcagcctctaaactactttgtatctctacattttgcctattctggacatttcatataaactgagtcatgccatgtatggttgtttgtgactggcttctttcaattagtggagtgttttccagggtcatcaatgttatagcaggtatcagcacttcattcctttttattggtcaaatactagtctgttgtatgggatacattgtgttataaaaccatatggtttctagagatggaagccatgctgacccctgtagagttttgtttatccatttatcagttgatgaacatttggattgtttctgctttttgcttctatgaacctctatgtagtacattcttgtacaggttttggatgaatatgttttcatttctcttgggtttatacctaggaatggaactgccagactgttttccaaagcagctcaccatttgacattcccccccagcagtgtctgagggttccagtgtatctgcatcctcaacaacacgtgttgtctgtccagtgatagccatcctactgggtgtccctgttgttttatgtcatttaacaccaaacttagatcacatttctgcattactgactttgcacataccgtccccaacttgcaacttatgctgtcattaacttgtgtagatttatccatgtttggctgtttatgaggatttgtccgtgtcaacatgtatgtttcataaggacattcttaccttccattaccagacctacattctcaatttaagatttgatattttttcagtgtttaggttttcaattgaatcaactgacaagtttagtggcttaaactttcaagcttaaactgaaatgccaatcttgttttagaccaaaaattacatcctatctcttctcttaattgaggaacttaggtggtggttcctatttgtctttatttatttatttatttatttatttatttatttatttatttatttatttttgcctacttcagtttggtcggtaaactatcaattgtattttcagtatgtatatatatatagagagagatttttaaaattttattattattttttgagacagagtctcactctgatgcctaggctggagtgcagtggtgtgatcttggttcactgcaacctccacctcctgggttcaagtgattctcttgcctctgcctcccaagtagctgggattacaggcatgtgccaccatgcccagctgattttttgtatttttagtagagatggggtttctccatgttggccaggctcatctcgagctcctgagcttaggtgatctgcctgccttggcctcccaaagtgctgggattacaggcatgagccaccatgcctggcccattatatatacatttttaatataatgtgaaatcatgtggtctctactatagactgaatatttgcatcctaaaccccaatgggatggtattaggaggtggggcctttgggacgtgattaggtcctgagggtagagccctcatgaatgggattagtgcccttataagaagaggccaagtgagcttgtttgctccttctaccatgtgaggatacagcaaagaacgtgctttctgtgaaccaggaagtggccctagtcagacactgaatttgccagcaccttgatcttggacttcccagcctccaaacctgtgagaaataagtttcggttatttataaaccacgcagtctgtgatagtttgttagaggaacccaaacttaaaggcagcttctaaaggggagcaatgctgaccactctgtagtttgtatatttggtcaacacactttattgagtgatgagtctgaatttcatgcttgattgatgatgattggtgacagagtagacaaggtccctgcttcatggagtctatggtctagttggggaaatatacctttattgaatcaaggcagatgtgattacagattgaacaatggctgtagaaggaaagggatttccaagagagaattagaggggcttctcaaattgagtttgactttctctttggtgggggttgagtcaggaagaacctttttgaagcaggaaaaatctttagctgagacctaaaggaaggggaggagcttgcctgggaaggacactgtggtggctggagggggtttcccaggcagggacactctggcaggttagagtaactgaaataaagtctgtattggagtgcagagactgccagaagaggggctcatagaggccaaagtgggacggccttacggacgagctgataatttgggcattattcaaggtggggtgctatgtcattgaagggttttagcagaggggaggcattatccaagttaaagaacaaaacaaaacaaaacaaaacccactctagttgctgtgtggcagatggattgggattgggggtgtggggttggtggcaagaatagatgcagggggacaagttatgaactgagactgggacctcatgggtcttcctgcccaagaccgagatggctgggggtgggggctgcggctagggacatggggcctcagtgcccacgttttctgtcctctgtcccctgtgagggtatgtgcgctctcataccagccgcagggcactttggctcgcttgtgttgctgggagagatctcctttaatgtttttttgtgtttttaaaatatgaacgtaggccctggcagagacaaggcagaaaccacttttgagtttttagggctgtgcattcttaaacaactctgagtgacccttccatcattacaatttatcaactaaattagaaaacgtgctttcacagatttctagtacttgggcaaatttgttatttacgctcaagatgaagtgttgcttctagtgacagttttgctgttgggcagcatttgtagggtaaaggtgtttttgcttttggaatccattgggagatttaaaaaaaatatatatatatatacacacacacacacacatacacacatataaagtaaaaagcaggaagtctatgtgtgtgccaggggatcccaaactcttggcctccagcagtcaagagattttttaaaacctttatcttttaccgtcttgctttttaaaatccattatgtctttgtatagtacttgacatacatgggcagaagtcatgctggaacgtgaagatgggagcatggactttctggagcatcccaggtggggttcctgtggctgctgtaaggcaggaccgcagagcggtggctgcagacagcacggatttgctattatatctcatagtcatagtcctgtagggtagagtctaacatgggctaaactcaaggtgtcgccagggctgtgttcctcccggaggctctaggagaggatccccttgcctttcccagtttctgcaggacatctagcagcctcagctcattgtcccctccctgcgtttgcaaagccagcagagtagcatctctgtggccattcccacatggtcacatctcctcctgggatgctctccttctgcctccctttgcactttcaggagcccttgtgattccattgtgtccatctggataatccaggataatctccctattttaaggtctgctgattagcaactttaattccatctgcagccttaatcccccttttctgtgtaagctgacctagtcacaggttctggggattaggactcgcgcatcttgggggaccgtcattccacctgcggcagtggtaggctaaaccttaggcgtctgatattgtgaacctggctcactggcctttgtcttcttgccgactcctattggtatttgcctgagatgtttttcttttctttttcattcttttttcttttctttctttctttcttttttttttttttttctggttctgggcccacagtaggtgccttagaatcatgcagtccagttgccttctgggaatccattgtagtaagtggtagaggtttttaaagcagtcctaggggagatgcaattccctgcttacattcaagtttttcgggaagacaagtctgtttaggaaacataggaggttagttgcattttcctcattaatgcaggatattaaaatattcagagggaacccaggagtcctctcaagatgagtttaaatttctcatacttgtagaagttgttgagaaatactggattctaatttccagaactctggtggggaaggagactgtgaacctggtctgaattgagaatggggattgaggatttttggacttttgaaagattgacttactctctcccgttagttctgttgaactgctgcctaaatggagggaatgtgcatcctctcttgtgttgcgtggcaatgccgtggggacacttgggacatgagcaaaacgttgacagaggggagcacttcccggaatattattaatacagagttagttttaacttcaccttttgggcaaacttgacattatagatcttgaaataggattatttttcccttgctaagaggttagcaaaatcagtacagtgtgtctctttgacaggccctagaaagcactctacgtttgttaactttaaatttgggttggaaattgatgttattggaggagcaaggcgtgtttaggcctgatgatttttggatagcttgaccaaacccggtacagccaactttcccccaaaactactgtgtccttctcagtggttcctgtgcctgcacagggacccagcgcacaccccagtgtgcctctttgggctctggctaacttaggctgacatcctgatagaacgcagtgaccacctggtcatttgattacagcgcttcttccagacagcacgctcagaattgcgtttcttacttccagtcttcttgccctgtaagcattttctgtgagtcaccaaatcaaccacaaaatcatttaattttatggaagtggaaaatcagacttattaaaatactgtgtttttgttttaaagctcagaggtatagagatccatgaatttttttccatgccttctcataatgtggttttggtttagctgcggttcttccaggcgcagatgtcagccctctgcagatctgctgcatcagagtttatggcctgggtcttaggaatttgcaatttaaaataaccccttcaggtgcatctaatgcagcctaaatttggggccagctcatgggttgtaaactgtactgccctatacaatagtagcacagttctaagctcaagggactcaagggggtccagtaagtacagttcaccagagaaataacaaaagcatttgcagagggagaccagagcatcacctttgttagggcaaaggcttaacatagaggaatgtggcttctatgtccaggccagtgggcttcctaacacagaacatggaattaggcagacttaacatcccccatgcccaccctggtgagtgccagcatgggcttgagctgctcttcacactgatggtccatcctttggactgatctccttgtgaaggatacaatacagcaaggagcgggggcttggcttggcacagccagttctttacccaaactcctgccaacgctggcccttctttcttaccctggttggagtgtgtgagggtgggagaggcaggaggaatatgctggacagagcttcttccatctagaagggaatttcaggagtaagaaagcggcagctcttaacatacctgggagaaagcaagtgcttagtaaacagttgaggaatgaactaagaattttcagacagattttcctcttctggagcatttgcagaaccgcctctttgctcaagtgctttgttttttacagattcatttaaggtcatggttccagacactaatgtaaatcagcacatatattattgaagaagtagaatacctccaggcccagttgccttggagcaggaaaaacagttcatcctcattaccatccagaataatttatgttcaagataatacacaatcaatttactttttatcatattcgaatttgatttgtgtacacttttgagtttggatgtcagccacattgcttctgtgtgttaatctttatttacgggaataaatgggaagcagtcagccattttttattctttattggggaactgcattatagtggaaaaggtaaaattataagctaatgagaccagaggtctgtatgtatgaaaaccatcctcatcgcttcacagttgtctttgaaccagtttccctgggattgttcgaaatgtagcaaacgtggctcacggttaccctgacggggccagagcaatagggggaggtgtgggcacctgtatacatttgcgttgaaaaacgtacccgggctattctgaggtcctttcctgcccccagcctggtttctgggcctctctggagtctctctttctggcacttgtgttctggaatcgcttatcagcctggactccttcagcagcagctgccacagtgtctttgtagtcacactacacacctatatactccatgacatggagtgtatagaatgtttcccgtcttcctcgtcagtgctgccaggagaagcttcccctctcctaacctctggatagtggctggctctgctttattcaggcattgctttcattttttagaacagctgctccagtatgcttagtgtgaatgacagcccagcagaaagaatggacttgacactggagactgcaccactccacatttctttgtcccctcaagatgacttaactagctcccttcccttttccttcatgcaatagcctcagaagttcggctggcattgccaagttgttagagtgaaccttaacctgtgtatgactcagaattcacatctgctgtgtcagtgaacacacagatctctttgtgaacacatggttatcaagaggcacatgctcttacgtatcctgccggattctggtaaggcgctgcttgcgcagtaagcacttgtcaggtgcttatgatgcctgtgacgtgaggctgtggatgcagaatttgtggtctgaacatttttgagtagataaaaatggaaactgaaattggtgaagcagacttgtggcttttcctactttctcatctcttttatggatataactagaagctctgcttcatcaatgagtgtgttaaaacaatttgtttacaaaatcctatttattaaaataaaaatctgaaagaactgcgctgctgcttttcagtgctcgccactctaggaacctgtattcctcctcttgttcatcctcccttttcttcttttttgttttgttgttgtttgtgtgtatgtatgtgtctgtgtgtgtgtggcggggtggtgggggcggtgttgccaaatcttttgcactgaatctttgaagatgtcctgaaatacttaccaaatcactagagcgaagagaagcccatctgatttcttcatctcccagctgaggaaggacacactttctcccactctccagtgaaggcccctgtgtcctgtgggtgggggagctgacatcacactcagtgctgacatttcttgttccctgggtgggcgctgattccagatcctcttcagccccccaagcggaactgggtttctcttgaaggtcctaacctcagctcacaccacctgctactacctttctgggtcttggattttgttgttacccaggcccggggtcatcccaaaggctcttgatagccctgtggctgtgcttgctaatacactgtcctcatcttttgtcagcgaagtaaatccaataatgtttccttctctttaatccaagccagtcttcattgaaaacacttcacctctaagctctcaggctctgcagtctgccctctccaaccttacccctcatgccccaccttccctttgtccccatctttcccttctgttccagtcattctcatgggccatggagagtggaagcctgggagggctttgccaaagtccagttgcctgctcactttcctaagggccagagcaatgggggaggtgggggaggtgtgggcacctgtatacatttgtgttgaaaaaggtacccaggctattctgaggtccttttctgcccccagcctggtttctgggcctccctggagtctctctttctggcatttgtgttctggaatcgcttatcagcctggactccttcatcagcagctgccacagtgtctttgcagtcacactacacacctttaccccgatccactgacctgctgtcccggatggccctggtgtgttctctctgttcctagccgcagaaattgctggtgaatgtcaccaagctaagcggtgtagctttataacaagtggattcagccagctgggactggacgactgagagtttgcagcccctgctggaccctgtgtaactgtccagttcgcctagctattggtttggcccagtggttttcaattttggctgcacatcagaatcacctgggggaggtttaaaaccctacctctgcccccatgccctggggttcacacccagaaatgctgacttcattggtcgggggctggcaggcctggacattggcggtttgcttagaagctgcccgggtggttctcatagcagccagggtcaagcaccacggcctgttcttacctggctccccaggccccctttaatctccctctgcagcagattggcacacatcgcattgtggagcaagccatctgataattttccttaatcgtctttgaagatcttccctctgcctgttgatcctttaaggaagaagtctccttgccaggatcggcctctccagtctctggatcccttcttgcttgctctcttcagaacccatgaaacctctctggctgccttgacgtcagccagattggactgcccaaatagaaaggcagaaatccctgtcgaggccagacagcaaggatggagagaggcagggggtgaaatgaaacacaccaggaggagtaaagacaaggccttctgatgtcagccaagagcaggtgtggattaggcagtcctaagtttgagtcctggttctgctattcattggccaattgtattctccctccctctacgatgaggttaattacagttacctcactgggatttgtgaggatggaatgaggtaacaaaacataaaatactatcagtacaatgcccagctcataatgagtatgcagcaaatgttcaccctttaaaaaatgccacggccgggcgtggtggctcgcgcctgtaatcccagcacgttgggaggctgaggcgggcggatcacgaggtcaggagatcgagaccatcctggctaacacagtgaaaccccgtctctactaaaaatacaaaaaaattagccaggcgtggtggcgggagcctgtagtcccagctactcgggaggttgaggcaggagaatggcatgaacctgggaggcggagcttgcggtgagccgagatcacgccactgcactccaacctgggtgacagagcaagactccatctcaaaaaaaagaaaaaaagtccctttctaccttcaaattcaggaaaattttctttagtccaacagatgtaatccccccctcactcccccatccccactggtatctttatgtactcaggtgactattaccatattctgcttggactgtttttatacattgcagataatttgaagatatggacttaggtaggtttttagatttataataattaagtgatgtttttctatcaagtgatgaaagtttcaacagatatttgttaaaatttaatttagccaaaggaagacatgccatttcagcaatgtgatctaagtaaaagtgtcttctcttttttttggtggggagaacagagtgtagaatgatattagtaagatagagaattgatgaccaaaccataactgctagcattttagtagaggacacaacagagaaaggattctcacgtgtgttattaggatcttggatgagagtgggctgttattttaactttggcccctgttggagcaaattactctttgggggcccggttttcctcaggaactacctacagaattcagtgagatagattttagaacccttctgaatgctgctggtttggagattctgtctgtagcaagttttgttcagttgttaatctttctttcttttctttcattctttttttttttgacagggtctctctatcacccaggctggagcagtggtgtggccctggctcactgcaggctcaagcgctccttccacttcaactgcccaagtagttagaaccacaggcgagttccactacagctggctaattaacaaaaattttttttgtagagatgaggtctcactgtgttgcccaggctggtctcaaactcctgggctcaagtcatcatcctgtgttggctttcgaaagtgctgggattgcaggcatgagccaccacacctggccaactatatgttttctatgtttattatattttctgatttgtaatgtattttaaaattatcccaataatatatgaatgcaatcttgaaaaatactcagcatagcctttaggctaaaaagtggaggtctgccatttctctgccttgccccattcccttcccatgtcttattttccctgttaccaggttggtgtgaagcctttgagacctttttctctgcacacccatatatgtgttttttctatgtttttatagtattgtttcatttaaaaaataaactaggtttttgaacttgagaaatttcaactcgttctgctcagagctttgttttattaaaatcagtgcctgggagttgctcaagtctttgggctgtgtcgccctctagtggtagccctttggcttacagctgccattcagccctagggctggggtggaagtgatggttttgtgcccacaacctgatggcagggttggcagtgtttagttgctgacaagtgtctaaaactcaatctaggccgggtgctgtggctcacgcctgtaatcccagcactttgggaggccaaggcgggtggatcacctgaggtcaggagttcgagaccagtctggccaacacggtgaaaccccgtctctactaaaaatacaaaaattagccgggtgtggtggcgggcgcctgtaattccagctactcaggaggctgaggcaggagaatagcttgaacccaggaggaggttccagtgagccgaaattgcgccattgcactccagcctgggcaacaagagcaaaattctgtctaaaataaaataaaataaaataaaatataaaagtctagtgagccctggagtggtgtttgccttgctacctccaacttgccctttttaagagacttaaaatttgtggtaacttttcacatgatgtgtatttagatactgctgagttaatgaggctgctatacctggctgatttattgtttgctttcaaagcagttgtcaagtggtgtccacccatcccactgatggattttttttcttttttctttttcttttttttttgagacagagtcttgctctgttgcccagactggagtgcaatggcatgatctcggctcactgcagtgtctgcctcccaagttcaagcaattctcctgcctcagccccctgagtagggattacaggtgtgtgccaccacgcctggctcatttttgtatttttagcagagatggagtttcaccatgatggccaggctagtctcgaactcctgacctcaagtgatccacccacttcagccccccagagtgctgggattacaggtgtgagccactgcacctggccccactgactgtttatatctgttatactgttcttgcctctgtgccttttgggttccctgtcatggcagcatccttacatcctccaccacctctgaatgttcccgcaccttctctttctaatggaagcctggctggcccttgaagacaccgccaaccctgctggtctcttcacagagtatccacccagcaacctggcctgtccattcttgacttcttctggtccagctgcctttctgtacctcattttagtgaccctttttagatctcgtccaggttagcctctagaaccctgatctcaggcaccccactctgcgatcttccgttcccagccttcccacttgaaatcgtcttggaaatcccgggctttgccaatctgtggacccaccaccttttcaccccctttccggtgccctcaggggactttgctgtagcctgaacctcatggtcccttcctgtaaacagccaaactgctttgtactccctgaggagacccccgccccggttggacaccccaccctggttaaacccactttcacaattattccaaaactgccaactggacttgcatctcttcagatcggctgtcatcactcattgtatcaggaatcttattacacttttgcagcagtttcatcggttcagagaaaactctttcataccttctgctcatcagttctccacacctcccatcattctcctagctgatgactttgctgcttgtctcattgaggaaagaagccgttcagtcagtgagaccccctgtctcgcctccacctttccacctgctgccgcggaggcttccccagctcctgtctcactcaggccccttgccactctcaaggcatcttcttcctgcttcccaaggatgtcactcctgcagttaaccctttttcctcctgcattattcgtttcttcctttcttccgcatccttccaaatagcgtaaaaacaggccttatgacgtttctctttaaaaatgtccttaccagtgccacgtccctcttggccactgcccctgttgtgcctcactccgtagaaagaagtgacctgtagagactatatcctgccctctccagcgcctgccttccgattgccccttccagtctagtaactgtgtccattccttctccaatgaccgggtgacaagctgtcccttctgagagtgccagagggtgttccccgggtgggaggccggatgtcagtgccagctgtgctgtcctgggtgagcctgtgagggaccacatctggtcagactcgtgcatggcctgccctcatcttcctccgcctgcggtgcctttgagagcattgactccactaagcccgatttctgtgtggcatgttgtctgttcctccccggcctcctcgatggctcctccttggtctccctctggctactcctctgcttctccgcctccaagtgctgctccaagtgccttctgtgctctgtctccatccatcccaaaccttccaacactctctgtaagtggaatctcaccttgaggctgccagcccagcctcctcacctctgtgctccatctttagagagtcacctctctgtctggcattcccacttgaggaccaagatgattctcaaccttaacgtggcccacatggagccgctgctcttcctgcccagatctgccttcaccaccacatcctgcccactgtcactaaacggcaccagctcttacctggtttctcagtaaaaaaacaagtttatcatcttccctcacatactttattcaataataatcactaacatttattgagtgcttcctatgtgcctgacaccattctaagcatttgaagaagagtgtattaacctgcatgaggaaggctattaaaacctatgtaattaattaattaatggatggatgtgtggatgaatgaatggcagggtcttacactgtcacccaagctggagtgcagtggtccaatcatagctcactgcagcgtcagactcctgggttcaggcagtcctcccacctcacccttttgagtagctgggactacaggtgcttgccaccagctaatttattaaaaaacattttgagagatggggtttcatgatgttgccaaagctggtctcgaactcatggcctcaagcgatcctcccactttgacttatagattgtggggattagaggtgtgaggcatcgcgcctggcctgtaataacctatttgtgaatgagaaaggtcaggcacagagaagttaaataatttgctcacagaaatttcctgtgttgcagctgagatttaaatccaagcaatgtgtttctaagcctgggctcttaactgtaacactgtcctcttccccccaccaggagggtgtgtgtgtgtgtgtgtgtgtgtgtgtgtttctgaatctgaccagttgaggcaggctttcatgtcattctctctggccacccccttctttgttcacctccagtctttggaatagtcagagcgatgtgttcatggtagaaatgaaatcacatcaggtcctcagtggcccatcacatttagaactaaagctcactcttgcctcatgcctctgagcccttatatgatctcgagtcaccaccccactgccagcctctccactctcttctctgtgcctcatgcagcccaggaaatataatttggaccgcaaatgccagccacctatgtacggtaacttttctagtagtcacattaaaaagaaaacaggtgaaatgaattttaatgcattttagttaactcatgatatcaaaaatatttcaacatgtaatcagtattcacacaaagattaatcaagtatttaacagtcttttcttttgatccggggtcttggaagtttgacctaccacctctcaatttagccagccacatctccagtacttggcagctgtttgtggctacctcatggacagggcaggtcccgccacccgggcatccgtctgcctggcgcacgcaccaaacgggttccagtctggggaagttggcatctgctgttgttacttttggtcagaacgcccttcgcctgcatcctcacggggctgactccctttagccttgaaggggaccgttgctccgagaggcctgaacactccagctgcagctttctccccagtcattattgagatcaaattcacatctcttacaattcacccatttaatgggtgatgcaatgcggtggcttttagtatattcacacacatgtgcagacatcaccatagccaattttagaaaattttcatcagctgaaagagaaaccttgtatcctttagctatgactttgtgattccctgttcccctgagcccctggagaaccctatctttctgtctctgtaggtgtgctgattctgaatattttgcataaacggaatcatactagggggtcttttgcttgggacccctttcacgtggcgtgtttccacggcccatcccgtgtgatagcatgcatcagtacttcattcctttttatggcagataatagcccattgtatggatataccacctgtttttttaaaaagtccattcatcaattcatgagcatttggtttgtttctactttttggtagaatgatgctgccatgaacacttgtgtgcaggtttttgtgtggacatagacttaggaatggaataaccatggagttacggtaactctacatttaagcatttgagaagctgctggcctgtttttcatctgcaccatttgcattcccaccagcatcaagtgggagctccagtctctccatatcctccccagcagtcggttacccttttctgttgcctctgtagcactttctaaacataaaattagcttatttctttgcaggtgtctattgcaccccccaggaatgtcagttccctgcagactctcactctgccttggcacccgcaccccagcacccaggggtgaggtgcctgcctgggcagccctgtcctcacctggggttgactgaccagagaacccttgcgattctctctctgggtttctgtctgcattcctgtcgggataggtgagactggggtgacggctgctcgcggcttggtcaccttcttctctgcttacctgttcatgctctcttgcttttctctgatagcaaaccccgtctcctgtcatcctccctcctggcggaccagagctatactggcccataaaggagtgctggccacaccaggctagattccagttgagcttaaataaaacaaatagaaaggtcctccgtggtattagccatattgtaggtgctccgtagacacatgcggctagtggctgcccacaggacgctgcagatgtggacgtccccgtcatcacaggacgacattctgtgggcagcgctgtatttgtttacaaacacaattctttggctgtgtgaaggttcaggatgatacctttgccattatgattcatttattcatttcaggctggacacggtggcccacacctgtaatctccacactgggaggccgaggcgggaagatcgcttgaggccaggagtttgggaccagcctgggcaacatggcgaaacctcacctttatttaaaaaaaaaaaaatcggccgggtgcggtggctcacacctgtgatcccagcactttgggaggccgaggcaggcggatcacgaggtcaggagattgagaccatcctggcctaacacagtgaaaccccatctctactaaaaaataccaaaaattagctgggcgtggtggcaggtgcttgtagtcccagctactcaggaggctgaggcaggagaatggtgtgaacacgggaggcggagtttgcagtgagcggacatagtgccactgcactccagcctgggccacagagtgagactccgtctcaaaaaaaaaaaaaaaaaaagaatcaacatttatttatttattatttatttttttgagacagagtcctgctttgtcaccgaggctggcacgatcttggctcactgcaaaccccacctcccagttcaagtgattcttgtgcctcagcccagtgccaccatgcccggctaatttttttgttttgttttgttttgttttgctttgctttcctttgttttgttttgtttgagacagggtctcactctgtcgcccaggctggagtgcgatggcacgatctcggctcactgcaacctccacctcctgggttctagtgattctcctgcctcagcctcccaagtggctgggactacaggctcccgccactgcacccggctcatttttgtatttttagtagagatggggtttcaccacattggccaggctggtctctaactcctggcctcaggtgatccacccgcctcggcctcccagcgtgctcatttatttattttagcatcccttcttgtgcctggttgtaaagttcttcttatcccaggaaacttaataggtcaaagaaccctccccttgaggcagtttggatggggctggctgggaattggtatgtccagctgcccatatcgtggcctctgagtcaattatacaggcatctcggcaacttgaagttttgagtcccaaaacaacagattccatagcataaacagtgcaacgtaaatcctgtcccttgtccctcttccattggatacccttgtttatgttctctctggcaactgcctgtagcagggacggatgctggaaatcctggcggggccacgtcttctgtgctttgtaggcagctatggctggatggtgcttgctgtttctgtgtgtaaatgttgggatttttaaaagcccttccccgcttaatgcgttgtggttgagcagcacatttaaatgcctgaagtgtaagatgaagtctcttggatgtagagggcagtggtggtttttatttttcctggttgaacttggcctttggattgctgactgcgagctcacggtactgttaatgattttctacctacagtcacctgctgtgctgagcacatcaaatacagtgcggttagttccaagctatgaagcgcctgtcacgcatgtataacaggcagacctcattaggagggggccttgcataggggtcacattttccagcatttgttttcaaaatggaaatccagatcacgcatttgagattaaaataaaaaaagcggcctgtgctttatcattttctttaacttacatgcctgctggtccagcccagattgctgatagaggaagccctgagaaagttgcccagccagttgcccgtgcttcaagacacaaagggctgactgaggcatccgtgcctacccttggctgctgctgctcagcctgcttgcatctcgtctgttttcagttatcagtggtgtgagaaaaacacaacggtggattcagaaggagaaaccaagtcatctgcatttggcctcagtgtgtggactgtcctgaaatttgagtacagatgtgttcatgtcctgggattagataaaaagagaggaaacacacagtgaattcttatggtctgtcatttcagttggccatcttctgcccctctgttctggaagcttctccactaaaatagagcacccaacttggttttctacttgctttctggcccatcttgagttggaggaatcatgtgtttcagaactcttgctggtggtgggtgggagatcaacaaatgatttcattaagctgtgtgttcttaataaactttgggaagatttgtcgcagagaaactcagatagggaccttgtgatctcagcagtcatgggtttatcttgtccacacgcgtggcttcattgggatttggttggttgcgatccagcagaactgtggcggtgttgggctctgttggggtttggttgtgagcggaactgtggtgttgggctctgttggggtttggttgactgtgatctagtggaactgtggtggtgttgggctctgttggggtttggttgactgtgatctagtggaactgtggctgtgttgggctctggagtttggttgtgatctagtggaactgtggcggtgttgggctctgttggggtttggttgactgtgatctagtggaactgtggcggtgttgggctctgttggggtttggttgactgtgatctagttgaactgtggctatgttgggctctgttggaatatggctgtgatctagtggaactgtggcggtgttgggctctgttggggtttggttgactgtgatctagcggaactgtggctgtgttgggctctgttggaatttggctgtgatctagcggaactgtggcggtgttgggctctgttggggtttggttgactgtgatctagtggaactgtggctgtgttgggctctggagtttggttgtgatctagtggaactgtggcagtgttgggctctgttggggtttggttgtgagtggaactgtggtgtcgggctctgttggggtttggttgactgtgatctagcggaactgtggctgtgttgggctctgttggggtttggttgactgtgatctagcggaactgtggctgtgttgggctctgttggggtttggttgtgatctagcggaactgtggcggtgttgggctctgttggggtttggttgtgatctagcggaactgtggcggtgttgggctctgttggggtttggttgtgatctagcggaactgtggcggtgttgggctctgttggggtttggttgactgtgatctagcagaactgtggcggtgttgggctctgttggggtttggttgactgtgatctagcagaacagtggcggtgttgggctctgttggggtttggctgtgatctagtggaactgtggcagtgttgggctctggatttggttggttgtgatctagcggaactgtggcagtgttgggctctggatttggttggttgtgatctagcagaactgtggtagtgttggcctttgttgggatttggttggttgtgatctagtggaactgtggtggtgttgggctctgttggggtttggttgtgatctagcggaactgtggtggtgttgggctctgttggggtttggttgtgatctagcagaactgtggtggtgttgggctctgttggggtttggttgactgtgatctagcagaacagtggcggtgttgggctctgttggggtttggctgtgatctagtggaactgtggcagtgttgggctctggatttggttggttgtgatctagcggaactgtggcagtgttgggctctggatttggttggttgtgatctagcggaactgtggtagtgttggcctttgttgggatttggttggttgtgatctagcggaactgtggtggtgttgggctctgttggggtttggttgtgatctagtggaactgtggcggtgttgggctctgtgggtttggttgtgatctagcagaacagtggcggttttgggctctgttgggatttgcctctgatctagcagaactgtggtggtgttgggctctgttggggtttggttgtgatctagcggaactgtggtggtgttgggctctcttggggtttggttgtgatctagcagaactgtggtggtgttgggctctgttggggtttggctgtgatctagtggaactgtggtggtgttgggctctgttggggtttggttgtgatctagcagaactgtggcggttttgggctctgttggatttggttggttgtgatctagtggaactgtggcagtgttggcctttgttggaatttggttggttgtgatctagctgaactgtgatggtgttgagctctgttggggtttggttgtgatctagtggaactgtggcagtgttgggctctgttggggtttggttgtgatctagttgaactgtggtggtggatcccaggagctgctgctgcttgtgcgcttcaaagttggattttgtgtttttttgtgtgcagatgcttcttggaatcttgcgttgctgtatgcctgtatacctggaaagttgtgggaagaacagtttaggattttgaaccaattttctcaacaagtttgattatttcatttgagacatgttctgatgtaaatatattctgaaaaggtgagatcaaacatccactttttactctaaagcaggtccccttgctcctctctgctagtgcattctctttcctttacagctccttctcctttctgaagcaggaattaagctgaagtaactcagaaagtgccagcgttaaccccaggggacagaaaccacagacccagggctgagggattgagtttttaatttccattattctccatcagtgcgttcagtcttttgaacttaccctgactggtattatcacttgaaagttgttgttattgttgttttgatggtagtggtggtggttttttgccacccacaggagagaggggattagaagaaaaagaccaatggagtttccttgggattaattaaatgtggggctgccacacaggaatgggtccaagggctgctgtatttaaataacttacttattttgcagaggtgaatttaacctaacatgtactacagtgatggagtaaaccttttcctgtttggaatatcacaaaaggtacttcaataattgcggatcttcctttgaaccctcctattgtcagatggggtagtggtacagttccttgttccagaatgctcttagcaagttgattaatctgtcacataccccagtttcctcattcacaaggtggcataataataggacatagagttgccgtgaagtttaatgagttaatgaatgtatgatgcttggcaaatggtaggggctcaagccaaccctcgttgtcttactgagcttagtgggccttggacctgctgaggactttgaactgagcattccgtgttattctcttttttgtttttttaaggggatgagagtgtgtgtgatagagttcatgacaatattttgtttaagaaaattgtcaaaacaaagttcaaatgcaatggatggaaagtgatttttaacatgtagggtaactagagatttgagatcagacaaatcgattaggagatcaccaaacccaaacagagaatgtgagttaactcatacttaggagatgaatgctggctttgaagaccatagcgtgcattttcaaaccagatggagaggccacttttctctgcagatttcagttattcgtgatgttaaatgactcgtcttctcttacttgcctggcatgtggaactgagtaaattctgtcaaaagaactcactgaaggaatgtgtaacagctgggaattggatgtcttttcagatttgggagagcctgtgttttgactgtgatggaattcttatggtttaatatgcttaaatattactgtttaacattttgacgtttttgaggacagggataattaggagaatgcacttataagtttctatgccaactgtttccactgctcccctgtttacatggtcccagggtttatacagggctctctgtagccccacaatgaatcttgccccagtgagcgggctctcacctcccttctatagatgaggttggagctcagaagaacgtagagaaacttgtgcatagttattatgccagatttagcagctggaacaggactggaacctaaatcttttttctttagataatatgttctttgcattaatgagtttgtgggttttttgggaggcgagaaatatgtgtaaataagaagtaaaaatcctaaattgcaagttgattttttttcttaatatagtaatacgtataatgcagcaaacatatcatttcccctccatgcttgctagactggcttatgcacacatcatgatgataatgaaggagctaacatagcaagcactcactctgtgctcattcataaggtggcataataataggacacagagttgccatgaagtttagtgagttaatgaatgtatggggcttggcaaatggtagatgctcaagccagtcctagttgcctttctgagcttaggaatcatggaatcatgctctaaaaacttcatgagtatgagctcgcttcatcctcataacaactctaggagatagacgttactttatgtccattgtagggatgagagtgtgaggtatgaaaggaagtgagcttaatattctttatttttattttttatttttattttttgagatggagtcttgttcactggcccaggctggagtgcagaggcatgatcctggctcaccgcaacctccatctcctgggtttaagtgattctcctgcctcagcctcttgagtagctgggactacaggcgcgtgccacaatgcccagctattttttgtatttttagtagagacggggtttcaccatgttggccaggctggtctcaaactcctggcctcaggtgatccatccgcctcgggctcccaaagtgctggcattacaggcatgagctaccgcacctggcctaatattcttaatttggctgtcggttgtcacccaaatcagttaaagcaattccatttcttttttttttttttttttttttttttttttttgagacggagtctcgctctgtcgcccaggctggagtgcagtggcgcaatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccgcaattccatttcttaagtgaaaatcagaatttcttttcttttttccccttctattttttcttttaatcttttttcttttgtcttcaagcaattttcatttttttttttttttttgatattcagggaacctgaggcgaatgaaggggaggaacctgggagctactgactgtgattacagtggatattcacaaaggaagacctttagtacagtgtgaagtagtgagggaaagggtgatggagtaggagggatttaagtaaaacctttcaggtttaaggtcaatggaaagcgaaggaacattctagaaggacattagattttccttagagtgacattagagtttctataattggactataccctttagtgcctcaaatcaagggtttcacattttctttttttttttggaattgcctgctcatgtatttccatctccttaaaggcaagggctggccgccttatttggctgatactagcctccatggcatcagcctgtccagtggaagtcctgaaacagtacacgatgaatggataaatgaatgaatgaatgaatgaaatgaatgagggaagaaatgaatattgtgcttccagaacgaggagagaagggacgatgatgtggcaaattggcccagcatcttaagatctgtattgactttagcctcatgtggtagtttaatgatactctctcttactcagggaatgtgccttttaagctgtggtctgtggtcaatgtgtgtgcacatctctgagagtcccggctgtgttgtgtttgtgatggctcccatatgtggtttgcctttgtatctgattgtaaattaaccttgacatgtaggtcacctgaccgtgtatggagagagaaaaatcactttcttcttctctacaagctatgtgagtgcagactgaagttccgagttgtgatctcaagcagtctgcagaaactaacctttggcatacttggccacagccgacttctttctagtaaattcccactggttcttggcttttcttgtagaattaacacatttgcaaagttttcttcattggtattcctggtacgtggactggaatattcttcatcccttgatttgtgggatcgttttgtctttgggccacacttgtcatgggaggaaagtcacagctagactgggtatggaaagaatggaagtgtgtctcctgcagggagagcccaggttattgagacagtcttggcccgtgtttcttgaagaagacgttgggttttgttttgctcattgatggccataccccttgtgtggttggccaggaagctgaacgcttgctcccccttgtggctaaatacaggtggttctgctggcaaacccagtggtcaattactgcatctactaaggattaagaagattataaatgtaaactcagcttgaaactttttgtggcttagaggtaaagcaagaaaataattaagataacaactcacatgctgagggtgtttcatgtgttattattgtggatgttaataacaattttggctctctgtgggaagccatttccttcacttaatcagttggcaaagaaagtacatcagggaagattgttcaccctgagccgagaaagtggagtttcaagtaagaaccctgctccgcatcctgccactgagttgagatcatctatttgatttagctactgataaagtgtttttaattacaaattgtctgcttgcttggggactgacacaaacacttgttttgttttcaaagtgccttctctccccagtgttgggtttggagtagagcaagagtaacacagaggttgcagtcagtgtcttccagcgattttcattttgtttgatatgcggggaacctgatgctgatgaaagggaggaacccaggagctactgactgtgactacagtggatattcacaaaagaaggacctcagtacagttccaagtagtgagaggttgatggattaggacagatttaagtaaaacttaacaggtttaaggtcaaagaaaagtgaagcaacattccagaaggacccagaaaagccgagcaagtcaggcactgagatcttgggattgagaggagttgctggttcagacccagcactgttctggaactttcttttgggtactctttttttgtttcatcgattcctttaaaaggtacaatccagaactgaacatgtcattccacaagtggcccaaccagtgttgtatacactgactggacaggttttgccactaaaatggaataatagtctggttttcctaattccaggaaaggaaaatcaaggaatagatacattttttaaaaaagcatttaaagaatttctcctgtagacattatcttaaacttttggtccctgccattgtatgtgtatcttgaatccaagggtaatacgatacgcagtctggcaaaagcagtcattttaaatgattgagcaatatttagtgactttcaaagagcagagagcacaacttggctaccatagaagctccaccatgatgggcgcagggtttcctctaggctggaagttgtgttctggttatcttgtgagagaaatgcatcctgtgtgatttatgtggattcatttattcttagaggaaatcagacaaaaaccatattgtaaggcccaacccatctaaaaagacaatagaaatgagttctgtgtgtttttcagcagggcctcatgggcaccactcgtaagtccagaatctcttccaagaacaggaagtttctggagggcagtggagaaggggagtgagggctcagctctgtgaggtcgaggtgggtgtgaggaccagcaccagtggggagtgggtttattagctgggaaaggccataaggatgaggaagagaagcttgtgtttgagaggataacagggaggaaaagtcgccgagcccagagtgtgtattcactgcctgtcctttggaggcagccgcttccacctcttttgactgattcttccaactcgtatcctccagtctccaaaacatgattgtggtgcagctgcttgaattttcaaacttgatttccattttagaagttaaggatttgtttttttttccaaccacataccctgtcgctaccacatcaaatacaggtctctcatactgttggtcagctccgttttccgtgttgacctctgatactctgtaaacactcttgacacgtaagctgggtggtttatataccaccattacttttcctgcccagctttgtctttttcttccttgtattttatttgtaaagattccccagctcgtccccagttgtgtcagcctcttttcagtgtgctcagacccattaggtataaggcctatccatgttcctgaggaagtctggcccagagccgtccaggtggtctgacctgggcagggccccgggtctgccatcacccagcccatgctctcctgccgccccactgaagacttccttcacctctcttggacgttagggtttctctttgatggctcctctctttttctgggtttctgatattttctggttgagtggtgctactttcttctaacctatttttatttcattaaataaataggattcactaaatggatttcatacccatgggttgtatgatttgcactttgaaaggcactgggctagatgaccactttgagtgatgctgaggcattgctgggtgacttccttctttccttcttccctcccctcctcctcttccctcccctcctcctcttccctcccctcctcctctttcctcccctcctcctctttcctcatgaagcatgtgcacaggtgcatccctgatgctttgatcaggtatggcctgaaagacaagccattttagttagtttttggatacattacgaagtaatgtgggaagtaagcatcttcacctgtccctcttccctccttggctgagggacagtgtcctagtcagttccatgtgctgtgacagagtaccataggctgggtggcttaaacaaaaaacacttaatgctcacagttttggaggctgggaagtccatgatcaaagtactggcatatctggtctggtggggacctgcttcctggtctgcagatggccgtcaccttgttatatccttacatgatgagagagagagaaagcaagctctgttgtgccttttataagagtactaatttcattcacaagggcgccatcctcatgacctgatcacctcgtaaaggctccaccttccaataccgccatcttggggattaggatttcaacacatgaattttagggggacgcaaacattcagtccctgacagggttaggcactgtgtggctggcaatcaccaggatgcctggagccctgccgggaccacgctggctcatttcagccaccctggatgaatcatccagcctaaggcccatgcctggacattctgggtcctcagtgccttgcctttattttacctgatataatccttcaaacgaaaatgcttgtacttatttttgatcacttaatatacttggccatttaacaatacagattaaacttgtgttattctgttagaaaactgagagattgttttctgtagtctttgactgcatgcatagtctagggctggacttgggaggagaggataatttggccacaataggattccttattatctgtctcttccctcccaacccctcccccgacccccgccccatacaggagaccctcagatacatttctgtcagtaccctcagttacatattttgggagacgtaatacttaatgaaccaaaactctgttttgctggcagggaaaaaaattgggctgatacaatcaagataatcctatcaactttaggattattaggaatgttgataatcctatcaacattctatcaactctgtaatcagaaaaatatatttaaaataatgcgtgtttgttaaaaataaccaaacaaaatacagaaaaaagcaaagtaaaaattgacaatcccctaactccagtgttggtagttagggacagaagaagggtggaggatgtcttttgtctgtgttacatgattacagcaaaagtcactcttcattctggtagagccatagaaatctggccgagcagaggcagctgtgcctgaggagcctgacttcgaatcagctgtgttcatctttgctaacctcccaaagggaggaagctcaggaaaccaccgtagaaccccctaaaaggggagggtggctacagctgagcaggtgggggcaggcaggagtggagagaggcgaggatgatgagtcaggcaggtgagatggaggctttgtaggttatgttaaggctcctagacttagcatttcaagcagggatgtgacctgatcagatttatattttgcttgcattgctgatggacattggttcagtgctcattgtactggaagggtcaagaatagaggcagggacccaaatttagggggcttcccagggaatccaggggagaggtgttgtggcctggactagatttggaacatccagaaggggactggagcaatgacatacctatacctagtcccagagggccccgatctagctaaggacagagcgtgcagtcttgcagctgctgggctgatgacatgctggggagtgtatacctgggaaattctggtacagagataaaggaaatgctcaagcctccacctatgaatttgcccggtgattaaaatcatacttcctaagctttatagaagccctctgtttgtcccacaaagggccactagcacaaggaaatacataatgtgttcccttgtcattttataaaaatgtagactggtgctgcctgctgtgaaaatgccagtcaggttgcttccttcatagtaatcagtggattcttttgtccatttctgagtggaggattagccctctgtttgccccacaaagggccaccagcacgaggaaatacatagtgtgtccccttgtcattttataaaaatgtagactggtgctgcctgctgtgaaaatgccaatcaggttgcttcctttatagtaatgagtggattcttttgtccatttctgagtggaggattcctttccataacgtggtgtatttgcagtgtttctctcagaggaggagtgcctttctggtgtcatctgtcctgttggtggcagagttctgctgctgaattaaacctttgtgtcggccttggccactcttatggctctgctggaggtaaagccagtggagtggccttgtggcatttgctccgagttgttgctcatttttctgagattatcagaggccttagagggaagcattgaggatttcattatgctccgataactatcacgttgtcaataatatggttctgataagagtgatgggggatggtggggttttgtgagttgacagacgtgggtttgagacccagctctccagtcagaagccagcgccaggcctgtttctcttctctgagttttagtgtcctcaggtggaggggaggataatattaataacaccttcctgatgtggctccactgggctggccacgccatgggcacccagcacggggtggctgccctggttcgtgttaaccatcagcagggttggagagctccctccttactggtgttggaggtcattacctggcgggggatgtggtagataaagcaccttttattgtccctacacaaggcaactatggggtaacacaatataagttattcagattagagaaaatatgaactcttgctacagtgttactgcagacaggcacatatggtttgttaggatgacatttaaaaaaagtcattttgagttacagaataaaatcaaacaaaattcctttaatcaccagggctgggatggaaataaggcaaaatgttgattcctttgaaagacaatttttgggttttttttgttttttttaaaaaaactttatgtgttgatggtttactctggtctagtcgttcctactcctattctgaaagcaggccttgaattcctgattgtgtacaggattgtgtacaaacaatcaggaattcatcattcatatgaagaagttctgtttttccctctagtgtcatgaatattttcatgtacgtatatgttatgtgactagtgttaagaatgcatgtattatatatgtgcatgcgtctcatttacattgaatgtagctaaaaggaatatttgaaaagtataccttgcctgtctccaatttctacatttcaagttaaaaatgggaaataatcttagaatccatgcctgtggttcataagcctggctcagctcttgcattcccagctactgtctgacgaaactcacaggagctgtgaaaacaacgctgttttgtgaaagccaagtaggagcttagggtcatggcaaacttcagagaaatgtaaaggagcaggcactgtgggttttgtctaccccacccccccgaccccactgccttccaaaatttctttcttttttttttttttttttttttttgagacagagtctcgctctgtcacccaggctggagtgcagtggcgtgatctggggtcactgcaagctccacctcctgggttcacgccattctcctgcctcagcctcccgagtagctgggactacaggcacccgccaccacgcccggctaattttttttttattttttgtagagacgggatttcactgtgttagcccgtacggtctcgatcttttgacatcgtgatccacctgcctcgacctcccaaagtgctgggattacaggcgtgaaccacaccgcgcccagccccaaaatttctaattgaattttaaccttccacttttaagttctgatcctgatttataaaaacctatgtaattttcttcagctttcagaaggaatgtcttgtatcttcctatgggggtgttgggggaactgatattgacagaggtcctctgacatttcatagcctggcaaatagggtcaaacatgttccagacctcagaggattgctgaggatagctttctacttaagctagataaaagaagttttgggaatcttaccttgcactctgttttgtagaaacaactgtgtttaatatatttctcattactggctgaatgacctgtatgtgttcaggagatctctctatttaccattttaatgccacatatttaatgagcattcaggcccagactgtgaagactctggaaatgagaattttgtttcctatctcctgtgctgtaacaaaagcagcttaaaaattgcagtgtatagacctcaccttttatcatttttttttcagatttttaaatattaatattaagggtctttatctcagatgtttgttgtttaccaggtcatcagtatctggacgctggcagggtccttagcaatggagggagggagagggggtccagcctgcatgattggatcagtgttgaacctgtcccacctgtggggaagaggcagcatgcggagcagacaggtgcctgctgcaggtcaggagagctggccccaagtcctgacttggagggaaatggtacatattaatatcctttagtgccaggtactggcgtagctagtttatatgcatcacttgagcccccttgcatcactggcatcacttcaagatagacaacaaggggaatgcacatcatatccctgttcactggcgttcactggtagggaaactgaggcttaataatgtgcccaggtacacacagctgataagtaaccctgcaccctcgatctcacagcatgacattggaatccacctcattagacagagctcagctgcctattctgtcaaaaatgttgagttgtatcagctgcagtgtctgtagtattcctagacatttctgtgattctcataagtatctttgaaagggggccatttagtttatttctgcatttctcccttatctgccgaaatgcccgcaggctctgggacaagctataaagctctccgtgttatgctgtgttgcaaacccagtgccagtaatgatgcagtgctatttaaagataagaaagcaagctcacagagggagagcgcagcaggatgtttatgttccagaagcataaacacatgacctgtgcccgtcccgtgttcttcgtccatgtccccagagaggccgtggatgggatggtgcaagaaacccatcaggaagcagaattaatccttcctgcgggtggtccctgtgaagaagacacagcggccggtggtgtgacagcccacagctgtcgaaggaagggagaagagagggaccgagggccagcaatagggttttacagctacgtgctaggttgtctcacatctaggaaagagaatgttcatattatatagaaatgggcattttccctgtggcctgatatgaggtatccagaatattcctttaggtattattaagtcataggtttctgttttgatagaaacagcaggtggttcagttagtatttttgttgttgtaatagtagcttagatctcttgtttttgttttctttggccaagacagaaaacattctgccacacacattacttcatgactctggttatatattgagagcagcattaaaaaaaaaaaaaagtccaatttagtccattaggccattcttgcattggtataaagaaacacctgagactaggtaatttataaagaaaagaagtttaattggttcttggctctgcacgctgtactggatgatacaggatgcatgatgctgacatctgctcggctactggggaggcctcaggaagcttctaatcatggtggaagatgaagggggagcaggcatctcacgtggccagagcaggagcaaggggttgggtcacacacttttaaacaaccagatctcatgagaactcaccattgccagaacagcaccaagccatgagggatccacccccatgacccaaacacttcccaccaagccccacctccaacaccggggattattgtcaacatgagatttcggggacaaacatccacactatgtcgtagccttaaggggatcttacctctttgaagcattgtagagttcagagagtaggacattttgtctactgtattttaaaatactaaatgttctgattatgaaagtcagtcctggaaaaaaaaaacaaacaaacctgaaaccatagatgaagataaggaacaaaataaaaattacccacagtctccctcgcgtgtaccgcacagttcatgttttagtgtgtctacacaccatttaatgcaggtggacagactcaggggcaggtgtgccttgctactgctatgtccacagcacctggcagagtccctggtacagcataactgctcagcagttgggtgctgagtaaatattaatacatgtgcatgtggtaccagtcagggcccaacagagggacctcatgcgaggagttgctccacaggtgtggaggatgaaagaacaagttggggcgaccctgagagagcccagagacaagctctgcaggaagcaggcaccaccactgggccaggtgacctgagcccaggagccaagaggatgccctccactgctgatactacctctgcagtgggacgacatcggaccccactgccgatgctgggacggtgcttcccctccccctgaggctctcctaccagggccccctctcatcaggccccagctggaagccaactggaaagggagtcggggaagtttcatttgtgggggtccaagtgccaagggacagacaagggaagggtggatttgcagctaagaagatagtaaataaccagactacgtgacagtctgaagaatcaaatctggaatcaaattctactctgctctaatgtaaccctgtattttactgtgaataatataccgtgggtatcttccttgcttgatagatacagctccacataatttttctcaatgaaacagattctggagtaggcaacgttttcctcttttgtaaaatacaccaaattgtgaggattgaatgagataatgtcaaagtattagtactttaccctagctgattgacagtatccagtgatagtgataataagggtaattactactctagccaattttatcaaacctatggatgtttaaggaattgggttttttttttttctcctttgatgaatactcctgtaaatacatcgttgcctgcttgtctgagtctttctgtgggtttttagaagtggattacttgcagcaaagtctcctctacttaaggctctttgtatattttgcctaattgtgctctagaaatacatcaatttccagtcctccttctctcagaccacctgtccctcacattttcaccagttctgggtcttgttattctttttactctttgtcagtcaggtgattgaaaaatgtacatttttttaaaattggcatttattagtgagcttgaggatgtttttcacagtcatatggaaacagttctgcttcatgaacaagaccttggggccacatactggcttgttcacctggatgcattttctcagtctccctcagaatcatttttctccctgaaaaatgggattaaaagtattaatagtagtgcccatctcacagagtcattgcgaggattaaagagaggatgcatagacgtattgaatcagtgctgggttctggtagatgttcagctagccctagagattattagccatttgatgtgctgatcctcaggaaggtgggcgggagagacctagacgcagaaaaggagctctggagttgaggtttctagagatgaggtggtgcgtggtggcccagtccagcatggctctgggtgtgagcacaacataggcagatgtgtctggggctcagggagtcatttgtggattgagggtgctatgtgggtgtggaccacggtgatccaggatgatgcaggatttgggccggggaggaagatggcaacccaggttccatgccatctttaaagaggggatggttactagggtgtccagcgaccagccaagtggcctatagataataaggaggactcttcatgacatggaggctgaggtggaaagtcccagagtagggttcagccctggccctaaaaagtgccggagaacctgtagcctcctggaagctgtgctgtgtgcagagccaagcttcagtgtgacaaggaatggtgggagcaatttggagaagaatgaagacttggtgcagtggctagggaggcaggcagggtgaggatgtggaagggtgcagagctgctcaaggtcagcttcctgagcctgcggtgtggtcccttgctccacactcagaagggccctgtgtttgatttaatgctcttttcttgccatcttgaaattcttgatagtttttgaacaagggatccatgttttcattttgcgctgggcctggaaattacgttgctggtgctgaggggatgtgcagagcagaggtgggcagagaactgacgagaaggacacgtgggtaagggtgggatgagctggcctcaggactccgagcagagctcatgtaaaggtgtcgggcccaggatgcgttctcaggctggggtgggaggcctgatgatggcagatttctctggattacaaatagaatatgtagcagggtggttcatagctttgtgtgtctgtgtgtgcattggtaaatttttattttgaaactaaaattatttcaaaattagagaaaggctgtgagagtgcggttgagctggtgttggctctggaacagggtttcccaatgcaggcggcactggtaactatgtgccttgggtcattctttgttgtgggggctgtcctgtgccccatgggatgttccctgcacccctggcctctgccttctagatgctggggggcactttccctggtgtaatattaaaaaaaactctccacgttgccacatttggggggcagaatcaccccctccccgaattgagaagctatgttattggaacacagtgtcctgtattttgtctagcctctgctgcttagagctgcctgagcttggagaagatacttgttctctttccaaagcttcactgtccctttctgtacattgtcaggacggctgttaataccactgctctaaatgtgctcagttttgcataaacatcagctaatgttaaaattttaatctctgctaatataagaggcatcgaagtacacgcgagttcagtttgcatatggtgaggagggagacggcctcatatcgtatgtgtgtatttgccgattctatattttgaggttgattcctgttgtcttttgttcagttgtctcattctgttttcttttggggctttgaatgcccttttcatgcctgtgacctctctttactatgccttttcttctctgccagcaaatctcatccattcttccagggcaccttactgtcccccaaagccccagtgactgctctgtccacagggcttggtttctgtgctctggctctgcaatgcaatgcagaaggggctgggctcggaatggggttgctggtcaggccatgtgcccaggggaaggacacaggactcaaggagtccacccctctctcccccccttttttttttggtcactgaaatattttctttccaattctactagtcctgctagagctgccataacaaaatacccaattggtgacttcaacaacagaaattgattccacactgttcttttttttttttttctttttgagatggagtcttgctctgtctcccaggcaggaatgcagtggtgtgatcttggctcactgcaacctctgtttcccaggtttgagcagttctcctgcctcagcctcccgagtagctgggactacaggcacatgcactgtgtctggctactttttatattttttagtagagatggggtttcaccatgttggtcaggccggtctcgaactcctgacctcaagttatctgcctgcctcagcctcccaaagtgctgggattacagacgtgagccacttcgcctggccatgatcccgcacagttctgaaggccagaagccaagatcaggagaccagtggattgtttctcccgaggcctctgttcttggcttgcaggtggccccttgctgtgccctcacatggcctttgccctgcatacatccgaagccccagtgtctctctgtgtgtccaaatttcctcttcttagaaggatgccggtcatattggattagggcccacccgtgtgacctcattttaccttaataacctctttcaagtttctgtctccaaatacagtcacattccaggggctagagcctcaacatggaagttttggagggagacacacttctacccataacaccaatgtcatattaagctttatggaagaatagaccaatatgtggtgggttcaagaatcttttcttagaagaaaagtgaatctgtatatttgatactttatgatttccttatttgctctaagcagttggtaagaatctcagtgtcacttggctgaatgatacactactggtgcctccagacctatgaggagcctaatatttttgtcttggttgaaacaatgcaaatcctgggttttggagatgctcacagccagctgtctgattacgtaaactgaaagtggcagagaggaagcttttgcgtggggaaaacatgaaatatacaagggttcctttaaaacagagtgtacttctgaacatttactgaaggtggagggccctggacacctgatgttatcttatgtggcgttatctgggtcagatttcattgcatttcctgaagaggtaacttggcagtcctgggagaggctggaccttggtgtccaggtggcaggttcataagacacctgtgatttgatgtgagccagtcacagaggaagccatttccagagaggaacaaccgtgtagactgctttcctgggagtcaagttaaaacttctctcctgtgaccttgtcacccggcagtggaaagttacccttggctgcagccacgacttccgcatactcttcagaacttgctctgcccttgagttttccagctttcctttaatgcactaaacctttaatattgtattctcctaaatgagacgtctcgcatttgagcaggtaacactcggtaagactttgcttggtgggggaggaggttttaccttcctgtggcattctgaagctctccacatccacccatagtcatgattgtgttatatgacctcaagatgctggagctgttaattcatctttaacataattgacctgattacttctttcttgaaggattttgttcaaacctcagggtttatttaggaacaagtctaaccaggcctgattggttttcctaagtggagatacagctttgggaaacaaattaagaaaatgaatggattgaaaatgtaggaacaccgtgacagttgtgaatttttcttattgagggaaaaaatgggtatctgtgaatatttctcttacactatatagtgagaaatgacactctgctgtggatggtaatttatttctgtgtaattgcctgagtaaggctttctctgatattgaatactgagtagaactcttaggttatttttggaagaattatggattttctcttaggttttagaatattttttgtttgcctgttaagagaatttgatgaagatttcaatgtaattaaccaaataaaatggcattctgtatcctgggaagggacgatgtctttcagttctaaatcaatgcatcatttacctcctatctctcttgctggccgagccatcaatcctgtttttattaaatgttgttgttattgttttgagacggagtttcactcttgttgcctaggctggagtgcaatggcataatctcggcccactgcaacctccgcctcctgggttcaagcaattctcctgcctcagcctcctgagtagctgggactacaggcatgcgccaccacacctgactaattttagtagttacagggtttctccatgttggtcaggctggtctcgagctcccgacctcaggtgatccgcctgccttggcctcccaaagtgctgggattacaggcgtgagccactgcacccggccgttttttgataaattttaaacttctggtttgaaacttttgtttcctgtgcttaaaaggagacagttaaaagcactctgatttggactgacagaatgttttttagatgttagatagatctagctttgaatatttatttgcagagaagtagcagggaggactaggaatgactttttcttaggaggtgctatgcattccatcaggtgactgacccctttgtgagctgtagtggtcagtcccagcatggcccacacttgttggctgtcttagccatggtggctgtggttggcgggtacccagccaagggttttccatactgggggccttggtccagtgggggcgggtcgtaccatgacccccttctcctgtgatcttgcctcactggcccaggtaaattggtggacgtccttgactcctccacctccaaaaggactttgaactttggcactgttgggagtttaggctacacctcaaccaggcagagattgggatctaacctacttttaaaagccctctacagggaatttcataactttcctgagtgttaggttttggtgattaacagccttgactttttggagcccttcctttgccaaacatgcattcctcaggtgctcacttaaaatgattcttcacaatatgcctatagacgggtctgtctggtacagcatcctggagtggtgtcaagggaaagaaacatgctgcttctgccacagacggaccatgcatgggaagaaactatttgagaatggctctccaaggggctggtcggccttgtctgcccactccctctacctttctttgttctgtttctttctcttcagtgttggtccattttccttttttcactgttgcgtgttagcacatcattactcttatctccatatatttggatgtttcctgcattatggtgagcttcagtaggtcttggatgagttagttgttctcattccaaaatgtaaatttctggtgtttagtaggtgctcagtaaagcttgagaaagatgaaagtgagagcccagaactagtataaaaatacggataccaccatagcatggtaattaaaggggtgattgtgaagatgaggtagctgtgcctggatatgtgtgtgtgtgtgtgtgtgtgtgtgtgtgtgttggctggagaaggcttaccagtggtgctggacatgtcgggtagagtgttaatagtaacttaattgactgaatttggaaataatctatggggttagagagcaaggaggtagctgtgtaagaggatggggtcggtgaggtggggtggtgtgagcaatggagctcaagggctcaaggtgcaggcctggactgatggccactaggaccaccccaatggcttcagggtcaggacaggaagggaatgtgattattgcagcaggtagtagggcagtatgagtgtattattcatatatgtgtgatgaaatggagcatcaaataatatatcccataggatagaaaggcacagtgagtgagtttgggttaaaaattcgatgagataaagtggttgtacctcctatgaaaacaatttaatttcttattatgaaggcattactcatgttcttcagaaaaattaaaaaaaaaaactcagtcattatagacaattgtactcaagatgaaaacaccctgtactgtcgcaaccctgaagcacctctgttaatatgtgtgtgtcttctcacggggtgattcttgatcagagttactgaaacagcaaggcttgggaacaagaggcatgagagcaacactcaggacctgttcagttttctgcacagttccaggtgctcttccagcattgctggctccgagagagtcaaatattcagagcttgcaagctggggaggaaaggggttagggtggagataaagatctgggagatctacagatacggatcatctgcatttggaaattttgtttgcccttctcacacatttccatccaagttttctttggggaagaggggatccctgcagaggagtccagttgcaaatgtgaaatcttaaaaatgtatgtaaatagcatctttcattccgtgatacttgtttgtttgaatagaacattgttaaattttattgtgcttcagaatcatctgggtgcttacaaacatagattgctggactctggttctggagattcggattctggaagtctgaagtggggcttgggaatctccatttcttccaagttgccaggtgatgctgatgctggtagtcgggagtccgtactttgagaaccactcttctacctaccaagacttccaaaaaattggtactctgggaagatgccctgtgccagtcctgtggcttcttaggcacgtcttccagcatagactcctgcagagaacctggtacctgcaagtacctagtacctgcaagtacctgcaagtccagtgtgggaaatctcatgacaggcatcaacattccgtgacggttgacagccacctatggggtggctgatgccttttctgtgtcttcagggttttaggggaaggagagtctaaatgagtggttctaggttgtgggaagagcctggctttgatgttcagacacctggcctcaggctgcatcgcccatacttcctgtggaatggttatcatgagtcctgttgtcctgtaggtgaatcattgattcctgtctttgcataacctctcacttgatgataatttttttttaaaaaaatgactcttggaaacagaacacacagttgtagagggacctacagtacagcagaagctttgtggaaggatacaaaatgtgagggactgtagtcgaagagatttaaattggaagtgggaagagattgacttttttttttttttttttttttaaagacagagtcttgccctgtggtccaggctagagtgcagtggcacaatcttggctcactgcaacctccgcctcccaggttccagcggttctcctgcctcagcctcccgagtagctgggattacatgcatgagccaccaggcccagctaatttttctatttttagtagatatggggtttcaccatgttggccaggctggtctcgcaaactcctgacttcaggtgatctgctttgtgtctcttagggagcatgtttgaagagtcaaaggaaaaccaagtctgttagctttgcgaaacgtttttttttttgagatggagtctcgctctgtcccccaggctggagtgccgtggcgcaatctcggctcactgcaagctctgcctcatgggttcacgctgttctcctgactcagcctcctgagtagctgggagtacaggcgcccgccaccacgcctggcaagtttttctggaatttttagtagagacggggtttcaccatgttagccaggatggtctctatctcctgaccttgtgatctgccctacttggcctccaaaatgctgggattacaggcgtgagtcaccgcacccggcccgaaatattgttaaaaccaagtacctttgtgaaacattgttacaaaaaccaagtaccaagtgtgtggcattaaagtcaaacattaatttcaagttacagaaagaaatagcactgagtagaggttgatcgataaaaaatgaataaaatattcaacagaaactccttaaaaataatgtgagaaatggacatgtgacatgggcgtttaaaaagtctgtaaggccaagaaaagtttcctgaaaggaacactgtgcccaggcagtcctggctttgcaggattccctgataaccgagctcaggcatcctgcagagtgagtactgcccagatagaaccttctgcctctttggggtaaaattcgatttcaaagagagagaactgtggggtgagaaaggaagactgggacccaaggggccaaactagtgacaatgactgctgtcatgaagggtgacaaggacaatacctgctgagctcaaaccaccctttggacccagtgaagctcagatgttgcttccagtgcaggagtagttaagctcaactgttttacaatatccagttatttggctgggtgtgatggctcacacccacactcctagcactttgggaggccaaggtgggtggattgcctgagctcaggagttcgagaccagcctgggcaacatggcaaaaccccatctctactaaaaatacaaaaaattagccagacgtgatggcaggcgcctgtaatcccagctacttgggaggctgaggcacgagaatcgcttgaacccagggaggcggaggttgtagtgatccaagatcatgccgttgcactccagcctgagtgacaagagtgagacttcgtctctaaataaataaataagtagcaaaaacaatccagttattcctcaaatatttatgagcacctcttaatgtttcagatactattgtgacaagagaaaaatgtgtttgtatatgtaaatagaaaaacaaaagtgatgtggaagcgattacttctgtcattttggaactaccttcctcccatattaagtccatattatgacaattcaaaaataatcaaacttcgtaagatattacctgtgaaaggaataagtatttaaaaatgtattttagatggccttttagaaatctagtccatggacacaatctagcatcatgcagttgccttcaaaaagaatatcctaaagttcttgtttgttttctttttcctttcaaagtgttctgttgaaaataacccatttgtcattttagttttgatatgacttagttatacatgccttttgaagcagtacccacccacatagtaattgcgactatcttgtgttttgaaaaaaaaaaaagcgagacaatttgactttgatagttttaagtttaaaaaaaaaaaaaaacgctccaagttagactcagatgaaatcctttgatcttaggtattttatgaggaaactggaaaaaataatttcacaaatttctattttgctttataaaaattttatgattcataacacagggaaaatccagtaagctaaggcgttttttagtcgtttatcttgctagtgagtctttgatgtgagttatttctttgcagtaaggtggcatggatgggtgatgaggctaagctgaaaagttaaatctcataatggacatttgcaccctttaaggaagtaaagctctcaaagcatgattatggccaagatgaggccggtggcatgtttgtctcctaaatgtgcagccaggaatctcgacggtttgtctgaggtatgcaggtgcctggtagataaattagggtgagcatcattcatggcgtgtggtcagttgcaggccttcagccacagggatgacagaagggaagtgaggaatcagggatgggtttgaactgcggcgttgctttgggagtctctagacatgatcagttgcacatggactgttctccttgtcaccctcaggatctactgtcctgtggttgaagcctgcagctctcccaagttggtggcactgggcttcctgacttctcgatgtctaatgagacagtggaattctacttgcagccccactgcaggccaggtttatctctcctctgtcttcccagctggtgatttggtttctcagtgctgttttgggtgcacttttgtggaggattcaagctggtccttagccactccttgaaccagaacctcttttactttcttatgtagttcttctcttcctgtttttatatcctaaaactagtccagcgaccagcccatcgtagtagaagcgttggtgttcttcctaaaaggaattgtaaacatctcttggagagtgggttagccaccataaattgttttgggacttcggatacccccttccgcaaatgctatctagagatgctttaatttcatttcaattggttattgcatccctgatattttgaaactgttatgagtagaaaacattctggtttatgaacaatctgcttttcactatgaaatagatgggaattttaattattcctgcgctttgtcttagagaagttacatgcatgcatcaagtgttgtgtgatcttgtgcagttcttcttagattgagtttttggctcctcgtgtgcaggccttctaaaatcacaatgtccctgagttgggaaggaacatcaggggctatctggttcattatcctaccccccaccgcctcccacgccccacatttctggcagcctacgagtctttggtccctccccagtgctacctggccagctctcagccttgggaggccacctgcccgtgtttgacctgggttaatgaccgccagttcctttcccttaatacgactcatctgctctcctgcatgctcgctccttgggtgagagctgccctctgcaacctcataacggaaaatggcttgacttctgccacctcctcctccttcccatgaaagaatctggtcttttctatctctctattatttttaaaaccagattaggttggttgtggtggctcacacctgtaaaaatcccagcaccttgggaggcagaggtgggcagatctcttgagccccaggagtttgagaccaacttgggcaacatggggaaatctctaccaaaaaaatacaggaaaaaaaaaaaaatagctgggtatggtagcacacacctgtagtcccagctacttgggaggctaaggtgggaggattgcttgagcctgggaggtcaaagctgcagtgagccttaatcactccagtgcactccagccggggcaacagctagaccttgtctcaaaaaaaaaaaaaaaaagaaaaataagaaaaagaaaccagattaaacattcctaagtaattctttttgtgacttttgcctgtttattaactatcctggctacttaaaatgaatttttgagttatgaatacatttagatggttttaaattcaaagaagtctcttttttctcagtcacttccatccccagccaaacagttcttatccccagagtcagccagtgttaccagtttcttgtgtccatgcagtttgctttatgcatttacgtaagcaaatattcgtacacacacacacaccctttctatcttatggaaagttatttcagtggcacatgtaagtatctcagcatctcttgattgtcattctttttggattttttttttaaatttcatacttacaaatgatcatgtatcttaagttttctgaagtagccttggtttcgcattttgactgtatttattatggtatcaggttccatatttcttaaaagtaatactgctgctgcacttacattgaacaagtcaccaagctctgttaattttgcctgaaaaatgccactgctttgacatgatagaaaacacaggcaatagtctttatttctcactgctgtttttttcttattttgaagaatcaccctaatacataggaaaatctcgacttagcaaagagataaatgagactgtaattattgcattgggtttttcacaatgtatgttagttatctattgctacataacaaattagttttagagttagcagcttataaagcaacagatatttattatctcacagtttctgtgggtcagtaaatcaaaagtggcttaactgggtggttctgacatgtgcctttccacagtgactggtatgtagcaggcaagggtctcttatgatcctgagtgaggatgtcagccaaggctgctgtcatctgaaggcttgactggaacagaaggacccatttctaaaatgagtcactcacatggctggaggcaagaggcctcagttccttatcgcagagattgctccttggggctgactgagtgtctgcacatcatagcaacagctggctttccccagagggaatgatccaaaagagaggtggaagcctaatgtctttgtgacctaatctccaaagtcacacactgtcacctcttccatattctattagaaaggagtcactaaatcagatacccatgggaaattacccacctcatgaagaaagaaatatcgagaatttgtggacctgtttcacaaccacacagcatcattttttgaaggtagagtcaccttgagcatttaaattagggttctgctctagaagtctatccctttattttttcccatttgttaaccctttttctacttggcataatagcagccattacccagtagaagttgagagatttaggttgcacagaatcccacagtcttttcagaaactcatatccttagggtgtctgcttgttgacagcattgtagaattgaaacttgaccacgtgttatgtgttgggctgccagtggtgatcagagatgacggtgagtagtccgttgagttagggctgtttgcctctgtcggtgtcatctgggtttgaactggacttgcagtcctggtgggctgctttcatttccaccctgggtctttgttctcaccactcacatcttacgcctctgttccaccgctgtgtaagaggcaactgtgcacacaccttgcctctgccattgactgtaagctcctgagagagagagagaatgtgtctggttcagttttgtggccctcctttttgctagcataatgcattgaacatagtatcattctgcatttttgttgagtgtataattagatggtcaatgtggagaactcagagggattttaggagggggtggcagcaatataaggttgtgggacccaacattaagagagggaaaggtgtagcgggaaggcgttggtgtggggagggagaaagtcaagaggattcaacctggcaagagttggaagtgacaggaaggtgtgtggggtggggacgcaataggacagtgtgtgagattttttaaaaaatgcaggcaggggagggatgagtaggcagagcaccgaggatttttaggacagtggaaatgctctgtatgatactataatgggggatacatgtcattatacatttgtccaaactcaaggagtgtacaacaccaagagtgagccctaaggtaaacgatgggcttcgggtgatttcgatgtgtgaattgtaacagggatactactctggagcaggatgttgatcatgggggaggctgtgagtgtgtcgcggtagggggtacgtaggaaatctccgtgccttctgctcaattttgctgtgaccctaaaactgctctaaaaagagtagtcttaaaaatgcatgaaataggatcctgtctcaggactggggcaagagcaggtagaaatggaaagagtgactgtggctgctgtgtagcaggcacacaatggtcatggtccatatgctggcttagttattttttaaagcaactaaagttaagtaatgtatacgtaacttacatatttatgtggtccaaaaaacgttggtcatctgttttaatgcagagagtgtttaccctaagcctgcaaatgtgatgggtaatagctagaactagaaaagtactttagaatatggaaggtaaatacatgaattaatttaggtaagagtgacaaaacagtggaagtccctgacttagaacttattgtccgcttggacagctggtgtacgtgatcctggggtcacagtggctccctgtgcaggaagaacactatttcccagctcagataccatctttttaggagagtacctgccattgtggaaaataaggataaaggataacaagttacctccttaagacaaagtcataggagaaatggtaaattccagtatcttgagttttgagttggaacttgaaatttattttactacaagaacactgttgatggattatgtattttatagactttgtgggtggctatgggactctagtaactgcttatgcacagtttatatgagtagggctgctggatttccagatgacgctctcgcaactgaaccttcagattaccacctgtttctcagtttggacactattagtgtgctgctgtcacttatggctcgggcaccttatcttgttttatgtcttgatgaagaagtacaaaattcagtgtaaaagaaaaacatatgtaaagcttatagaatcaccccgttacacaataacaatgcagaacaatagattaataacaggaagagatgctgaaacttctcttcagcctagtttctactctgatgtttcttctctgcattgtacagccaatctatgaataatattttcattgtctaagcaaagccaccttagctaaatatttaattctactggagtcataaacagggttcaaaatatcacacatagattttatgaaagtaataaaatgcttcagaaagtacagtggctcagcctggggccactgcatctgataatttggttacacaccttgtcatgtgcttcctaaaagaatgacgatggttgcttatctgtgtttaagcttgtcattttgaggaaacaaagataagacatttttgctgtgtctctaaaatgcatgtgtaaactatttttatagagtaatgcttcagagaggagtaaaatttcctgcaacaggtgcttgtaaacagtaattgctcagtaaaggtcacctcccagctgttgcctacatgttacataagagaaccccttcacgtcttccgtatttgcagctctgtacctttgtgagtcacctaaaaaataactcatagggttgtttttctttctttcttttttttttctttgagacagggtctcactctgtcgcccaggctggagtgcaatggtgcgatcccggctcactgcaacctccgcctcctgggttcaagcgattctcctgcctccattctcctgcctcagcctcctgagtagctgggactactggcatgcatcagcacgtccagctagttttatatttttagtagagacagggttttcaccatgttggtcaggctggtcttgaactcctgacctcaagtgatccacccatcttggcctcccaaagtgctgggattacaggcatgagccaccgtacccggcaggtttgtttttcttatgacaaaatcaagcctggatggtgtcgttaacggtgaagttaaaatttttcttttgctttccttaatatttccgtgtttttttctttttctttttttttttttttttaacaaaagtctggtttctaaaccaaaatatttaatgcttcaaaaggaaaaaaatgtaagtcagtccaaagaaatgattcattttgcagaggtaattactactaacatcaacctttccaggtctttttctatgcagctatatcattgacatttttttaaaaatgagattacattgtactggagggtagactgctataacaacttttggttttggatgggaataaacagaaaaagtctcatctccagttcatttctggggagggtcaggaggtggggggtgcttggctctactcccttcgtatgattttcccttctttatctccaggatcttccccatttcacggagagacatggatgagaatgaaggctgctgagtgggaggggcttatggggccagttttgaagaggcctcataccttttggccacattccattggccagaactttgtcatatgactgcacctaactacaaggaagactgggaaatgtagtcaagttgcatgtccgggtagaaaagggacacttttctgtggaatagctagcatgtttgtaactcaattttataaaccattttgaaacttttcttaaaaaaagcttatttcagacacatttttatatgctggatctgctcacctctccatctctatcttattaaaagttcttgccagaatctctctccaattgttctccctccttctgccctgttccctcacagcagccacagtggttctttcaaagactgaagctacattttatcactcgcctgcttaaaactttacagtgcttctcccccagtggtcgggataatgttcaccctctcctgaagctggcctctgtggtccttgaccccatcaggtcccacggtgccctgcatggactctgccatccttcaccagcttccccccatcaaacggggcttgctgtcagctcttcctgcaactcccagccctgccgcaccactcagccttcttccttcaggcttcagctccagtgtcttttcgccaaagaagcctttcctgatctctcagccataaatgttcctctccccagttgctttttccctctccctctactcagttgctttatttaactgtcatattgtctttggagcacttgccaccatcagaaattatcttgcattatgtatttatagtctgtcttccgcctactagatggtgggttgcttgaggcaggaactctgtcttgtttacatctctatctccagataacgtgtctggcacgtgatgggtactcagtaaatacttgtggaataaagaaatgtcattacaggttctctatgcattaaaattttttagaaatggggtgttgcagccgggcgcagtggctcacgcctgtaatcccagcactttgggagactgaggctggtgtatcacctgaggtcaggagttcaagaccagcttgggcaacatggtgaaaccccatctctactaaaaattcaaaaattagctgggcatggtggcacacacctgtaatctcagctacttgggaggctgacgcagaagaatcacttgaacctgggaggcagaggttgcagtgagctgagatcccgccactgcactccaccttgggtgatagagtaagactctgtctcaaaaaaaagaaaaaaaaaaaagaaatggggtgttgctatgttgcccaggctagagtgcagtagctttccacaggcttgatcgtagcgcactgcagccttgagctcctggactcaagtgatcctcctgcttcagcacctccccgagtaagtggtactataggtacttcaggttttgaagcatgtgtaaaaaatactctagcagggacatcatttgagctgagcaggccatggaggcatcaaatgattagcagtagggtacaacttgttgtgagaaccagaaaagagccttggcctacataacatcctgtgctaaagaaagaaccctgactcccagcctgtgagtgttccagagatgggcagagcagtccctgcagcgcttcagggaacagggaagagcagctaacccctgatgcttctgtaagcactttgggacgccctttgctatagtagggtattgtcttagttatccctgacttgcctggtgttactttacatcccttaatcctttcatgaagactggcaaacacattcagtcctgccaaccttgaaagtaggccatgtttgaaagaacattggcacaaacgagttgcgaacatcttctttaccatattcatgttctttaccgtattatgaggacactgtgcttgccacatgggcagtcttgttttggggatacgtggttggatggacggttgggtggatggaagactcctggaatgtgaagtatctcaaggatgtgtgtccattgcatgaatgtatgtttgatctatgcagctaattgacaaagatccaccatcctgtaccctccatgttttatttttgccctgccattgttctcttcgacattctataatcatcaaggttttcttgccctcacctaggtcttgactaaacatgttgggtaaatgaatttcatattactactttcttgcgccgtcagccattgtgttcttggggactccatcctgagccttttttttttttttgagaccgagtctcaaaaacaatgaaggcaggtgactggtgaggagagttccagccactggattctccctcagtcccctgtgctgtggcccttggtgacccctagatttcagtgcccctgcctgacactggagtccctctgtaaagaagcccctttctagctagctagctgttccatcccgcacacccccacagaagccttttgctgccattagtaagcacgcctctattttctcacctgtgattgatcatgcatgattgagttttgtctattaaagcaattgataagttactaaaatggcaccaaaagaaattagaaaaaaattgttgcaaatgtcagtgaaatcttttaaaaaactgttgtactttagaagtgcctcaagtgactaatggaggcttaggactctgagtggtgtcgcaaggaaccttatgcacagggacactgacattagcctgtactgtcacccaggctggagtgcagtggtgtgatcttggctcactgaaagccccacctcccaggttcagcaattctcctgcctcagcctcctgagtagctgggattacaggcacatgccaccatgtctggctaaattttgtatttttagtagagacagggtttcaccatgttggccaggctggtctcgaactcctgtcctcgtgatccgcctgcctcggcctcccaaagtgctgggattacaggcataagccaccatgcctggcccctgggccattttttattcttactcttaggctaggtttctgcagaccttggttataactgcaggctctacattgataactgctgagtctctaagtcagaccagacgtttctccccatttagtgatacatgttttccatccaattgcttcatagacaccttggacacagtgctctatttcaatttatgttgacagttcatgacccaaactggaaacctgagttatcctggacttctgcctaccaccttctcctaagccatctcacctgtcctaaatgtcgtagcccttatctagttctctaagctgtcatagggtcctgtactgctgggctgcttgttgccttcaatagagcctggagcatattcatgtcggtatttcaaggccccgggcataagagcttcttaggaaatattggaatgaatgaagaaatgatgaataaatgaacaaatgaacagttgattgatttccaagacactttggggtggtttccatttagaatggtttttatttctttttactggaactaaggaattatttttgcttgttgatgccaacattttaaaagcactataattttgaaatgttttaccacattaaattcttttctattgcaggtcacatacaaatatgtcaacttcaaaggaggaattatttgttatatttatgcaaacttgtggcacttggacatttatctagatggttttttgtgtatgattgtgttcttggtacagctggaaaagctaactttaaatggcaggagtaattgatcttaatgacttactttgaattaaacttgtgcttaattaacttggatgttctaagtttctagagtcacagacttttagagctggaatattgctttttgaacttcagtgtaaggaagaattgcttatgcttgaattgatttgttctgtatcccaatactggactgagtagaaatcaattactttaatattggatgagagagtgattgattgaagatcaggtttttaacctctttttttgttctccttctcacagggagagtcccgtattctcagagtaaaagttgtttctggaattgatctcgccaaaaaggacatctttggagccaggtatgttggctttgtttttatttctgtggacttctgaaaaattgacaagcagttttcaaattcaggcacgcatcagaatcacctggaggacttcttaagacaaagctggctgggccccacctgcagggattctgatttaggaagtgatgctggcccagctggttccaggagcacattttgcacagggcttctggacttaccatcacaacagtagttaacagcctgttcttcacatgggctaatttcatgagattagcttctggtgttgtattctcagtagaaaaatacagtgtgggactatggctctgcttattttaatacttcaaaaatatttcaaacttagggaaacataaaagtgtcattttgtttctaggagaactggatattctggacatgtataatctgaattaagagatttggtgatttggggcgaatgtggtgatttctgtggcatttctagaagtaccagcagggaagcagccagatctttctgtgtgtttcagcttttttattttcagtattgcacttggatattcctgtatctttaaatcagcccttttgttcctattggctgtcagcaatgtgttgcatgaagttattggactgggagcagggtgagggtagccgtagagccacctggcactagaatttagccagattttggagaccaaagatcttttgaactttaaacctggaatggacatgagtgatgttctagtccaacttttccattttatggttgacaaagcttgaatccagaaaggttaaactacccgtgcaggtcacgtgggccagtgatgtttgaggagtgacaggaacactcctcctgcctctacccagtacattttcctttgtggcatggtggcctgaaaatactgtgaggtttaacaatgaaggcaggtgactggtgaggagagttccagccactggattctccctcagtcccctgtgctgtggcccttggtgacccctagatttcagtgcccctgcctgacactggagtccctctataaagaagcccctttctagctagctagctgttccatcccgcacacccccacagaagccttttgctgccattagtaagcacgcctctattttctcacctgtgattgatcatgcatgattgagttttgtctattaaagcaattgttaagttactaaaatggcaccaaaagaaattagaaaaaaattgttgcaaatgtcagtgaaatcttttaaaaaactgttgtacttagaagtgcctcaagtgactaatggaggcttaggactctgaatggtgtcacaaggaaccttatgcacagggacactgacatcagcctgtactgctggtaatgtcagagtgaagcaaatcccacctgaaggcactctcttgcttgaccgtgaagtcacactgagtggttcaccatgtgcctgcactcatgcaaggataagccaggtctctaggcatgggcagaaggatccctgcttcccttctcttccctttcagagcaacactttcagccatttccatgagtctgggctcttgttacccagctgaaatactgtttcctgaaactcacaaatttgctctctaccaaagcccaccttatgccctgcctttccatgaagcctgtcaagaaaattccaagaaagaaaatttgatattcctatatcaaagcgtgtgtctttcctctcctaatccctcttagctgccacagtaggtgcaatgtcattttagcttttgcagatgcattacctggtggtttcctagtatttctttcacttgcctcgattgctagattagaaatttactggggacagacatctttttaatatctttctcctactacttaacatatagagcatcaagggaaaaaaaaacaaaaattatcttttaaaaatgtaaacatttgcatgccaaccggtagtggattgtgcccttaggttaaaaatatgtattctcactctttccaatgaaatctgtagtgctgaaggagcaagttggatttagaaaattttggtgatttctttttaagcttgattgggtttttattttgttttatccaaaccctcccattaaaaaaaaaaattgattcacattgtatttctaagccagttaagtagatttaaggttgaatttgtagtggaattaggctccaaatcttagaagagcaaattcgtaccttgaatttttactgtttagataataacaggcagggtcatttctaagggacatggtggaaaggaatattcgtgatattaattagaaagataaatcgtattgtggccaagtggaccaagtgccagaaaatgcatcagaaactctgaatcctgcttctggtcttcattttcctagattgataagtaggcgcacacattcctgctttcctccctcctctgaagtgctgtagttcttcagggacacatttgccagtgggtctgagatgcaagtgtgaggtggcagtgtgggttaatgccatcttggtgtctcactctggagtggcactgcttattgccttaaaattttgccagatgacctgcattctccctccatgctatcttgggcaaatcccttaaaaccactcatgccactgtgtattagtgtacaacaaggataacagtttctttgtaactggcatgtggggaagaaccgcaaagtgacttctgtcggcagcagctctgtcgtgttttaaggtagtagtcaccgggcagaggtttggtagtgtcctctctggcagccaccgtctccttcagagccctgggccactgacatgccttttctccaagtctcttccctgagtattccccgcacctctgtgcctgatcagacacctcattctcccttggcagcaagaaggccgggtaggcattggagcccttgctgtagacaggataatgggtgaaagaggaagaacattttcatgagaggcatttcagagcctttaaagtggtagaaatcctgtagacaatgatgtttttaaaatcagggatttgcatctctgggactccaactcctttgtttccaagggaggttccctgggccacttggcctgaggttgggaaggggtcggtggttgcggggcagaatgtgatgcttggggatgcaggatggtcaggggcgggtgacggtgggaggagtggagcacgtcatctctccttgcccttccgaggtggtgatggaagctgcctacagctccagctcagaggccgggggtttctggggcccttggtgtggggcctctctcctagctggaggtggccccttcccccttgagaggcctgtgcctgaaatgcccttgtccttttcggccgtctcggccccagtccagtacttaggatccaagatcattttcagctccttccagccaagtctcactttattaaaggagctttgtttggagcagattggataaccgagccgcgactccacttgaacatctgagcagttggcccgcgtgggaaacagcaatggcagagaaagatgcttccctttggatcataccttgaggacaaaaaagggcagtcatataatctgtcatctaaactgggacgcttttgtctggaatgaatgcgacattgaatgggacgtccggacataagatgtaaattgagactgcagtcaccctaggtcagagacatccagcctttattatccatatcatggggaaaaaaaaatggaatgtgattttttttccccaaaacagcttaaaaaaaatgctaagtaaaagaaaaaaataaaaagcaccttttaaaaaagcaggcctcacataaaatgccactaagacatcaaaagagaagtaaaaccctgctcaaaacgcccacgtggggcggccacagctcacccttgtgtgcctgtgtcccgtttctgctgtcccctttcccaaggacgtactctgccctaacctcaccctgatctggtgactcctccagtggcccatcctatgttcaccagctgggtcctcatctcctccgccacacaggctgctgctctgtcccccagcagacctctccctgtctctggggtgcccatgacccctgggaagtgctgtgcatagttctgtgagtgaggttgacttgttgttttatgcttagaattgtttctttctcctgaaattagatttttctcctctgatgactaggattctattttatttatttatttttattttccacgacctattatagcacatagcacctaacaggtcctcaggaagtatttaccaacagattgcatgaagtgagcccgttcttcctccctctctccttccctccctttcccaccccttcactggaaactcacagtcactaaatatgccatgaagttgctgccttttgcctttgcctgtgttttcccctctgcctggcatctgctcacatcctttctggcagctgccctctcatctgtcagggcccagtttgggtggcaacactcctgctgaggcccctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccacctcatcaccccagcccaagaggacagaacactgctgcccgcctcatcaccccagcccaagaggacagaacactgctgcccgcctcatcaccccagcccaagaggacagaacactgctgcccgcctcatcaccccagcccaaggggacagaacactgctgcccgcctcatcaccccagcccaaggggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaaggggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaagaggacagaacactgctgcccgcctcatcaccccagcccaaggggacagaacactgctgcccgcctcatcaccccagcccaagaggacagaacactgctgcccgcctcatcaccccagcccaagaggacagaacactgctgcccgcctcatcaccccagcccaaggggacaggacactgctgcccgcctcatcaccccagcccaagaggacagaacactgctgcccacctcatcaccccagcccaaggggacagaacactgctgctcctggggctgtagtgggtgtacaagttccctcctcctacggccccttgtcttagcggacttgggaggctcagaggctccagccctgtcttattggtgtttgcaggtccagaaatcttggcacatgacagttgcctggtaaactgccttgaattcattgagtggaacacaatatggtgtgctgtggcacctgatggccagcctggaggaaggggtacttttttctcattgtcatggaggtcccattggctcttcaagcatctctcccaagcatctaggagaggtgcttatttctcattctcacaaaggctctttgtggaccagcacaggaggcctgcctagggcttggatgaaaaatgagcatagagagaagaggttaagggaagcaagctcaagagtgggctcagagaaaagaatctgggggagtcaatgagatggcaggcaaggaacagtctccatttgatgctgatgtttgtgcagaatgactactttataccgtacagaaggagcacactctcttcttgtggtaggtcccagaaacttctacttgggggtacatgaggcccagctgggattgggcacagaaggtcatggctctgcacctgaccctcaccttaggggtcaggagagagccaaggacacgagtgatgggttagggaggactcaggcaccatggaatactgtggccatccttccatagaggagaggtacaggtggcaaggcaggcaccaggcagccgccgacagggtcaggaaccaaggcaggtccaggaaacagaggccaaaacagcagcaggaacctcaggcttagcagaccactgactcttctggaagtcttttagacgctctgtggtggggcagggacaagtccttggtgtttggccatggtgaagtgtgcagttagggtaggtgagtcgtgctttcttaacacgggggtaaggaaggagggtagagtttcagcagctcctgtttcttgagcacttgctatgtgccagatgtggtttgaactgtttacatgggagctcgttgggtcctcccagcaaagtcctatgtagcaggcactgttgttctttttgcagataaagaaactgaggcaagaaaggtttagtaagttgtagagctaggtattgaaccagggcctaacctcttaacagaagttagttagaggctgttacttgagtgctcacaggttggctggcaatgtgtttggaacccaatgtaatgtcattagattctgcctaaatcttgtgaagaggggatttccatttgataggtaagaaaattgagaagttatcatcagcccgagatcaaacaagggtatagggagtggtgggacctattaagttcagatttatgtgacttctgatgctatgctcagtgatagatctcttcttgaggataaaagtttgagaattagggttcctcgaaaaccctgagatttctttttttgaggggtacctttttgtgttagcagatttaaagagctggccattgtatttgatggtagctgagtaagccttttgaattggagtatcatcaactattactagatttagaataggtatattttgctaagtaagatcttaaagtttttcctttaaacttaggatagctgttcaaatttcaaactcttcttttttcagtaagctggagacatttaaccttttgtcataggcttaaagcagtcattaaacagggtcatttcaatgcaactttttaaacctaactcccctccctttttctctttaattatgatcagtggcttcatttgagcttacctaggatgcatttgagactataaagctgctttcttaaaacttagcaatgttggctgagacaaaccaactgtagaatctcatgctttcataaagaaggttgcaagacattcattcacagttgctcctttattgtgtgtgatgagccattggcatcaaacctgcttgggttgctatcagtttttcttttgagagctggtgacttgcaatttaaaacacacatgggctcaagggtattggagggacatggcagaaagtccttctgctgcctagtgattgagagtgatggggtcctaactctgtgtgaggatagctctttgacacagctcacctcagtcccctttacctaggtttaagttgttagcattttgctgtatttgctccatctctttttttccctgaagtattcagaagtgaattacagacatcatgacgtttagttcctaaatacttcagcccaaacctctaaaaaacaagaacatatttctatataactgtagtactgttctcacaaaattaagactaattgcctaatggactctattacctgatccattatctgaacttccccagctgaccttaaaatgccttttacagctggactgtttaaaccaggattcaattgagaattctgtattgcatttaatcggtgggtctattaagactctcttaacctagaatagtgataaaagctttttactcttggtctctttgcaaactggggacccccagccagcaacaccccacctgggtctcactctaccatgctggggtgccctagctcacctgtgttatagcttgtacctgtgttcagtggttcccgagttcttgcatggcacccaagaagaatgaggatatgctggacattgagggtgaggagggcagagtagaattttactgagcaatggaatagctctcggtggagaggggctgcagtcaggtgcttctctccttcccgtgtggctgggcccagggctttttatgggctcagaataggggaggggcaggctgcaggtagtattggaaaaggcaacatttgattcattaaaaggcattgttcagaaagaatcaatcaggaaagggtgggcagacaggaacagaagttctcgctctggttcgtgggtttcatccgggaccagcagtccagtctttcagccttcaggctgtttttggctcgaaggtggggtttcaccggggacccgcccctatctgcccaggcgtttggctgcttcctatcactatcaatagtccctccccttcaatgacttgtaggaaggttgtttcgcttaccacattccatcgtgtcctggtggcatcatcaatgaatgaacatgttcttttccccatgtttcttgtacgtactgtgtattagaagttggactaaaggcttgatcaaatttgaggcaggaatgggtgaggctacgtgcgactggagggacacaatgactggttctgtcataatcagtgattgaaattgatccctgggtaaatgtgggggcagtctcatccctccactatcaaaaattaacattttttttccccaggtgaccagcaagtaacgaggggaggggctgttttggtactgtatacctgcaaattttaaatattgatttactttacttccaaaggattggaaattttgtttttttctttctttgcccttctttgaaaacattgtacaaagtaaatctccttattttactaatggagaagcttctgaaagaggctgaacatacattgaagtacctttgctcagctgtaagcccttctggatcagaggtcagaccaaagtttgcagacagttggtccctgtggaccagactgaatccacacatgagtttatttggcctgtggagcactgaaacaaaaaaatgcaaatcctgagccaacgtttgggagatcccatattataatacaaatcgagatttccaggtttctggaaggtcttggcaatctagccacactgggcctgcatctgtgcaaggctgcatggctagaggtgtgtgctgggcacacccattaggtgggactgcgcctttctggccactagggaccgtacacctcctgttgttttcatgggggctggtttcccttgctgacacgtggccatgaggtccctcgaggtacaggccggcatttggacttgtcctctgcactgtttctttgttagcatttagaactgacttctcctcttcctcccccattgaggagcagcaccttactctgtgcagagtctgtctgtgatctgcaggctctttgcagagctacatttctctctggaggccttgctgagcagcctccgctgtctagggcattgctgagaatcacaacatcaacactaatggcccttgggccaaaaagtgtttgtcttaaaatccctgcaccaaatagcttgtgcaattactcctgtgagagccccagaggatctaactcttaggctgtttgttctagctgtattccttcccaccccccaaaccgaaatgacattaaaggatagtaactccctcccgcaaatatagataaaaacctctttactccctaacaaccacacaactatttacatttttgtgtttacctcctagtctttaagtatatgaatgcatgcaacgtcctttaaaaatatgtcaatcctagcctccacaccacccagccgaccaggtttcccagagggaagcattggaaatggcagatgggtaaatgcacagagctggcaaatctccatcctgcgggaggcttgctgtgcaatatagccatcgccacaataatggatcttcctaatgatccggaatcactgagcaaagactgtgccatgttcagtatacactaagtgagcatgcagggaatatatgatcagtttacaaacatttccacataagcttttttctctctcccactccagttttggtaaataaaagagcaaactagactgctttctttctctctctcactctccccctgtctccctccctcttcctctctgtccctgtccctctctcctgcctccctcattcactctgtcgtcctctctctctcctcctcctccttccttccttgcttcatttcccccctcatctctttcttcctcaccctttttaaaaagagacaggagtcttgctctgttgcacaggttagagtgcaggggtgcaatcatagctcactgtaacctcaaactcctgagctcaagcagtcctcctgcctcagcctccccagtagctgggagtacaggcacactaccatgcccagctaatttttaaaaatttttatagaggtgttgtcttgctgtgttgcccaggtgggtctcaaactcctgacctcaagtgaccctcccgccttggcctctcaaagtgctggaattacaggtgcaaaccactgtgtccagccagacaggtcacttttctgtaccattccctgatgttgtgagattagtgagagtccctactgtggaaccttgttcacgttgccctgactcctgatgcatggatatttgctgttaccaaagacacctctgctttaccaaatgttttgacagagtggaaggaaaactacaagacttcttgccctcgttttcttcagtctagaatgatgctggggtcactgagtctgtagggcagagaatctgatgtgttttttaggaagcggaaggtgtctgacacatagatgtttgacaaattttgttaatctggtcagagtgcagcctagaaaattaaattaaaaagctgctgaggagtgctggggtcgcattttgacatagatacaaaggcaccaatagggccaaaagtatgaggccagtcctagggctggtgaagtttggatcccctgcctgacattgtgtcctgagcccctggccccttcctgcacctctccctgagccgccggcccctcctcattccttcctctcctttatcttccgctgtctttgcctgtgcccccacgcccgtgccctgaatctggagtctttgtcttagtgcattctgtttgtctcactgtcttcctctgttttccttttcttgcggggaggaagggtagaaagagtgtaagaagctcaagtatgaggaagggaaggttgccaacttgagatgagtcagaaatgaaagcagttctgaatgtgtaagacaatgaggaaaatatttatttaacaaaaattgaactaccaaccaagccttcaggaacacagccagaaggtgctgtgtagggagaggtgtgcattaggatgctggagcgtggctgccggcgtccccaccccaggaggaagggactcgtttctctgaacctttttaatggttctccccattgtagacaggactggctctccaccccatcctcagtgggtgcagctttggggaggccgtgaggctggtgatcttggggcctcctatgcatctgtgttattgaaggtaacaatgtcccttctacccaaaagtcatgcttttaaaaacgcacaatcttgaatccatttgggaaacatctttggaactccagaatgttgtctttctcagatatgtattgccaatgttgtattacttcatttgctaagaaacattgtcatcgactttataaaattattgaataattctatttgggggagaagcctggaagctaagattttaaaggttatttataagttaagtgaactggcaaattcttaaatggtaaataaaacctttcctacatgctataggtagttatctaaatcagggcctaacaagcttttttatttattttcctgtaaaaagccagatactaagtagttgaggctttgcaggactgatggtctctgacatagcactaaagcagccttgggcaatacataaacaaaggggcatgtgtgtgagctgatggtccacgggctgcagtctgccaacctctgatgttataagccattgattttctacaacatatatatttagaactgggagtgtcagtgttattttgtcatggtttccagattaaactctttcatattgctgctggtcggtcaatgacagactaattctgctagcattgactttctgggccagggaatggggttcctagatagtgcagtagctttttatttattttcttttccattcaagggtaacatgcttaaaatacagcgtatgaagcttttggatatccactgtaaattttaaggtctaaaaatgaatgcaattgtgaaatgtttttgccttagagaaaacgctctcactgctttgtgttaataaatgcatgaaaaataatgatgaattaaatgcatacaaacagatgcagacataatttagatgagtgtattgcaattaaaatattgcccactttcgctacggaatattcgttggacttatgaccttagtgtaagtaatgtttaataaggaatctgcaatgtgtaactcctccgtaaattattaaaaggattggcattcctactgtataatatcaacacaagggatagaaaatatgcctggattcttaatgaatgtgtgctaacgttccttctggcataggaatgtgtagtaaatacacttcaagggttgttcgcatttttatccgtcattgttataggcatgttattgtgtgtctcctattgaagaactgattgatcattgttttaagcttctgtgacttttagatgcttaaaactaggcgttcatggtaggaaagtagagaaacatgtggaagaaatactgcgtttcaagtttgggagtggctggtagaaaaatcaaactaggctagtcttacagctttatcgcagttttgttttttttcctagtaggaaaatgatagtattggttgtatattaaaacactctctctgacactgacttgaatcacgtgcgagttcattgagaacagggattttaacttggttaggtgattttaaacattatttagcaaatctggaaaacaaaagagaaattttcaaatatctttcttccacggtggtgtactggctctctggagtgtgatttcatcttctcttattattgcatatgtagtcaagccatcatgttcttgaagtgagacttcacccactagtggggccgtataattcaggatcttttgtttgtccttacatagtttcttggcctggaatgtcaaaatatcagaaactgttctttctctggaatcatctgtctccgtgatggcctcgccatctgtacagtcatccaagctagggaccaaagagtcgtcctttactctctcatccttccggctgctgtgtctggctttcagtgtttgtgtagagcaggggtctccaactcctgtggcgtgttaggaagtgggccgcacagcaggaggtgagccatgggtaggcgggcaaaacttcctctatttacagctgctccccatcattcgcattgctgcctgagctccgctgcctctcggatcagccaccagcattagattctcataggaccatgaaccctattgtggaactttgcatgtgagggatctaggttgcacactccttatgagaatctaacgcctgatgatccgtcactgtcccccatcacccctagatgggaccatctagttgcaggaaaacaagctcggggctcctactgattctatattatgaagagttgtataattatttcatcatctattacaatgtaataataatagaaataaagtgcaccataaatgtaatgcccttgaatcatctccaaaccatccaccccactcctccaacccccaaaccagcctggagaaaaattgtcttccacaaaaccagtccctggtgccaaaaaagttggggaccgttggtgtagaggactcctcaagcagcgtccatatctgccctattctctctattcctccacctaggtatagaattctatctctaagacacagacagaccatgttcctgcctgggcaaacacttcggtgactgtgcattgccctgggtgaacgggggcacagaggtctttgtgacgccctgcaacgcatccctctccgagccgcggtgcgctcttcctcctctgtgccttgccggtgcccagaaccacccccttctccctctttgcacaagtgccttgctctctcctttctcaggccagtgctgctgatctgtctgtgtgacccgggtgcacagtccttggctctgtgcctggccctgtgcaggtctctgtggtggcacttagcacatggcgctaagattctccctgataggccttcagcgccgcaagggccagcaccgagtcctgcacatcctgctctagtcccacatcggcccaggggcgttgagctgaggacagctccctaccccctttctgtgcttggccgactccagcgcccgttcttctgcccctccttggctctgcctccccaccccttggctgaatctctcctcctttcgtgcttgttctccaggtgacttattcaggtccatattttcattacccttgcatttcattaattttctcatttattttattctgtaggtttaggacattctttaaaacatgcaggttctgaattttaataatataaaagtcctctgtgagtgcccatcacattgcttttctgtcctccctacattaaagttagcactgatgcctttatgtttaaggaaagcctcctgcagaggggctctgcccatgtaagttcattagaaggcaggtgtacagcctctgtcacctgaagacctcttaactgtctattccttctctggtctatcgaggtgtattgggggggttacagcaccctctggtggccatattgtcaggtgcatttttgttctagccaaataaaatgataacacaaatcgaatagtcgattggagttctttttcagaggacatcgttaattcacattttgtgtgtttcagcaggtatttaagccattctcgaatagtgacacttatgcagaggagagaatgattttatttaagtaatttctactttttacatagctagttttaaagtagataatacatagtcttaaactttatttcccctttatttctttcttttctgttagagaatttcttaaacttgtagaattatccaaataaaatgccagtcactaaaaaggaataattaaccaatagtgggaaaaaattaaattcttcaaatgaagagtactgtattcatttcatatggtcccagagataaaatgtttaggattcatttttagttttaaatgtttatgaatcagagcttcaacactcttccaggtttctttaagaaaggaatttgtctatctacggattaattaattagcagagcctctctgtgtcaccaagactggagtgcagtggtgcggctgtagctcactacagccttgaactcttgggctcaggcaaccttcccacgttagactcccaaatagctagaccatggtgcatgcaaccatgcccagctaattttttttttcaatttttctgtagatacagggatctcgctatattgcccaggctggtcttgaactcctgaaggcctcaagcgatcctcctgtcgtggcctaagaaaggaattcctcatatagaaaaatattgttatataaatagttgcctatatccatgctgtttatggtaacagctaaggttcatctcaaaagtatttgtatttggctttggaataaaaagaaatttcccctttcatagggttctgagattgtaggagtttttagtatttcaaaaagttaatggaacaatctgtgatgtgaaagagactcagaagaggctagagaggtctttaacacattttttttttgtattgttatcccttccctatagctaactttgtagatgacattgtttttcagactgatgattgtgaccctcaaccatggcagcaacaatttttaatttaaattttttaaaatgcaaggttgggtgattttctctctggatgcttgagttttgtttttgataaaaattctaccaggggcaggggaaagagtccattaagtcttggtgcaagaaacctgggcagattgcctaagtggatatgcatagcaaagaactgaaaacagagtagggggtgctgaaaacacccacagaggaaaatgcggggcggggggtgaatgcatctgctcattttgtaataaagtccttcctttcctttaacatctcttaatgttacaaataccaacaaaaaccatgcatgtcttcagtgggacagaaaatgagggaggcatttagggagaaaaaagtgtcagttgagatgatgttaaaataaagtttattttctttgcagttgcagacattaagatttattctctgggctgttaggacagttcctgcagctctctgtggtgacctgagcattgataccttttttttttttttttttttttgagatggaatcttcctctgtctcccaggctggagtgcagaggcgcaacctcagttcactgcaacctcagcctcccaggtagctgggactacaggcacctgccaccacacccagctaatttttgtatttttagtagagatggagtttcaccatgttgcccagagtggtctcaaactcctgtcctcaagtgatctacccaccttggcctcccaaagtgctgggattacaggcgtgagccactgcgcctggcctgtgccttggtttctcagatatttctgcccctgtagtgttctatgcactatgcagggaggtcctgactgctcagagcttaaattctgcattcctttattttaagtgtttggccaactgcagaggaaggtaccttgaaagatactccctctcctccctcactgctgggtcccctgtcataggagacctggcctgaggaggcttcttcctggtaacctgggttgtcactgggaccacatcttcccataagaataatgcttcaggttgtatgggagaagtgtctgacagcagtaacttcagcacaccctgagaatggcccttatggcagacacaccggacagcagtcactgaagaaatgagggttgccacatggaggttgctgggaggagggtgctaagtgaaggtgctgtataagctgcatgctttttacaagcagttgaagttctgctgtccagcccagagccactgggccaccctataaataagttcctccaaataaaccctgtgccttgtttgttagctctgggtctcttctttggcttcttcaacttggtgctgtccctgttgaagttaatacgggtttggcacaacacaggagtagacggttacctgtaccctgccagctgtccttgctacaaacatttgcccagagagtggcatttgtgccggttcttaaagtgtttcttgagctgcacattaataggccatgtcctctttcaacctgccctctggctaggtagaacaaacactcttcacctgggctctgagccagctgctcacctgcagcacccccctccttggagatagcttttcatgttgcatcttaccgagttgattgagtattcacttcgaagctccatgctgcacgcatgggcctgtaaatagctttttcgtgcacttgtgggtgtcatttcagatcgcttcatcggatttcagttacccaagtttttacttggttcaccgtaagtttgcaaaaaagactttttgttatcctgaacattgcaggtggaaacatgattaattttgaattatttttgtttaacttctaccctctttcctatttacagcctattttgtttattttaacttccgctgttaagacttctaaggccaggcatggtggctcacgcctgtaatcccagcactttgggaggccaaggcgggcggatcatgaggtcaggagatcaaggccatcctggctaacatggtgaaaccccgtctctactaaaaatagagaaaattagccaggcgtggtggcgggtgcctgtagtcccagctactcaggaggctgaggcaggagaatggcgagaacctgggaggtggaggttgcagtgacccgagatcgcgccccattacttaaaggaagtgggcttgtgttttgtggccttaaggatgtaatctgttggtaggagggaatgagggtgtcctggtaggggtgtctggctttagtgtcaccattcttttaggaattgccatgttgtggggtcacgtcttgtaggagaaacgttctgaagtcatgggattctctaccctcctctcctgctcttggtttccaccagcagcctgcttggtgtggttttgaaatggagcagagatggttttgatgagcctcactggaggtgctgctgagattaccgagaggccttgctgggcacctggagtcaatcatgccgggcactagcatccgtcatcttaggaggtagggattattatcctacctcctaagccatccctggagtggtggcttggagaagttttccagagaaaccgatcagttcataaagtttctgtaaaggattaacacagtgtagtccagaaatgtttgttcatgaagtcttggtttttgggtttaagaaagtgttctcctgtctgggcgtggtggctcacgcttgtaatcccagcactttgggaggctgaggtaggcggatcacgtggtcaggagatcgagaccatcctggctaacacggtgaaaccccgtctctactaaaaatacaaaaaattagctgggcatggcggcgggcgcctgtagtcccagctacttgggaggctgaagcaggagaatggcgtgaacgcaggaggtggagcttgcagggagccgagatggcaccactgcactccagcctgggcgacagagcgagactccatctcaaaaaaaaaaaagaaaagaaaagaaagtgttctcctaagtgctgctcaccctttttcccaaacaggacctaaccctgcgagggagggtgaattaggccatttgtttacagtttcatcagcaccaggcgtgcacggcgttagtcatggtccttgctctcaaggcactgtacttgtagcagcagtatggtgcaaatgtgcagctgaacagtgggcacaggttcctggttagcatttagtgccacaggaatgacagggaggtcttaattgtgatcggaccgtgggagctggattaccccagactgtagctcacttccctgtaagcttttggtagggagcttggaattagcaccattagtagaaacagatttcaaagggaatggtacctgttccgtatgaaacactttaaaactcccggtgtccacacgtctttaaaaaagaggcttaaagcagtttcatttcatcaaccccaaactggagaagctatgaatgaccacactgatgaatatgtcctaaaatatatgtatattatataaagtgtaaaaacagacaagcaggaacatactaccagtttctatcacagctacccttaaactatcattggagggcaattaagtgggtacaacctctagctatttggaaatatgtataaaaatgacaaatgcctggttgggtgctgtggctcacgcctgtaatcccaacactttgggaggcctaggcgggtggatcacctgaggtcaggagttcgagaccagcctggccaacatagtgaaaccccatctctactaaaaatacaaaattagccgggtgtggtggtgcatgcctgtaatcccagctactcgggaggctgaggcaggagaaccgcttgaacccaggaggcagaggttgcagtgagccaagatcgcgccattgcactccaggctgggcaaaaagagcaaaactctgtctcaaaaaaaaaaaattaaaattaaaattaaaaagtaaaataaaatgatgaatgcctgtactcttagacccagcactcccagttctgccagttcatgcagcagatacatatgcacacgtacagagaaggatgcaacagatacatatgcacacgcatacagagaaggatacgtatgcacacgcgcacagagaaggatacatatgcacacgcacacagagaaggatacgtatgcacacgggaacagagaaggatgcacaagaacatacattccctagttgacggagtcgcagattagaaacaacagaaaagcgtccattaaacaacagtttgaacctgtgttgagatatcagcatcccttctccttctctcccccactccctctgccctctgcaggttttattctcagcctgattttctccattggatgtcagccctaggctggcatttgcagagagagatggtggctcctgcagagcttacggcctgtattttattgggctgtgttgagttacccctgatgtgagcaggagaatgggatgttctgtaaattggcctctagcccatcgtgaattcatattttttttataatgtgtgagtcacactcccttctgtcttcatttccttttcacactcccagcagatcattctctctattctgattggcattttatacatgaggcagcagaggctgagacctactgtattttgagacactgtagctttatatggcataattaaatcatagtcatcaatcactcttaggaggtaaggattattattggcattttccagatgagaaaaatggaggatgagaaaggttaggcatcttgctgaagtcacagcagacttaagtgccggagttggagctgcattccggtatgcttgcatagactctcttctcatgctcactgcacgccagtcacctggtatgctgaggccctgcctccaggccttctgattcagctggtctgcagccctggcatttccagctcctcaggtgattggaatgaaagccagagctgggagccactggtctacaccaccagtaaatgatcgggttctcacttgatcatttaattaagaatcttggttttctgctttcagtctgagaactcttgttcatgctctcaggcatgtcttcacgcaggaaagtgatctttgaaattaataggtgcttggaaagcctttaagcagcaggcagtggagaaggccactcagtaggaagggccgagcatgaagcctagtgcacaggcttttcaagaccccacggagggtcttcagaaaaatgggtttcctttgtctccgtggcttccagtctttgttggccagcagttgtctgcttcaaatgagctggctctagaggggcctggagaacccagaaaaactttgtgacaacctgacaagtttgtgttaatcttctgggcattggctttctgcacttattgctcactgaatcactgaaccaggcaggtggggtactggagggcaggggatgggtaagaagggcagcctttggaaggaaaatcagcagagggcagggaccggggaagaaggagtaagaagggaaacccttttcttaggttcttataaatcagaagagcaataaagaaaaaatttaaatctaaaaaatacatgattaggatttaaatccagtatgcttcactacatataaacctttgacttaatattttctgtagtagatgaaaataggactttttttgtttcccttctcttcacatggtattgagtttttttgtgttctgaatacgtgttaagattaattatgaaaatcaaattaccagatacttgaaatataaaattagtaatattcaaagacgattaaatgcacgatttccttttctcataaaaaaagaagcagacggaacttgtattctgaaagcaaatatggaaatcacattaaacatcagaaaatcctcaccaccgccccccacccccatgtgcatttcatttctctttggtctgagttcaagggagagagaatatgaatgtgctgctagctcaggaagcttcagttctaactttctaaagagctgggaatgtgcttgagtctctgttatctcttgtttgcatttttcgctactttgcctccatttgatccagggaaaacttcatggtagggatgtgagatgtattttgagggctaaaagaaaggagtttgaacaaaagcaggacagtgttccgaatgtggaagcaaagagtccttccaaggggatgtgcggccaacgcctctgttcagcgatagtccagctgagaccaggacctgtgcctgatgcccaggccaccccacaggacggtcctccagcacctcatttctcctttcatcagacacctcttcactggcctactgggagaaaacggagatgagtgaaaatgtgcatacagaacatagggctcaaagggaccctagcaccagtggtgctcaagcctgtcccagcattagcgtcacctgggagctccaaaaacatattgttgccaagactcagtccaaagcaagtgacttccttggcctggagtgagatgtgggtggccagggtcaggaaccaccgagtgggtccacctctgcagactgggaaacagagtctgggaggctgcttgttccctcaaggacctgaagtgacatggggaggcaggactagccttggggctcctaattctgtcctggcctgtattccagtgtgccgtggggcacgtccaatttgaatctggccccagtacactctcaagccctgtgttgctctggtcccacagcttctgcccttgactttccatataacagcacattggtggccagggctttgcactaacctacctgcctccaacattgtttttgaataatggaagctaagcagatgcatattaatgcctgcattatggagaagactgtctcttcacaagtaatctgtagttgttagtcttgaaagtgggttaattcattgtctttctagagaaaatgattgcatggcctaatagtcttccagttagtctgtgtggcaaggtagaagccaggagtggattcttagctcctgttgagtgctgctgtcctgaatcagagatcttctctcccgggccactttgtcatgagcaggattttgaaccaagaccttttcttcacctgtctcgggggccctcaccgtgggctttgtgatcccctgacagctccaaaagcctttatcctcatccaatatctggtgaacagctgacacctcctgggaatctttaaaattgttctctaaagaaaccattctgctgcaagtacctatttttagactttataaagcccacgtcaggttgtcgctgcctttctctctgtctgctaagcctgctgtttgaaaggcagcaacaggatcttttgtctttaaaagttcagccccagctggtgcattccccacttgaactccatagccctggtgtgctctggggtacgtctcttccaaaacccaaaccacaggcagtgacctttacaccccaggagttgaaataatacacctggaggaagaaaataacttacaaacaatgagcctagagtataaatggctatgttttaaaaccctcgattttccttgggcgtatttaacctgtacagctagactgttgtgagttttgggctctctagggggctgtccaagttcagaacaagtgcatctgctggcagccagcttaaggagttagcagccctcaattccaataacgaacgtgtaaatcagaattcaggtgtgttgaagtggggccttgcacacatcctggtcagcccagggtgatttcctctctggccagtggttttggcttttttcttcacatgaactcgtctgttgtaactaatggtcactgagtgacttcagaggtagtgatagctcatagtgaagcccgtgacccacagcggtgggcacacctcgatcccttctccttttcctccatccccacctttctctccttacttcatcccttcctctttccacattgggtcatccagcagaagtcaagtatcttctatttcatatctttattgaataagagttttacgatccatgttattgaagtaggtaaatggaatctcttatgtggaattctctaaaaaagaatcaaaactccagtcaaaccaaaccaagtattccaaatcgtttttagggaaaaaagatataattagaaaagactggagagggtatgcaaactattaacattatgttagggtggtaggagtataagttatatttgctttttctgaatttctaaattttcttcaattaacatattgttcaaagtatcaaatacatagaaggaagagtgtgcatgcctgttatctagcaaacacacttcatgcaaagaaactctcctaaggaagtatgaagttatattttatccttatctatccaaagagagtgtgtgtgtgtcatatacctaaagaattagttattaagatgaagcattgcttataatgaaaaagttgaagcaccctaaattagcttaataatggtatatctttataatggaatagtataaagccattagaaatgatgttacagataagtatttaatgttatggaaacattttataacatagaatgaaaagtaaattgtagattggtttgtataatttgatctcatcttaggaaacacataatacagaaatatagatgaaggtctgaagggtattaatgccctaatgtattagtagcattacctcagagaagcaggctcatgagtgtttattttcttttcttctaactaagtatgtatatgtgtaagtgtgtctgtatacaggagatgtacatacacttttcttgtgaagaatctgtattgcatttactactggaagggtttattttagatttagatattaaagttacctctttgagtgagaacctatgatttatacttcttagcttaccttttacctccattatttaaatctgtctttttctgccatcaaatagaagatgtaacagtgataaaaaggctgggtatggtggcttacacctaaaatcccagcactttaggaggccaggggttcgagaccatcctgaacatagcgagtgacgtagtgagaccccatacctacagatttttaaaaggttagccagacgtggtggtgtgtgcctgtagccccagctactcaggaagctgaggtgggaggattgcttgagcccaggaggtcaaggctgcagtgagtcatgaatacaccactgcccttcattcagcctgggtggcagagtgagactctgtctctaaaagtataatattttaagaaagaaaaaacaaaaactagtgataaaaagaacttacaacaaactgatttttaagtggactgttgataattataagggctgagatttttaattgccatttgtatcaatgaataacccccctgtcaccacattttctggtgtgtttggtgtgtctggttcctataggagagacagtaatgctatttgacatgctttattgctgattagaagcctaatctctgtaatgggaagctcagtaatgaagcttgttcagaattgtgttacagtcggttaatgaagctctgagatcaagggcctggcttttgatggcccctatttcagaatccagaggtttgtttctatttatttgggttttgcaggtaagttccctggactgaggtgatctgaaggttagctctgctataataggtaaattgctaagaaaaacattggccttggtatgtacaacttaaaattttattggtgagatagtcttcttacaaaacaatcacgttaagctagaacttggtgtataaccagcatatatactcctgccaccctcagtgtgtagccaaagttctaaattgcatgcacagataacaggcatataattggtgatttcagaaaagttatacctggtgggcagtgagaggcgagccctgatctgggaaaccccagtgcttcaaggggaagcaatctgggaaactccaatgctgggattgggattcaatctgggaaaccccattgctgacaaggtacatggtacaagcttgataaaaacttgcttaatgtcacacttgtaatcccaacactttgggaggctgaggcagctggattacttgagttcaggagtttgagaccagtctgggtaacacggtgaaactccatctctacaaaaaatacaaaaattagcctggtgtggtggcatgcacctgtagtcccagctgctcgggaggctgaggtgggagaatagcttgaatacaagagttggaggttgcagtgagctgagattgcaccattgcactccagcctgagctacagaaaataataataatttaaaaaaacttaatgatgatagcatttgagttggcttttcaatcctggtttcctaaatatgtgagcctggaccattaaactgtgtgacaaggagctgtcatcttaacatacagacattaaatgcattgttcaagatcagaaatgatttcttggttctgaggcactattgactgtgtgtaaggtgccatcgttaatttggtaacaaggggagacagtggcagggaagttccttttgcctgtcttgtgataggcttcctattttctcaaaggttaagatattaaattgtcttaagcaaagaaggtatttaagggcatcatcagggttagttctcaggtctcctgactcctagcttctctagtttagtgattcacgttagcctttcaacagtttaccactcactttaatggtttttcctagctgtcttgacatgcttttcactgcttacatggttagtttcctacctagctgaatgcaggcatctgtgctgtctgctgggttcgctggtggtgatgcagcccacaggtacctgtctctttgaggggagttcagcaaccatctatttatgtcgaaaggcactgtactgggagatgaagaacacggagttctaggcctccaggaatttatcatcacttgctaatttgagctgagcctggcacttgaacataggccatagatttccagatgtgaaaaaaagaaagaaagaaagaaaaggatcctttaatgtatagacttgtctttctgcctgctttcttcacttctgatcattttaaacaattgatatcctagtttgatgtgatcatgttccaacacagcacaacagcatgtatggtttggggcatgcacctgtggcagttgcaaaggtttttacagtgaagtggctaagaggagagtaagaccagtgtatagggtgtggatgaggtcagatgcgagactatttcttgatttgagtcattttatttaggagctatttctttggaatactttatatatgagaatgtacatctgattggaagacagaaggtactgaaatttacctcttgatcaaattattctggtataaaattaaactaaataccaatcatatacttaatttcacagacttgcaaataatatactggggtaatggaagtaagagacaggaccttttatgtggtatgaagttctgtgataattcctatcttcctttgtacctaaaagaggcaccctctcctgtcatactgcaaacagatgccactactacgtgtagacacatttgccagctgtggcacagctggtcacccagccctcatcacagctgcctgtaaaacccagatttattctcttccccctgatcttttttagaaattactggtatcgttttagagagtatccaacttacacacaccacttaaataatgaattttccaatttcttctttttccacggccttcacgtcatctggtaaatattcccctcaggttggcatctgcttctgagaccctgctggacctagatatattcaccagagatgggtgaaggctttggaagagtgaagtggttaggaacatttcttccgatatctcctgctcattgtttttgacctctctgatttccagtatattctctgctccagccacacagctttcctgaacagctaagtcttgctcaccggtcagggaccagctcaaaggctgtccctttttaaatttatttcctgggtcttctcctggcctcttccctgcactcggtggagatgcctgccccatggttttcagtagcgttggctcttagctgttccgttctggggaaggattggttgttttccttttctccatgtgtgttgtgaatttcattctgtgtcaccgcatcacgcgaggtgggtagggaagagaacttttacgctccccgctgtcgttagtggcctgattacgagtctgtgggaaagaatgaaggttggaaagaagatctggtgggaaagaacaagggttgtccttgagcagccaaactaagctgtgagaatgttaatgtaataaagcaggaattctaatgggctgcctgctacagaggagatgggagatggtgtagggctcatgactgtaaaagcaggagccaccagcagatggtgatgcggcctcttggcccaggtgggactctgcatctccccctcccatggacattcacttgataagtgttgggccggtcacccatgccctggtcgttcttatcttatcgaagagtgctaatataagtaagctgtaaattcccattgtaagaaataattgccttttaaaagggagcattcaagaaaaagaccttttggctcaataaagcctttattttcctcaacactcttggctcttaaaaggggcctgttcttggccttgaagtgggaatttacacagaactttaaacagaagaaaccccctctgccttgggctgcaacttgttgtccctggcagggtttgggctgtggggtcatgggatctgtttgtttgaccgttgtatgcaaaaatgattttaaaattggcctccctaatctgttggagacattagttaaaaaaaaaatgagagtaaagaggaaaatattttgctattgggtatgcactggatccatttttaaactgagagaaggtgaaaggggtgactggttgatttcggataatacttttagagccacggggaactgtgaaagacaagaaaatgtaaaaaaagcacattgattgcaggaccggtggtccaaaagcagcacgtgaaagaggtttcaaggctgtttctacaaacaagaatgccaaaggtggcataggacaagaagcttgctggatgtcacgaagctagtggagatgggatccaaagctagttttggctgatgaaaatacaggctcatgccctgtatgtgaggacagtgggacagtaatttaaacagaagtatctagaggagtttccttattgccttgaatgtatttctttttaacattgctatggacatttaggaaaagatgccactgatattccaaaaggggcattccatacacctgagagggatccctgaggctccagggtcacctgggccaagccaggtatacacacgtctcatggcaagttggctgttatcataacttccctggtgttgttttgttgcaagggcagcattgtggggctgtggtttgaattgcttttttgtgtctcttgaatacacgaagttcctcctatagtcattggctctgctgcctctgatgaggcacttccgtgttccccacatttgaatttgctcccgattcgagagaggcaggcatccgcggcagcagcttccaacccagacaggcttaagccgcgcgagggtgcccgggtgggtggctgcgcagggccctacctgggcgggagcggctgcagagccctgtccacgcggtgcctccccagcacggcacctcccactccaggctgttggttacctggccgggagctggcggagaccaggatttctcctcgccgccgttgctgttgttaggggaaacagaccatctgcatctagacctgcctctctctccgctccccacttcagacgagctcttcctgcctggaatctggtaagtgccaccgaaggttccgttccctttattactcgattagtgcccacagcatcaggagaaggttctcttaccgtgaatccattagctttaacccaaatgtgcaggattagggggattatttgaacttgcttgctttctttttaaaatgtccattagacattttttgcatgcttcaaaatcaaaaggcggcacagtgaaaacattttaattatttatgtatttaacctgtatcatcttgtttctaacctcttttcttcagggtggtgcagtcaaaaattacacagatattgctctggtgcctctccccccttttatgaataaattactgcatttgtcatcactgtgttcctgaaatgtcaaaaatagacactgtaaaggaatctgtcatgcagagttcagaaattttcaaatttctgagttcaaaacctcaggtgaagcgaggacagatcttttatttaaaatttgattctttgtaggcttcactgtgaaataagttcaatttagagcatagctaatgcaatcagccagctcgtgatgggacaacatacttctctattgttttacattcacctcctgggatacacacactttctcatttgttttataacctttacaggaaatacctggggatgagtcacatgacccctaacttgagtttttacatgtattcacccagattaaatgattttgctcttatatggataaacgtgcagttaggaactttaaaaaattgtaaatgaggtgcataatcattactcattgtcttttactgcattcttcagccctcaaacatacaaactgttattccctttaagagaaaataaaactggcacttatgttttcctttactctaagttaaagactacccatgttatgtgccattaaaaaagatgtagaaattaatctgtctttttctttgacctaatcttactagcagggatattggggctctacttttctgttctctctctctctttttttttttttttttttttttttttaacatcagccacaggtaatactctccggtaaacatataacttgaggaaaattgaattaggttaatgctggaatagaaaaccagggcagtgcctgctctttttggataattctgtataaacaatgagaaagtctcaggctgccttagacaaaggctgagacgggagctcctgatgatttttatgagggggaagaggagatggtgaggaggagggcaccgagcatgtgcagcaacttgaagcatgtcagaaagaacatttgccagaaatcttcaaaaccgtggcggtcggtaatgtgtgaaattgaacttctgaccctgtttgcttgatggcgacttctcgttgtgcttgagaattgtgtggcattgggtccctggatgtgggaggaacagcaaagtggtttagtgaatagagagggaagtgccttcctttttgggtgggacttgccatgatagagcctgggccgtcaccttaaaaggagatgtttctgttgagagatggaaaattggcaagacaaacgccataaaccttaacctgtttataattccttacctgtaagctgctttgcacgcaaggtgatggttctacctcataagggacaaggaagaacagcacataaacctgactgaatggagtgtggctgtgggtgggagcagctttacaagaaggaaacggggccccctcctgagacacccccgtttgcttctgctccttccttgccattgacgtgcccttctcctgaaactacccacacctgtgtttggcaacaagcggctgccccttgatccagatgcatttttcaacttgcgaactctcacctcttgccaagggacccaagtacctggttaatgatctttaatgtaaatgatcctttgccttgattaacattgagctgggttagtatgcacaggccgagctccgtggtcctcccatgtgatcccttccctgccagccacctgcccacctgtaggaactgggcatggccagttttctctttatgtgctctctttgccttggatggaatttgggctgagaccgatgaggtggaaccaaagctgttcagcagaagagagcaaccacagagcagcctgttgggtggagggcttgagggttcctaaaatccgaggactttctggtgagcctctcccctgagcctgcttgagggaaggcgctgccccacacactctccagtgggtgggggtgggggtggcagcggctgatcaaaggttctcacctgcaagtgataatagcctgggtctctttggcaccaacctgccttcttcacctttctgctcagacaccccttggcacccctacataaaggaaagagctgtctggatggcctagtaactgtaggataggtaaagttagccctttcttctgacctagatatgtgatggtggtatttctacatgtttatgtcgagaactttacaaagatgaaagaaaagctttcagagctgcagaaatgagaggtggcccttttttaaaatttattttaagaaacaacttttaaaaccactttttgaaaaattggatggcaccgtcctgcagcctatcagatttcccttgagaactggggattattggcttattccttgctacctccaatcccaaattaagcagagtgacatgtaagaagctggggacatcagcctaaagcgtttatcaggtatgtccttcccaactggatcttttctactccctggcccgaattttgttttagggaatggggcctattttaattgctggtggcaaaaggatccctgggcagtgtttccatggtcctctaaggtggagtgtcggggagctgggggacacacagtggtcatgggttatctgtcttggcctctttcccagaccaagttctgtctcatgcttattggtttggctgctagttatagaatctgatgtttaccttagaatcctaggatggaaagggttaagaatgatttctaatttgaattccttgtttctagggagttattaaacctatacattacatttattattttacaaagcactaattcataatagttgacaggaacaactattttttggcttctttgtttttttgtttgtttgtttgagatggagtttcactctgtcacccaggtctggagtgcagtggcgcaatctccgctcacagcaacctctacctcccaggttcaagcaattctcctgcctcagcctcccaagtagctgggattacaggtgctagctaatgtttgtatttttagtagagacggggttttgccatgttggccaggctggtctcgaactcctgacctcaggtgatccgcctgcctcggtctcccaaagtgctgggattacaggcatgagccaccacgtccggccttctttggttttcatattgattatattttctaagacaaatactcatttatatagtctaataaaaattgttaaatctaattaaacaactaataaaaatagttgtttaatccacgttgacatcttcttacattgcgatagaaattttgatatatttatccatatgatgaattgcttaaagccggtggttctcaaccagtggcagttttgccttctctcccacaagggacatttgacactatctggggacatttttcattgtcatattcaagggagtggggtgtgaagctggcaagcatctagtgtgtagaggcctgggtgctgctccactttctacattgcacaggatggcacctgacacacagcctaaagcgtcggtactggttgaccgtatacgaaatgctgtggaagaccaggcacggtggctcacgcttgtaatcccggcactttaggaggctggggcatgaggattgcttgagcccagcagtttgagaccatgtttaagtttaagttcatttaagttctctgaagtggtgattctgatgctatatagatgaaggttggtttgttgaagagctgactctggtatttcatccaacatctgcatttttctttagctaaggatgccttgagaaacattctagtctcttaggcctgaatttgttattagcttaatagctttcctctggttgaatttaaatggaaagaaaattgcatcttgcaacaggaaaaagaacattagggaaaaactgaggaaatggaaataaagtgtggaattttgttaataataaatcaaccttgatgcattaattgtgagacatgtcctgaaccaatgtagatcttaataataggggaaactggcttcagggtacgtggggactctatcttatctttataataattctctaagtctgaaacaaaaaagtttattaaaaaagcacaaacaaaattgctgccattttgttaacagatatagtagtcaatcttactgttttctttttctttgaaataggctgctgcaaaggtatgttctttgttatctcggctctctgctcagggtagccatgaaatggctcttttaaaaatgtgcccagataattttgaaccatttcaaaccatattatgtgattttaatttattttccattgcattcactaactcatttctgggtgatgtatttaaaagactagacagaatttacaggtttaaaaagttacattaacagaaaatgtaggtgccagttgaaatactgtcaactggaagtgctaccagcaatgtgctagctggacaatgggacttgtcccatttttttgctgggtgacagctatcactgcctcagggaggctggtgccatttaatgcctctgataatcagaataagtaattgtggcttctgggtctgtaaagccacaaaatacatatgcaggcctattttaattgacggtaagtcatgaaatacactattttcgtattaagataacgtggatatgttatggaacaaagttaaaaattagcatgaatttttaaagtactactggagattgttggctttaggttcgtgaaccccattcctgcccccacactcctgttagtggggctaccagaggggccagtatgaaaagcactggacttacagatgaaagaacttgagctggaatcaattcgagtcacttaccttcgctacaggtgggcagaggagtcatcctgcaattttgttgtgaaaacagaatgagataatataaaatgtatttgaatacagtgaggtctgtacagatgctgaatctattactttgcagtaggagtcaggtgtggccccacttccccaccttcttacctacttgtatcttagatcttatatatattgatattcacttttggtggactaaggttgtctttgttgcaaacctggactctcttgtattcttgctcagtgtgtgtatgttaatgttagataaagaagattaggctggtgcattggctcacgcctgtaatcccagcactttgggaggccaaggcgggcagatcacttgaggtcaggagttcgaggccaccctggccaacgtggtgaaaccccatctctactaaaaatacaaaaattagccaggcatggtggtgcctgcctgtaatcccagctacttgggaagctgaggcaggagaattgcttgaacccaggaagcagaggttgcagtgagccgagatcacgccactgtactccagcttgggtgacagagtgtgatcctgtctcaaaaaaaaaaagcagaagattatttggaaagctatttttaaattattcttcaaacatatactaaaatagagaagacaacatgagagccaacagcataatgaaaccccatcactcagcctcaaccgttgttaacagtttactatctttattgctactgaaggcagttaaatatttcttggcagtccttttttatttagcatataccatcagggatgacaatagaaacaggtttcaaagtcaggtgaaataatgtttctctatatgagttcaccactaacttgatacacagtttatgtttatgtttatgttcttattacgttcgtgtattaaggcaacctttcatccctgccccctaataggtaactgtctttatttgttctgggattatccctcctccattgccctttttggaaagagtatggatatatttgtattttctcttactcataaagcagcacatggtacatactgttctgagccctggttttctttttttcacttagcgttgtatactggaactcactcaatgtcagtatacagagaattcccttgtctttttataactctgtgctatacgctgacggaagttcattcagtcagtcccctattgatggacacttgagttattctcagacttgtccattgttttgcaaaaatagccctgggcatatgtcatttggtatttttgtcgaggcattccaataaaaataagtaaattcttcctctcttgccttgaagatgtgcagctcctcagtaaccttttattctctaaagctctgtcctcatctttaatccctgatttaagaggtgcagctcttcaggccataaacctttcttcctgcctcaagtttttaaagctacaaaggaacctaagagatcccttgtgttttacagctaaaggacctgagagcaggaaacaaagggagagccggaagcccaggtctcctgttccctgcctggcaggtgatattctttctgttaaactctctttccctctgcctattattggccaggctgtcctccaggacagtgcttgccaaagtgtggtccatgggcagcagcatccccggagctcatcagcaatgtaagatgtcaggccctgccccagaggggctgagatagaatctaccaattactggattccctgggtgactcatgcagtcaggttggagaagcgctggactggtggctttctccagaggagcatgcaacagcggtttgcaaacagggatgatgcagttccttttcaacacgtggatttgcacttcctcctggtgcgctcctccagcctgcctgtcctgttcacaccagcctccccctggaaggggttcagacttgatcctttgggtggtagggtctttcttggatagcgtagccatccattcctgctgcatacattcaacacagccttcatccttcgccatttgtctctgatacttcatactccttttgtttgacaagtgccctagctttatttattttctcatcattgttgttaagggcctattgtgcttgctagtatacacaccactatgtaagttactagttcaatttatttgttttactgctgctgtgaggtgtgtccatgtgtgcacatctacctgtgttaataactgataatattgggcctcagtacatactttttttttttaagagacaaggttttacttcgtcacccaggctgaaggactgagacacacttgtagctcactgcagccttgagctcctggacccaagtgatcctcccaccttaacctcctgagtagctgggactacaggcacacaccaccacaccaactaatttttttattttttgtagagacggattcttactgtgttgcccaggctggtctagaactcctggcttcaagccatcctaccacctcagccttccaaagtgctgggatgataggtgtaagccgccgcacctggccctcagtgcatacttagatatacttaaataaaaacttaattgcaaattacttcttaaattatccacaaactgacttggcagtaaaagtgactacaagtagtgaaaccactgtcataataaaaataaaataaaagtctttattgttagatttagtatttctaatctcttacatttcagttttggaaggtaaatttttgggaagtggtggtaataatctaagtatgtctagattcagtgatgagagctagtacactgtaggaaagagttgagcataacaatatggaatgttggtaaatgtataggggttttctttttaaagacagctttttatacaaagaaacagatgatctttttttttttttctaaactatgttagctatacccaaattagtaaatgttaaaataaccatgactggtaaaaacattttgcaattttttttttttttactttttatataattaaatgaaattctccatttggtagttgatctctcagacaaattaagtcttgccagactgtctgagaagactagtgaatcagaccctaaaaacttgtagcatttgaaaaataacaactgtgtactgcaatttataagggcataattagagaaagtttttcagtctgaagatttatactaatcttatcaccctggttttagagaacccagttcaatccaactgctgtgaaaaagccactgcttttagccacagaaaataaccagtttcatagaatgacttataataaaaaattcaaccattttgactccaaaaacctagctggccgggcgtggtggctgacgcctgtaatcccagcactttgtgaggccaaggcgggcagatcatttgtggccaggagtttgagacctgcctggccaacatggtgagacccccgtctctactaaaagtacaaaaattacccagacgtggtggtgtgcacctgtaatcccagttactcgggaggctgaagcaggagaattgcttgaacctgggaagcggaggttgcagtcagccaagattgcaccactgcactacagcctgggcaacagagtgagactccttctcaaaaaaaaaaatgcctagctgactttatcatagatctgtctgtaccttgtaggcacaaatatatttacaaaataattaatggtgatttacttattcacaataagcagttattttgcttttggtgtatatgtgacaaggtggttatgtggagtacataaaggagtaaagtaagtccgttgacttcaagatgcttacagtctaatagaaaccttgaaatttccactgggcaaggttttcttgggattgctccaaccaaatgaatgagcaggcgtctaagtaaatgaaaatcactctcatcactttccttcacattatgttaactggtgacaggtcatgacgcatgggaattgcttatgtttctgagctttagtcttataaaaataagtttagatagtccatcatgttcttcacattagacactcattacataaaaggttttatatgtattgtagatctttctgaaggtcatctatgtgaatgtttaaaaaagaagcaagaaggtgctataaaaagcactagaggtcaaatggtttgcgagacaaaatgaaactctccagctctgaaatccaacagagtatgatgaagaaagggaaaaaatagatcaaaaaatggcaggggaggaactcggcatcaaaccagtgataaagcagaatgtggatcagatccagacatccagtagtctgtgagcatctccatatgggatgtttgggttgagagttcaacccatcaaatacattttatccataaccataagatattctaagatcgaccaagtagagtaagcaatgtagaatcaaaggtatcatctttattatgtcctatttgtgtttcctgcttttgaggttcttagtccttttaattaactcctaatgaactaatatagttttgtggtcaccagcttctagctagcacaggcacagttgtgggttggtaggagaatgagcagtaaagccatgagctaacacaccaagtaatctgttgtgttcacaaatgtccaagagaataacaagagattaaagaaaggtaaatacagccacctggcaaaaatatttacccttttaaaaatgatggaagttatatatagaagaaatgtttttggtaaaaatatttgaaagtttgttttataatcataagtagcttgggctattttgactagaggggtgtgtgtgtatgtgtgtgtgtgtgtgtgtgtggttggcagggagagaaggagggaggcaggcagctggtagggactgtggagtgtgtcagttgggagagggaaatgagatgattaagttagtaagaatagaaaacatgtaggaaggcggatcttgaaatttcagggctattcattagtcattagttgaagatttcacagagaaatggtagaaaccaccagtaattaagaaatagcattattgtttaaaaaaaaaaaaactcccaaaccctttccctggcattctggtagaatatagttttggtctaacccaggtcccctctcatttcctttcacatccccttggttctagccacgctagactgtctgggttgtctgtatatgttctgatgtttgttttcctctgtgctgttcttctggcctcagaatcctagtgtccttgtgcctccttctgcatatgtctggactttgcttagctgatgagatccagttcagataccaccttctgtacaatggcttccttaattcctttaatttctcttaattgcaataaagctatttgcagaggaatgaaagatgctcccctttgcgctgggtacaggatgcactgaaactttcagcatggggcgcggtggctcatccctgtaatcccagcactttgggaggccgaggcaggcggatcacctgaggtcaggagttcgggaccagcctgaccaacgtggcgaaaccctgtctctactaaaaatacaaaaattagccaggcatggtggtatatgcctataattccagctacttgggaggctgaggaaggagaatctattgaacccaggagacagaggttgcagtgagctgagatcgcaccactgcactccagcctgggtgacatagcgagactccatctcaaaacaacaacaacagcaacaacaacaaaaacccaaactttcagcaggagagtagcttggttcagcccagcaacatcggtttgaagattatatcaagttttccagtcaaatgtaaccagtttatggatctgtgtttgcacggggtctgaacctcccctcctcaggtataagctcttggaggacagcctgctgcttaaggtacaggagaattcatctttgctggacatggtaacgtatgcatagagatctggcaacagaacttaagatatgtgggagaaataataggcaaaaactggaaagagcacaagtaagagaagagaaatccgcagaagtgagaagtaacagaattgctggctgaggttggaggcatttaagtgtgtgggtgtttgtgggaggcatggtggtctcctgttccttgacctcattgggcaaatattacagcacgaacatacaggtaggaatcaattgcatccccacctgggcaccttcatcggcagttggggtacaccacttgctacagtgtgatggaataagggacgatccaaaggcactcagttctcccacccatcctgtgcacaaggaaaagacctttgaattgtgtctcccctgttgtcctttgcattgtgaaagtgcatattagttctcccatccactcttttctctttaagctttagcatacggttgtttgccaagaatggaatcagatagaatattttcaccaggaagtgaaatttaaagtgcttgaaaaagatacaaggatatctggaagttccagccggcgccattttatattttaaccatgacaagtagctgtatatcacggaactttggaaaacagagattgttccatttatttttcacagatgaaaaccagagtgctgtgtggggatcaggaagagggaaatcaaccaaataccttattttagagatttttttttttttctgggaaattggaaacagcgcttgtttatagccaatgcagcgtccttgaaactctcctacttacagttctggagctgtagtttcctccctgaagaatgggttaaggcaggcagggtaatatgcatagatgggagtgagagtggatggcctacagggttgttaacctctaagtgcaacaggaatatatgaaggtacttgggctggtggtcctgtgcagaaccagccagtaggttctaagagacttgatagccgagggtccctgggccctgttccacctttttaaaatttctatgacttagtagcggcagtaccatgcccatcccccagtgatgtttggggtacagctgcacccttggccgtgcctctgcactcactcacgagtgtcatctgacatctggctcttgaccaggatctgaggttgcttaagcaggatccaggtcaggttttttgttcatggaaggtggaattttggacctttcccccaaaaagttgacataacttaaagagaagggaggtatattgagaccagcctggccaacatggtgaaaccctgcctctactaaaaatacaaaaatagctggcatggtggtgagtacctgtgtagtcccacctgctcaggaggctgaggcagagaatcagggggcggaggttgcagtgagccaaggtcatgccactgcactccagcctgagtgacagagtgagactctgtctcaacaacaacaacaacaacaacaaagagcagagtggtattatttcctcactggaacattaaatggttcctactaccaaagagttcctgttctaatgggggtttaatataatagtaacattttattcactgggattcactcgggggaaagtattcaggaacaaagtgacaaaattaaatataggacatatgaacattgtcattttggttctagttcttacaagggcttatggtagcaacccaaatagaagtatttgctctaactaacagataaattattagcacttcagttgtttacatatgagtcagagcatctaaaatttgttcccataagtaggttgagcctccttctcagcctgttgtcatagggtctaaaaatggaaacgtgagtccaaactctttccagatcataaaaataattctgaattagtagagcataaatgaatgtgattttattcgagtgtcattttcaagttccccaggccaggttgagaccttttaagtcatccttaattccctgtagggaggattaagaaaatagtagcagctttgcttctggccttagacagttttctaatgaggtcagagcctctcaccctgggagaagcagattcactttgccccttcagcattggtttttgagaattgctgtctgaagctattgcatagacaaggtcactttttaaaagggtacatttctgaacctggagaaggagtgggttctgaatgactggctcttctgattaacagagctgtatatcaagctgtgctgaaaatactcctcagatatattaggccacatttatgcatgagtgagctcactgtcgtgacatgggagtgaacctttgctgattcgagatcctggggtgccttcactttggaacactgattctagatatttaatagcagttttaaaattaggctaaatgtagtctgactgcaggattcattccagtgggaatttttttgtttgttggttagttttttaagcctgaatatgaacccaaacatgtttcccaaaatgtgaaatgaagagtgaaaacctaacaatgggataagaaagataatagataagagattatagttcaaactcggcctgcactcaggaaatggaagcaattcctccccctcatcctgcccccctcctcttcctccacctcctcctcctcccttacctcctcctccactacttcctcctcccccactwcwtcctccwcctccmcctcctcttccacctcctcctccccctcctcctccccccctccccctcctcctcctctccctcctcctgccccctcctcctcctcctccacctcctactgcccccctcctcttccacctcctcctcctccacctccccctcctttacctcctccttctctttctgcacaccccacccccgcccccactagggacagtttactcttccccctcctttcttagaatgccaacaactagcctgttagttccattacttacttgccttgtgactcataacgttttcatcttgggtatttttctttcttggtctttgtaaaaattctaagcatcatagagcatatcacctggagaaaagacctacgttcattatttaatgaaggtaraaagtaaagaattgagtctgcatcctaagaaattagaaaaagatcaactaaktaaacaaaaaaatataaaaactaggagggaggaattaggagatgataagatttaaaagttatgtaaatccaatagctggttatttgaaatgaccaataaagtaaactctcattatcctaactcaggaacagaaagaaatcatcctaacttagaactagataataggaatgagaggccgggcatgatggctcacacctgtaattctagcactttgggaggccaaggcgggcggatcacctgaggtcgggagtttgagactagcctgaccaacatggagaaacccatccatactaaaaaaatacaaatttggccgggcrtggtggcttatgcctgtaatctcagctactcaggaggtgaggcaggagaattgcccaaacctgggaggcagaggttgcggtgagccaagagcactccattgcactccagcctgggcaagaaaagcgacacttcgtctcaacatcaacaacaacaaaaaagaaagaaatagataataggaatgagaaaggagacacaggagatatcaatagactttataagaaaacattacatgcagccctgtggcaacacatttgaaaatatagggctgacttcttaggaaaatacaaattaccaaagttgatccaagaagcatggaaaagttgaatagaccaattactatagaagagataggaaagatgattaaaagttaccattgagaaaggcaccaggaatccatggatttccagctgaggttttgtttgtttgtttgtttttggagatggagtttcgctcttgttgcccaggctggagtgcaacggtgcgatctcggctcactgcaacctccgcctcccaggttcaggtgcttctcctgcctcagcctcccaagtagctgggattacaggcatgtgccatcatgcccagctaattttgtatctttagtagagacggggtttctccatgttggtcacactggcttgaaaaaatagaaaatttgagtgttatttttactcttctaggacatactaaaacatggaaaggttcccagttcattttataaaaccatcaaaattttaatttaaaaatctgatgaattaaaaaaataaaaataaagatcaatattatttatgaaagtaaatgtagaaatttaaaataatttattaacaaatagactcttgggtgcatcaaaacacgaatgcactataatcaatagttattttccagtaacaccagggtgattaactattagaaactcatcactatcatcagttgttacacaatcatattgaaggagaggaatatatgaataagtcagtagaggctagaaaacctatctgaaaaaagttttagtagccattcctaatatagtacaatctgagaaagtgaactaggaataaaaataaattacttaaacataaaggccatgtatgtgtacacaaacacacacacaccacagcaaatatttcctaaaagacaaaacactaaagccattcaattaaaattaggaatgagagaagaatcttacattttacaattagtatttaatattatcttagaggttcttgtgagtggagtaagacaatgaaatggtcatatgtctagaaatcgcaagagcatctagtaaaaaatctaaaatcaacagaaacaattgtcaaggaagctggataaaagataggtctctagaaattatcttttctgtgttttgaagataagcacctagaaatggaagttaagtatttctttcacaatagcagcagataacctaggaagacattgaactaatacctgtaaccttcttagtgaagaatgtgaaatataatctagagaaattaaaacatgtaatgcctgaatgtatcttattacaaaattaataagtaaatttcttgtgattccggttagaatttccaaagtggtgggttttctctttaattgaataaaataatcctgaagtctttgtagaagaataaatacgtactacctgagaggagccaagacaaatacggaaaagaaaaccggtgagggcagatttggcttacaagtaatgacaacatgctaagaataagaagtcttcgtgatcaaagcaatgtgttattgatccagaattacataaatagttcagtggaacagactagagaatctaggactgtgtctcattaagagagtgaatgtataacaagatgatagttcagtactgttgaggttggtactggcataaccagttctttatctggagagggggttgggggaacagcacagacctctatctaatatcataatcagtaataaatcccggaatcttcaaacacttaaatgtaaacaagcaattcaacttttacaagaaaatataagaacctattttgtataatcttggagtgagggagaatggttaagcaaaatttgatacgcacaagctataaaaagagacacactttgaacatatattttatttttatttgtttattttttgagacggtctcactctgtcaggctggagtgcagtggcgcgatcagggctcactgcagccttgacctcccagactcaggtgattctcccacctcagcctcccatgtagctgagactacaggcaagactcactgtattagttctcatactgctaataaagacatacctgagacggggcaatttagaaaggaaagaggtttcacagtttcacatggctggggaggactcacaatcacgggtgaaggtgaatgaggagcaaagtcccatcttacatggtggcaggcaacagagtgtatgcgggggaactcccctttataaccatcagatcacatgagatgtattcactatcatgagaacggcatgggaaaaacctgcatctgtgattcagttacctcctaccgggtccctcccgtgacacgtggggattattacagttcaaggtgagatttgcatggggacacagagctaaaccatatcagctaccatgcctagctaattttttgtggagacgaggttccaccatattgctcaggctggtctagaactcctgttctcaagtgatctgcccacctcagcctcccaaagtgctgggattaacaggcatgatccaccatacctagcctgaatatatattttaatatatttttaaacttttctgtggcaaaagatacgtgattaagtcatagatcaagaagggatatttgaaacagacatggggttactatctataacaaaaaacttacaaattgttaagaaaaccacagaggaaaaatggacaaagaatgttaatatagaatacacagataacaaatgcagacagctggcaaacctatgaaaaatagttcatttttgctagggaaatgcagattaaacaatgagacgttactttaaacccatcagattggtaaaaatagagaaaaataacactttttgatagcaaggctgtgatagaagtggcaagtggtattatttctcataatactgatggaaatgtgaattatggccttatttgaaaagcagatgggcaatgtatattaaaataaaaaactattatacctttcttctcagcaacctttgtccttggttatttgtcttttagaaataaaaaatgtgtgcaaagtcatatgtataaggatgtttattgtaccattatagtggcaaaatactgaaaatatgaaaacccatcagttgaaaaacggccaatttatggactgtttgtaccatgtaatattatgagagaactagagctgtatcagagttggctgtggaggaatttctatgagaacttgatgaatgagaagcaatgtgtagaaaaatgtataatatccagttctaaaagacgcattcctcctgtgcccatatgtgtaaatgtgtgagtgtggagaacaagatgaaaatgctcattctgtatgttaagcagggagtggggtggaggttaatgtggaatggtgggagagtggggtggcagggatttccaagtcactcaccagtgatgagtgttaaccacatccccatttttttcctatgaaggaagctgcaatgctgtggtttgaggttaaacatgctggctccttctcttaggtcttgatttggtttttgttgttgttgttttttttttaattctagtctgttcttgttttgctgtgctgatatcctcttagattttacgagcatattaattgtgagtatttaacattcttctttggtttattctgagaccctgtttgcctgccctttggcatcattggcagtttttggtggtctactgatcttcctgtttgtgcctcctggttgcccctttttcactggaatgtgccaggttgaagcaatatgtagggcccggtgggatgggccaggaggtggcctttcagatgtggctcctgcatggccctgcttgctctcttgagctgcctgcacttcagtcttaggaccacattcctgctttggcctacatgaagtgcaaggtagcagtgggaaaagcagagaggaaatgaagcttcctgataaatcacctgatcttgccccagggttagtcactctagcccccttggatctcttgactccatctcttgatttgtttcttttgaatttctggcatctggagaatacttgtacagcagcagtcccctggttttgtttttctttcaacccaagcccatcccatatctgttcatgtgatgatactgtgtcttattttcccaagattcttatgactgtgatatttagtctgaatcagagaaagagagagatagaaatgtacactcagagggccacttggatggaaacttctgttgatctctccaagccttagtgggatcatctgtagagcagggataataacagcgcctatctgttactgttagggttactgcaaagattaaaataggtagtgctcttaaaagacttagaacagtgctgtcatatagtaaggattccataaacattagctgctccggtttgtgtgtgtgtgtgtgtgtgtgtgtgttttgttgttgttgttgttttaaacgggatcatgctggaaatactagattgcaacagagctttggggttttttctctaattaatagatctaggatgttttctgaggtgtgtcatgtttttaaaactctgtggtatttctgtttctttcttttctttcttctttctttttttttttttttttttgttgttgttgttgttgagacagagtctcactgtgtcacccaggctggagtgcagcgatgccatctcagctcactgcaacctctgcctcccaggttcaagtgattctccttcctcagcctcccgagtagctgggactataggcatgtaccaccatgcctggctaatttttgtatttttagtagagacagggtttcaccatgttggccaggctggtcttgaactcctgacctcaagtgatccgtctgccttggcttcccaaagtgctaggattacaggcgtgagccaccgtacccagcctggtgtttcgtgatatatctatgtgtgagggtttctgtagttaagttcccatagttgaattcctggttcaaaggacaagaacatttacaatgttcatagacgttgggaaattgccttctgtagtactgcactagtttccactggctgtagtggttgatgagagtaccagtttttccctagttcaatattaacccattttatcagtcttttcatcttcgacattatggaaggtaaaaacatctcattttatgtcttcagtcagtgatgccaagtatctcttcgtgtatttatatttccatttttatccagtgccaactcacatccttctgtcaatttttcctttgacctattaatttttcattgataagaattctttgtaaaatgggcagtttaactccccatctgtcatatgttttttctcatttgatttattgtatatcttgtatagcaccttttaattttaacattgtgtttttaagtgtcttttctttgttgacttggaaagctaacaactacttacagcataatttccttctgttaaaaacaagaaggaatttccccactctaattgtgaaaattatacatttttctaagtctgaaaaaaaatagaggaaaaaacagaaaaaacaacagaaagaacatcagtcactcttaatattttggcattttccttttagttgttttttaggagagaatgtatatttatacttcgcatcataacatagatatagtctattttggtgatttattccaatatgtaatatatgtttctgaatgtacaatatcctatttatcatatctgttaagtttgtttccagtttttcatttaagagagaaaggtactgggaatatctttgtgcaaaacaagaatttttttttttttttagtcagattctgttcctatgatacctccctggaaatgggattcctaaattaaagaatattaggatttaaatacttttggtatagattaccaatttgctttcctaacgtgtggtactagtttatgttgcaatcagcacaaagactcactgtgccatttatcatcagcagagaagagagcagccctgaagatttgggggaggagtgttcgtagagaggcacgtgaaagtgagtttgagaggtttgagaagtagccaggtcagtggggctggggaagggagagagttcatgggggagggctggagatgaaggtaaaggagccatggttctttagggctgtgtgggtgctggggaatttgcatttcctgcttaaggttgtagtaagtcagcctggagagttgcaagaaaggagtggcttggtgtgatttattgtagcaagtgagagtgacccagaggccagttaggaggctgttgcagaggcctgagtgagagaggatggtttaaacaccaaaatggtaacattcagaatggagaggaggagatggtgggagattaaatttggggatggagcccttaggatctgttgattgactggatgtgtaagagagaaagggaagagcagaactgaggaaatgtcctcttgaactggctgggggccactgagatgggacagatgcgctggggagcagtgcagaggtggaatcactgctccttttaggtctaaggcatcccttagatctccaagtggatgtgtgaagatggtggtagaggagaatggagctcaaaagaggggccagagtcatttccatttagatggtctttacagcctaaggaactggcagatttcactaaggttgaggtctttctttcctgtgaaaatgtgatcaggatcctaagtaacagccctaaaaattaaacattaggaatacaaggtttcataaattaggaactgtctgtagctgccagattctaaatgttttgaggatacaaatcttggggtcttttcaaatgctttgagcagaagaggtacatcgtattgtgtttactgaatggcatttaaagatgcaggagacctttcccccttcttgatgaaacaatttgcctttcagatgttttgttatagtgagttgattgtaggaccttatgaagcatgtaaaccacacatttgtaaggcattggaagaaaaagctaacgcgagttaggagggatttaagaccatgtgaggaaattcaagcctatgttgttatcaccaaaccttgcctgtgttatgtaaagatttttgcctgtgtcatgttatcttcgttgtgttcaatttattcctccccctccaagtgtcctagtttctcctctactgtagctggttttgaacaaaatagagaaacttgggtctcctctaccctcagcatctgaattcttctcatttggttactcactgtgttaaaataagtgatatagtacattcttagcagtttgtttatttttaattttattttttatggggctagggtctcgctctgtcacccacgagtgtggtgccatggtgtgattgcagctcactgcagcattataactcttgggctcaagtgatcctcctgtcttagccccacgagtagctggtattacaggtccatgccaccatacccagctaatcttagcaatttttaaataacttttttttccgactgtaacagatttagatattggaaaagataaaacataaagcattgatctacctgtagagccaaatagccacagggatataaattggttaccatttaattttatttccatctatatttggctatatttatatttatgtatatggaaatagtttatgaatttggaatcatactatgatattttttagaatgatgctttttaaactattttatggtaagcattttagtaaagctcgtaatgttatttttaaaaacgtacttttcgaaggctgcagagtatggataaattcatgatgactttttagaggaaaatatcaagatgatctcactaacaaatcctgagattaaaaacaaaagcaaacaaaatacaaaaactgaaccatctttggaatactatgaaattcagattttatgttcttaataaaatcctgttttgaatgaaatcagtcttcatgcttttcttattccttttggctatgttgcctacagttgaattaactgtttttatatacttcctttgctttgcattgaaacaagctgatccattgtgaccctttgtggtatttcataaaaccacaccctaaaaactgcccagtcttttaagtgtagtcaacagtattgagcaattttttgggtaatattgtcggaagtgttaggtgccaaccaacaatggcttattgcttacatttaagtgtaagcaatatacaattaaaggaatgtcatgactttttaacttcattttgaaatttgagtttagaaaaaattccaagggaaatgagcaagaatgaaagcatcctctcctactgccagacttctgggagacccacccctggtcttcccaggtgtctcttcagtgaccttgttggtcgttgagggctgtggaaagttctagggaggctgctcgtccctcctctccttttgcttattgcattctgcccagattataaaactgaagtatctgtcttaagacgccagcagctttttcttcatcaaatccagacacatcgaatcccccacaaccaagaattacccagccacaaaatgtcagtagtgccccggtaaaagaaagctggttttagacttagtctcagaacctggccatcttccatggattatcccgatatgtgcatccttgtctcttttcactatagcctggtgatcatggtggcagttttcattttgggacactggtgaggttagaatagcttcaggcttccctcagtgaatagtattgtgaaggcagtggtggtaacattgtgttggagctggtgtgagggttgaatccactctgtagcctctgtccctagatcatggcgctgctttgattgggccctaattttctcctctagacaaaaggaattgaactaagacttccagcattccctccaggcttaacattttttcatttggtgctgatggtcactgactattttccagttacattttaatgcaagtttggcagaagccgatttctctgggttagggaaaatgacataggtggcagggaggtggatgtacaccatttgtttgatcagtttgcctagaatggaggtggcagaattggagttggaaagatcaaatgtagcctttttttcccttccagaatgaggcttttctatgcatttttttgaggcatgagaacctcatgggacaaggcaaaagtgagcatactcaagtgaagtgtatggttatgggggaatggttattagaaatgtcaattgtctcaattttttagtattaattttcagagggaaacagtggtgtaattagtgttgtgattgtttgccagggcggtcgtaacaaagtagcacagactgggtggctcaactaccacaaatttgttttctcagggctctggatgtccggagcctgcgatgaagcaggattggcttcttctgcggcctctcttcttgacttgcagatggctcctctgggtcttcccaggtcatttgtgtacctgtccgtgtcctaatctcctgttcgtataagggcacagtcatattggattgggacccacctactaacctcattttaacttaattacctctttaaataccctctctctaaatacagtcaccatctgaagtattaaagacttcaacttaggaattttgggcaaatacaattaagcccatcacaatcaggtaacatttggtaggacgtgtagatgctttttatctttaagggaataggcaaatcgttaaaaataaataaataaaaacttgttcaacttgtttaggtacattttcttatagtgtattgtggttcttatttttttaaccttccagctagtgtgtaaattggaaacattgtagcttaatttgatagttttagacataatcaaagatgatggaaatcttgttcttaaatttagtctattcagcaacttgtgtatcctcaactgctgaagactgaaaacttgagatgttcctcttgttttaagaatttgactgaaaacgggctgggcacgatggctcatgcctgtaatcccagcactttgggaggccgagatgggtggatcacttgaggccaggagttccagaccagccttggcaacatggcaaaaccccaaaaatacaaaaattagtggagcgtggtggctcacacctgtggtcccagctattcaggaggctgaggcacaagaattgcttaaaccccagaggcagagactgtagtgagatcgtgccactgcactccagcctgggtgacagagtgagaccctatctcaaaaaaaaaaaagactgagaatggttggcactgtccccctgtctccaggagggtgaatgtttctagctgaaccctggtgcccacttgctcctctaagccttcagcagtcctcccagcctcaaggggtgatccgttcatcttccccccacctctggaaatcccagcgtttgaacgctggggggaactgcaagtctagtttacagggtttctcagctgagatgccaggaacacttcgcgagtgagcatgccaccacatttgagtgcatatagaaagttagctgtttgctacaactggggacacttcctattagagccgcatctgctggggtggagggctgcatgggttggagagtagggtttgacttcttccagcaagaggaaagagaagtagcatagtgcccatcgcaggcagagagcacatgtttgacttgctcttggaatttctgtagagtaagggtggtcaggttgccagactacccctcaaacgaggacatggtgctgctgggtcccagggtagagctctgctggcaccttcaaggccctgattgacacctaagggcacacaactgggatctgaaaactgggatgcacagtagagactgcagatttacagggcggggctgtctctcatctgactattgattcttttgggggctgtagtttgtatcatagttatttttgttcctgtttttttcccctcttcaagacaataagcttttggagggccaggaagttgtgttcccatctgccgagtgtggggcctcgagggccagaacctcagaatgtagaagctgagttagtttgtggaagggtgaagggctgacctctactgttggcaatgccaaaatctctgatctggctttaaataagcaaaacagccccgccagacatagagtcatccacaaaacctcatgacctctgttattgcccatcctgtgtggaagcccactgagaacttcacttgaagatgctgcaggaacaactgcagggaacctggagataaggccccgggtgatgatgtaagctaattgctgagctgggtggagctacttaaccttatggtaggaggtggagcttgacgcaacccccttttccaccaacaagcatttctttctacctgtcattgaagtgaatcatcagtataagtagcgagctgggcgcattccttctcagttgtgttaaaacttgctggtattcccccggtatcagcagaggtgtgtacgggcactgctttaaaactgggaaggaggaagacgaggccagggagccggagggtcaccaaggtagatttccagcagcgctagtccagctgaacactttccagccttgtttttcagcagctttgaggaaaagtataggtaagaacaaagacaccactgtatgtttgctatatgaatgcataacatctgttcttggtgtgagtttgatgttggtttaatactgtttttatgtcagcggcatagttctagcatgtaccaagattttcaattttaaatgctttttggtgttcacagttagctataattggtcaattttaccctccttatgcataaaaagaatgataggcaaagaatgagccttgaaaagaattgggatcagaattctggtcccctgttctttgcttcttaactttcttgactgtttagaaaaatgtacagcttttcttccaaaaaccaaagcaataatacttttatttttgaaaataatggaaaacagagaccgctgaagttctatgaatgaggttttctgttttagaaatcacaggttgaggaggaaagaattgtgttttccttcctgttctgtttcgagtctcagtatttctgacttagaaacctcctggagaagagggagataccagctctcctttgtttcaagtaaaataccatcttaggtcataaaatagtttgcttggagaataattcatggcttatatctgaatttttggttggtagcaacagaaatctttctcttcctcggtatgagtaatttggcttacatgatttagactaaattttattatattttttgtttgagaaaacaaaagtcaaagcccagaacatattcaactgagtttttccctcaggttttctatcattttcccacccttcctactcatacatgtgaatatgctgaatgattaacatgtgatttttgtttctttactgtctcatttcatggtgattcataaactgttagttaactggttatagtgtatccatatgttggcaaagcttgaaataaatttgagtgcaggccacaccttcgtattaatgagccgggttactttcagtaaagtatttctgatgtcattaagtgttaagatggccggaattctaaagacaataaaaatgtatcaacaatatgaaatatcaaagcctacagtcaaatctgagttattctttagtacttatttcatagctattctttaatgtttattctttaatacttatttcacattgaaaagtactttacacatgtttgaatgagatattgccagtgaaagaaacaaaaggaagcaaataactctataagggaagtgggattttaaagtatgtttgtgctggccccaggctggtgagtgagtttcattagcaatgaccaggaagagaaagaatgccacagtggtgggtgctgaccttttttttttttttttaaaccttttttaatgtctgtaaggaatacaaggtgaactttaattcttgtgggaatctagtaagatggaagtcaagaagagaggaagaaatgctttttatttagttaagaggaagtaaacatctgtattagttacagatgttatgccttaacctttttcattcctattgtttttatgaaaatttgcctttttaagttaaatgaaggttttctggagtcctgtattgcaactaaatgttgatttaaaacgacacatttgggacattttcttggcactgtggaatataatgaaaagatataaaatgaaggcaggtatgaaattatatgaattctgctacttgagtgtagaagttttattccaaagtggtttgtttggtggatgtggtgatggtggggggctctatgtacttattaaggcaagtgatgggacaggagcttttgtggtcttgacggttctgttgggatgggagggttgtttgggatttttatgtttcattccaggcaatgtttattttttcagcctgtacttcttagggttttaccaagtcagttttcatgtgaagccattttcatatcgtttctttcaccttgatggaaatcttttccttcttatgtgctttgagttaaatatgatgtagtgtgctagtaacatagacggggctttcctttctacaggtgggcttgaagtgagcctgccactcgctcaccttgaatcacggaggtttcagaggccgttggccttcagggcaatggcagaagcacgtgttctctgctgttttctgagtctatcttgactgcgttcccagtggcccagaattccccaagggtcactcgacacaccctcgtctacagtaaatatttgtgcttcctctgtgcctggccaggttttctcagtacccaggagcacaggtgggcagtgggatgtgggcaggcggtacccagattccctcccacaggggccacagggaggacttgggcgcttcctgccaagagccccctgcccttcagactaagtggaaatagagatccgggagtccagtggactcagtggagttccgtagaatacatggaaaacgcaccgctttcagtgatgtaagctgtcttgttcacttcacgattactcatctggcctgcagcaggcaagaggatcacatgcaattataaacagcctcctctgactaaggtcagtgactggaaaccctgtcacgttatcagaggtcatttggtcatagattcttgggcagaagccattgagagtcctgggggtgtgatctttgagcctttgccagtgctggggtttgactccttttagctcaattaactgggtccaggagtttattttggccgagatctgccatgctgagttgacaggcttgacactggaaggaagtctctaacttcattagaggaaatggcgtggcaggctttaaggcattttgatatactccactttttctctcttgccctgttttgttgaggttttcagggccataactgtacctgaggttaatcagtgagtgatctatctgaaaggaatttgttctctttgggcaccgtcctgtgtggtattatatttgtataactatttagcttaactcattcaaaaggctatttaagtcacttgtcctgttcattacttgaaaacaagttgtcttctctttattcttttagtcagttaccaaatatttcctaagtgtctcttgtgctagacattgaggggcactccttctccctttcacttgtctgtttttgttgagctacattttttccttaagtcagattgttcttttcctgttacttttgttttcacttgatttgtttatttatatttaaatatgtactttaacagtcttgaagcattggtctgaatgaggatggtgatcagattgggaaaatgccccttaattttggtttaaaaaaatttattttctttaaattatttgtttattgataggggccacccactccaggtgagtggtttctctgtgtgtatggcagaggaatgctctgtgtctggaaaaaaaagtaacttagcttgaaatattatgaaagctgaagagtacaaatgtcagagagaaaggggtattgggggggaagagaatgagaagacaagacacagtcagggccctgtcacccgtgtggaactgcaggtccctggagagatgatgcatttatccctttttagagatattgccattaaaagttgatcttgagcatgacacagaactgaaagctttgatgacaaagccagcatctcagtttagcaataaccaggttagggaaaaaaccccagaaactcaacagtgagattcagctcagcaatgtgggcggggaacccactgtcccgccttgggcagggagcagccccagccgcttcatctgcctgatgctgggttttatccgcttcacaattaaatttcctttttgttatgcttgcgcacatctatatttggctttgcttcttattttttaagaaaggggagaagtgtgggcaaggtaaaatacagcttgcaagctcccaaatggcaaatgcaccagttagagtctaagtggcctcccaaccttaaggtgaaattcatttattcagcaaatatgtaccaagcccatcagggaccactgctcgagataccagtgcaaatagaagaggccaagggcacattattaggtccggagccagtggcctgcacagccatctgtgaccacctacctctctgcttgcagggaacttcagcctggtggaggagacccataatagccaagtcaactgataatagaattagtagtagtgttgagggacataaagaaatcagggtaaggccatcacaattacatgggtggatgatcttttataaaacagcacagttttgttttcttctgccatggtatctcctaacattttccagtgtgtctacttgcagttaatatggaagttgtggctgacactaggttagtggttaaaagaaaacaagaaaaccaacctttagaaatctttagaaacatcattatcatagaaagctataaactcttatgtatagaagggtaagtggccattgatcttggagcagaatgaatggatatttcctggaaaaaaatatttccactcatcaaagatactcaagtctcttctcatctaatcaatattggaaactaggaatatccacgcctatggaatatcctcagagagaattaattattctgccctctaagaggtatagtagtttagacttagaagtaaaggcctgaacttgaggctgcaccattgaaggcaaaagtgccctttatctcccagctttgtcatttgccaaatgcaattgtgttaaattaagtgacctccaatagcagagctctatgttttctcccctgacccccaagtcaaaattgattatagtaatccatcaaaccaggcctggcaaatgatgttttgcaactaaaggataaatactgagggggagggacgaatgtgaacgcatatgcagagcaacacagtcatgagaccattctattacttcctaaatttcttaaagcttttctgtcttttctgagcttctcaagtgagcaggggcatgagttcctgtgttgtgcctggggacagggaaggctttgggacctactgtggtcttaagtctgagcagtgctaatagcctcggctcaccaagtgctgggcactgtgctagagggtgtacaaacctctcatctcatgaggcctcactgctacagctgacagagatgacatccagttgtttttagttgcacatggcagagctagaaaacaaaccagggtgtgtcagactccagagcgcatgccctctgtgatgccgctgtgttaccctagatcaaatcaaccaagccatccgttaaaaaaaaaaacctgatctttgtcaaatatgcaattgttatatttttgttagagtctgcctctccagtgaacgatattaatttccttcaagtggctactcacagatatcacaaaatcagaaagctgcctaccctgcagcctgtatctgcctccacgtccccacatcagtcctccttgctgtgcttcttgtctctttagtttttatcatcttctaacactgtgaactttagtaatttattctggattgtttgctacctgtctccccttggtaaaatgtaggctccgcagaggtagagcgttttgtctgttttcttcactggcatatctcaggtgcctaaaacaaccatgccagacatgtagtggtgccagtgacaacttgttgaatgaagaaatgggagaaaaaagcagaccccggagacttaggagggtctgtgtgagggagtctccaggcagtcttcctgggggtgtcctgaaggacctcctgatcgtgattttggtcagtgtggaggctgtggtttcttctgagctgtcagtatttggaggtgagttgtctgcaggtcacctcatcccacctttacacattagtaaaatggaattcgctgggtgattcttaggactgtgcggtcagtcgaattaccagacgtccctgcatgctctgtctaagggctggagtctgctttccttgcttctcttctagaaagaggtctgtccacaagggattagcctcgcgaagttcctactgttgttcattgacccagtgagtgttttacccactgtgggtttttcctacctaattgcccctctggttttcagtgttcaacatgctgcagttacgtgttctgctgtggttttacttagcttaatcctttattaattgcctcttgttcaaactccctaagagcagattatccttctctttcagcttctgctccattgctgttcaacttttttttggtcgtgacatcagaaaaataaccaagtgtgagtctgataatagcaagtaaggctattttatttctatccaatttaccccaaatttgagcagtattacaccttaaaataatagagctggctcttgcccagcatgtgaaaagtgtatgcttcacgatggacagatattttccagtatctacttaccataaatctcaagatttaccctaccccactccagccattacccccatgtcccatttgcctaacctaccctagggccatctcagtgccctgtaaagcaacctttgttgcagacccactgtgtgactggaatgtgatgcacacaagtgaccaaggcatggccctgttcgcaaggagcttccagaccaatagcagaaatggacacataagtaaacaactgcgctacttactgataggcttctgccaacaggagtggagcttgagggccaggaagccttcccagaggcactggagcttgttttaacgttacgtgcatgccaggtggggggcaggacatttctagcagaggagaaagcatgctgtgctctgagaactaaaagtcatagtcactccacttggtgacatgtataaggcacatggaatagggccagagctgggcagaccgtgtatgccacaatagttcgttgtaggttacaaagaaccagcgcagagtgggtgaggcataggctatttgattagtaatttggctgcattgacaagatacggaaaaggcgaggttgggactttccgtgaattttctcctcaaggtgcttaatcagtgaggttgggatggccggagggggccctcatttgcttgcctggtgctcaagcctgaggtcctggcctttaatagtcagggaggtctgtgtggaggcagggtgtgagccccctccaaggaaagctgcagtgtgaaaagaggcccaggatgagcgttggggagtatccacatacaacttgatattcctaaagtctcagttctttatgggaaagcccatgcactcttccacacttggaattctttgcaaagtgagtggtatttgagcttctttagagtcttgctactcccaccagctgtactggcatcaccagggagcttgttagaaatgcagattctcgaggcgcggctcacacctacccaatcagactacattttaaccagatcccaggcaatccctgtgcacgtgaagtgctagaacactgctctgcggtaaaacgactttgctaaccatggaagagggtgatttttttcccttttcctttggtgttttttataggacctatttaccttttagaactttttcagaattatggactgcagaatatatacagtgtcaatgtcattagcaattcattagtgatatttggctagaatacatagttgtttccacacaaatgccatttcttacattgagatggcacatgtctggttaggacttgggctcatttgtaaggagccacgctgcaggtaggcatgctcccgggacattagttgctcaggtttcaaactgctgccccagcatagcagatggcacggtaacaaagtccctgtgcactctcacattgcctgcggtgatctatcacacattttgtcccctaataacctagctgggggggagctcatgtctctgtttgtgctaatgctcgctgtatccccaagtggcagtacttaatataattgtcattgctctttaatattatctcattccacgtcagctgcacaaaaatttgaggttaattacaatgaatgttttctcaatccttgttattgagagctgtttaatagtttattttgtatgtgaagattcatatctagattgaggcttatacaacttcatttaaagacagctttttcggctgggcgcggtggctcatgcctgtaagcccaacactttgggaggccgaggcgggcggatcgtgaggtcaggagatcaagactatcctggccaatatggtgaaaccccgtctctactaaaaatacaaaaattagctgggcgtggtggcgggcacctgtagtcccagctactcggaggctgaggcaagagaatcccttgaacccaggaggcggaggttgcagtgagccgagatcgtgccactgcattccagcctgggcgacagagtgagactctgtctcaaaaaaacaaaacaaacaaacaaaaaaggaaagctttttcaggttggaaaaatatcaacagcagacatgaaaaagcatgaaaaatgacgctaagaatgtaagcaactggaaacaaaaattataatggtgcatagtgagctgaagatctatatttaaatgtgaactcccctttcagtttataagtgattactaacagcctcatcaggtttctggaattaaatgttttattatttcctcccaaagttagtcctcaaacattagtatttcttgtgttcttttgatttgaatatttgtttttaacacacaaggatatcaactctataagtcagttaatatgttcatgcttttagttaatcgaaagtggcttatgatttcagtttttggtatcaggtgatgttgcggaagtgaggttcactgtgaagcttyttttttttttttttttttaatcattttgttgtttctttgagtgtatgtgagcttataactcaagacactatgtgagatgaccctgtgtttcctacattatagaaaagaggaaaatgaagcaagttcaagaaaattagatttatgtggaaaataaattctgcatctagttattcattatcaggtatgagattacaggcatgagccactgtgcctggtgacctcatttgaactcgattacctctgtgaagaccctgtttccgaagaaggtcaacattctgaggtgccattgtcaggtatgaggtaaacacactgagaataccacattttggcaactcagacaagactattttccaaacatgtaaccctgggaagaaggtaacctatcatagtgtgtgttaagaagtcaagcaaccaagaaatgttaattgaaggcccgttttgtacaaggttgcatggaaaagacacactagcgtaagacatggacagtggccctgagtgacgtgcagccggtttaggcgaattaagatttacatacttgaaaagggacaaacaagactggacgtggtgactcatacctgtaattccaacactgtgggaggtggaggcaggaggatcccttgaggccaggagtttgcaaccagcctgggcaacatgggaaaacccctgtctctacaaaaattaaaaaaaaattagccatgtgtggcagcatgcccatagtcccagctactcacaggggctgaggtgggagaattgcttgagcccaggagatccaggctgcagagattgtgccactgcagcctgggtggcagagcgagaccctatctcaaaaaaaaaaaaaaaaaagaacagaaaaggaacaaacaacagacacaagattccatggaaaagggagtggtctaagtattcagactgtaggaattcagagagtggagaggctggttattatgggggtgcttcatcctggggagttgagggaaaggaggtgctggctgcctggagcctggaagaatcaaagatttccaaggccatggggggaaagcatgaaggatgagtgaatagacaccagggctgagcaccctgaaaactgtgaagatactagattgacctgtttggaaaaccaaacagatgtggctgttattgacaagtggttttgtttttgttttgagacagagtctcacttggtcgcccaggctggagtgctgtggcttgatcttggctccctgcaacctccgcctccaaggttcaagcagttctcctaccccagcctcccaagtagctgggattacaggcatgagccaccatgcccggctaattttttgtatttttagtagagatggggcttcaccatgttggccaggctggtctcgaactcctgagctcagatgatccacttgccttggcctcccaaggtgctgggatcacaggcatgagccactgtgcctggccaacaagtggtttaaattggattctccatttcccacctaatagaaagtcggaagcatagggcaaaagtaaaggaaggaccctgtgttctgctggttaaatataacagaggtctgactccatggctattataggagaggtgcattttgttctatgtaaattatcttgatgtgtcccctgtgcctttaccccatgattatttagcacttttttctccacggtcacagggacagggagaaacatacacccataaaacagtgtcatctagaggcaaaaacttgtaatgctcaagcatgtgctctgacgaggtggggcagggtgtgtaggaacaacactctactaattattacagagcttgctctgcactctctgtagaagtccttcaatatccagtctaacacacacagtaacttaagagaggattttaaaaaatcaaaatgccctcagatttgtattggatttaaccaggctgagatccctgagatccaaatataataatgtgtcctttttggatgaatcagacacaagcaaaaatctccagtgtgtgtccagctttagaggtgtagtaatggattccacagttcacgctggtgggccagccaagacttttaatgctgatgtgggcttcattctttctataaactccctttcttcttgatagtgcatgggcccaggggttccaaacatgggtacctgccgtaattgatctgtgtaaccggagaaaatacatatcctatggcacccttgtgtcgggatatagggggcatcaccgtaatgtggctctttttctgttgattatagttttttctttctgtgatcacacaattgttttatcctcgcttaaaaaatttcaagaagtgcagcactcagaataacagaaaagtcaggtgtctggtggtttgtttgcacatttaatagacttgaagataaactctctaaacatttattttgcaagttgtttttctgagagttgaggggaaacttttgccctttggtctccctgaggaattctaggatttcacaggtaggcgtgtgttttggactgcaggctgaacgtggttggcagggtggcatgaggagggcacatattttaagcgttcttagggcagatccacgatcctttacctgccatttctaagtccaaaaagccctgaaaaccaaagacatctttacaggtttgtaggaaattgatttgagagcaaaacctgatgtgaacagatttgagagtattctaggcatcttgtataacacttcattcaatgctttgtgcctcttgctgcacagatattaacattcacttatgggccctgccccatcccctactggggatgctatgcaataatcagaataatcaaactaataataataataataattggaataatcactgagtcattgtattagtccgttttcacgctgctggtaaagacatacccaagactgggtcatttataaagaaaaagaggtttaatgtactcagttccacatggccggggaggcctcacaatcatggtagaaggcaaaaggcacatcttacatggcggcagacaagagagaaatgaaagccaagggaaaggggaaaccctgtataaaatcatcagatctcttgagacttattcactatcacaagaatggggaaaactgtccccatgattcaattatctcccactgggtccctcccacaacatgtgggaattataggagctacaatttaagatgacgttgggtgggaacacagccaaaccatatcagttatctttctaaagtgagacaaaaagtcagaattctgtaacacattttttcccaagcgtttccaaaaaaggttggctttaatggagtattagtttgctaggcctgccatggcaaagcactatgaactaggtggctaacaacagaaatttattttctatcagtgttggagtccagaagtctgaaattaagagtcagcaggattctgctgtctctgagccctatgggaatatcctttctggctcttctagtttgtgatgtttgccagcagcgttctctgttccttggcctgtaaatgacaatcttctcgttgtatcttcacatcgtctttcctctgtgcctgtttgtttctgtgtctacctttcccctgttcataagaacaccagtcatattggatcagggctcaacctaatgacctcatttctttccttttctttccttctttctttctttctttctttctttctttctttctttctttctttctttctttctttcttttcttttcttttcctttctttttttctttctttctctctctctttctttcttctttttctctctctttctttctttccttccttccccccttcctgcctccctccctcccttccttccttctttcttttaaaaatatttttcttttttttcccttccttcctccctcccttccccccttctccttccccttcctccctttcccccttcctccctccttctctccctccctccctccttccctccctcagggtcttgttctgtcatctaggctggagtacagtggcatgatcatggctcactgcagcctcagcctccctggcctaagcaatcctctcacctcagcctcttgagtagctgggaccatgggtgtttgccaccatgcctggctaagtttaaaaaaaaaaatcgtagagatagggtctcactatgttgcccaggctggctttgaactcctgagctcaagggatcctcctatctcagcctcccaaaggggcgagattacaggcatgagccactgtgcctggtgacctcatttgaacttgattacctctgtgaaaaccctgtttccagagaaggtcacattctgaaatactgggggaacatagttccccatcttataagatagagaatgggtcaagacgagcagctccattctttgtttgtgtttaggccagtgccaagttcaagatcttgcctcagggctttcctggtaccactgggaatgtgtatcagcagcagctgtgtcactgaatgggtgtcacctggggggctgcctgttcagacattagaagctgccacatcccctgttaactccatgtgagggaggaatcccttccctccgacaatttcgcctgtgagaatcaagtcagattttgcagtgagaactgagctgagtgtagaataagttcccgaggtacttctggcaccgttgttactcccccaccgagaagcccagaatgttcccaggtgtgttcaaagattgtacaggaagggcttaggacggtgcttggctcatataaacactcagtggcaaagaggttgatgcttaagtacacaggaagtgtttaccagtacacataattgtataccagtgagctccaccattcaatttactgtctgtccggtacattgaccctgtgaggcccactctcggtgtggcactgctaacaggttttattgttgcttactcacttgaaaagtgtctcccttggctagacctttgtcctgagcactggacacacataggtgctcagcagcgtccctctctgcaatgcaaaagggataccgtgtgcctctgcatgtcagctctagtcctggcatcccaggagcatacggtgacagtggaagtgaagcgcaagccgagtcctgggaaccaatgtctgccagaattaattaaaagtaagcatctttttttccctggttagttaatccaggatgaattccctgagtagttcctgccgttagaacttgctctcttttttagttagacacagctcggtgttgcctccactttgacatcacgttcatagttcctgtgcttggccagaactgttttcagttgcaaggacatgggtgagactggtcaaggaattgactttcagggttccaagggaataattagttcagaagtctgtccccatacttgttttccagtacaatctgtgtctctgtgggtgtttttttttaaggctgtgtgctttataaactggagatagaaggagccagtttattataaaaaggataaaatataattaaacaattaaaagtatgtctttatagccgggtgtggtggctcacatctgtagtcccagctacttgggaggctgaggcaggagaatcgcttgaacctgggaggcggaggctgcagtgaactgagattgtgccactgcactccagcctgggcgagagagtgagactccatctcaaaaataaataaaaattaaaataaaaataaacataggtctttggccaagtgtagtggctcccgcctgtaatcccagcagtttgggaggcagaggcaggaagatcgcctgaggctaggagtttgagaccaacctgggcaacatagtaacctcgtctctacaaaaaatttttaaaaattagccaggcatggtggtgtgtgtctgtagtcccagctactcaggaggctgaggtgggaggatcgcttgagcccaggaggtcgaggctgcagtaagccatgatcttgccactgcactccagcctgggtgacagagcgagaccctgtctcaaaaaaaaaagaaaaaaaaaattatgaagtactggttgggttaacgtttcttcttctaaccatgagggaagtaggtgggctgttctggtgactcagtgtcctcccagactcactcatgagaggaagtattcatggactgtggtgatcttgtcttggaatttttttctttgcctcctggcctcacagccgttccttttcttttacacatccatgaaagggagactctaagatcttccttgccaaggttgttgtagggatgagcgatgatgtctctgaggcaccaggcacggtgctggcacccagaagctgcttaatgatcctggaagttatgaccattgctcatagtttttcctacgtttcagatcatccttaaataattgtcattggctgggcgctgcggctcacgcctgtaatcccagcactttgggaggccaaggtggttggatcacctgaggtagggagttcaagactagcccgaccaacatggagaaaccccatctctactaaaaatacaatattagcccggcatggtggcgcatgactgtaatcccagctactcgggaggctgaggcaggagaatagcttgaacctgggaggcagaggctgcggtgagccaagatcatgccattgcactccaacctggacaccaatagcgaaactccgtctcaaataataataataataataataataacaacaataattgtcatttatcagcttcttcgagctcttaatatatatatcgccacctttcacaaaggcaaaatctgcattagaaacactgttgtctgattttggttcctctggaggaggggtagacagtctttttgtgggcagggtctgtgtctgtttacctctattccccgccccccactttggtgcccaccacagtcccttgtgtatggtggatgctccttgactatctgaggaataaaatcaattgtgaaacagtttccaaaaagggaacagtttttgtttccccgtttccttatagccttgtgagttgctatctctcttggctctattattaataacataaaagaaagagctgttttcttaacctctggtagttgcacatatatggggaaaaggcataagaaaatatgctggcaataaagacaagtcacagattgtcattttctttggtgactgaagggatatttcagtaatttataggtgaaattttcctttttgtgacttccactgaacaacatttttactaccaaaagataagggaacaaaattttttaacaaactatttaagcttaaatatttaaacatttttattagtcaccacatattaataaaatatttttattagcacatattttattagtcaacacatgtgtacacagctctgatgatctctatgtagatgcctatataaatagctatatagatatcatcagagctgtgtatatatgtgttagagatagatgcacatacagttgccccctggctcccaggggtctctgtgacccaaggttgggaccctcaagtgactagttatggatggcaaggggggctgcaaggccagagttggtcctggctagagatttcaagactcgagtggctgggaaggttttagaatgcaggtaaaccactagttgctccaaacaacaaaaccatcataatggaagcatccaaaagtgtgttcttcggcagcttcagagactatgcctaagcaataaagttttaatgtcttttttagctgcttaggtgaagaatgcatttgactgtaatctaacccattaatctcttatttttgctggaaaattgtacatatttaagagtagtggctcattgccagtattggtgatgagtgatatattttaaactatgcttcatacctttcagtaaatcttgggctgaaagatatgtacgttttgttttcaaaatttttcagtagtcatctatttaggtcaattttctgggcaaacaaaatatttgaagccctttagggtttgttattgagatgtaggctggccatgaaattacagggttcagtacctcattgaagtgtatatgtaactgttattagttttttgataacttttttatactcataaaagtagtacttgcttacagtagaaacttaataaaaaccataaatggaaaaataaaccattcgtaaacatcatatagagagaatgactattaacattttagcctaacatgattttaggggtttataatattctagtatatgattgtaccataatttatttaattgcttcccaattattggactggaagtttgcttctaattttttcttattatatatgatgctgtaatgagtaccttttaccagtaaatatttacctgcctctctgattacttctttgggattaatccttagaaataaatttgatgaatcaaagggcatcaaccctttgaatgcttctgatacttctgccatgttgtttcccagaaaagatataataattattccttccagtagtagagtattaaatgtctgcctctctttagaaagtattaccttttttcaatctttgacaatttgttagataaaaacttcagtttcaatattattgcatgtattcatggatatttttcttctcacacattctagattttttctatattgtttataaatgttttataagtgttattactttattgaggatagaaattattatgtaacacttgatggtaacagtaaaagtggaaaagaatgaggaaaccttcaacaaaacaagttgtcattaacaaaacgggttttgcaatgcaactttctgtgggttagctaaatctttagtgtgtcaggtagtcgaaagaaatttgctgtatagtgctgtgaaatatgctgcatccatgagccctctgctctttgcccatagggagcaaagacacaatctttttgcaagctgtcccagtgccatattgaccatcttccttttacagcaaaaagttattgcttcctttggttaaggttgggacacccctctggaggcacacttgaaaaatgaagacctttctcgttgttcttctgttatccaagtatcttggaagaacacctctatagcaggtagacaaggcagggtgtcattgcaatctggctgcagactgaatgtaatcccccttgacattttgccccttggaggcaactcctgccctcccctctccccttacgaccctgcccccttaaaatatgtggagctcagcaaagactatttattctgataaaaatgagcaaataataggtgtgaaagaacatggaacacttcagtgaatcaaatggagaggattaagcattaccccaataatgattggtcaggacgactgtccctgtggagtgtgtcccagaagggttgagcaatggagcaaacccagaaaatctcattctcaactctccctgacagatgacctcgtctgcgctgtcttttgcttcatggtgatgaaggtggaacaagacccttgaataagatttccatctctgaattctttaaacccatttgagatcagtgcaggcctgcagtctctctattagaactggagcagaggcccagaagagagaactgaattttcagcactggattctatgttgagaatgaaagagacaataattgaaaagcttttctcttggaaattttgcaaaatccgagtggatttttgaccaggatttcttggtgacctctcctgagagagcaggtgcctgaggatttcctgctgagtatcttggcatctgtcagactccttatgacttcattttagttcagggagtctgtttcctagccattgatttcatgcacaattcctggcatgtctcatatctctaaggtgatgtcacccttgtatttttttttcagatgtaatcttttatgtgacagtgaggtgtcttacttgtgagtgaacagctagaacctcccaagtggtttttcttttggattttgtttactcagtgaaaaatactgaggaacctgagatccttcagggtgcattttggtgtctgcccaaccttgacgtggatttggaggccaggctcaatgctcgaacttcttgttggagtggcactaaatcattaggatccctgaggacagacagttcaggccatcattatttggtaggcccttgcacacttacacacaaatgcatcattatgaacatggagcagaagattctagggccccgtgtgtaatggcacaatttgtcaagaggcctgagacctggagttggcatggattccctctcctcctgacccacctacctgctacattcagttggccaccctgttcttttgagtccacctttggatgagtagtcagtcctttctgatttttgccacctctgcattcagagctttcttattctttggcgcggaggccttccgttgggtcccactgcctgctgtcttatctccttctaatgaatatgatcatgatttttctgtatttagacaactccacgatgaggaggatgatggcggcagctaacatttatgtatgtatgtatgtatgtatgtatttagagacagattctctgtcacccaggtgcagtggtgcaatcttggctcactgcaacctccccctcctggattcaagtgattctcatgcctcagctccccgagtagctgggactacagatgcacaccaccacacccagctaatttttgtatttttagtagagacgagattttaccatgttgtccaggctggtccggaattcctgacctcaagtgatctgcccaccccggccttccaaagtgctgagattacaggcgcctgcctccacacccagccagcagctaacatttattaagtgctcactatactcctgccaagcactattataatttgtcttctaacaggctttttataagggaagtgctactattatacccattacatagatggtaaaactgaggcacagagaggctatgtaagtttcccaaagtcatgtactaattgatgaagacccagggtttgagctctgcaggaatggccgactccttgtggtttgagcctgggctccctctccatctgtcttttctccctgagccctcctgactgcagccatgccagactttttaatgttcggaaccccagctccagcctatcactggagctctgcctgtgatctttccacctcatgcctcccggttctatgtgtgccttcagactccatctggcagtggtatctggatggaccagtgcatagaaatcacatgagacactcaataaatgtagactcctagtgattctgactagcaggtctggggtggggccaggtacccagatttgcacgggaagtcagggaccactgctgctgactgcttttttcctgggtgcttggcacactctgtcacaatcatgttttcatttctctatgttccatttcaactggatagggacagtgtcttttgtttctggaacctgtctcttttcttggcatatagtaggcactcaggaaatagaaggttgcttgaatcctagtaccagtttactatctccttggaaagactgaaaggtagaatgtgatgtctaagcagcttcactaagcagagaaaacaaaaagtttgcaaaatagaatttgccttttaatctacatgttcttgcgtagcattgagcttcctagagagtcatattcatgctagccctgagttgtatcagcagcagccactagaacggtaacttgatgccaagagtgaattctgatgagcttcattgtgagtaagagtcccagtttgatcatctctacccagatcccgtgggacctccacactgtatgaataatgaggaggcgtggaatacgtagcatgctgcagttggccagcacccagtgtgtactgtgaggtaatcagtgtcaaatcagtcattgtagcaggagttgggagtcaaaaggagcaacagagaacctcacacgtctcctaagagaggaagcaaagaacagttaccttggctgtcatttatggggcatttgtattgtatgtttggaatggtgctgggtactagtattaatacatgattttctctggtctttgcaaaaactgcctgagttaggaattgtcattcccatttcattcatgagcaggctgaagttaagagtggttaagtaccagctgaaagggaggaggagcctgagagcaatctgggcacccgttttccccctcttcttatttatgaatgaatgaatgagacagggtctcactctgtccgggtctcactctgtcgcccatcctggagtgcagtggcacaatctcggttcgctgcagcctccacctcccaggctcaagcagtcctcccacctcaacctctgtggtggctgtgactacaggtgcatgccaccacacgcagctacattttttttgtatttttttgtagagatggggtttcgccatgttgcccaggctggtcttgaacccccgggctcaagcgatctgcctgcctcagcctcccaaatggttgggactacaggcgtgagccactgcgcctaaccctctcctcccctcgagcttacatcctactgtcctgtagccagctccaagatgttggcctttggttctctcttcaggctgaactctctttcagggagtttcctaatcgatcttccatagagttgatcataccttagtacctacctggatgagcactcaagatgttacatattcccacctgacttctttattactgctggaagatatggtgtcccacacctaagtatcccacccacagtccccagctgcatgtgtcgattattgtgagctattcaagggatacagactgaggtaaatgaaccaccagtggaagaatgtggatgttctattacaggaaatgcttagctctttattgcagttatataatagccggcaatcatgatagctgaggggcataaaaatgagagcaacagagtttatggtattctttataactgtggattctgtatgtgcgtgtgtgtgcacgtgtgttctgcatgcatctccatggcacattatctggaggtaacatgatcatcaggccttgagctcttttataagcaatagattgtggctggaaattgaaaatttagtttctgtagctagaaaatactcttttcaaatgccatctagaacagtagttctctgagtgtggttcctggatcaggagccttggtatcactgggaatgtgttaggaatgcagattcttgggttctaccccggattgaattcagatttgggagcggggcccagttatctgtgttttaatcaccccctcccccaggtgaatctgatatgctaaagcctgagcctcactcccctggaaagtgacgcctgattcatctgccagcccatgggcacccattaggaaggtgcttatgggagctttgacctgggctaggtgccggctcttgatccatctttttctaaggctgagggtgtccatagaaggagaagtaggtgcatccgcttagatgttatttaaccaaataaaggtttacagcttacagaaaagaggcaagattaactaataccttccaaaccataatgtaaaaaaaaaataaaaaatatttactcataatggagtataactgaacaaatcatgttgctaatttttttaggatatgttttgtagcttccattcagatacatcttatgggtaataaatacacacgtgaacacacatatgtgtactaacaatatttttcagacttttgattttttagatattgagtatagccttgttactaccaaacttttatcatacttccttgtgaaaggatttcatggcatatttattatttgaaatatcaaatagaaatcagtacagtctcagcattttaagatacttgatgtaatctttggaaatatcttagacattgcaaaataagggttaatagtcatagtatagccactaacgggcagttctgaaattacataaagttaatctgatgaactgatagcaatcagtttttggctatacataactagataagtgagaatttagagattgaatacatgagagccctttttatgattttcaggatctcaagtatgaagcaaaataaattcttctataccctaaattcttgcaaagtgatattttgattagatttgagctaccccagggctagcaatacttgaaagtgtgactagacttaacctcttagtagatagtccgcagtaatcctttgaaatatacatcttgctaaaagttcctttcccaaggaaataatgattaaagataatgtgatgtgtacaaggaattctgggacttgtagtcgagaaacctgaactttaatctagactactcaaatttataactttaggcaaatctctcttgactttctaggctttagttttctagggttttgttttgatttgtttttttatgtttttgtttttgtcttttttttggggggagggtgttgagacagagtcttgctctgtagcccaggctggagtgcagtagtgtgatcttggctcaccgcaacctctgtctctcaggttcaagcgattcttctgcctcagcctcctgagtagctgggattacaggcacctgccaccatgcccagctgatttttgtatttttagtagagacagggtttcaccaggttggccaggctggtcttgaactcctgacctcaggtgatccacctgcctctgcctctgaaagtgctgggattacaggtgtgagccacctcgcccagccttgatttggtttttaatctgtaaaatggtaggtgttgggtcacatgaacaatggctcttttacctccactaattacctagacgctagaaataagaatatggataagacctaaaccaaattttctagctaaattttgtaaagtctgaacaaaatctgtgtgcgttttaatctttcttgtaatcttttcagactctgcctttaaaattatatatatttttagcttctaagtaaacattattggcactggttttgataggaactttactccaagtgtattgacttagtagatatgttaaaaaggttattgcttagtttgtggaaataatacaagctgagactttaactgatatttgtattttcatttaggttccactgttttctcttttgaatgaaaagtataatcctattaaatctaaaatattttcttacagtgatccgtatgtgaaactttcattgtacgtagcggatgagaatagagaacttgctttggtccagacaaaaacaattaaaaaggtaggtgtcgatctttaaccaaatgtcatttaagtcataatttaatccaattatgcacagtcatgtgctgcttaacacctttttggtcaaggatggactacttacacagtgatagtcccgtgagattataatggagctgccatatacaagtgcatcactttttctcttttttttttttttttttttttttgagatggagtctcactctgtcgcccaggctggagtgcaatggcatgttctcagctcactgcaccctccgcctcctgggttcaagtgattctcctgcctcagcctcctgaatagcagggattacaggcacctgtcaccatgcctggctatttttttttttttttttttttttgtatttttagtagagacagggtttcaccatgttggccagactggccttgaactcctgacctcaggtgatccacctgtcgtggactcccaaagtgctgggattacaggtgtgagccaccgtgcccagcccactttttgtcttatatactgtattttcattttaagtgtctttcagtattaaatctagaagttttccaagacttttcagtactaacccaagatagtgattcaggtttttacttaaatgcctacaaaaacagtatcctgaaaatgtcataagttgtaacattttttcttgcacagaatacagcaaaaacaaggatttgtttttgtctttgtatgtttcacaaagaaacttaaagtaatttaaaatacatttaatgaaatggagctgtttgtggaattggtaggcacaccaggaatagtttaacttgaaacaactatttctaaaggaagcaaaatacttggttagctacatggaaatagagaaaatatattaataataaagctatacatgtatgtatatatcttattttcctaatttttatgtacatgatttcattaaaaatttgtttccataagtatgtcttttagttatttatttatttagtttgagatggagtgtcactctgtccacccaagctggagtgcagtggcataatctcagctcaccgcaacctttgcctcctgggttcaagtgattctcctgcctcagcttcctgagtagctgagattacaagtgtgcaccactatgcccagctaattattgtatttttagtagacgtgggattttgccatgttgaccaggctggtctggaactgctgacctcaggtgatccacccgccttagcctcccaaagtgctgggaatacagatgtgagccactgtgcctagccaataagtatgtcttaatttgcctatctccatgtatttatagacatatgtaatatatatatatgtaaatattttttaccctatgtttggaggctttattgaagatgcatgttctctattaaaaatacttggctgcatgtggtggctcatggctgtaattctagcactttggaggccgaggctggcagatcaattgagcccgggagattgaggttgcagtgagccataatgatgccactgcactccagcctgggtgacagaatgagaacctgtctcaaaaaaaacccaaaaaacaaaacaaacccctttcatctgtgctttagtatcatttatgggttactatgatttttttccttacctcctttagaggcgggtgcttagatagtctaacactttgctgaacattttctggacagcatgagtgctttaaaaaattgtaactgtcttgctgccatgcaggcacccaggatgcagccaggtccttttgtgtaaggaaccataactgcagcgtgtcctccacttggcattgggactggcattaaaaatctgttttgcaaacaggagccagtcttttgattagcagggaggatgcctgcgagggacggaggccattgttccctgtgttaggcgttgttgctggttcagtttctaggcgagtgggggctgtctagaaggctcaccaggaaggcagctgggaaacttggttgcctgctggatgtggagccaggaccaaagccggcgactggggctattgcaatggatttccttccaaagggaggtcctccttctccctccctttccacggtcgagggagtagctctgtagctgggtttgccctccttctcctgctgcccgcctcctggttcgtatgcctctgctgcgttcgggtgcgagaactgccgtcatcctcctctccttgccgtgttgtgactggttcttcgttcgttttgtgctctcctgtcttgttgctttcctcgcctgtcatttcccatgtgcatttccgtgaagctgccctccgggactgcagccagaggcaggccaggcctgctgctccactcccctcccctcgttcattaccatccttggcttcattcacatgggagaaaagtatgaaccccattatttaggattgaattcactgtggagttgtgggtggggaaagtgctgaatttttgaaaacgccggttcagtgttgtgtgagctctgtgtttcctatttttatgttgcactctgttgatttttttcatctccctgaactgatctattaaaggaaatagattgataaatatcattttgactaaaaatatctatcgctgttggtgaagtgatggtttttttttacatacaatttttaagagctgtctttaagaggcttatgcatgtgcagatgcatgaacacacatgtacagtcttccttcctagaacatgcttcctcttagttttgggggatatctggcagtcctttcttccatgctgaggacaaaggtatccttgccatctccttgtgagatgtacatttaagtagactgatagaaagatgaacagatggatgaatggagaggaagactgaaggataagtagacaagtattttttaacctggttatttttggagaaaaaaataattcacttttggcttacatttggtctttttttttcttgttataaaatctgatcctcagtatagctctctataaaatgttaagatttcaatatttatataaacaatgaacacattcaactttcccccacctgcttcgttactctgcaaaacagatttgtagcagggagagggaagtcaacaaagttcatttccttcttggtagaataatcatatcaatgctaatttcttgaagaaattttttatattttcacgatgactctttccaggcctttgatgtttttccttctggtgatagaagcagtttaaggtttcatacctatgaagcatctgtttcataaatacattccctccccctccagtttggtttgtacgctagtctcaaataatcgactaaatgcttctcttagctttttttaatcaaaaaaatgtttaagcaaatatgtagactgtattgtactagtaactgctaatgtttgaaagactaaaagatactacaagataatttctcttccagacactgaacccaaaatggaatgaagaattttatttcagggtaagtttttcattgtttggtaataattgttattgatacgtctaggtcgattatcacagtcaattcatcttgaggaaaagctcttaaatttttaatgagaaattccttttaggatgaaaaataggtagcctcctaatgtgtcccaaattttgaaagactagaagtagtggtcacagaatccggacttgtttcattggggagtgttttggagaagtcaaaagaaaattatccaaacaccatgtttcatcttttgaattcaaacctttctttaagaaataagataaaatatcatttatgagaatggtttaattctatgagacgggttatgaaatattttagagagagtaaaacagtagaggcaacatgattaaaaagaaacataaaaattaccagttatattctagttagggaggaaaattttggtaactgaaacacttgagtgcatatatatgtgtataatataaaattacttctggaatctaaatacaacagaattaggtaagatttcctcaaccttttatatttaatatttagaatagctatatgtcgaaatgactgaaatatgtgaattagcagagatttctgcttattgagcatttgtggttgttgctgtgttttactactgacattttggaactattagactgcttagctatttaattgaaagaaaggactttctcttcaaaacgaaggtatcttaattgtatagctgtgatattaaaaactgcttacctccagaaataatccaggtcttctgattagtgcagatttccaaaggccagtattgccatgaggcccattataattggtcctgatttcttcctctcaatcgcaatagtattttttgtgctccaaacttgggctactacatacagcatctgctttccattatgctcagagctgaaatatggcactgatactttcatacttggtgaaatcctatgtacagcctccaccagatgtatttgaaattaccctgtggaggactgaaaagctaataggagctgatagatgctgttggcctttagttaactctgaaatatagctttccacttgtgaacctccaaagtgtttagactcctttcagtggaacactggcacagttacataaatgttagtacatcccatacttttttcagctaatcttggcagtaggcacatttaagttctatttttgttagtttttctgaaggttttttttgtttgtttgttttgttttgttttgttttttgagatggagtctcactctgtcaccaggctggagtgcagtggcacaatcttagctcactgcaacctctgcctcccaggttcaagtgattctcctgcctcaacctcctgagtagctgggactataggcccgtaccaccacgcctggctaatttttgtatttttagtagagatgaggtttcaccatgttggccaggctggtctcactctcctgacctcgtgatctgcccgccttggcctcccaaagttctgggattacaggttcttaaggtctttatatttattcattttacaaatctttattgagtgcctattatgtgccagctcctgcctgtagcctgtggttgcattggtaagcagtgtgacccttcatagagcttctaaacagatagcaaaccccatctgcttgttctgtcgctaggctgcaggcagaccacggaccagaagcacaagctgagctctctgtgcttgtatggtcaagtgttatttcatgcattccccaccatgaagcagccagggcctaggtcacctctctgctttaccatttagagcctttgtgaccctggaaaggccacctgaggactctcaatctccctgggatccttatctctcacataggagagagaatccttacctgcaagggttgtttcaaggatttgcatgcgatgatgtaaatgagaacatgtagctctatgtcacacatgtggttggtgcttagtaaatgatacctgagtttggaagatgatatgggccattttggtgaggttttggtaaggaaaaggaagcaaaaagatatggaaagcccagtagctctgtcattgcctcacagttccaggtctctctcatggttcacagtctaggctctggagacaggcagcatagacctagtgcaggtcctggttgccctacacattagctgtgggctcttgggcaagttacttcactgaacctcagggtctcggctcctcatttataaaattgcgtgatagttatacatcataggattgctgtgaagattaaaggagatcatgggttgttgatatttaataacaagacagtgcctaaaaagtaagtgcacaggctgggcacagtggctcatgcctgtaatcccagcactttgagaggccaaggcatgcagatagcttgagcccaggagttcaaaaccagcctggggaacatggtgaaacctcatctctacaaaaaaatacaaaaattagccaggcatggtagtccaagctactcaggagagtgaggcaggaggatcaattgagcctgggaggttgaggctgcagtgagccatgatcatgccactgcagtccagcctgggcaacagaacaagaccgtctgtctctgtctgtctgtctgtgtgtttgtgtgtatgtgtgtgtgtgtgtctctctctacacacacacacacacagatacattcttaagctattacaatcttcttcattatttttatcattactattactgctatcaggactaagaaatatcctcaggcatagacattttttgatctacagtcatgagaatgccttgtacagaggttaggaacccgtcaaagctatttacaactatcctttcttattttagaaaaacagctaagagccttctcttcttttgctatgcctaaatttggggcagaataatatagaaatcagtaataggaaatttgatttagtacacatagaattcccagaacagcaggataattgttcatattatacaattgagttgggcgcattgtaaagcagtatgttattctttctgttccttacgtcgctgttcaaatgattcctgctcgtttaaaaaatactatttatgttccttataggtaaacccatctaatcacagactcctatttgaagtatttgacgaaaatagactggtaagtggatgcctgtatttgaattttgcttcatttttttattaactggattttcagagcagcgtctttaaaactctgtgtttatgttgtacttaggacagtatatgtgcccaaaaagaagactgtttttgtcccactacgagtttgatgttcaaaatcctaacaaatagttttatgttgctaggataaaaaatgtatttgtatgtgtgtgtatttttattgcagttaatgttaacgaaagccactggtgatagtaccagcccatatgctcatgctctaatgttgggaatacagaacggtaggcagatctctaaagaggccatcaggaatgtaaacaaactttgctcggtttttaaatgaaagccagaccatagtactgggatgcattcagtagcaaatctatggtaattctctcagggaaaataacaatctaattaacaacgtatagcaaaaagtgaatctacatttagcaggtaaagtcaagccgtattttaaaagtggttgccttcttaagagagaaccttagaaagtacctttaaagaaacaaagatatgcctgaggcatataacagatacaaactaacattttatatatagattgatgtatatataatatgtttatgaagatcgacacatacataactgaaataattatgatttcattttattgtttttaattttttttagagagagggtctccctctgtcacccaggttggagtgcagtggtgcagtcgtagctcactgtaacctcaaacccttgggctcaagcaggactccaggatagctaggactacaggcatgagccaccatgcctggccagatttcattttagaaatggaatttttgaagcacattttgctttcttaactatctaaaacgtgtacatcaagtagctgtgaaccaggtgttccaactatgaaaatgaacactatcatgttaagtccttaaggtaccttcagaagtgacactataattcagtgcagtcattctcaaatttaccctctctgaaacttgtatggtatgttgactggttaaaaggtgttcttaagaatcctttctgcagtattcaattgaaactccagatttttaatagctggacgttaatagaatatttgaattggaggagccttatacaataactcatttaactcctgttgttgtcgatgagaagaaccaggtctagagcctttacataaacacatcagtcagtggaggtgccagggtctgacctgctggcctcctaagtcccagcccagtgatagtttgggaatgtattgaatctcatatgattagtattattctccaaaagagaaaactaacagattttggcctaccatcctagtatcttttccattaaagtatgggatgttgggtgatgaacagtcgtacagtgtaactaaaccacttttgttgtatgcatcaaagtactttcttgtgaaattggatgagagattctgctgagggtgcagttcttgtctctccatctctcatccagtgaggaccaagtgagttaagcagtattgagtagtataacagtacgttcctggagacatgggtgtgtctgtagactaatccttattctccaattattgagtataatatctcttgtttaacatgaggaaagggtcaaatttgaggaaaaatggcctcttgtttgatgtgttttagttcattattctcttctattaagagaaattcactgttaaaaaattgtttcccatttccgtatctgaaataatgactgtagttgaggtgatcttgccctgggtctgaaatcatacttccaaaccaaaaaggactttgaatacaaaacttttaagaaatcttgtatgaatacaagctatatctgaaaaattgtgttttataatattgatgcctagttttgccccaggccatctgcagtgtggttactatgcaaagaatgctggtgttgctgtttttttttttttctttgttggctattaacccagcggagacaatatgtggctatggtagtacttggaagttctagcattacacagactagcttccatttctctcatagaggtcatttttggcatttaaaacacatacttttagaaaacagatttggatgtatgtaaacacagggttaatccaccacactctggatgctagagctgttgacaaagtcatgctttgcagattttaaaataaactttttgttactcttacagcttggtattttcccctcctattttttttacctcctctaaataaacctctttgttaaataattgatgtttctggatcatagaaaatagtaagtttaaaatacagaatatttccaagctaactacaaatctgatgacagttttttgagtgtgcacttttccttttatttcttaggtcctttttggtcctttgcaaacatagtaagattccatatttgtgtcccaactgtggtaatattgctgacttcttactggaaaacagtcagctctaggtagcatttcttctgtgtggtatttaagttaaattattaccaaaaaaaaaaaaaaaagcccacatgcactcccttcttcaattttccttttgttttttgttgtttgtttgttttttggttgatgttttctttcctacctgacatatggaagacaatgttttctccagctgggttcattttctctttgaaaaggagacactttggtgtctgccagtcatatgtgagctacacacacgtatacaccttttaaatgaaaaatgctgtgatagcaggagggctctgcttggctaactggttgtgtagcaagtcgggccccctcccctccacttagctattttgttacttgtagatcctgctctaattatttagcattaaacggacactaactctgtagggtgtgtaatcgtatttatagtactgttttattagtttgcattccatcctggtagtgtagtgatgtcataaataacagaaacatggtatccgatgcttgggaaggggtttacccggtggccaagccatattttacttgtcattattccgtgtggcggaaggtgggaaaataccgtgtgtgctgttaaacatgccaggtccctgcactggttagacagagtctcagtgctgtgctccgttgggaaagaagtgccctgcgaatgtgatgtggtttagatttatagcagcgtggcttgcagtgtgtttgctgccttagtcagctgtgagagcgccctgctggggccactcctttctgatggaaaagcagcagctgggaaggtgctgtttcaggctcttgctgtttaaaaaaaaaaaaaaagagcgagtaggggaaaactcctctaagtccagaagctggcattggagggggagagcgcggtcatgtggttctggccaccctaccctctgtcacagtgtggatgagagctttttgctcttatccaatcatgcctggaatgccgcttgccacttgggttatttctgctatctgtcgctcccggtgccgcagtgctaacagtttggcagatgggacactttttcttggaatttgtgtctttggtttgtgacttctggcagtgtcgggcctgttgttgccgcttgccctagccctcctggcccccctggtcaccatggcccatcggcttcggtttcattttggctctggtcgcagcaacacagcccccgaatcagacatcctagaccaggagagagaagacgacttcttcatggcattccacaccctaccgcggagaagcagcccgcaccccttcgcccagaacggaggggaggacggcggcggaggcctgcagggaggcgtgggtgcgcttaagcggagctcgtccatgttcatcccgcagctcttgaccagcatcgacgcccgccccacgtgcagctcctccgtgcagatctccctgcagcgcaaggccacggacggggccacggacgggtgcgggccgcccgagggcgccgacgatgggcctccatgcgcaacgcccgaccccagggaccaggcctccgccactgccaccacgagggcctcgccccagagtggctcccgggagccctcgccgagggacacccccgggagctcccctccgagggcagcccgggacccagggctccaggtcaacggcacgtgcggccgccgcgtgcggtgctccggccccgtggactgcgcggaggaggctgccccgggcctgcgcatccagcaccgcgcctccagcgccgacgtgcgccaggtgaggctgctgcccctgggccccgatggccagggcggcccggccgcggcagagcccaggcgctggtccctgcagcacgttccagatgcttctggaagctctgggaagcggtgttttgtcttccagttgcagcagccccaacaaggcgcttcggggccaggcagcgacctcaactttggcttcacgggcacaaagggggacaggttggtgaggtatcctcgcattcggctggagaggagcacctcgtaccccacgcagccccgaagcgagcgagggagccccacggaagatcggggagccctggaggcatcgcctcgagctggcaggatggctcctgaaatccgcaggacgaactccgcggagaggactccgcagggccaggggtgcacatttaagatcaggcaggatcagaacgcggggcagcagcattttagaattcttgtaacccgggggccggaagaagctccccagaatcccgaggagaaaagcgccaaaagccctgtttccacgggagctgacaccacggtaagttcacagcgtgtttacatgtgtttactgatttcaacttcgatgcttactctgcggcgtaaccaaaataaaacctcaccctctgttccgggagtttccctgcttcaggccagtggatctgaatgtttggccgagttctggcacgatgatttgtggtccagtgtttggtgcgcaaaacaccatttctgcaaatgtcttctgtaggtcctcaaagtcaaagaactttgtctgtttgggaaatccaagcagaataccttccccctgcttttatttttctgaatagaagaatttcgaataaaatttgttactttaatctgccaaaaaacaagtcagtgccccaacgtgctgatgttggaattgcatttaaaaagctgcttgcaagtatagcctgtgacaatactttaaaaataatcagttctcggaaaggggaggatattgcaaaggacctcagtttcgtctcttaatagttaaaataggtgaacgtaaagtttagtggaatttgattatctgtcatggcctgtttgctgcaggaggtcaggtcagtcgttgttatataatttgctttgttttttgtcccattaagtaatactgaacgagagtagtgtcgcttgtgggatagtgacattactatcccacaagtgacactgctaagatactaacagatgcgtttttaaagcacattgggttatggcccaaggctaggggaaactgatggggactcgttaatggtcagatggcgtggcgtgcaatttaacactgcacacttgagtgtccaagccgaggatgctgtatatagaatagtaagtaccctgtctccagctgtggagaaatagacgccttagaacagattctggccttcctgtttctctgttcctgatcgctccctttcagtgaaatggagtacagagagacagctgcacgctccatcgaccaggcatatgatgactctgcagctttcgtggtttagagtggaatgaaaagtttaatcagtttggtttcttttactagtaatcactagccagtgagtgctttctcctaaagaacaagctccttatattttgaattggatattttttctcctgaatttcatgtgactgccagtttgaggatatgattagtaaatgtctaggggacttttctttggatttcacttatgaattgtgattttgaaaagtgagcgtctgagtattactctagaattgtattgtcttttattgctttgtgttcagaaataagtttcgtctttcaagtaaactttatcactcctccatcattggttttcatcattttgaagatacaatcagatgcttaatgaaaatttatttttcttcttctctttccttccatgtatctcctttctttcattatctcaaactcttctattgggaaatggctaagtgctttgtcttggtctgaacctataccatgtattacacaatggtacaaattttgacagtgttttgatattatagtagcctttaacgcccacccccaacaaattaccatcatgacagttgtaggctttgttcggcaacacaattccatgctgaacttggaccacttaccgcatgaagattgacaaaaatatatttcctaaagtttgataactattcaattatattaccaaaaactgaaatcactttgcagttctgatgtggaagacaaagggattcatacaattaaactcaaaaaagttcaaactcatttggtttgaactttcaaatgtaattagaaaacaaatcgaataaactaaaacttatgtgtaccttgcttaaaagggctattctaacttgcaaggcctatgtagtgaaaaacaagtttctacacatttggagaatataaattctggaataaagagcttttcttggagagtaagttccaactagtgaacagtagtttgcattttcatatgtatgtatttttgtacacatacataaattagtacgtggttagtaagctggttttcttttaagcaaaacgatttttctttaaaaaacaaaaccaaaagtggtcacataacaaaaccgcctgctccattcagcaactttgtatgcatttcatatggacaggggtgttaagtttaggcagcagctgccaccgccttacttatcttgaaggaaaacattcacttgggcgtctacagtctggctgctaccaagcttttttggcgtttcaaatatccactgctttggtctaaaggacattacattattaagtttacatgaaaaattagctagaatctgttgcagtttggtggtggaatgatacggaaagtcttcctagatttgggaataccgctcgggattcatttgacaacgtgctcatatttcataagctatactttagaccttattacaagattaagttgttgagtacttttatctgtgcctcccaaacatcctgccatgactaaatctaaaatgtaacatgactccaaaaaatgttaaccaactaaattattagaaaaaatacaggggaaacccttgtgatgataagttttccacctggtttatcactgtcagtagacaagaattcattcgtatagtcatcaattataatcagttaaaaattttcagaattgttagaatttaccaattcagtctgtggcagtcattagtagtcatttagtgagtaatttagttattttcttataaattagcttttgggttattgggttctgagcctcttaactgcattagagttattctgaggaagttggaccaccatttaccagcatttgtgccccctttgtatgccattttgtgcataataactgtgaaactctgcattttacaagcttgaggagccagttttctaatctcattggtgttatgaagtactcaaggtctactgtgtatataacattacacccgttacttggaaagtttcagggactatctcagtgtcttgctgacacagtttgtaatgtaaacggagggaggagggcccacccggacaaaagcagtaattctaacacctactgcatatgctattggagaagagagcgttctttcagctcaagctctctggactttcctctgtgtcctctcagtggacatgtacttcacaggagtagtgtagtaatctcttgtgattaaaaattacttttattgaacaacaaaggaatatccaaattcttttcatttggggaaactttgagtctcttttacagaagatgtttttaatatacggccattttttgacattaatattagtcgatgtctgttttttaccctaaaaataactcgagtatccaatgtattaaccacccctttattttaaaacataacaacacaggaggcgaaggtgggaggattgcttgagcccagtagtttgagattactgtttctaccactgctctacagcctgggcagcagaacgagactttgtctcaaaaaaataaaaataaaacttaatattgtaatggagaagaaaaacaagtagcattcttttcattgacctggtttctgactggattcaatcaactttctttcttctctcatgtatagtaatgctctctcttaatgtggtcttagaataaaccaatatatatcttcaaatgtcacctgttttcagggatgttttgaaaagttggataggcagtgcacaaggttgacaccgatatgtttgtaaacagaaactgtacatggcttgcagcatagctcatgtttctgtagcacttcacaatttgaagaatgctttgtttatttcacttaatatgtatggctgtcccattgtggaggcattatctcacttgaccctcccatgtgaaatggtgttatttcactgtttcagatgaaaggatgtgataccgttgttaggtgtaataacattatgtgtatatcatagtgtatactatgataccactttgtgcacctataatgggaatgtgtatttatgtgtgtgtgtatatttgcacaattaaaaagtatatgttaagccaaaaagcagtttaatgcttcctgtgatgtgacagtttttttgaaaagtttagaggtggtaacttcatttggtgtcttgcagcactggcctgaaggtgagagaaggcttctttggggatccaaagagccgtcctctgcagttcactagcttttggtcctggagtacttagactccctctctaagcctattcctcatcagtaaatttgaggataaaaatgtccagctcgtaggttcatagttaggtttaaatgaaagtgtccatggaagtacctgattcgtgaaccaggtatctgtaatatacaagtctttcccctagcaggttaattaggttaattgtgaacaaccattttttgatcagtatggcatttaacacaaggacaacatgcatttatggcatacctggaaatgtttaataaatagctttgatgctctttctgttgtcatctcatctttcctaagtatttgtccctaacaccaaatacagtcttggaaaagaattgcttcctgaactatgcattatagaaaaggactcgtaagtatatttactgtgttgattttgaagccttataacattttggcttgcatttctagcatattttttaccatgtcattttgctgtaatatttccagtggttgagtctactgttactttggtatagactgaggcttttgagaaacaatatagtgttatatctacaaaatttccacattgtatgactttaaaatgagggagatatttgggaatatgtcttattttaagatactgtcttttagagagttttcataaatggtgcagctgcttctcttggctaagcctggtggcagaattcctagtcattgaaataaatctctaattttataatacatactgataagaaacttgctatcttttcataaaagttcacagtaccagaataaatttactttatgtaaaaactaccttaaaatgtaataaatgttggatagttgaatggtttctggatagttgatgaatatttctgtatagttgaggcgaatctaaatggtaatgctacttgtgcaactgtcaatgatctagcacattttttaataaaaataattacctgcatattactgtgggcgatacaggttagctatcctaaatacgtttttacctttttaactcttaattatattaagccatttgcaagaatgggaaaaagcaggaaattcataggtggtgttgaaattgctgattctacacaaaggtaccagaatgtctacatttcctctgtaaatgtcaaaggaaactttggaaacccacctcagagttggctggtgattattcggacaggttatcatcctgtctgcaaccttctagttctcttagttcataccaagcagcatgatattccatgtcctgcacaaaggagagcaccaaaacagccttcacctgctgcgtatgccccactctgaataaaaaattaagaaggccaaaggacggaatgtgttaaatattagcaaaagcaggtattgcagaatgtctatacatttctgggcaggtggagattattgaaagatttttagaaataatcttcgttcaccacattccagtgccaggccctggacttctgtctctatttgtgataaaatgtttttaagaaatgctggctgggcacggaggctcatgtctgtaatcccagcacttccagaggctgaggcaggtagatcccttgagcccaggacttcgagaccagcctgagcaacatggcgaagcctcatctctacaaaaaatacaaaagctagccaggcgtggtggcctctgtctgtagtcccagcttcttgagaggctgaggtgggaggatctcttgaacccgggaggcggaggttgcagtgagctgagatcgtgccactgcactccagcctgggcgacagagtgaaactctgtctcaggaaaaaaaaaaaaaaaaggaaaagaaataatcttctctcagtgagcatgttgaatgcataatgttctcactggagttctcctttggaatctcttgattattctgctgtggcttccttgggccaactaggatacagaggagaataaatgagtaactcagtgagacaggtggcctgatgtgagtgggcctgaaagttagcccctgatgtcctggctggtttcatgccatgtcctaaacgggcactttcctggtctctaggcatcaccatgttttgacaggaagcttagtttctttcaggatggaggatggaatcaattgtatttcaagaagcctgttttttatgccctgacaattttagaactgtggactgttctgacacttttgagtacagtgattaaatctctgtgaagggacaggtcatgaaatcaaataaaaggacggatcataattgagctggaagggggccctcctatctctgatttgactcgttgctacactttagtattcggaaactttgtattggttcccttaagtgcctgcacaacctccctatttctggaaaccattcagtgtttgttttaatgttaagcattatgccacgtatccagtaagcagagcagagtaaattcctccaggtagcaagagactggaatttcccccaggcagatcagcttttcacaggttcctagagtttacacagccttcttttgtgacttaatttttgtagatcacagtgagtcaagtgacttttacttccctgtataataactttgaggagcctacttgcaacccacatcatcactcattatcttgcttacttctttcttgggggttagatgggctggggttgttattaagcatgttaaatactttgaaattcaaatcgtttctgatagatttagtttctctttcttctattggtcattcgagagctggtagacttttaaaatacatttaatgactcacacatgtcataactcatccatttccttgacattttcaagcgtgataataaatcctacttcttaggcttttttttttttttttttctctctctcatgttcttttcaaatgcatggctaggtagttacctcatgaaggaaaacaactgttttggtccagattttaaaaattgagttgaatagaaaaggtggggtggaaccagcaatactgaaatgtaataatgtatattaagctccaaccaaacaatccagcacttcttcaaatccaggaggtcttctggctaggagttacattgcagaactaacatggactcttaaactttgtaaggaattagtcacagttgaacactggaaactatcagcatacctctgggcttcttttatatggtcttcctcatgagttaaaatgccttaagcatagtatctgttccataggaccttactgattatgcttttccacttgaataaaagagatattaagttgtaataaaactttaaatattttaatttagtttgaatatttaagttgaattaacctcttactacagtaattatcttttttgctaacttcttactacggtaattatctgcttttataaaatccagaaacgatgttatacacattagcacgcattattaggtgcccaaatgccaaggctaaggacacagcaatgctaaggacacagtgtgcctccggcctgcttgcaccctcggttgaaattatattagcaagagaagctcctgcattctgcagtatggcatttttctccagctaatgtatgcaggggtcatttttaaatgttagattcacagatttgcagagctgtgagttgccttggaaattatgctggccagttcctttaattttaaagatgaggaaagtgagaactttcctttttaaaaattgatacataatagttgtacctgtttatgggggtacatgtgatatcttgtacctgtttatgggggtacatgtgatatcttgttaacatgcatacagtgtgtaatgatcaaatcattacatactgaaatatttgaaatatctgtcactgcagacatttgtggcatctgtgtgttaagaacatcccaaatcttgtcttctggctgttttgaaatgtatagtaacttcttattaactgtcgtgctgaggacatggactactgtgctgttgaacactagaacttactccttcatctgaccgtattttcgtacccatttaccaaccctgagagctttacttttgcagtaatagagaatattctttttctctcttgagtatgaaataattatttttgaataatcactgcacgtgaagccctatccttcttgacgttggacatataaatacttagggagcttggtttatagagctttcagtttcacaaacagatgggacagctgaggtgaggcctgcctagcagaagaagccttactcctgatagcaggcactaagcaaggcattgggtgacagattgcttatgcacaggctgggagggacaggaactgtgtcatggaggggcaagactggaactagatgctgaaaagtgaccagatttgtctcagtgggccagtgggaggggcctcattgaaagactgaaggtagatgtgcctcccagggatgtggtggtcatattggctccaaattgccttgttcattattagcaggtttcagcaaattatttttcatgtgaaacgttgctgaagatttcttgggtgtggctgtcatcacaaacaattcctctcttttccatcaaatgtggagttggatgaattccttccttgctggcctgccttccacaaagctgctctctgaacacctgccaggccatgctgaggacaaggaaataccaaggcacagtttctgccatttaatggtggtagcagagacccaggaatgcaatggattattgtcactttcattataacatcatagtgtaggatcataactggaacagagaggatgaagctttcttttggggaattgagggagggagacagggtcttgctctgttgcccaggctggagtacggtggcgttaagatggctcactgccacctccacctcccaggctcaagtgatcctcccacctcagactcccaagtagctggaccacaggtacatgtcaacacacgcagctaattttttagtttttgtagagatgggggtcttactatgttgcccaggctggtctcaaacccctggcctcaagcagtcctcctgccttggcctcccaaagtgctgggtttacaggcatgagacactgtatccaactaagtaggctttatttatggagtttataaagaaactgtttatatatacaggggagaatttatcctgaagaaaaactaaagaaatgaatattttaaatacgtctctgcatttctttttgttggatatgacatttatgtaagaaaaatgctaatctgtatgatatgagacatgttaactacatatggtacatgtatttaaatatcatatgtatttagatttagcaacaaaaagggaaatataaaccatatttccaccatttgaacaaatcgtattcaattatatgtgcatataaccatttgaaaaaattataatgttgatcggatctgcaacctaaaccaagtgccaaattaaattcaggatagataatttcatataaaatgtttcacatcatagaaaaagcagtgaaaattgagtatcagatctgtgaaggatgatacatttctaaccttagaagcagaaaaggaaatcacagaagttggatagattcagccatataaaagtattaaatttctgaaccatgaaaaaaataactaaaatgagggaaacaccagatgtttttcttagtgattcatgtaagttctcatatgtatatcttttcagtgtctaattatgtgcatattttggccagtgggtttgtatctctgtagaaagttctagtaaatactggagaaatactgaggttcccccactccagctctttcactccattgcaaagtaggtcttgtaggtgaatcacacaaacggattaacaagaaagacagaaatgtccagctcccaggttcttcattatgctgttgtgttgtgccaatagaaaatacaatatatcaaattggagaacttgtggttgaaaataatatacgcagattgctggtggcagtattcatggatgtgactttggagtgatttttaaacacttaagtatttttaaatgataagaccaattataggtactaagagtcttgtgttttgaagtgttggagttaatccagataaagttacaaatctaagcagacaccttattaagagatcattattctctgcttttaaagtggacttgctttctatatagttatgaagaacagatagaaatgttttaagattactggctactattaaaataatctggtgactgcagttatatctgggtctaaggcattagactttttggaaaatggatgcttttaatctttccttccgtatatttgtcacccagaattttaaaatttggaaatttcattcattcagtccagatagagaattgatcaattggcaattttattatttttcgctcatgaaaaactctagggttacaagaatatgctcctgaagttaacatagttacttttaaaatgtctcaattctaatggttaggtattagtcctctgagttccggttgccccttaacttggatcttaatttacagtttacagaaaattgttcatatttcatattttaagatcatgtactctaaaaccctattatacttgaaatgaagacctgttatgttatactagaatctctcatgaaaatgtaacagttcccaaggaactttgtttctgtctccatggaccacactggctactgtactggatggagggcaaggcgggagctgagaggcctttgcagaagtgcatgtcgccttagtatgtggcagatcctggtccctggcctgtgctgtttccctccacaccctttgatttctccgaataccaggaaagatcatggattgtctcgggagttagtctcccccttagtacttagcctgtcattttcatgttgcaatgcctagaaaatgggaggtttttcttctccttaaggaagtgttatttcaagatgctttctgctgcctgtgtcatgccagggcgtgggtggatatatttttgcacttttgcaccagcgagtctgcttctgatttgcagattgcatttattataaggcgtattgtccattgatgaataatgtgtgggcattaaagcaaagctgatacatagcttttaaaatagctcatttatcctgcaagcttcacccccatagagaggcaggcccttcagaagcgttttccaggagcaaggtttctgcatgtttccctcagttttggaggtcgtcgttttttgcccctactgtgggggccaagggaaaattgctctgcgccttctgatggtcccctgaaaaatcaactgtcaaaagcccaattaataggagaaatgccatacaactttattaacgtgcatgcagattgattaccccaaccccccaatggggttcagaagcttctataccatcctgaggttataggaagaatggggctcagagcccgaccaaaaacagggtatggtggtcaatcaggttatggtggcaagaaagattcttggagggagagaagaggaggcctggctagcagaggtggccctgtcgtgtagatgaaacctcctaggtagcagccctcacagagaaggcagatgtttctttcagacctttaaagatgtcagactctcagtcaatcttccctagatctggacaagggagggccttagagaaagatggcctcccatcagtgcagtttcctccccaggtgcaaatctccccccacagacctgccctctgaacagccattcacagtgtatcaaagaggtatattttggggtgaaatatttttattcccttcacccctagtttatcatgtgtttccttgatgggctgagggcttgacagatgagcagatagacacaatcttagttcatggaaaaggcaagaatagagatgggtagcggagagtttttaagttgctgctgatgaaatagacactcctttgtctcagctactgaaaattttctttagaacgtggatttaaataaagaaagaaatagacatcatgctttgtaatgaagtcctttggtcatcgtaagtggtcacagccagtttatcctcaggcttctttaagctgaccagttaaggtttctgcagaggttctgtgtttgagggaggagaggaagtccttggggaggaggggagcgttctatgggagacagtgctcttgggagcattctttcaatctgatacccatgcaagggtgatgacaaggtatggccttcagtgactcagagcctcctcccttcccaagcctcaggcctgaaagtctggtgatcctccctggcagaaacctcactggggatcattttttccttttgaacaagtttcttactttgcctcatatgttccaatcttctgtcagtggccaggtgacatctgttggaatgctgatgtgtgttgccaagcaaaacagatgcaggaagacttgcccaaagtgggggaagagagggaggaaagggaggaaaagtgggaggtagagtgtgttagctggggagatgggatcgagcagtatttatctatagaacagatatcaaagattgggtaaatcatggcttaaaactctcagaaaagcattctaggttcttgcacatctttgcaaggctactgaatggctcatttttatttcccaccctgcattcagagtggtgtttaaatggctcaccgcagcaaaaaggattaggtattacccttgtgctcaacacaagtctgtggtcagcagatttttaactttatcctagtttattgatatttctgtcagtgaacaggtttaactcttaatggaatagaatctaccaagaataatgcagactggataaaaagaatttcactgattttgattatttagcacattaaaggggacccctggccagaagtagatgtattgcttgggagaggagggcatagtacccaggtggtacttcagggattctgattttcagtcagggttgagaaaaactgatttaagaagttaatgctactgacagcctaaatctgggtttccttgataacctttggtgtaaatactgatttaatgcatttttttttctcacccacttttccttcatcaccccaaatctatattgtgttcacatggttcctctttctagaggtgggagatgtccttttatggctcagaggtccacaaaataaacttgcattcaagcaaggttgtaaaatacctatgcattaagagcagtccagttgaatatttgcaaagcatatataaaagagcttctctcctaaaataactgaaatgaaaggattaattttctctcagtttcctactgcaagaagtaagtaaaagtggaattccaattctagccatggcaagaccggtctatcctgttatgtcttgtgtgccaataccacttttctccaggtaactaaagtatctccctgctgtaaatattaactgagtgcatttttgtggcttttgataactccacgtcttgaaagtgttggccagttgtcaaattttcatgaatttcagagaagtgggtaataggtggtttctctcacggtatttaaagcgaagtagtaaagaggaaaaagactgaaaaggtaacctgggtcaactcattttttagggcgcacagaagtgtggtagcggagcctggacatcaggtggcctgaagcccaaaccaccagctcccccagcacaggagagtgcaaactgtagctcagtcttggccatctggggtcttctgcaaatgaactgcccatagtctggccaataattcacactgttgtattgctgtctttcattctctcttaagtagagcgttctgaacatatctgaacaattttttgacacgctgttactctcctagccacaggttattttatgacctagtcattctttacataaataaacatttatcaacagtgctgtctgggcctagtaaggtatatagcctgctttgtttctgcattaaagttcttagaccgccatgatcttgagatgtttgctgcttattgtttctttctgatttctattgcttatcagttactactacttgtggctccaaggatagaggccgattccctcttctaatttgctgcctctcatctgcttgaagtttaggtgtttagtcaagctcactagcattgtttataaaggaagaataaataggttgagagtgaggacttagggtagttacctttttaataatgtatatgatctgtctttggtctttctccctcatatctctgctgattttactctcgtcctcctggaacgtctctggaatcagatagatttttgtaagatttggatgtaagaattcagtggaataatatatactagctgtttagcctagtgctcggcacatattaatgcttaataaatattagctcttattattattgtttttttccctgaaagagaaaatcttaattcctcacagatgtttaggaattggatatgtagcaaccctatccactgaattagtatcagcaaacacatttttagttcatcttgatttttatcttcattgggattatgcctttatccctttagggttaatgaatttcctactgtgctgttatggaagaaccctgaaaaaaaaatcaataaaatgtatccttaaaaaagattttatgattcacttgactaccaggtgtcagcaacttttatttttattactttaagttagaaggactttgaaggttttcgtggtttattctcttcttttttaaaggccaaaccaagatcactttttggtaatggctacctttcctctgaatgtatttctcaaaaagttctttctggtttttattaaacccaaacagttgttataatttaaaagttacccttttagagtgagattagctagaaaagaaattaaggattattttaagtaactcagcaagtaaccttttccaagaagcttagattataaattaacgaaaaccaaattataaaccaaggtgtatggttgattgtgggttaagaattttatcagcagttttgctgttgtatgtaccagtcgtatattttactactgagtagaagaaaagtctaaaaatcaagtgagtagtttttccttagctggttatcatgtttgtttgatcattatattaaagtgctccttttatttttcgttttgtatgttttcccaacttaataatgaatgtgcttctctctaatctaatctctgaatttcaggagtctattaataaagtatgtgtaaagaattagttgctaattttggatgtggctgaaatgagattgtcagcaaattacaacttcaagcctaggactgcagtgttatggattgcacctcatccttggttattgaagatatattcaaaatttatgaaaaatagaaaaaccttattacttctgaatttttccacgttagactttccttatacttttaagatagtgaaagtgttattatataagatcatggaaagtttgtctttccagacctattttatgttaggattttacaatattaaattcataagataatagtagagactagaaaacaaatccttactgataatgaatttcctaagtagtaaatacaaatgaatatttatttgtaaacctgggtgattaagtaatagctgttcagtgatattgatggaaggaaattaatattatttaatccaaaggccaggcgtagtgactcacacctgtaatcccagcgctttgagaggccgaggcagatagatcgtttaagctcaggaattcaagaccagtctgggcaacatggcaaaaccttgtctctccaaaaaaatgcacaaattggccggtatgtgcctgtagtcccagctattcgggaggtaggaggtaggaggatcacctgagcttggggaaactgaggctgctgtgagctgtgattacaccactatactccagcctgggtgacagagcgagaccctgtctcaaagaaaaaaaaaaaaaattaatcccgaccttcctccaaaaaagaataaattaagtccagaaagtttggtgattcacttgctgttgccgcctttataaacataaaagtcagtaaggctaagggacacttctgaattccttctgctgtccaggtactgtgccgagttctagagatgcaagtagtacataagacagatgtggcccctgcccttgtggagactgcagaccatggctctttttcatgaatggtagagacactcataagtcaaatacctacatattttcatgaatgtgccaaaggaggcaaacaggaagctgagacaggaaatcatggggaggcctacttgaagagaggtaaggaaacccccaactcaggagtggcctgtaagctgaggcctgggaaggacccctgtgggcatggccagtggaactgcagcggaagcagccgttcgcccaaaggtcctgcgctgggaagagagtcagagatgctggaggatcagaaagggatcatccaagtgagtggaggagacagtggtccaggatgagcttaggggaaaacaggggcccctggattgttcacgctgtagaacacagtcatgcatctgatatataaatagcatggagataccctcagtagatgcaaaactcaaatgagctcagctgctctgaagcctgggaaaagtcagcaatttgtaaaattatgacacatccacattttcacaggaaaaacaaagactagtatgtaacatctcctttggtatttacagaggatctctctctctctcgttgttactcttacaaatgataggaggccacttttgttttccaaattctgtgccagatatcttgcctttcctgagatatagacacggtagagtcaggagtgtgtgtgagggcttgcagacttctttttatgtctttagaatccttcttcacttttgaatcatttggtccaggagtttaaggccttttcatttggcttttgttgttcatttgttgaacataaataactgtgttcattatgcagacaccgtgtcattgttgacaatttggtgttgtagattaaggcaacgtgaccatgtaacaagttgggcaaatgcacttatccattcagtgagctctaacctacaaggtgttttgcttcagaccaaaatggtagactgcagaaccttcggcgcggattttaagtccatctttaaaaccataggtagagattgtatctgtgtgttcttctttaattaaaaatgggaaaatgatgcgacagagcaaagacggccatggatcctttttatccctctcccatccacacagagagtttatttcccctcccctggagtccaggctggccttggggttgccctgaagtgatgccgggccctttctacacgtagcctttcaaaggaccggcagcttccacttcctgccttttggaaaagcttgctttagggatccagctgccacctaaaggccctgaagtgagccacgtagagagaagttccagctttctagcccatctccaagtgtcaggcaggtgagtgaagccatcttggatgctccagcccatcaccatccaactatagtcacataagaggccactgatgggcccagtccgtctccacactcctgagagttagtaagtggctgtcgtgttgggctctgaagttgtagactagtttgttagtgacgtataaccaaaagagagagtgtacttaacatttagtcctgggaggttcgaactttgcctccgcctgtacgtgagctccctggagtgtgggatgaaggagtgcttggcccagttgttttcccggaagctggaggagggcagccatgcacaggttgtgtagaccaaacagttcagggatgcacttggcttagacagctgataaacagctgcagtgcccccaagggagttgaaagagtgggtattttgttgtcaggagtagaattattcaccttgaagaacagaagatttgtagtttaaggaaattcaccatattcttaaaagttgcagaaagagaagagttcttagcctaggcctgcaccaagatccagttcttttgaatctgatttaataggcagatgatgaaggacttattcccaataataaagaatagttcaataggaggttggtagtcttaattgtcttccttgtcaaatcctagtgaatttcacatcagatactgtgccctgtgaggctctatcaccttagcacttggtaatacgttttttatgtagtaggtgctcaataaatgggttgagtttctataataatttgatttctgtagcatctgatttaactaagtttgctataaaacgaaatctgctattgtattcaacatcacaataagcagtatcagttgtaaatcatacctacatacagttaacccttgaacaaagtggaggttggggtgtcagcatcttaacacatagttgaaaatcttttgactcccccacaatttaactagtaatagcctactgttgacagaagccttactgataacttaaacagttgattagcatatatattttatgttataaaaattaaatactgtgttctcataattaaactagagaaaacgttaaaaatcataagagaaaatatatttgctatttattaagtggaaatggatcattgtaaagtctacatcctcactgtcctcacattgagtaagctgaggaggaagaggaggggttggtcttgctgtcacaggtgacagaggtagaaaatccttgtataataagtgagctcatgcagctcaaacccatcttgttcaagggtcaactgtacttgaatggatttttttccctgtcataattaaccacccacacacatcttactagaccaaaaaagaaaacttgtcaagcaaataatctcccatttcttgggtcatttggagagataacacaggccaggttatatgtcctgaaagatgagaatgtgtgaagtccccagagagtctgatacgaggccttcttccagggagtagtggtctggccgctgcaccagcctgccccctgccatgccctgactcacctgcacatcatgggtgctgctgatgcccaggcttcatggctgagcatggcacccttagcacccctcacctgttacccaacggagatttgtgggtcggaggcatttcaattggagaaggatggaatggctgagggcaggaaaacttaacaagcaggaagtctgcaccctggctgcacatgagaatcccctatgggcattgaaagtagggggagcattgaaggtagggggagcattgaaggtagggagcagtcaggtcccatttcctaagcggctctgcaggcctctaagcttgtcagtcatctttttggtgctgtgtggactagtgccagcaggaacaactggatagttttaagtctttggcctcttctgacactttttaaaccaaacattttattaaattctcattcaagttaatagcaaaaaaatattactactggtttcatgtttaggtctcatttgcagtttttaaaacaacctaacggagtatagtaaatacataaagtactcgatttaatgtgcattttctttttcgttttttttttttttttttgggtggggaggggggtggttttctgagacggagtctcactctgttgcccaggctggagctatctcagctcactgcaagctctgcctcccaggttcaagtgattctcctgcctcagcctcctgagtagctgggaatacaggcacatgccaccacacctagctaatttttgtatttttggtagagatgagttttcgccatgttggccaggctggtcttgaactcctgacctcgggtaatctgcccacctcggcctttcaaagtgctggaattacaggcgtgagccaccgtgcccagccctagtgttcattttctgacaagttctatgaaacctggtgacttaacttttctgaatctgtttccctatttgtaaaatgacaatacttgcagtagagtttatgtaaggaattaatgagggtaactttaagatctttgtaaatgctaatgtgttttacaattgtggtcagctataataataataataatattattattattattattaataaaatgcttactaggcgaagttaatagctcccacccattcccaaactacttaaggaggattttaagctggaaacagttaaggaaagccgcatgatgccatgagcaaagccttccatctgcacttggcatattgcagggtctgtagttgctggaatgcctatgtccctggttacgagccttgatataattgtgctaaggcattttggtggagtggaccgtgtcctgctgttgatcaaacatttctgtgtggagagactttgtttttcccttggggagtatttattttttagtgccaggctccgggttggctgtgctacgcttggcttttcagtgccaggcatacaaccatgtcagaagggggactgcatggtaggtctcaggaagaagggccgaggaagaggagaaaagggacttggctgtggacggggaatcctgtgtatcttggcttctctgagccagctgtgcctcagaactttgatttgatctttgtaccccatgggtcctgccaggtgaggggcacaatggagaagagaaaaaaaaaaatgctctgctgaaaacataagcaacaaataaaatagaggatagtaatcctaggataatccccgaggcagcagagaaaggcaggctctggcttgcatgtcaggagctctcttcctgggggcaggagggagggtggtatagtcccagatgagtgcttggcaggttgtttagagcctggtagcagtgaaaagaaatgtctaccaggcatcttgtgcatgtggtagatcttgacttgtaattgtggcagtctcccctaggctcctcggcagctaggtgacagttgcctgctagttcatgggtcaccttctcaccctctggcttgcttctcagtgggtgtaaatggggtaattaaaacctgtggttttagactttgctccaaagactgtttctggggttctgcagacattgacctgaaatttatttttattatttttaacgtttaccacctgtgaagatactgtacatttaaagatataaaatgtatcatattaaaataaaatagcctgtatgttttacattttgagattctgtttatgctgtttttttaaaaagttagaaatgtattggcatgttctgatagcttatttttaactacttctccattgttgtaatatcataacaggttggactggctaggctatggtcagaaattgcttatcagtgttttatacatacccaagggattccaacacagggtgtccatggtcctctttgagaaaactttgtctaaaagatattgaatgatgcctcattaaaaactcctcagaaatattttctcctggcttgttttcgccttccgtgtcattgtttgagaaatatccaagattccttcttttttacaattaaaaaaaaaaattgaagcaagtccataatcccatgtagttctttgatagaattaaacttcagataggcataagaaattcagataggctttctctccactggaaatgcatgcctttcgcaatcaacagtgtatgatgtggtctgttctggagttggggtctgacccttcagcatggccagcgctatctgaagatcagaaaccctggaagccccttgtctctttttcatcttccacatctgtctggcgtccaggccctgcccctcctgcctcttcactgccactgctgtggccagcctcttattcagaggagtttcctacctggttagacacttaaccacctccagacggaccccttctaatccacgtcccacattatagctcaaaagtatggattttacagatcttatcctctcctgttctgccaaaaccccatacggtcctccattaccaatcaggtgaatcgacttgcatttcccagcgtgtccccacacccacaaacactccatgacactgggcccgtccctgcctttattccctgtggcccctgtgtgcaggagccttccctcccctttctatcagtccctcatgtacaggccttcagccccctcactctgccctgttcctacctccatcattgtgccatttatgtttgcatgtctgtcttctctgtcatatctatctcaaactaggttgaaaattttttgaagatggagattttcagaataaaattgttcacacagttatatatttgactgattatacttttatttttatttatttattttttttgggatagagtctcactctgtcacccaggctggagtgcagtggtgcagtctcagctcactgccacctctgcctcccaggttcaagtgattctcctgcctcagcctcccaagtagccgggattataggcatgtgccaccacatccagctaatttttttttttttattatagtagagatggggttttgctgtgttggccaggctggtctcaaacgcctggcctcaagtgatccgcccaccttggcctcccaaagtgctgggattacaggcatgatccaccatgcctggccgattatacttttttaatgtgttagttaagatgagagagtccaaagtgatacagcccctgcccccacagagcttccagtctagtgagagaggcaggtaccaaaaagataatgcacatgacagaatggactgaactaagtagtagtagaagttcaaacgaagaagaagacacaaatggataaaagctatggattctcaggattgaggatgctgaaaggctgcacaggatgaaaggtgcgaccaagggtcctgaaaaaggcataggatttatacttgcaaaggagggcatttccagcaagaaggcttggaaatgagaaaatctcgggagaataggcccatgttagaaattcggtacacgattgagaggaaaagaagtgccactggggaagtgtgttgaagcaggtatgtggatgttcttccctatgcagtggtgtcagtaggattttttgttggtgtgagatctgggatctgtttgtaagccgtctgagagctgggactttgcattgatcaactttttgttgcacatgctctgtgatgtaattggcactgcatgatttgacaagaatgtgttttatgctctaaatcaggagtgagcaaactttctgtaaaaggccagatagtaaatattgtggcctctatttagtccattctgaatgtacattagacacaagtttctaaagtacattcacaaataagcattatacacttagttgtaacatacacggatgttgagcagagaaaaagctctctgatttttgtggttcctgctctaagccaggctaagtggtgttgtcagggttccaaccccttccatccctgaagcacactcagcaaaccatctagtttataagccacagtgtgctagtaggtgctgtgtgtatttttttgcggggggaggaggtatttattttgtttttgagatggggtctcactgtgtttcccgggctggtctcaaactcctgggctcaagtgatccttctgcctcggcctcctgagcagccactgcacgtggctgcatcgcttttaaaggtagatgatgcctctggcctttctcattgcctctgggtgtgtggcccatgtcactgtccagcgcattccactgtccagcccattccactgtctggccccatgccactgtccagcccatgccactatctgtgtgttccgtgccactgtccatctgtgtagggaagagagcaccaggggctggcctggtcaggagagctttctgggcaggggttgagggaggcacatgtgagctggactttgaataaaaagaaaggaaagccaaggagcagaccgggcgaggctgggtgcgaagaccagatgtggcagggatttaggaggcagagcgagcacaagtctgtgcatattggtaggttctgtagacttgtcggttgtgcagtggattcatggagcctgtcgaggaagctacctgtgtgcttccactaggaaagcacagccacagagagaagatgaggggcagggaagcagtattgtggggaattcggagtgagggctgaatctgagaaatattattttaaaattatgttaaatacattattttttgctgtctccaaccatcattactatgttcactgtagaaaaattaagaaagtcagtatggcatatagtcccataaccaagaaattcattcattgtgacctatttgagggtcatttaaaaatgactgatgtaatttggtaaaagattaatcaacaaaatgacttatttcataattaattactttccactaatgtgacatttctcaaactctgcagtatttccttttcttcttttcttttctttttttgtttcctgagacaaatctcactctgttgcccaggctggagtgcagtggcatgcaatcatggctcactgcagccttgaactcctgggctcaagcgatcctcccgcctcagcctcctgagtagcttggactacaggcatgcaccaccatacccagctaatcttttatttttttgtagagaaggtgtctcactatgttgcccaagcagatcttgaactcctgagctcaagcaatcatcctgccctggcctcccaaactgctgggattacaagtgtaagctgctgtaagctgctgtgcccagcctatattttcaagtctctctccttttttttttttttaaactggaaaggatagctgtgatggaaatggaagacttccaacagaataccttagttagcataactgtgtagtacaagatgtaaaaacctgttgaagtgggtggccttttgttatcattctgtctttctgcccaccctgtggcacagttctcaccatattttctgtcaactgctaccacatggtccccttctttatgttataaagtctcttaattattaggtaattattcattgtcctagctctccatgcttaccaaacaatattaacaagagcacggtattacaaatgagtcaataacaacttccctaaccttatggcagctgttttggaggaaaaagaaaaaagaaaaaaaaactgcctttttttttcttaatgcgttacaattggttgatcagaacagttttaaaactataaaatgttggctgggcgcggtggctcacacctgtgatcccagcactttgggaagccgagacgggcggatcacgaggtcaggagatcgagaccgtcctggctaacattgtggaagcccatctctactaaaaatacaaaaaattggccgggcgtggtggcagtcacctgtagtcccagctactcgggaggctgaggcaggagaatggcatgaacccgggaggcggagcttgcagtgagccgagatcgcgccactgcactccagcctgggcgacagagtgagactccgtctcacaaaaaaaaaaagcaaacaaaataactataaaatgtttaataaacatattttaaattaaaaggtaaggttgtatctagttatttatacagctatttttaaaagtcaggttggcaaaggtctctctgagtttcacaaaatatccgcgtgagtggtgcatagtttgtgctggcttatttccatttgttcgttgtgtttatggtaaccagtagcattttctaatttggttaaaatgctgtcattagctggtaaatgcagtcctgtcacctcagaggaggaccaagaaggagctctttctagaaagggagggatcgaattactcctcatcctcatatctacttatctatctgattttaagacaccactgaaatttatactgcgagcaatttaaatgaatgcagtaattgattgcaaaacatgtttgtaatatcagtgactgaaattaaataaaagataagtcagtactttttggatcaatccagttgatctaaaaataaaaagtagacatccactgaatggttgggtaaatgaacgaatgaattttggcttatgtcagtgaattggcctgactgggccttgatctacatagcttctcttgacattttgttgcccaaattgagtttgagtgtttttagtaggcagggtaaattagcggtatggatgggtataacattctacatttagttttgctactgtatatgagctcatgaaatggacttcccttccctaataatatagaaaaaagaaaaacctaaggaaatatagagaacacattcaatttgggaaattcccgtctgtatcagtcaaggataggctagatcatgttgatcatgctgcgtaacaaaccacatctcagtgaattaacccacaaagggggattttcttgctcacaatacacaatctgtgtcagcaggagagcagaattatctgtcgtcaccaagagtccccaggatgatgacggctaatcccattgtgccttcaccatcactgccataggtgaaaggaacgtgataaattgcacactgccttatttatttatttatttatttatttatttatttatttatttagagactgtgtctcgctctgtcgcccaggctggagcgcagtggtgcgatctcagctcactgcaacctccacttcccgggttcaagcgattctcctgccttagcctcctgagtagctgggattacaggtgcacgccaccacacctggctaatttctgtatttttagtagagacggggtttcaccatgttggtcaggctggtctcaaactcctgacctcaggtgatccacccgcctcggcctcccaaagtgctgggattacaggcgtgagccaccatgcccagcccatacactgcccttttaaagcctgtgttcgaaagtggcacacatcactgccgctcatattttactggccaaagcaagtcacgtggccatgccatttcagattgggaagtacatttttaccattgcccagaggaggagagagatatgcaatatttgagaatagccctaaagactacaatactatcccttagtccacctaaatgatcctgaggctttggggcaattaaaaaatccttactcttaaaaaatgtcaggcagtaagtcttgaagccagctgctggtgctacacaacatacccttgtcttgtgaggaatcccttttgaaaaggtaacttacctcgtcacttcattattgctcattgctacaggttgaattgtgtccccccaaaaagatatgaaatcctatcagtacctctttgaaaatagggtcttatttgaaaatagggtctttgcagaccctcttgcagatggagtcaagttaagatgaggccatgaatgtggaccctaatctgactggtagccttataaaaaagggaaaatttaggctcagaaacagacatgcacactgggagaatgccatggaaagatgaaggcagagatcaggtgatgcttctacaaggcagggaactccaaagattgctagcaagcctccagaaagcaggggagaccatggaatctccctcagccttcagaaggaaccaccctgcccatatcttgatcttggacttccagcctccagaacttttaaaccatcaatttccattgtttaagctactcaagtttgtggtactttgttacagcagttttagcaaactaatatgctaatcaatatatatttttatcatcttctttggacctagcaaggagaaccacttgggaaattgaaacaaggaccctctcgggaaagtttggtgaaatcctggccaacatgatgtctaatgctgattgcagtgaaattgagtactgttgttgatttttttttcctcaaagtagcttttggaagtgaagagtaaaatgtgttagtgggaactcagtattagcaagaagagaaataagggactatgctagcaaaatgaataatctgtctttattatactgtcacaggtggttatcaagatacagtttttgtgagacaaaaagctttgtggggctggagttgcttttgatggtggtggcagagggagagttatacgaatagaagtaacttctggtagtgtcttcgttgcatgtgggtacacccagtcttatgggcaagctgattttatataatcttgttcttttcagtaaattttgtatatcaaatatttaatcttacttctttgaaaaactttttagataagtgatgtttgcaaattgaagcatattgaaccatatatctggatattttgtttgattataaacccatttttacaccatggccatagagaatggtgccgtgggcactatcaggagtgatgtcattaaccccacagggctatgtggaaagcgtgtggctgggactgctgtgtggttgggacaaggcagtggtgcacatgggccagctcctgtaactttcctgtttgtggctctcatggggcactcctgctcctgctctccccgtcctcatccatgatacatactctctctcatctacgtgctcataaatttcagcagcaagaactgcatgatttttaaaattctgagtagatgcttccagcaaaggatttcctaactgatcactgccagagaagaagaggtgctaaatagttgccaagttacatgctttttttcccaaatgaagtacttcccctgctactgcttgtaaagccctgattgtgtgtgtgcgcagaacccacatctggtgctggaagcctaaggactagtgagtggtcatgtggtttatggcccctataaaaggacccattctaaaagtctccaagaagctaactccacaatgagatgatgtgtgtgtgggtgtgtgtgtgtgtgtgtttgagatagggtcttgctctgtcacccaggctggagtgcagtggtgtgatctcggctcactgcaacctccacctcccaggttcaagcaattttcctgccttagcctccccagtagcttggattacaggcatgtgctaccatacagaattcagattcctctcctacccctagcctgatgccctttcattagctatacaaaggtggttgagtttcaggttaggcttattatcgtttaaactataaactaaatattcaccaaagctagcccagcctaagcccaggaataatcaaggcagcttgaaacctaaaagcaagaggaggctgggttagatcagctcactgtataatttttgcaaaggcggttttattgaaacctgttcttttttgaaagctggctctttctaacattcaaatgcttttgatcaacctattttatggaagcagaatattgaaattaaagtaaatattagatgaactttatagccagttgttttcctcattcttcaactctttaccctacaaatcttatttaaacaacatgttcagcaagctgttatcttgcttacgcaatgaatagaacattatgttctaaggtacagtttgctcacctatgaaaagaatttaacctcttttctaagtagctatacacaattgtcataccttaagttcttatttagatgcatttcagaatattagagcattttgcaagtagatgcacgttcttacccagtgtcagtgtttatagacagacactcaccatgggtatatttaagtctagggtggtaaagaatataaatgtcatttattaaaacctgaacatagccaactatgaattatttgtgttaatgtaaacgtcgaatgagcactgcaaacatttgatgtttgaatatttgcctgtagaagtgcagtactatgttctatagaatataagtagatgccaccccagtcctcaaggtttcagtgaggtcatcattgtatcaataaaggtgccaatttaagatgctaactggacattggaaaaaatttgttgtcctattctgaaggatgagtcttatataaagtgtttttaaaatactttattgaaactgccatgttcttttcaattttggtagctcccctgcatttaatttagtcataagcgtctgtattaatgggtgtgttcaatatgaaggcatcctgtgaagacaataaattatattatacttatctagtctgcactctttatcatgttgataaaatggcccttaccagtaaaaaaactgtttcagaatgtgtaagagtaagtctgtctcagctgactgagggcttcatcatgttcatgataatcccagcagcttgaaatgtcaaactggtagctgatccatagcacacagtagtgctaattgagataggttggtgtggataattgaatttaattgaatattaaacaccagtaagtgctatgctagatgttgggaccacagtgatgaagatgacattatcctgacccgggaggcgctccttatgtgcataaactgcatttggaaattttcatgatttgagagtcatctatggttgccagtagagattttcccagaaggccaaggatatatggggacatttgactgttctttgaccataattatataactttttatttgcccagaatgaacaactgcagggtaacatttatacaaaaggaatttagtgtgcttagagtgctgataatacttgagaaactaggagagctgttttaaagcacttctttttcttttttttttttttgtttttttctgggctgttagaatttgacattgttcaactttaactttgtcctcattgaactttatcctgctaaggagctaaaaggtccatatgatcaaaaaggcagctctcaacccagcacgaaattccaggctcattcatttctgtttacctactgccaaataagtcagacaactgcatacttgctttacagacgaatagaggacagcatgttgttcaataattgtagtttaaaaagtatatgtaagatacatccacaagaagaaagatagagggagcataccgtttgtcttctagtataggctccctgcccatggagatcacagagtagaattatgatgggtgccttagagaccagcctccctcaggatcatgagttcacctcggcaccacagctctgggcaacgtggaattagacaaattaaagtagttaagttctgacttcatagaacagaatctaaacttttgttcatactgcttttgtgttcttgggccatctgaaaaccttttctgtgtaaatattcctgcaatatgcggtcttacagaggtcttatcacgcatatttccctttgcttccttatgtatcgtattttttcaagaagaagacattcaactttagagacaatggaatcattgtgttgtggctttaattcaggtcattgtccttttgtgagttgcacgtgtgcgcgagcctgtgtgttttgcttggaggaatagtgaataagcaaggagcagagtccatttgctttttctaacagagggtggggcagattctctcacaaccattaaagaaaaaaataagataccagcaataatgataatcagtcatgcatatgtaatcgtgtgtagttcatacttcagattcatatacagctgagcatgatcttgaatgttacaggaagcaagcttgaaagtcaagttacagtagaactttgctaattcatgcctgactaatttgcccacccagtagttcctactgaaatcttgccattcctctaccaactttcagaaaaatttgatagcaggctgtggagaaggctcatgttacttcttatgtcaaggtgtaaacaaaaaagagagcacaaaatacatccatcagaccacatgaagtgaagagagagaaaacatctgctgaaagccggattctctgtttttttcctgctttttaaatgtaatctgtctttgtttatgcatgccctagaaacatgcataagtaaccctgagtagtctcttagtttaagaaaacctactataaaatttcttgttgtaaaataaaattctaaaaggaataagttgtggacatataagtatcagacagtttatgtaagactttacataggatgcaaggtaggctgtaatatgatttggtaattgttttatgggggaattttgttttgtatagcgaaaaagaacagtgagtgatttaactgaattttatagtttaatattgttgttgttttaactaaaatctttggtttgctattgcaaaatgaaagagaaaggaaattttggttaatgttaactttatttccaaaaccatgtgatggatgctgaccaaatgtgtctcaatagtgccattgaggtctgttgcctttttttttctttgaggtgctaagtgaaatgtgttttgaaccttgaatgttcactatgcgacgttctttgcattacaggtagatactgcaaacttgcttcaaaaaaactaaaaagttcaaataactaatggttcatatttgttccagttagtaaatatcacgacaaggtatcatcgtcatcccatgttctcagtatagattcagcttcatttcttacaccacacacccagaggcataaatggaagtacacagacaaaaggaaaggcctgagtttaaatgtagttatcatgttcagtgttcgacattttattctgagaatttaagtgtttctactgcagtaccacaaagtcaatctgcatattactttaactagatcacttgtaaaaataaatgaataaatgaccagtgatggttcagtgacaagagtgccagatgagtcatgaatgttttcacaagtaaatttgatagtaaactgctaagtttactgtatacgctgtgtacaaattagctcagattcaagatttttgctctaacaactgaacgctcagttgcatctttaaactttttgttgcgtgagaggggctgaggggcaggtgggaggaagtcagcctgcaggtgggaggtttctgtctcaggattgctgatacccctgcatgctgctgggttgcagccttggagtatatggcctatgagacatggcatgtctgatttgatggaaaaagcagcggagaaccagtgaaccaagctattccgtgtattgactttttaatacgtttactacaagaatctgccttttaaagtacttttaaataaggttataaacataagtgtgttttggacacagcctgcagcaagcatgcctctcgaggtgtaagatgagtcagctacacatgctcaggggcccgttccaccagccacagtagctgcagagggtgagagcaactgaagcaagagaagctcttagtaattaggggaaggaagcctccacttgacaccgcatcaccaccaagtatttattgagtgtctttggtgtgcaaggcaggagaatatcttctatggattctacatatttcagggttaccagagttataaaatcccttgaaatctatatgcacaaattgtaaatacgtatgcagctttttataagtgttcaaattctgccttaattgtgctcaaggatcagggtctcaccccagcattgtataccattgatagtcatgatttccttagtgaaattaagtaaattgactttcagagactttatttcatattcatcatgtccccatgtagccatgaaaaaatgatatgtttaatattcatctatttgtatgattaaaacaaaatatccgcaactactgacgattcaatgatcagatattttactgtaaattaatatcatcataaactcctagacattagacttcagacattcagagcccactcgatgcttggcacaaagcaggcaccaaatacatatttatagaatgaatgaacaaatgaaggaagaaacaaagaaatgactaatgcaccaaccatttatcaggaagtttaacagggaaattcaacaaccagatttgttttgctaatcttattaaagtgcaacatgtttgttgtaaaaatagatccacattcgtcattgaactggacagtctaaaatagatccaaacccattgtttgattatacttcacaaatgtaaggtaaaatagatgatactatctgtcatttgcttgatgctattggcaaatgtgaagcagttaccagcataaaagcttggaggttactccgaatgtatataaacaacagtggctaggctcacaagtgggagagagtctaaaaataggtaggcgatcccttatctcgttgcattgaaaattgtgtttatatgaagtgatattattagaaatgatgtgtgagaaaagaatttctgcaatgtctcctacaaattagagtgagaaaatgtatctttatgcagctttgaacaaaggattattattgctggactgtggaccaaaattttagtcaagttttttttttttaatgaaaagaaactgttcatttatgcagacatacaactttgtgtcctatagagacttgtcatctgctcttaaagttagcaccctatttttcctctggatgtccactgagaaacccactggtgtgatggtcattgaaaaagagagttgtgtcagaatgccctaaaatcttagtggcttaacacattcccaagtttatttctcattcacactacccgtcaacataggttggcaggcattcggatccacagggtcacacagagaaccaggccgaccgacccacatggtcacacagagagagaaccaggccgaccgacccacatggtcacacagagaaccaggccgaccggcccacatggtcacacagagaaccaggccgaccgacccacgtggtcacacagagaaccaggccgaccagcccacatggtcacacagagaaccaggccgaccaacccacatggtcacacagagaaccaggccgaccgacccacatggtcacacagaaaaccgggctgactgaagtttgcactggagcttttcacagcccccactcagaggtgagataggtcaccttttctcattagccaaaactggtaagatggccttccctgaccacaaggatgctggaaagtgtagtcatcttttctctgtgcctggagaggagagagaagtgaaaattggtaagcaatatttctattttaaaagtggttagagtgtagtgcaatgaatctagcaccattggttcagttttaaatggactgattccatttgtctaattctgtgtcagttggctgttttctgacccctgagaataatttagtctggctgattcccccattgtaccaattgcctggcatatgacaaatgcatttttgcctgtaagacaatctttgcctaggaaagacagccctaatttttatgtcttgtattttccagtaagctaatctggagtatcagtttttaagactgatacacaattagatgaaactaattaaggacaaaacatttgccacttttaactgttgataagtgaaactgagatttccccccaccccatggatacagtgaacatttgtcagcagctggtaatggctagtataaagatgtattcagttttcccttcccttcctctccctccagccttccagccacccccttcccaaacatttatgctcatttagagaaaagcaaccccagttctcttacctgggatatcaccagtataaaaagcacattcgtataaattatctggcctttaattacctgtaaccctgtaggaaaccccctgggaaaagtaccacaaaagaacgcagctctgggggtctgtgagaggcacccagttcacacccctaaaggacagagcgagcactcaaacgcacagctttgggtgccctctgaggcgtgcactggctggtagcagtgggtatcaaccatggaagttcacggaagattaaaccgcaagttcccacgcaaggccaagtggctgccaggacttggaggcactgaccctgagtggtccttccacctcagcgtatactgtcactgctgcctctgtggcttcccctttccctccaagtagagatagatgtctcgaggtgctccccaccccagccaggttgtcctctcctgggtggcattttcctttctactctctggggctttcaccaccccactgggcataactctccacaggctcccctgggaggggtgcccccacctcacccgccacctcacttctcagcccctcttcccatccaagggctccctgggctgcccccaccctagtcatgatctcgctcctctactctctcgtagtactttatttttaccttgctcctggctcacattacatgcttatcttgtatttatagatggttctatatatgtctccttttgcatccttttctcaaagctcctgtgaggccagggatggttttatgccttttctcatccccttatgttctagtataatgctttacacatgattaaattctcatcagatgttgaatgaatgagcttattatgcattacttcttagtacaaagaaatatttggaacgtaagctctgaaatgttgatattctgtggatgcttggattacatacaatgtcctaaataaacacttgcctgttaagtagaagtcagtctggaaagtgatacctttagatccataaaatgtttataaggcacatgaatggacaaccacgttgttttttgctcttttatatgtttaggttactgacagcccaaaatacattagcttgtctttgggctaggaggtacctaaagttgtatttttctctcatgtcattttcctgggtttttatcatatccaacttcgtaatgtccaggaatttgagattaattcctcaaacttgtcgaataatacaagaggaaacttgtataatacagagcaaacttggttggtgagtagctgctagagcaaacttggttggtgagtagctgctacagcccatacatggatttgtttcttcaggtggggggataattgtatccatggactcttcttcattaactgacggatcaaattattttatgaagctatgatgagaaatggcttgtgctcttgggaaaatcaatgccaatgtaggtaatgcaccagtctagctggtttttaatttaaagaacaaattatcttctggaatagtaataaatccccctttggaacagcaagaagacatcagctgttggatatgggataattaccatatgtttcaatgtcgtaaaaaggcacagcaattttaataacccaggttgtgtacaagaaagctcttgcttctgaagaatccatcaaccatgcgctatgatcatgtaataataaactgctgtttacttaatgctgtttttatttaaagttttaattaacataacctagtctttgcaaaacatctgattacctttgactcatatgttttctggtatataataatgggttctcatgagttgtttattttataagatctcctgtttactcagagtaaggaaaattttttttgaggtaaggtcttgctcaggctggaatgagtggtgcagtctcaactcactgcagcctctacctcctgggctcaagcaatcctcccacctcagcttcccaagtagctgggaccacaggcacatgccaccatgcctgactaattattgtattttttgtagagatggattctcactctgttgcccaggctgatctcgaactcctgggctcaagtgatccttccaccttggcttcccaaagttctgggattacagtcatgaaccaccatgtccaaccaggaaattttggtattaggaagtttcccacataccatttgtctttggtgccatgaacacctattttgtctaatttttataccaacctttcactttggcacatgggaaatctgataaggaatcgatatcacggaaatacacatcacactttctgcttgccgctttcttccaaaataaacccaaggattccccttttccaccaccaccttaatttagataaccaagataaatgagaaggttttcgcacttgatggtgggatttcttatcaaggctattgaatttggtttggcactgactaattttggtatccatgtgtttttagagtaagttgtagctagggtggtggggtgatggggagactctcactgaatggctctaaattcagtattttattctttaaccaagcagctgttcacacacatttcttaggaagtggctaaataaaaatatggcagccctggaatgaacagaaactgttgctctttcaaaaaccagcagctacaaatagtgatttttcttccacaaatacttgtttttctgtgacattctcattggttttattcagttaaactcccagaccatgttgatttttttttgccttttattttttgagctcataggtaaaaggagttgaagtgattttattttctgtttttgtcttttttttttaagtttttgtagagacaggggtcttgctatgttgcccaggctggtcttgaactcccgggcctcaggcaatcctcctgcctcagcctcccaaagtgtacccagtctcaagtttttttcacattatatatttgtgttctttctgtttttaactaatggtctccattatttaggctatattaaaataacaataaaaacagagcagatacaacagaattcatttggttggccattggagctgtttttcctttttctttttaaactgactttatttttttggagcagttttaggttcagagcataattgagaggaaggcacagaaatttcccatgtatttcccccacacaggcacagcctccctcatcatcagcatcccccaccagagtggtatgtttgttacagttgatgagccgacattgacacatcatcacctaaagtccatagtttacactagggttctctcttggtgttgtacatgctgtgggtttggacaaatgtataatgacatgtattcactattatggtgtcatacagaataatttcactgccctaaaaatcctctgtgcttcatctgttcatccctaccatgcccctaaaccctggcaaccactcatccttttactgatttttacagttctgcctcttctgggatatcatgaagctggtggaataatgcagtgtgtagccttttcatagtggcttcttttacttagtaatatgcatttaaggttcctccgtgcctcttcatggcctgatagctcatttctttttagtgctgggataatattccactgtctggaaatgccaccgcttagttagtcattcatctactgaaggacagctgggttttggcaattatgaataaagctgctatgagctttttaaaaaatagtttaaatgacatatcaatactttaatatttaaaatatctcagtgatcatatagcctcagaaagcacagttttctgagctgggtgtcatgttgagtggtgttagagtgccgtgctgcgtgctcaggtgctatgctgtgaggaaggatgataggagcagattctgtataagcagatgcacaaccacccacagctgcactttggcacccagggcagacaaataaaaagggggcaagatttttttaggggagattcctggcagatagagattttcttcagaatgtacagatttatttttggggggtggttaggggcgacagttgttttaagcagagttttgaaggagtttggtttggtgagtgaaacagtatttcatttttgattgcctttgtttttccagcaagaaagctactcctggtctgtgggcacacaccaaaaggctttgggcaagacagagggagcttgtgtgtgcccccagaccggagtcataccacagttgtcagatcactttgcggcagaccttccgacagtctcctcatctctcttgtggaatttgagtttgacccacaggtagcattttttattttggtggtaccctgatgtttataaggaacctcaggacactattagttaggaaggagattgtcccatgcaattcagccttgcaagagcttcctggttgatctagggacaaggtgtctatcactcagccctccaggtcttttcttcctaaagcaggctttggagcaaagcctgaccggtctttgcctatgactgagtttcccagggctgctgtaccaaactactaccagttcaacttaaaacagcagacatttatcctctcacagttttggaggccgcttcaaagtaggtgtcactgaactgaagtcaggatgtccgcagggctgcgctccctccagagcctctaggaacacatctgccccttgcttcctccagcttctgggggctgccagcgggctccagcctctgccgctggggctgccttgcctcctctgctgtgtcagatctccttctgcatctctcttacaaggacacttgtgggccgggcgcggtggctcacgcctgtaatcccaacactttgggaggctgaggcaggtggatcacctgaggtcaggagtttgagaccagcctggccaacatggtgaaaccctgtctctactaaaaatacaaaaattagccgggcgtggtggtacgcacctataattccagctattcgggaggctgaggcaggagaactgcttgaacctgggaggtggaggttgcggtgagctgagatcgtgccactgcactccagcctgggcaatagggcaagactctgtctcaaaaaaagaaaaaaagttaaacgagtgtcttggcactcccctgtaatcccagctacttgggaggctgaggcacgagaattgcttgaacccaggaggtggaggttgcagtgagccgagatcacgccactgcactccaccctatgcaatagagtgagagtctgtctcaaaataaataaataaataaaaataaacaaggacacttgtgactgcatttagggcccagaacaatcccccatctgaggacctttcatttaatcacatctgcaaaggcattttcatgtaaggtaacattgaaaagtttcaggggtcaggacctaataaattttgtgggccagtattcagcctactgtcccctagttacaacaaggatggggcagcttatggacgagagtgtcaaaaaattcagattctgtcctacagggatcatcttaccctgcagctgagttgagtgattattgagaacatactacgtgttgatgctgttgcttagcactgtgggagatacagagatacataaaactctgcttgccctgaggttgcttgtggttttgttggaagagaagacgtgtgtgtaaaattagtcataagaaaaaggtcgtgtttgatcctgagagccagatagtggtacccagcagtgagcatccaagcccttatctcccaaataggagactttgtaggctgaaaactggcccacagatttattaggtcctgacccctggaacttgtcaatgttgccttacatgaaaatgcccctgcagatgtgattaaatgaagggtcccggtatggggggttgtcctggatgagctgagtgggccctaaacgcagtcacaagtgtccttttaagcccctgctgtgcctgttctcttggtaaatatctctgtgtgctggaacatttgaccagagagagatttcggtgatcatatccatatttgactgtttcctattccataatttgtcgctgtctccgagtcttttcccgggagccccctattacaagtgcacctttctgtgtttattcttccctgttgtgcttcgatgatcccttttttctattcttcagtggctttttctccagtaaaatagcagtttaaattcttgaagggaaacaattaaaaaataatgtctctgggttaacaagtatgattaccgtcatctaaatcagtgatactacatgagaataaaatcaaggagacttacatttacagtagatgaatctatgataatatgccctaccgttttgggtaatttaaatttatgctagttcactctgtctacatgcatgtcatatcttagtgtaaattatagaaatatccctataaaagaaaaaataatttttttctgtatatgttttaagggaacctgaatttctttgggttgtaatgcatgatacagatagtattctgggaatacctgatgttcagcttatccctggaaccctggttatcttagtaaaatatgcagcaacaatatagaagacaatcctgaattttccactgcacaattagggcttgattcaccgtatgacttgggcaaattacttaactttttgaaaccacacttttctgattttcaaagtatgactaataatggtttctgccttaggattattaagatgattaaatgaaataatgcatgtgcaacgtttagaacattgctttaaagtttgtcttttccttctctctccccttttttcctttaaaataaacaaacatgtttgagggctgggtgagttgatgggctccctttagtgcagcacttctaagtaatcactgttccctctggttcattccattgagcacatgatgacaaacatttctggggatatcatgtcaccttggatatagaacagtctcctaaattacattttatatgacaatgtctcactgtatttttgtcctcttgaattgcagcatagcatattatttcttccagcaaaaagcaaaattaaataaataagtgattaagttgctggctgtagctgatactgattcatgacatgactgaggattacttagccatcacattgaattgatgacagttgccttctaaatattatctttagtcacatccctcgaatgtttgcagtgactttcactcctcaactagaatatgcacgtttacatacacatgcgataaggctgaagaaggcttgtggctcagcacaatttggtcttgtatgcacagtaatcattctgaagtgtatgattctgttttattagaaattctcacttgaatttcaagagaataaaggaggattcctcctacacaaagctgctaattgtttgcacactgtgtgcatcccgcatgataagcgatttgagaaggtgccacatcagtgcctttatccaagtgtcacctcctgtctacagttcccttagcagggtgaagggagaaagatgcaaagactgagcttggctgaaacaaatgtatttttgtccccttgacttcttgaattgcagcagagcatatgaataacattgatatatttcttggctgctagacaacaacgtatttctttgacagattgacgcctgaaaagaaaaatacagtatgtactaatgaatcaaaacattttttagtacctttgtgtatactaaatgtattttagtttctatctggagctagttgccatttactgtgggtttgaaatgccctaagttatcttctatggatgggtagaatcaatttacccacacattgacagcttttacagataggtcctgtaacagatataattaagttaaatttcataaatagataggcagtctcctccccagtcctggtccctttgggagatgaattggggtgggggtaatggagagtttcaaaaaggctttccggtgtctttttttaaaaataaggttttaagagtttcatggtcagctgttctcagtttttctcaagatggtatccaaagtaaggctcaaatttatgatttatcttccaaatgcttaaatggtccatttatgatctgccagatgttacaatgactagctccccaaggaacgatcaagaatgtgccaatgtctgggcaagatttttatccagcaaaaagaaaaaaaccataagaattggaggttttactttgaaaagtaaaatcagccacatccatgacttaagagactgtacatattgtcatgggtaaggatttagatcttggctccactgcttactcacagtgtaccctggacgagttactaggcctttctgtgttttactatgctcttttgtaacacagggagagatagcatctatcttctaaggttgttgtgaggattaagtgaactaacttgtgcaaagctcttagaatagtgctgggcacagagtggtgagtgccttgtatatgctaatagttgttgatttatagcttaaaattaattgtgtgatgcaaataggagggttaccaccccaagcttaaatggaagttttgcagcactttgcactcttgaggatgtatctgggtttaatagtaccttccatctagtgcacaagtcttctcccaggctcatcctcccaaacacctctcagtgttatccccactttgcaaatgcagtagtagctgggctcacagaagttaaataactacaccaagtcaccatccaaaaaggttcagggccaggaatagagcgaaagacagaatacggagccatgctctttttgctgtctttctcagtgctcagttctcagaattactcaggaatgagagcagagaaactctgtataggccatgcctggagttttttgcacatggcagtcagagagtcccagctcagttcagtgcagtattaccgaattccagtatatgctgggctctgttaggtgcaaggggttccaggatgaatgagtctgtggcccagtggacaaggtttgcccctgtgtgcagctctgtctgctttactagatttgctcctcagattctaaattggaagtatgtgcatattgcttgatttacagattattagagaatttttttcagttaaactttgtccaaaatagacactaagggcaaaggaaactggtattattacagcagggtgtatctcataagctctgagaattgtacatgtataaagaatctttgaatcgccgagtcttagccccacgttttatacagcacagaaatacagcacagaaagtttaggtgatttcccttgggttccatagtgtgttagtcaccaggttcctttaactctctcagtactctttcctttaaactatggaagagctcaaatgattaaatcaataaaagaatgacaacttatagagctcatttacgtgtctgaatatatcagtttagcccagagtatctgaactcctgggccattgctattcactgaccccttctcctcctcgtgttctagtaaaattcagcccatgaatatagtcattagaagttgaattttaatgaaattgggcgctttgatcaatgggaaggttgaaagttatgtgggaatcatctatatagtttatagctgtgactatgtaggccagctcctaagtgccccctgcaatgtgtcactctctctggttttctttccagggaggtaccctgtagaataacagtctaatttacctctcagacatctgctgtctttcagaccttatgctagttttgtgcatatcttcatgattcctctaataatgcattggattataaactattatctccacttgagagacgctcagagaggctagggagcttactcagggtcagagggagtgcagctgaacagttctgccagggtccctgtgattgttggagcagcctgtgttttttcaagctgttgtctgtgaatcgtacggtagaaacatggtgcgtatttctgcagccttagttctagccattgaaatatcatggagttgaatgcagaatacccattaatgtttaggatgaccaagaaactgtgcccaggccccaagcccagctctgtcatcagaggtgagttaccactggtgccatgccctttgattccctacaggcgccctatggaggtgcccaccagtgtgcacttggaagtgtgatggaacgcatcacatccccttgttgcccctgccccggtgatagggggattcactaagaacttcagcatagtgagagaatgtaactgaagctcctgggttagtgctgcttggtatcccagttactttgtagatttcggaatcttttgtcgtctagcctgaggattactgttgatgacacaattttctttgggattataggcttcatgtttcatacaaccagcatatacgtaaatggcctcttcagtgtatcatgtatacacattggtgaatagcttcccactagataatgttttaaaggggaaaaaaagaagtcatttctaaaatcccaagagagtgtccccgttttgaggttagagcacgttttttagccttatttagtcttgcatcttgctccttatgctacctttcattgctttatgaaattattttttaagggatactttctaacattccatcccactttttttaaagctcgttcatatactttgtcctcacagaatagaacagggtttctcaaattgcacacactgaactctgcaacacaacagttccttcattggagaccagatgaattgaaccactggctttctgaaaataactgtatgctttccagttggccagccttggcagggacccactactactcctatttctcaacagtcaatttaaaatattgacagtagcttaataacattcaatagtaacttgcagcagcctttcatataccctgacatctgagatatgtaccagtttatgaaatagaacacgcagatggtcttagccccattttccagatgaggacccttgtccagggaaggattggtgcccatttagctagctctttcccagcaagcaacagtacccaaggctccatgccccaggcctgggaggacccctcccacggcgtacttcagatctccacctgtgactttgaacacatgcttcgaggtgatgtacttcttggagatagtgctttcctcatctcttctaccttgtagtcgtcagtagagggcatgcagtgcaacagaagtggatttcaggactgagactagggtgaggcaagagaggcatcagaatgcaaaagtgaagtagacacctgccgtcagcactgggcagccctgctcctggggccttacttgactcatcctggtgggttttccaaatggcagaggtgcacaaccacttcttgcaagcatgggcacttgaattgtagcccatgtacacagtgattcttggaaaaagtcatgagtaatgaatggaacttttctctcccaggttgatcttgtgttttgagacataaagagtcaaaggttggtgcctaggatgctcattcttattgtttttggtagtttgattgttgaaggcttttgacacgacattgaagtgaatatttggggcattttgctcctctgggcttttctctaaagcaccctcacatttcctctctcatgttcctgggctttcctggctttgatgctagccccactgtgtgttcccattaatagccctatgtagcctggctccttattctggaaactgtgtaacttttgctcttgatcttaaacacaactcagtcttgcaaggagctccgcctttggtctcagcttcttggggatttccctggcagccacatagtgaactatgtcacactctctgttttacccgttaataaggaaatactctggtacatggagctttgcttgctgatctttttctaagtactctgggggcaaaaggaaaagcagctagaggataaggatgtaactttcttcaaattaagccatcctgacccgtggtagagattagccactaagaaatagtggaaacaaataactgtttcactcttgagtgttagcatggaagagattctggttgaaatcctagtcctgccactttctggttacagaaacttacattacttttcttttgatgtttcaaggagataggagttaaagaaaaatgaacaccagattactagaacattcttatagagttgcccaaagatggaatggacaccttccagagtggaggggagggagtaggattggacagtagtcctcaactgggagcacttcttccccctgaccccacacctgcctcccagggacgtttgacagttttggattgcacaaaactgaggggtgagagttgctgctatatctagtgagcagaagccagagatgcttccaaataccttacgaagcataggacagccctcacggaaagaattatctagctccaaatgttaatgatgctgactttgagataccctagtcagaagtaacatttattggccaggggcagtggctcatacctgtaatctcagcactttgggaagccgaggtgggaggatcacttaagcccaggactttgagaccacctggacaagaccctgtctctacaaaaaaaaaaaaaaaaaattagcctgatgtggtagtacatgcctgtagtcttagctactcaggaggctgaagtgggaggattgcttggacccaggagtttgaggctacagtgagctatgtttgtgacactgcacttcagcctgggcaacataacaagactctgtctcaattaaaaaagaaagaaagaacatttgttgaatctaattttcccatgtaatatcaatgactgtgcaagataaagtggtttttatcctcactttggatgagattggcctttggtgattgagaaaccttcctttggttgttcagctacttctagatagacatgggattttaaactagccatctatatgtccaaaacccatccgctttccacaaacccattatgtagttagctccccatcactgatgatgttcaaagctaagcagggtgagcaaccacttgaaagaggggatggatgaggatacaagcctaagatgtatgttgcactaaatgacttttgaggtccctcctaaagatcacattctctggttctattttgaaatgggatttgtctttttagccccagtgttagagtggcagccagatatcccttggctatgaccagtgaaatccctgtgctttgtgcacctgtgaatcttactcctattaatatcccagccctatcaggccagagggctagggttccaaacaggggcagccagaaatgcaccatcttctgcatcccatctgcattccgggcccaggcagttatttttacctccatatgtaaatgggcccagcgatttctcatttaggagaacagcagaggcacaagtgaggagagctggagaaggaggggaacctatcatatactctgagattgcaatttagtatcagtttgctgatcagtgtcactccactagtttaataagtaattatagtttctcttttgtgaacaaggaaaacgggtagcttcacccagtggttgtcagccctggccaaaaattagaataagatagagggcttttaaatcacacctacaactgagcccctccaccagaaatttaattttctgaggtgaggcctgggtacagtgatttgagaactttgcagggattccagtgtctaagcagcctgggcacctctggttaatggcacacctcagtgctgcgcattctgctccagtacctgctggtgttactctgtcataaagagactagaaaaagagtgagttgggccctgtttctcagtgtgtcttcagtggattgtagggagaaggaggcccatactctctgcctaaacgagcacctgctagactccgaaatctagttaatggaagtttctcctcatccttggacatccttgttttatatggtatacgttcaaaatacatataatctttggtttaatttgttttacaaagttttgaagttttcctctccacacttttctcatcaagctggtgtgtggaaagaaggaatttttcttttccattgctgtcatctaaaattgttgtgtcgggtggggaggggagggagccgggagacttagctttaactacttagaagttttcagggaaatgtttttataacaacctagttaatagtttgactttatgatagattctgttccttcattattttgtcctccagtggcttttatacccagcaaagtggaccatggtgtatgattcaagaagggtgaggagtatgtctttctttggaaaggcagggatggagggttgtgatttagaaagggtaagcaggaaaggagagccacagaaggcaagcactgttctcatgccagatcggtttcaaggaagaggacatatggaaggatctagaaaatgatttctctagaattgtccatgtccaagtcccctggaatgtctttgtatatccccagagctgtgcatgctccagtttgaagagctctgctttaaaagatatcaggggtgggggagatgaggggggtgaagtaaaagaataaaaactattcaattccatttgttttctctttcaagctcatacgttactcatttcatttttatattaaaatatagatgaggaaggaagggcacatggcaagatttggcaagcataatccatgaggggatatggagttggggatggcagcatagcatactaggatttttgaggagggattcggggagcctgttcatgttttccttacaactctggtacatgaagtacaggttctgcatcatgatggaagactgcctgggttcatgtttgggccactacctactagctctatgactttgggcaagtttctttcttcttttttttttttgaaatggaattttgctcttgtcacccaggctggagtgcaatggtgcaatctcagctcactgtaacctctgccttttgggttcaagcaattctcctgcctcagcctcccaagtagctgagattacaggcaaccgccaccacgcccggctaatttttgtatttttagtactttagtacagaccgggtttcaccatgttggccaggctgttctcgaactcctgacctcaggtgattcacccacctcagcttcccaaagtgctgggattacaggcgtgagccaccatgcccaggcaagtttcttaacctccttatagcagtgtttggcacatgatagatgagctacaggttggcttgttattccttgcaatcctgagtttctccccagagtaaagctggagcttttcccccagccaactttggtggagtccactgggcagagatgtgatttcagccaaaatatggctaaaggaggaaaggatctgagacagccaggtggaaatggctccccgggagagcctctgaccgacccgcacactgggaggagtgcgcactggggtggagccttgggaagttggagccatttgcagcagggaggagcctggcccctcgtcttcttggatggtacctaggattcagtctgcgaggcaggaagcacactagcaggactccagcttggtggagagttcctgctttcctttttttcctttttgtccaataaatcccatttttctcaccctttaaattgtgagcctaatttttcgtagtcatgtgacaaggacccctcttaagctgaactaaggaaaagtcctgcagcagatctaactagaatgccaatgcctacattgaggctgtttaatggagagtttatgaaaagttagctagtgtccttgtgtaaacctgtatgactctagttcagagtcttcagtaattgcaattcaagaataaaaccacacggcacttccattcccgtgtaatttgttgttgctggcttgtgacattctacatatcccaactatgagacagccaggcagtgggatggactgtgaggttcagatgcccggagatatgtgaggaagagcttttagcaataaatatgaaaatgaaatcaaagtaaaaactggcattgaacccctggtgtacttaggcttacttttacccaaatccaaatcatttcttgacactgcatggttaccaagagtgtctgtcattcaatatttcagattaagatctgttctttggtagctaagttcagagaaaaaaatataggatctagaagtatcattgctattttttccttgatacatttagtctggtattaaaattgcttgcagaagatttgattttaaaaaagtagaaaagagacacttatcctggtaaaattatcctgaaatcttaagaaattaagacaaattgcaccataaaaccagaagcacaaaattaaacaatattatattaagagaacccagtgacacattttccttctaaattaactttaaaattaacttttaaattaagttcaatctcatatcagtttgctttgtaagaaatagaatatatgtacatgtttgccttatttttagcatgttatattcattaaaatgttttagtttattcagcagatacatttagcatttctttcacaagaaagacagtgaaggaacaattaataacaggaccccagtaatacctggggggtttctacatctgcattttttgcaaaaatagaatagccttctaagaagctgtcagtggcatttgtgcagctggacagctttgcagcagatgtaacagtaactgtgatgccaggctgtgccccagaacctgtaagcatgacaagctctggcttcttccctcctctgaaaaggtacaacattgaagaggcagtcgtttcccctagatctgtaaccaagcacatctgggtgaaaggggaagacatctcttcacagagaggagaataaaggcggtgaccatcagggtgacaggcagcatggactgttgccacctgtgctgcaggcagcttcagtgcctggatggtgggacagctggtcctgcactcagcacgagtctcaggcagtggtctggccgcgcctcccttgggctgcttcttcctccaccgtcctccacttttgctttgtgtctgcgtcaagtcagtaacgttggtctgcagctcccatccctcctgatgggcactgcactgaggccttagtgggcattacctgatgccctcatcacccagtgtacaaacacctgctgacaagaagcgtccctgagcggaacccttctgctgggacaccgcaccttccgtgtcctgagtgggctgcaccctcgtggtgctcaactgggtgacttcacgctgtcctcagcgctgtcctcggcgctctctctccagtcagctctcctgctcaggcacctttttgggacacagtccttgccagttattccctgtttaatgtcctcatgtgtcttcacttgccctgggagactgtcgtcttgagagagcaagccatatgggaccctgaggcctggcctcagtttccctcccccccactgccccacggggccacgcctcatcagtggcatggaccttttagtcagcgagccctcagccggatgctcccgtgctggccactcctgtcatccctgctctgtcctccctgcaaactcctccctcaggaccctgcttgaagttacgtcctgcacagtcatccctggccttccctcagcagaatctgtcactccctcaaggtgtccctgcagtattctgctcatgcccctgctacgtggcagaactctgttgtcctatcctgtccagagctccaaagcccccagaccctgtggctgctgagcacttgaaccatggacagtccaggtgaggatgtgcggggagagatgcctcctgcctgtggctgagccctgcctgaaggagctgatgaggaaaagctcctaagcacaacagtgctgctcacctgggcatctagttggcagagtcctatcccagtgttcgcacccccagtttcctgggtcttaagttcagattatcaagtgctcctcctgcaaaaaaaaaaaaaatggtgattcttgtggcccttagttgaccccgcacctgggatatgcttatatttggtgtggggcagagaactgagtgttcttgaaccacttccacccaaccagagggaggtggggacatcaggtacacctcatcccctgatgtcacagccaagtacctggagccaccaggactccctctgagcccacaaaggagtgtcttctcaccaccccgattatactggtctagttatgctttggagttaaagccaggctgctattgcagggctgctgcaatccttgcaagcacagttgttctttgcttgcgcttgctctcgctctcgctctcactctcactcttgctcgctctctttctctctctctgacttcagtttaactttttaaacttcagtaataagtgttcctcaaaatagatggtcttcctggttgtaggtacagccagcagggttgagttctgctgccttccagcatggttttcctggggcctgtaaaagtcctgcaaatttgctctgacactctgttgacatggaggtcctgtttgttgttataaataaggaccagtgtctctaggacacaccttactgtgtataactgaatggcagattttgtcccttacttcttaaaaattaaatggcagaggagctggccatgtaaaagccctgtgtgtgtttaccaaaaagtacttgaattttgtttgggttttgtgttctggtgcatatgaagtgaattttggtaggagtatgtgcattcagagaatatgtcatcacacttttttctttatttgagtgccacttggctcttatttgattaaggtaatttcagtgacatattttgaagcaattcaccttgaaaggcagaggcaccacccaggtgcagaaaccttccttgggttatgcaacgcccagcgtgggtggaaccgcactcacactttacctcttcctgaaccacagccctgctctgattgcatgatggaagctgaggtgcctgacaagtggaaggaggaaaaggaacattctgccttccttttctctgtaatgccaaagctggcgtcatgcattactgagggcctctgtcagcactggggccggccagtggcacagctgatggggtagctgcactcgctagtcctctgggaaagcccaggttgtttttctggcttagtgttagaggcagctgtgatgtttacctcgatctgtaacatacaaagatctgatcatatgctgtctttgcttacattctattattgtctgacttatgttctaaattctctacaattcccttccatttgtcttaagaaactctaaacctttttttttttggaaatcactacttaatgattttgaggatggaaccccagacagttggtaatagtggatggttttccttttctgtagttgagtggctttctaattcttttgatggcacacaaaaagaaattaaccttagatcacaacttagtacacacatttatgtatgcatgtaactgagatgagttcctcaaaatagcacatacctgtgttttatgtgaggcactgatattttctgttctatattatgcttaactttaaaaaatacttcctgtgtaccatttgtgggactgtaactcacaacttaaaaaatattaccttgatacatagctccagaaatacacatgggtcattttaccaagaatcaccagtgcttttgctctgttaccccggctggagtttggtggcacgatcatagctccttgcaccctcaaccccctgggctcccagctgggggtctttaggaccccagcgtgacctgtgggtagcccttacttcctttaatacttttcttctgcacagtcaggtgctctccctttgagcttccgtgtgaagttcactgtgcacccagctctgctaccaccgcctctgtcttgaaaggaatagggtggggtggtcattggtgtatagtaagattcgaatcacatccttcaagtacagttctaccctaaagataagagtccctgtggtttttgttgttgttgttgttgttgttgtttgaatggatgtgttctttgagaaattgagtcttgagtattttgtgaggacatctaagcctggcgtccacgtcagtgctgacagatttgacctagtgtgcactcccatctggggaaatgcccataggctataatctctttcactggagcgattataatagcccccgggcttcaggtcccagtggcatccacccttggtggcagcatacccctctgcatgaacagggcgcccacttgtcccctccttcttacttagtagtgatttttaaatgggagggtggcagtcctgaatgtagttcatcagtatttaccgcacaagctctaaaggacacagtgttttatttgtgtatttatttaacttttccaatatcggaatatgaatttttgtttcacctgataggacacgtttcaggggctccattctacattgtcatttcacaggaaaaatatttgatggctcattagaagggaacatctattattttgttactgaaaggcacattttcaaccttgagaattggatggcaggtaatggcatggaaattgacagcattttaccacttaaaaataaccacaagaaataccaatgttcaccgaatgagttgaagttaatttggttttgtaggctattgtagttaacttgatgcttgatggaaataattttgtattggaaatcattaaaaattgatttgattaatgcttttataatgttggtatgaggtgttgcactgtggtggtgatgacctaagggtgctgtgatggcctcaaacgttgtccctccggggcccacaggcaggatcccacacactagcacacctcccagttcctcttaccatcaaacagaggtgttccttcaacatgcctgctcctccctgtggcataaaggcaccaggaggttctctgatcccctgttagatctcctctgcagggttgaagacattttttgtttgtttgtttgtttgtttttgagatggagtcttgccctgtctcccagactggagtgcaagtgacatgatctcaactcactgcaacctccgcctcccgggttcaagcgattctcctgcctcaacctcccaagtagctgggactacaggcatgagccaccaagcccagctaattttgtgtttttagtagagatgggatttcgccatattggccggggtgatctcgaactcctgacctcaagtgatctgcccgcctcagccttccaaagtgcttagattacaggcgtgagccaccgtacctggccatttttgtttttaatcggtgtagtgtccctaacctaaatatttcacatggcctgaactttctgtagttttagcagcaatgggatgctttgaaacctagatctctcaccaactacatcttccattgactggtgaaacactagtttaagccaattcattttgaccatgtgcatttttgtgcttgatccctattttatttcaaaaataatttccatctctattcatgtaaactaataagagccttccgtttcctttaatggcaaattgtctcatcttttactgttttaaaacatttttttaacatcatgagaaaacgtgataaaaggtataagtgtagtttcctggcttttgcagtacctcagggaattcggtaaactgtattccatttgaggaaaggaacggttggagtaggaggtagtgtaaagataacatagcaaataagcattttaccagaaccctctgaagtctcatcagactgtgccatcatcagaatccctcccttctcatctacaccgtttaaagccagcatccagtcagaagcccagagctggtaccgccttgctttcctgccagttgctgacatatggcacacagtgtcagttaagggcacaggctgatgggcagcttggccacatacctctctggcttcctatctgtaaaatgggcacaatcattgtacctgcctctgaaggttgttgcgagaactagacgcagccttggcacagagcccacagagagtgagcactgtgtaaagactggctgccagccattagtactgggtgcctgtcttttggtgtctgggcaaagctttaacctgttttatcagtttagaactcagatgtctccctggcaaaacaacacgggcatatagatattttgtgaagttttgttgaagtgcacttaggttaatgattcatgggcagcctttcaaagctatcaggctgtgaaacagctttgaataagattgtcttgtccttctgctgattgtcttttccttcccacattagcagccatagcaggaggaggagaggtggcaagtggccacactactctgctcacattaagcagacaatttatggtcaatattcagtgaatttcttttttttatagtacagttaactgatatttatattagctttaaaattatagcaaaatcaggccaggcatggtgactcacacctgtaatcctagcactttgggaggtcgaggagggcagatcgttggaggtcagatgttcgagaccagcctggccaacgtggtgaaaccccatctctgctaaaaatacaaaaattagctgggcgtggtggtgtgtgcctgtagtcccaattactcagtaggctgaggcaggagaatcgctttaacccaggaggcggaggttgcagtgagctgagattgcaccactgtactccagcctgggcgacagagcaagagactctgtctcaaaaaaaaaaaaaaatagtgaaatcagccaaatagagaactaacaggcacactgcctaattccttgtgtaactgtcattggtgtgagtcactggggcagcgtgattaggtctgtgagctggatagcaaggctgtcattattctcaaggccttctgctgactgctgctcttggtaagacatacagaatctgtgggcacctcctcttaccgctctatgctagatgaggcgtctccacgttgagttgtagtagatacagctaagaaagtaaaagaaatgaaaatctcatcaaaaggattggggctagtctcattcttcatgcatacccaaaccgtggtaactttgatttttttattatccagaatggattgtttagaggagacagatggctttccagttcatcaaattttttcaattatctaaaaggaattcctacgcttggagcaagtcatttcaataaatctcattcatatttacctgctgcttccttttaaatggaagaagagaatgtttttcaagtacacttgaaaaatagcatatgtgaattagaaacttcctctgtaaactagcagagtagaaataagaggccaggattcagcaccaggtaggctggattccttcttaggtggctcaagctggctatggggctggtctcatcatatctaaagaactgcccagcttttacgcattttgaatgtggtggaaccagctgcatctttttatatgagagcggcaaatagtcattgtgttccagtttgcttctcgggtcatctaaacatcagaggccatgccacaagcaccgtaactgagggatatagtgtgactgtggtccaggccagtgacctctggagctgaatcatgacttcccagatgactcccagagcctgagaggggactggtgcgtggctccaaatgattttcacctgggcctctttgaagtcgggagagatacgtgcagtatggtagatgtggagagggagaggcatggtgtgtccaaaaattagcaggacctgatccatatacgtggactctgaagcagtgcacttactagattaaaaaaaaaacaatgtacttactggatttaaaaagtgtgtatttttttgtgagatgcagtttgtaagatgcattggtaactaggccatatactttttccattggctctcagctctcaagaagctctttagtttttagcattggatcagtaaacacttattaaaatgttcccagcatttgtttgccatcttctctttcagctgggcccagctgtgccctcaaaccctgcctgtctcttccctgactgatcatgttctcccagcttctacagaggcttccatagcattgtgcttggtagcagacaccagacagaatctcagccagctcagtccaggcgcacgggctcctggtctcccggggtgtggttactcgtgtctcctcggggccttccgccctccacattccagtgccaggccctggacctttgtctctattcatgataaaacatttttaggaaatgctgacgctcagtgaagaaatggaaacactaactctttgttctcttcctaacagacacgagacgacttcctgggccaggtggacgtgccccttagtcaccttccggtaaggacagtctcatgtttgatgcttcgtgctgggggcgggggtttctaattgtttgtagtcagtgtattttcagagtggcatttcttcaccacggtgaagaaggctgaaatgaagcacgtgtgctgtggccttagtcctttctggaaagtggtcctttcaaggggatggaggagaagctggctgtaagagtagtcaagggtgcctgagtcgtgcgcttgcgttcccatccaccagctccctctcttttctgcacttcagctgctccacctttgtgtatcacatcttacaaaacagacacctccttcactcaggcctcccttcttgtcacttactgtctctgcttcctaatgcatcctccgggtcccatcagatgtttgcactctttgtctggaaccgcacatgccatttctcttggattcctgtacacaaactataatagcatttcccagattttggtgtctggctttctcagtcagcatggtgacaaaagaagagcctgttcattctgagtgaacttgtcagctgggaacacccaccctcgctggaacacgcactgagttagcatgtgtggtgctgccatttctctctgctcaaagtagcggagcagtcgaggctgttgactagtgacgggcattagggctgacacccgaaacaggaggcagggcagacagaatgtgtaggtaggacctggaggccttgagaaaggtggatccccatctcctattccagcttcagttttccggcttgtcccagccttcctcctggccttcttccctgggcagcacaacgacgtgatagaagtagcagctgagagaggaaaggcgaatgtttctgccttacaagggcattgtgttatttttttaaatgctttgtgtgagcaaatagcataaatagtctataaagtaaagtgactttactttatttactttatttaaagctatttaaagctagcataagcttcctgctgtgtggaaattatggctgtttccatatgttaaataccaagtaagctttgaagatgtgttgtgaaggaaggtgatagcaagtaatttactcttactcactcaggggagctgttaaggaagaaagtcgcaaggttccaaaggactggaagtgaaagtgatattcttaaaatatcttgtttgtttctttttaaaactctgaagctccagggtgccacgggaggggcctgcccagggcttctgcgtgagaactactccatgagccatcagaaacctgactgctcttctcaggtcacgttgggaggatgtgactatttcaccttttgtgagcaaagacctcgcatccttctacgttgggaggtcttctgtgtatgttcttcagactgaagctgcgatcctctcacacttttagacagtgttggttgatgggccagctcttttatggacgtctccagccaccctttccctaatgtaaaaaccacatcttggccaggggaaattattttctgagaggcaaatattctaatatttggtaagaggcaaaagtgacatgtttaaaaaccttgctctgacaagctcttttttgagtgagtgagcagcacctggtcttgaaaggttgagtagggctcccagaaaatagaaaaaggttcctcctccgaggccaagtcagtataaagaatagtttagctttggattctgagtggaggcagggtcagtgagccttcaaagaaggaagcggtgcagaggcacagggcaggaatagaagccagccgattgcagagggtagagccgtgaacgcccagagaggaccgagtgcccctagttactcgtgtgcccagagggtaacttgcagtggcgtgcttcacctccatacttcaaactgactagctgttcacagccaactatagtcagagagatggaccacaaagaaaacaaaaggagaggcgtgaaggtatattctttattcatgtggcagtagaacccaagttgcgtgttctgaatgcacgtggcttttgaatccaggatttcaggtgctttcgtgtgtatgtttggtcgtgtgtataggatgctcatggggaaattacagtggcattctgtttgtgtgatagcacacaggtatgagtgtggagagggcgggagcatatgcgtgtattttgtggatcgaattatggatgctgtgttcttgattagtatatgaccattgtcaaggtggattattaacagtctgtagattcaaaatttgtcaaaaccaccaagctttagataaaaggaaatagaccaggcacagtggctcatgcctgtaaccccagcacttttggaggccgaggcaggaggatcgcttgagtccaggagttaaagaccagccttggcaacatggcaagaccctgtctctatagagaaacttaaagattaacccagcatggtggcacatgcctgttgcctcaactgtttagaaggctgaggtgggaggatcgcttgagcatgggaggtcattactacgctgagccgtggttgtaccactgcgctccagccttggagacagagcaagctctccttttgcttttacattcagagaccaagagcaaacttagctgtaactgtaccatcattgacttttcaactgccgttttcacatttgtctgttaaattcagtgcatcgttgtctaaaaattcatatgatatttgataacttgaatctattaattatttagcgataaagagaactctgagatctgtattaacagagtgagatttctggcaggtgtctgcctgttgcaccttcctcttgacttgtcagctctgtctccccttcccctggagccctcagtaacccacctttgcccagccacaaaatctcttcgtccctgggaaaggcagtggggcagagagagctattttaaaacattaagttttcctaaatcccagtcatgtgccacttaagacaaccatggctgactctggaatttaaattttcctagatgaggcattttcagggtggtggtattatttgatgccctgcctgtacttggaggtagctcagctgaccaacatccttttaccccttttgtgtttatggctgatggctggtagcaggtagcaggctgatggttccctctcatgagtgcagaattcaaaatcatggagtttggagggacttcagagacactgattcactcccagtattttccaggtgaaaaagcctgggcccagagagggaggggacttgcccagcaataagcaggtcattggtggtgctggatctgaccctggctctccagcatcattgtgagcctgtgatgtggtcaagggtgtgggcatggggtggattcgcccaaggcttaggtgcttgcagtggttgcatacaagtttcgagtctagggacccagcagggccctctccttccatttgcctgcagaatcctttatctcactctattccatagtgaatgcacctaggctgaaagcaactactctctttcaagatgaaagtactgagtatccagattttcttctgaatgcttttaccctggaacgggggcagcattgtggctattgtggctaatagcactgtttgcagcagtgtgtttaagatggctttccaccaggagcaccctcaaagccatcctgggaggctccactcagctctgctccagggaacaggatcagattggaacctggatgaatgccaagaagaatcttgtagttatctataagatcatcttctaattcaggctagagaatttcaatgaacgtgaactttgattttaatgccctacctgttactgcttggcattgagttggcagtactgacaaatgatatgaacgtgtagaattggccaatgaacatggctagtgtttgttgagtgtgccaggtgcccttgagaactttatagaaaccaactcagttgattggtttgatcctcacaacagccctgtaagttatggttactgccccattttatagatgaggacactgaggccaagagagataaggaacttgcccttagccacttggctctgtaagtaacaaagccaggatgcaaacctaggagatctgccttttgacctgtgtgatatcagtcatttaagctgcatagggtatggacagactcaggttcagagtccctctaagctccgtggttttccctcttcactctcttgcatgttctctgctgctcctagattgctcatgcccactggtgatcctgtaggctttgtcatactgtttgatttcctatgtttaaactgtcctttctccctgttctctacctctcaaagccttcccatcaccctgtattgtattgaactccacatccaaaaccattttgatttccaagtcagagtccgtctgttccctgtaccccaagtctcacagaatgtaacctctctgtgtcagtgaatttgttctgccttagcttctagtggattgttgacatgatggctcttcccttgtaattgtaaccataaggactgctataccatgttgtatccacatgtcgttgacataatattgctctcaaattatttgttggttggcagggcatggtggcttatacctgtaatcccagcactttgggaggccaaggcaggaggacgtcttgagctcaggagtttgagaccagcctgggcaacataggaagacccccatgtctataaaaaataaaaatattagccaggcatagtggcacacacctgtggtcccagctacttgggaggctgaggtgggaggatcacttgagcccaggaagttgaggctgcagtgagctatgattgtgcccctatactccagcctggacagcagaagaccctgtctctaaattaaataaataaatgttggtttaataaatgaaaaaaggaaggaaaaccagcaatagttttcttaataaattatccatttatttccttatatatatttacaaaccttttggtagaaaatattttcaaaatgtatgtttattactgcctcataagaacagtctagaaaatataatttacttcattcaatgatagtgaattttgagagcctccatagtttactacagatcagatgaattgagtgcttacgtgagtacctacttggtgccaggcaatgttcttggcaccggtgatccaatggtgagcaaattcaagtctctgttctcatgggcttacattctggaagtgatggataataaactagtatgtcaacaaatgaacaggtactttcagaaagtgatatgaggaagatacagcaggacgataggatggggggtgataagggtggctgatggtgggaactctaaaggatgagacctgggatctcacacttggatggcaaagtggaaacagccatgccatccagagtcagctgtccagcagagggacctgcctgtgcatgggccctactgcagtacatataatctgcatccatggcagatgaggacaggatggttgggagtacaaggaaggaagagagagactggttcccaatgaggttaaagttgattcaaggccagattatagttggtcttctaagtcataggaaagaattttcagtttattatgagaggagtaggaacccattgagagtttaaagcagagaatgacataaacagataacatttcaaatagcagtgcatacgtacatacatgtgtgtgcatgcacgcgcacacaaacctgatacagtaaatcactggactggtttatctcttgctataaaagtatttgaaattgtgtttaattgaagggtctaggtatttgaggttttactgaatatatagtatttaaagattaatgaaatagtcacacttatttatccaatttcctgctttggtaggaacttcacaatgaacttcacacaatcaaatgaacactgatttctttaaaaatgaacaaatactccacttgtactcatagcttcatgaactgaacctcagcagaaggttgatcggtgctgcttcctggggtgatctctgtgcaggagatagtctttcaattctcttaggctgatgttgtgagattttaatacaaaaaacaaagtttccctcaaagattaaatcttgtatattcttctgtttactccacccatatgtgtctcgagtcacaggcatatagcaaggcactggtgaccgctggttctccgaagtgaatcagctctgtgagactgaggccacaccctgcagttagaaattttacttggcaatgacagctgtaattttgtcatttgtctgtggctggggctggctcctaaaacccggtgtggatagtgacatctagtggatctctagtagatcagttccgtggactgtcaggtaccaagtgaagaaacacattcagcggtgccacattctaaaattcaaaatatattctctgtaatccagttgcctgtatctaaaacatgaaattattaggaatattaaaatttaatttactgttttttccccacagacagaagatccaaccatggagcgaccctatacatttaaggactttctcctcagaccaagaaggtgaggcttgtgggtatgggtgggtggggatgcctgccctgcatgctgtaggtataggtggggatgcctgccctgcgtgctgtggatatgggtgggtggggatgtctgccttgcttgctgtggtgtgggtgggtggggatgcctgccctgcatgctgtggggatgcctgccctgcgtgctgtgggtataggtggggatgcctgccttgcgtgctgtggatatgggtgggtggggatgcctgccttgcatgctgtgggtgtaggtggggatgcctgccttgcgtgctgtggatatgggtgggtggggaggcctgccttgcatgctgtgggtataggtggggatgcctgccttgcgtgctgtggatatgggtgggtggggatgcctgccctgcgtgccgtgggtgtgggtgggtggggatgcctgccctgcatgctgtgggtatgggtgggtggggatgcctgccctgcgtgccgtgggtgtgggtgggtggggatgcctgccctgcgtgctgtgggtataggtggggatgcctgccctgtgtgctgtgggtatgggtgggtggggatgcctgccctgtgtgctgtgggtatgggtgggtggggatgcctgccctgtgtgctgtgggtatgggtgggtggggatgcctgccctgcgtgctgtggtggttggcttttgctgaagacgattttaagtgtcctttgaagtcacaacccttctaaccaattttcttccattatgtgtgcagtcataagtctcgagttaagggatttttgcgattgaaaatggcctatatgccaaaaaatggaggtcaagatgaagaaaacagtgaccagagggatgacatggaggtacgtggagggcgtcaggcctctctccttgccttgagatcatattcatgttttgctttcaaactttataattgaaggaaataaaagtttagaaagcttaaatataaaaaaaagtacatatgtccgcaaattggtcattgatagtctaccagtgcagttatttgttttggttaaagatgaactttttatgttacaacaccaatcctcggagagatttctctttagacaatgccatgcctattccccaattttagccccacagctctgtgaaagtctcctcacacattgaatcctattattgctatggacttgcaactctttgtaaattctttttagccattgaggtcgcttcaaagtgggtggaggaatttagaaaatcacttttttttcaagtttcttactcatttggccacaggtgacagcctgcagtagaaaaaccctcctctagaaatgccattctgcaacgagctgaccagaggaatgtcccttttttccttcctgacagagccgcctgtatttaccttgtcacagactgatgctgtgggtcctatcgtaccttttcagcacacacctcggccagtgctgttttgttaggcaaattctcatattctcaaagtgtccattaaacgagacctctctagggcttattctcacatctgtttgcagctgttgcatatatgtgatatgtgaataatgtgtgtggagaagttacccgaacactcctccgcctgagagatcatatttccaggcattatttcatttacaagccttcatctctctatatacatgaagaggaataatgtgggtgatagaaatttatgaatgttaacatttcataagcatgtcaacagattttcatctcaaatccaaggaattttttacactcttcttgccttccaaccgcagtaaccagtgaattacttaattttccagtgattttagcagaggatgttaggaagggaatacgtaatgcctaaaatacgtgctgtaaaaatggaactcgaattttggtaccatgaaacccgttggtgtggacacagccagtgtagagggcctgagtagaaggcagcagcttccagactcctggctcctgcggttgtttgggatgcaaagaggaggcttctctctcaaagcccacctccctggggagcaacaggactctgggcctctggtcccctgtgccatgagttctgttagaacacaggtgcagcgcagctcatcttccctgctccctttacctcatagaggtgttgaaaagatggcagctgtactgtgacgcacatcattggcagggctttgtacagccttggaggccctccagtcaaaataacaaacatgacgtatggaaagagtatgatgtgacttaagtagtgtcaccgactgtgcagatccaaattgtgttgtcctgcaatgtagtcatcttagagggcagcacaattaattggccattactcagaagatttttgaggcctttctagaattgatatatatatatttttttggccttcatttgggaatttactcaaatgtggccccgaagccatggctagagccagagagccccagagcaaggagaagggcatgcagggcatgctgtgatgacctcttactcacagccgaagaaccaacctcataatcgtccctttaactttattccgcagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatgtgagtaccgtgtgatggtcaggaacacgtgcacgtgcactgcacagctagggacaaatatggcggccctttgggacaaggctgggaaataaatcatatgtctataagagcagatggtggtgtggtacgagattcctctccatttacgtaaagcatgcaagagcagtcagaacttgtatattgtaagcgattgcctttgagtcacatgctgctgttttcaatatgagctgatttattttctatgtcttcttttatgtcgattcctgaaatacaaagcagaggttgtttttcttagacttattttacatttgttgattatattgttttcttcttcacctttctctcccaactgacccagaacagaaccggcaagcaaattggctttgcaattttctgttttctctaagtttttggatgtttactctcttcaatgcattgatttggaaacctcaaaaatttttttttctttaaggaaatttgagccccttaatataatcagaaacacatgggagagttgcaaaatcagaattagtaattactgtgtattttaatccctcaccagcctcagggatgcagtaaaccaattcctgttttgttccctcagcaatttgtggtttgattgatagcaaataaacactggttaaaactcatggactttaaaaatggttttgtagcagcttgtttttaaatcattttatacagcaagatctaagtctattgataatacatcgaagggcaaaaagaaagtcttaaggaaaaggaattttatcctagaaattatgagctgcctagcagccaagacaaggaggaaattatcaatgaactatacaactttcagtatgtaataaaagcaagtctactgggtagtataattttcttgtctactcgaactctgaaagaaagtggctaatgatacattttagacaacaagcagtatgaggtatcatgttcttaaaaaccttttatctttagaaattgtaaagatactccacatatgactgtgggtggggagggtggccaagttttctggtctaggttttgtttaatttagagataaagcttgcagaatctggaacaacgaatagcctgtcttctctgagcccccaggtgggttctcgtgaatgtcagctgttcgtgatcagctttagactacccagcacgggcattagctcaggacagcaaaatggtctcaggacagcaaaaatatcagatcagctgggtaatgctgaactatcttttgttaaagttttttcccatggaaatagcttggttgattctttttgttttttatattattgcttactatttccttttgattaatgcccatagctattagaattattgtaatccattgtagctatttgtagtaaaaatgaatttggacccaccttgggcttggttacatgtagtgactagaagaatcttggtagcaatatcctaagcaaagcaccattacctacctttgctcaacagcatgagaaaggagtactgaaaaggtagatcctcatgcgacctctgcagaaaagtggccaggagcaactcacaaaggaaaaaatcctgatagctaatatgtatatggaaaaatattcaacttcataaataaggatcatgcagattagagtttgtattttggggacaaatagggataattgctactttctctgcctcacagagttgtgaaactaacgagggaattcttgagaaaacacttcatacattttaaaatatatactatttttttgatggctccacaggtgtagacatgtccccacactcatcaagaggtgtacgttaaacatgaacagctttttaatgtgttgattagacctcaataaagtgatttaaaagagaaaccatctttggtggcagtgtcccagaaactaaaacaaaaggtataacttgctggctcttcactagtctagagcaacaagggaaaaatgcagttctttttctgttgttgtttgagacagagtctcactctgtcacccacagtggcaccatctcggctcactgcaacctccgcctcccaggttcaagcgattctcctgccttggcctcctgagtagctgggattacaggcgcccgccgccatgcccagctaatgtttgtatttttgtgaagatggggtttcactggcttggtcaagctggtttcaaactcctgacctcaggtgatccgcccacctcagcctcccaaagtgctaggattacaggtatgacccaccacgcccggctgaaaaatgcagttcttaatccaagttcgggctcacatgtagttttgggagaaggcagaggcataactgccccatcaaggaggcagcaatgccaaagggagaggctgagccaggaaggacacagcagaacttggcagaacctgaggagcgcctccttccaatgggcataacttctagaaggcaagaggaaagttagttaaggaaaaagagtttccaccacaacctctgctagcaggccctttattgggatacagtataagttcatttcagaatgtgtgagaaaactttaaaatttgtgttttagaaagagtctataatgagtgttcttgataagtactatatttcagttagtctgaaatgacgatttcaacttttgagatactgacttgtgacataatttttgttaatgggatatgttatcttattgaaagtatctgtgagagttttccctgaaaaaatttttggcatttaaacttgtacactaaaagcctctgactagtatttcccccaaagtatgattgcctataatttactgcaaaaaaaaaagttttcttttttctttttttttttgttttgttttgttttgagatggagtctcactctgctgccctggctagagtacagtgacatgatctctcagctcactgcaacctctgcctcctgggctcaagtgattctcctgcctcagcctcctgagtagctgggactacaggcacgcatcaccacactcggctaatttttgtgtttttagtagaggcggggttttgccatgttggccaggtacgtcttgagctcctgacctcaagtgatccgcccgccttggcctcccaaagtgctgggattacaggcatgagccaccatgcccagcccaaaaaagtcttccaaattgaatcaaaacttttagatcctgaattgagaacatcacatgacaaaaaatagggtatgatgttttggtgttcttcagaccccggctgacttgagaggttatctcctgatgttattaggatttttaaagtagggtaaaggaggtaaatgcagtgtcacgattttgagattctgttaaaatgagctagaaagcagctgaaaaaaattcatgttggatcaggtgtactttcctccagatgacagctttgaatttgtcccttttgtagtctttggtcaaattgctggtgaccagaagtagctgccttgtcattgctggacttctagaaggattgggactgcccagctgctgtgtgaacttgcctgtcacctctgcttccagaggtctgacttgtcactgtggcaaatgggcagaggactttgatttctgttttttattaaacactgaggtatattttgaaatgtatgcctgaaagatgctcaaaatggtgtgactaatgcattttaaaactgcatttatttgattgaaatgaaccttaacctatactttagccgtaatatggtgggagaattctcacattggatgaccagctagttgtcagtgtttgacgctgacactttagtggccatctggccctccgtgagcattgaactgtcatgaagggcccgatctcaaccacttctcttcttctctttccccctttcctgcatgctcagggacgtgtcctcggagtcggacaataacatcagacagatcaaccaggaggcagcacaccggcgcttccgctcccgcaggcacatcagcgaagacttggagcccgagccctcggagggcggggatgtccccgaggtacgatgtccccagaatggtgcaaagcccggcagctccttcttcccatgttctccagcttgattttatatcataatttcttctgtcagtaaacattgttgagaatgtaaagattacagtgacgcagggaattaattaccatctgaatattaatttgaaagtgcctataaaagccgtatttgacaagtataggtggctctatattatagaaatgggaataacattatttttatttttattttttttgaagacagtgtcttgctctgtcgcccaggctagagtgcagtggtgcagtcttggctcactgcaacctccgcctcctgggttcgagcaattctcctgcctcagcctcctgagtagctgggattacaggcacgcatcaccacacccggccagtttttgtatttttagtagaaatggggtttcaccatgttggctaggatggtctcaatctcctgacctcgagatccgcctgctgcctcagcctcctaaagtgctggggttataggtgcatgccatcacaatggctaattttttttttttttttttttcctagtagagacgggtttcaccatgttggccaggctggtcttgaactcttgacctcatgatccacctgccttggcctcccaaagtgcttagattacaggcgtgagccactgtgtccagcccacattatttttcagtatgaaaatcttaagatatttttagtttgtaatttatttttcacaggggtctaaccatgtctcattaaatgacagcttgtttttatcatagttgagctgttgaatgagccctgtaacttgtttgggtatcatatgcatatttttattgagtcgctttgacacttggaagagatttattaccatgaaaatataaatttaggtgaacagaaatgtgtaattttccctttaaatagttatggtatgttacttaaaacgttatgaattaaacaacaaattagccataatcttccatgctatcttaccagctctgtttatgtactaaaaatccattcttcttagacattgtgttaggaagatcatttagaatgcggcgattcaacttggaaaagcaaaagtaatgattccataacacattcactctaagttattttataccattgtttaattacagcgtatagcttctatgctgctcccacggtgaatcctcagtggttaaaagtaaaagaaaatccatgacaattgtttataaactccagagaagtgtgtgtgtgtgtttgtttttttgcccacacatatagtaacaagtgctggaaatcagtcactgttcagagaggcattgttataaagtggttaagagcacagcctctgtctggaaccaggcctctttgattcacatcatggccctaccacttgctagatgttaccttgggcaggttattttctgtgcctcagtttcctcatctgttaattgggggagttatcttcctcgtgattattgtgaaggttgtgtgcctcatgagaggcacttagtaggtgttaattatagttatcaccggggctattgttgttacatggtttcattggggagctgggtggatccctacttctttctagtgtttaccccagtgtctccttctctgaaagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattgtaagtagtggccttgtttgaaagaaaagctgagcaatgttttattgttttttgttgcttaaggagaatctcttacgtaaatagtgaccaagggtcttccagctcccagctaactctgacattttccaaagttcctccttccctaggagaagcaggactgtactaaaattagcccaagcaggtgagaatctttcttgatttggaaggcagctttgctcacccttataccactaacactttcttggtttataaagaactcaaagaagtaaaactccatgatgtctgtaattattagctttaaccttgtagaattcctttcccagccaaaaggctcatgaaaatagtacctttatcacaaaggtagtatttcacagtaacaattaaaagccatcaggcctaatcagtcctcttaagtcatattctgcaggatttatcattctcatttcgctgataagggcacaatgcttaagtggctcatccaagacaaggtttgagtgggagatagaagacgtctcctaagtcccagagtatgaacccttgagaaatgttgtgaaattgtgacactgtcatcaaggtagcattttaacttgggctctcaaccatcagaaacatactttctagaagcttcagatgggcccaatgcctgagcccaatggaatcagtcacaaagacttccatgtttgacaatacaaaactgtgcagaaataacccatagaaaatccctgcttgtgcatagaattaagagcaaatgaagtctcagtgtttctcaagaaactgaacttaaaaatgtacaatagtaaatgatgatagtcactccactgaaaatggcatcctaaaaaaaaatagtcattactatgaccataacaaacataaatgatagcactgcatacaatgtaaaagcaagtaagtactcacaaaattgttactttttggttatggtgtttttattttaaaagacagtatacgcctctattacatgagttgcatttatcattcagttaatgtgatttcactttgaaattctgtgactttttcaaaaagagatgtgtcgccaagatgaaaatgtagtactaaatggacaggataaaagaaattgcatatatatgggatatttgaactgataaaactgttgttatagaaacaacataaacaatgaagccttgtatttaaaacctataaaagcaaggaggtaccacagaatccatggcagtaacacacctttagtaaaccaagtaaacactgtaatttttttcagcaataatcataagttagcaattctaattatttaaatgaggagctcctgaatttaattattctagatctgtgaaatatcattaagacccaaatacaatgttgtattatttcacatcgtgttcactgttaaatgagagtcaaatcggatatttgctcattgggattaccctgtattcaaggggaagaacatgagctttggctgggcacggtggctaatgcttataatcccagcactttgagaggctgaagcaggcggatcacctgaggtcaggagttcaagaccagcctggccaacatggtgaaaccccgtctctactaaaaatgcaaaacttagccgggtgtggtggggtgcacctgtagttccagttactcaggaggccaaggcccaaaaatttcttgaacccaggaggcagaggttgcagtgagccacgattgcgccactgcactccagcctgagcaacagagtgacacttttattaaagactgattctttcacagtaaccaatgtgggaagatgtgaattgtcccccatccctaccacagagtcttcttgtaccatttacttatcacaaaatattaaaaatgagtaaatactagaaactgatactatggatgaacttgccgataaggagactctggcttctgtgtgtgacctgctgctaatcttcattcatatcaattaggcttcatttaaccacatagactaatacaaacaagttagaggaatattaagacactacacagtagatctttttttttagcttgatctcactatttttaatcattcctgtctttactacttgaagtccacttagaagctaaaattggctgagcatggaggctcacacctgtaatcccagtactttgggaggccgaggtgggtgaatcatctgaggtcaggaggtcgagaccagcctgaccaatatggtgaaaccccatctctactaaaaatacaaaaaattagccaagtgtggtggggggggcttgtagtcccagctactcaggaggctgagacaggagaatcactggaacctgggaggcagaggttgcagtgaactgagatcatgccactgcactccagcctgagcaatagaacaagactctatatttaaaaaaaaaaaaaaaaagcagctaaaattataaataccagatctttcagaagtaggccaagatgatgtctgagctcctctgaatataggatgcaactgactctttttcaattgtagtaatcaataaatgttgggtagccacatatcactatttcttccgtctattcacatcacattaaaatttaccctgagggtgcatcttagagcaaactacaagactgctagcttctttctgtttgtaaagcagcattagagtgttgcagagtcctgttgagtctcctttctgaaagttttggtctcctcttacttttgccactaattctaaggaccaatcaaaattccagtctgcttaactgaaaattaaagtgaaagataactttttcaaggatgactgtccttgctctgaaaagatgtttcatttaattaccttctaatatcgaagagcggtgaatatggttgagtgatatgtttgttaaaccaatgaaagttgcttttaagaatatatttcttgatgcttcctgtcttttcctctccttccagcaaagagaaccctcctcaaggttgaggtcatgcagtgtcaccgacgcagttgcagaacagggccatctaccaccggtaacccatgctaatttccagtcatcagttgactttttgggctttcatttacaatcatctgggctgctgcaaaaagagtttttcctgtgtaaatttatttacaatataaatgagatgtttttaaatttcctggccaattgccctaggaaagtggatttccaatacagagaattgttactgccacccatctctaaaatgctggaacctccattgctttagtttttcacccaaatttataccaattttgatttgctcacttaaacctcttggtacattagttagtaattccttttaccttattgagttactatagttatgctccagtgatctgtactttgaaaacacattgagtccctttaaccaggaagaactcttctgggcgaccgtccacatgttcctttcccttcctaatgacctgaagtctgaactctacagagcctctgtaccttctctttaaaggcagctctgctcaccaggaattggctataattctttccaactctctcatcattttaatagctatcagcaaatgtagaggtgtaggggattttgtaacatctgatgtatgtaaaaagaaatattcaagttggaaggcagactagaactgccctgagagatgtttgatgtacacattaaaaataagaaattttacataaatttgtttcttcttttggaggtgaaaatatgtggttttaatgtgttcaatttgatattttaccttgacagtttttgtctgttgctacaaaaccttgatcatttttaagactaataatgtattacgtatgaatttgaagggggaaaagtgcatttaaggtttttgaaatcacagtgtcttccctctcatgtatgttttttcttgacttcaaaggatattttggcctaggttttaattttttatgtttgagaaaaaattaatgagtatactagtcaaggaagcagtcaaaataatagagtcatttgtgacttacactggagagacttaataataagcttgataagagcgtcttttctgggatggtttcagtacagggctcccaaaaacaaaataatcaactgagatggactggtaggatccttctcaacctcatgatctgtgattttccttgaacgaatggcactttctctccttttcaaaatcaatgtcaattttatttcccaaagaactcactttcctggaaactggtgattcctccctctttctttgtactttctgatgctgtaaactcactgatcaccagtggcctcttgagttttgagtgtggggagatttcagaatctgctaattatttgacaatggcaggcaaaaccagaccagctttccatttttctctaaggcagctcactcttttgaggttgtcattgtttacccaggtcaaggatgagccaccttggggggcataggggtcacgcttcctttgtgctactggtcatcagcgactgctggctttgtctggatagggtgggtttcagggattctgatctcacgtcacctgccttacagcgctgccacagcagtgggccctgattcagacagcagggggtcatcccctaagtgcatttcactgatgtaaattatcattcacagcccagtgccccagctgggagagcgcgttcatcaactgtcacgggtggtgaggaaccaacggtaatgatccactttatcagacatcaatagcaagaggccgtgaggcagttttaatatttcgtgtagtggtgaatatacgctgtttttaaaaataagattagctcctttgtgctacagagctgcacacgtttcttattttaagcttctcatttggggattgtttaaaggtactgggtaaaatagaatgatttttttagtcccacataattttattaggaatactatcaggctttggttaagtgcatttaacttgaccatttagaagatatttttgacttttagtgtttgtactcctgataagtttttccatttcattagaatgcttggttaaagtaatttggttggaagatcatcaaagaagccacaggaattgcatgagactgtgtgtgtgattcctggatgtgcccagggcccttctcttttctgtatctcccctaccacatttttcacagggggttagacttttcttgtccccagaatgcaccatgaccctttctctgccaccaggggctctcctcacacaatgaacaagtttccgtaattccaagggaacataattaggatatataagtgatctagaaaaatttatgtttctctaagccaaccattcttattcattattttaaagaatgtatggtattcctgtctgtgggtgacttagatttgtattttaattgcctgttttctacttgatccctaaagttaggctagcttatagtttatgcttcgcactgcaatagctgtttaaagaatttcctggccgggcacggtggctcacgcctgtaatcccagcaccttgggaggctgaggcaggtggatcatgaggtcaggagatcgagaccatcctggctaacacagtgaaaccccgtctctactaaaagtacaaaaaattagccgggcgtggtggtgagcacctgtagtcccagctactcgggaggctgaggcaggagaatggcgtgaacccgggaggcagaggttgtggtgagctgagatcgcaccactgctctccaccctgggcgacagagcgagactccatctcaaaaaaaaaaaaagaatttccttattgtttcacctgtgttgtattttgctataactgctcattacttgtggataactttacgtatacaattgctgattttctaaagatgttgatgacttcaatttaatgctcagcattgtacagaggaaaatcaaactggcaacagtcagccccagaacactcagctcaggttaagatattgagctacagtggtttacatgagtcatgtgttgagtgaggcagcctccagtagaattcaagaaattctacatgaactattacccaccgtgcacttgtaatttctgtgggagtagtggagctttgagggggacaagatcgttttgtgagataattttagttgaattcagtgctgatgaacatcatctgtatatcacctatttttgtattttgttggccttgcaacctgcgtgtagctttggagtgttgtggtagctgatgtagtaaatgaagtaggaatggaatcattgcagtaccaggctccatggcaagggtcacctggcaggacaaggtgacccctctgaatgagatcccgggtggcacgttagactgccattttcttgccttcttgcctgcactctcccctccccgtcactgagtttcagcatgggttgggtgttttctttccacaggcttggcaatattcattcctaagtgtttcatctactttggtagatgtagatagatctccctacctacttaggtttgaaaggatcacatccatttctcttctgctgtatcctgcatgtttctcttccacatcctttgtcgtgaatgcccttgacttcctgtcttgcaattctgtctcttcaccccgtgtttccctctttggcatcccctctgttcctctgcacatcactcacactgaagtgtattgtcctgggctggagccatcatggtattatatcatctcactgcccctgtgctctcacagacatacggctgtgtacacataggtacacattgtgccactttattttattttttttaaatgggatgtcatgtatacattacttagcaacttggtttttttttaacttatgttcattcttctgggacatgttaataattacataatattcactgacgtaaaggcactacagtgttaatttcccgacattcaaggactttctgctccccctaccctgccctttcaactccatacgctgtgctgaagtaaccgtcctgatatagctttctttctatgatagtgctttcattcctgtagtctaataaacaggccctgattctcagccaagaaaccacagccctttgatttatggcaaataaacgcacagtggtgtcactttctggcagactaagccagggccctccccatgagataaaaggagagggatagcacagtcctttcagtctttgttgtatatattctaaaaatacacgagccaccattactgggttggtgcaagtcttggcaaatctgtgatggtcagtgtcaaagtggttatttttaaaagcatattgaaacttctgcagtttacaatcgtgttctttattttaatgaaagaaacagagccactaggaagttctagctaatggcttttgggcatcacaaagcagtagtgtattcagtctatgatgaaaagctgtgaaatgttaaactaagtcgccatttctaactggtttgattacttttttgttaagctagggtggaacatgctcacaagcacagaacagcagaaggctggttttggcttcctggcgggcccttccatggcctgtctgtagaagctgagtcctgccccctcaactagccgtgcagggtgggaggcagtgtgagttacaaatggaatcatcagcacaagttgttctatgtggtcatttggagagaacactgtaaagaaacatttccaaattgccctctcacataaatgtaactaaattacaggttgccagttcagttctcaatagaagagagaatgagcattttaatcaaattcagcgttctgtactacactcctaattttccacaaactgattagtgctattcactgtggctgtatgaaagtaacctgtatggccaggtgcagtggctcacactggtaatcccagcactttgggaggctgaagcgggtggatcaattgaagtcagcagttcgagaccagcctgaccaacgtggtgaaaccctgcctctactaaaaatacaaaaattagctgggcatggtggcgcttgcgtataatcccagctactcaggaggctaaggcaggagaatcacttgaacatgggaggcggaggttgcagtgagccaagattgtgccactgtactccagcctgggcgacagagcaatactctgtctcaaaaaggaaaaaacaaagtaacctggttgctagaattcatacagctttaggattcagttctgacatgatctatgacacatccatgtgctttcagttgatcagttgcctcccaagtgtcctttccagagattgagtaactgcaaaaatatccttaaggtttccagagctaaggtttccggagctgagttttccaggggcggggccagggggagtggggagttatgggagggaaggggcctgggagagggggaagtggatcaccagaacggctgaaggggagggctctgggcggagggagctgagaatgtaacacattccatttagtatttcattgattgtataaacaatagtaggtcaaaacttgatagcacatactttgccagagatactaattgaccaggaacttaggttacaacttagctcagcaggggagtccattgaagtgcccggatccacgtagctctttgtgttggcatcgctgctagaggaggaggggaggaaggagcttcactcaggctgcatgctaattcatcgatgtttaatattcatgaaaggattaagtcagtgttgatgtttcttctgctcctgctgagtctccacacctggatccagaatgctccatgatccgagtctttgtgtctttataaactatctttagggagctttaggactttatctcgggaaatattaaggaaatggttttccttaatatcagcagaaagttttcttacacataatctaaaatcagctgcttcatgatcttacccaggtgatctttctctgaattgtgcttttaagcctgggaggttaacatactgcttctctgtggatggggttttcaaattacccggcagcatccttgttggtgctttctctaatttgagatcctggagtccaccagggaactccaggtgaccaccttcaccaagacacattgctggtagtgcagggaaaaggtggcataagcacaggaagaagttaggttcagaggaaatttgtatcctcagcctaatagcaagaaaacgacaactgctaaaatatctggaagtaaaagaagcagcttccttttgtaacctcacgtgagaggttattcctaaggcatgggctcctcctatacctttagattcccctaggagccgtggtatagctgtggtcggcttctggtttctgagcttttccccacttagagctctttcacaggcacagatattacatgcatcgcctatgcattcaagccctcctgggacagccctgatctccagcatctgtcccagtgcctccatacatcatcaacacatccttgtttttcagtgtttctccatccacactgcatttcttagactgcttctcagggatcttcttgtgatcaaatgtggactgtctaaatacagtcactctatgcggggcaggtctctaaacccaaaaactgccaagctgtgttcaccttcaaagaaacacactcctatcagcagacttgggtgcacatagcctggggtgtgaggctgggtgctgggagacttcctgagctggggcacacagatgtcctgggtggtcctccagattatatgctactgtgtgaccatcagtatcctagagaaggggctctgtgttgggattttaaaattttgccccagaagctacagtgttagggtcctaagctgatggaggaatgtaggcctaagctagaagagcaccccatgggagctacatcgaggagaaactcagagcatccctctcatacacagtgacactggacagtttgaaatacctttaagctctacctggtttggcagtggaatttagtagctagaacagatgataatgtttttccctttccctgtgatcaccttggcagcaaccctttacgtttgagactacggggtcttagaaccaagttctgtaagaatttggactctgcaactgtatggggttcatttcacagtagatttgaagctgttggaaatctattgctgttttccagacacttgtaagtgttttctatataaatagatctaattaactttgtctagagaaaataattatctgggtgtaccttactttattaaactgatcagaaaacaaatgcaaggtattaaataataatgatttcttgcacaaacagccatcagtggcctatgtacataccacgccgggtctgccttcaggctgggaagaaagaaaagatgctaaggggcgcacatactatgtcaatcataacaatcgaaccacaacttggactcgacctatcatgcaggtacgaagattgccatccaacttaaaaccgcaggccatagaagccgaaatgtacatgaccgaactcctttcctggtgtttatgtattgttctgaaagacccgtatttgtaaaagctttctcacaaagaaaatgcattactttaaatacaataatagcctaaggaagaatttgcttttcaaatcagtttatcactgaatatattttaaaaatgtgacgtgaaagatattttcaaagtaagagcctattgcagtgtgtagcttcttccctgcccagcccccactttaatccgaggctagtttttatgctgggctagtgattcatttttatttcccttgaatgtgtttccccatcctttcccccatcattatgtttaattatttcctccacgctttaaataggccagacctcttattattgctgtttgcgttgttgtttgggttcgcgctcctaatcacacacaccgggagatcctcctatgaagctaacttgtttttgcctccaaaatagcttgcagaagatggtgcgtccggatcagccacaaacagtaacaaccatctaatcgagcctcagatccgccggcctcgtagcctcagctcgccaacagtaactttatctgccccgctggaggtgagacggctacctcatctaactggactcactctgtcctgtgactcccattctttcttctctcttaactctgccttcagtttcgcagccacctcctctctctgatcagtcgttgtgttggtagtgaagggtgttcttgtgcagcctttctacctgcttctctgtctcgcccagttaacattcctggactctctcacctgattctcattctcatttggctaaggccgtggtgacttgtgctggacgtgggcactctcttttcaggacttccaccgttcttgttgtgtttcgcttctttttgcctgttacctcattagaattgcgtaatccctctttcaaataccaagtgctgatattgagtttttaaacatagctattgctttgatcatttgtttttctttcgactgtagggtctttcattctatgggcatacatgtttctgatgcatatttgcatctctgcatcatggctgggcatctgcttgtgctctctgtcgtgtttgtgttgttttggtgccatcctttaacatcacaaatgtcagagttagagaagccaaagagcaaatgtttgaggtcctaagcttgactgtgtcttagagacagctgtgggcttctccacgccctgggtccagatgctgataagtgggaatggatgctgtctcttccacactctgatatgaatgtcatgggtaacaactgtgagattccagatcgttgtatttattgatgtagcttttttcctttttgtgctctaaaacttgccaatctgttcttcattagactgtgagctcattaagagcaagggcacatttttctccactctgtaaaatctccatccataatgcttagtacagaacagtatttttggtacatactgagtagctaccaaaggatggtggttataagtacttaatattgttgtgtgtttatccaacagagaaaatgtaaagttagattgatttaaaagtaaatgaatgacagaaaacaggttttggggatcttccctcttgtccatctccaaagagaagccttctgcaatacccaggtacattttggcacattggaatcgcacatgctaaagagttctaatcctcattgttattctttagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggtaaggctgctgcttttatttggctccattttcatcatgaagtctggcattaattccttgatttccttagtgtttgtagttcttgcagaggaattggattgactgtaaagggtgtctccagagggtctcctcagcctgcatctggcccctgcataaatgggcgggtgggagtgggtgggcacaggctggtagctgtctttggtattggtgctgggactgccttttctcctgggaagctctagtttagacagttcaccgtgctggatggaaaggtattgctctgtgctgttaagattttgttaattgatcccgtctcacaaagtaatctcactaggagttataatcattttcaaataattttttcttttgtcagtttcttcttgcactttcattggcattacccaccttcttttgatttgtcgctttagtttttaccgttcacatcagatcagtattcacacatctgaggtacattttcatgcccttgatcatggggtaggggatgtactgagtgactttggttgtagatctgatagctcctttttgtgatcattttgtcaaaattgatttttcttaccatgcaactctttcatgtactcctgaattgtacccaagaaagaatctgtacattgtattatcttgagcaaaatgagattaatcaaaggcaaatatgcatatctttgtatttctgtgtcagttctttatttctttagccgaatcggtggctttttcagtgtagtatatgattcttttaaaagtcagttggtattttcctgctatataatctctatacctgtaattacactgaaaccggttcccgtatttctggctatttttgatgctgcagatggtttttcttccggttatgtccccatcgaattgtctagctagagttaactcagagtttaacttggtcctactattatctgtgctacttgctcaaaactgatgttatgggaatgctggttgagttttggtgattgttattggccctagctcttcggtcttggaccaacaataaacatacaccattaattcccatttctagtgatggacattaagtgtgtccatttgatagaccagcagccttatattcaagaacaagccctgctcaaggggatttgggctgtcattttatgccttgcccattgtgtggtatcagttcagtccagcaggtctagagggcgtgctcctagaatcctttgaaccgtgggacattgtgtcttcaagggcattcttggcatttggggcagggacatttgtgccttcagttgcagactatttaacaaacttggcttctgctaccaggtgctaatggtcaccattacccccaccccaacatagtgatgaccagaaatgccctacacatttcaaaacagccctaagtgagaaccactctaagagctctttctattttgaagtaagggaatggcctgccaagagaatttctcatttagatctggtaacattcatttggtaaactgataactgcttctaaggcatataataacatgtaggtttgactcccctactagtctgaagataatgttacagatttgcaagcaagccaagagtaagagacgggcagctcgttggaaaatatagtaatggagttctttttttttatgtctttttacttattaccatggaacccctggctgtcctgccagtgttctcatttactatagcactgctgtaatttcaagtccttagccagaaacaggctccagaaataagggctaacgaatataaaagtagatggattagtgtaaggagccactgtggcttgccaacataagctatgattttagtaattttaactagcttggtaatcaaatcccctaaccttttcacagcctttcaagccacaacaacagtgtaattgcagctgggcagattgtattttaaattaaacagcactcgctttatttattgcttgcaacattgctgaatgaaacccatttttactttccagttgcgcctgtttacccagatacttgttattgactaatttactgatttaaaatcataatctgttagaacaacgttttgttgctgctgctagtgtgggaaatcctcaaggtaacaactggaaattccacaagctactccccttcttaagcgaattcagatgtgctgacacagctgcgctatcgcttctaaacaaaggcctaggcaggattttccctgcggaagacacactctaaattgcctgttcattttgacaaaagatgaagagccggagggaagcatgaagataatttgcaacttaaaaatctgttttgagttggttttagggtttgctttgttttttatttttgaaagaagaagagaacaaatgagagaacagcatagctattcttagatttgagcattagaatagaaaaaggaaaaaaactggtaggacaggttgaaaatgatgaaaataacactccttcccccatcggcctgggcactgttgattcttacaccaaccagaagcctgaaaagtatcaggacatttcccctctatacattgagaaattaaaaaatttattgaaatatgtcatgaaagatgaccttttacacacacacagatactgaaggcagtatgccatcagtatttggataaagtttggcctcattctcttttttttaattattattttttttgtttttgagacggagtctcactcttgcccaggctggagtgtaatggcacaatctcagctcagtgcaacctctgtctcccgggttcatgcgattctcttgcctcagcctcctgagtagctgggattacaggcacatgccaccacaccagctaatttttgttgttttttgttttttgttttttttagcacagacagggtttcaccatgttggctaggctggtctcgaactcctgacctcaagtgatcctgccgcttcggcctcccaaagtgctgggattatgggcatgagacaccctgccaggccaagtttagcttcattcaaagaaagaaagaaagattggctttgttccccgcctgggtcaggcctttcagggaatgcatgctggagaccaggggcttgggagggatgtaccaaggagggatgttccatcctcagcagctcagagcacgtgtatacgggttgagtatccctagtttaaagattcaaaatccaaaatgatccaaagtctgaaaccttgtaagcactgacatgacactcaagcaataaatgcttattggagcatttcagattctcagattagggatgctgaagtggtaagtataactgcaaatattttgaaattcaaagcccctctggtccccaagcattatgggtgagggatgttcaacgcatattagtatatttgtgcctttcacaggatgaagcatgggcttgcttttgtagcaagaaagattacaccctagttaagttgcaggtttgttttccatccaagttcaaactaatgaaacttattactctaggtgtggttgctggtctcttccaggatggaaaagataaagaaaaaaggcacttttttttaatccctgtgttttatatattttgaattaggaaagccctttttaaaaagatactgctctttgcatcctgtttaaaaacactttgaacttgatggagggaagcaattttatattacacatgaaagagttcccgttatgaaactgaagaaaggaagaaacaaccaggcacactttatgcgttggatcatttattttcattgcatcagagccctcgttagtattccactttctcatcagctctgaagttgtccataactaatagagagccttgaccgccacttcatgtgccctggcaactaaggagagatgcctgcctccttgagtggctcggtgggagtcagggcttgagggtgaccactagccttagccctcagagctgattcatggcagctgctgccattatttttattatttttgataaaataatattataagcctcaaggcctctggccctcacagtctatgaagtctaggtttccatgcaaggccactttagcagatctttgaaattcggttgactttatacttcatgctctgcccctcactgcctccctgacacttttctctaatttcagcttcttttcacgctacggccccccccgccccccccccccctttaaatcaccagccttttaccaatagggctgattgtctttcacggtagcctacatgtgattccaaggtcaggctggccttgtgtgtagcattcagaagccctgccctctttcatgtaatagcagtcctcagctttctctaaatctggcatggtcataggctaggtggaagcccaagggtcccccgctggttgtggcgttacctggtccggatcagagttagcattgagtgggtagcttttgagaaattcagtgctttcagtttttcctctgtctgtagttaaccagtcatgaatgtttaccagattatcagcttcttggaccttctcaatcctaaatcagcttagcatatttctttatgttctgtgtatgtcagtgtactgcagaatactggattattttgtgcctctgcttagccttttaaaaaaatatattgggttcagggtcaatatgcatcacaaactgctaacaggatttctttcttcatttaaaagttcacctagctagcaagaagcaagggttgatttttgttgttcgtggtcttttttgattcttgccagttcccaagatctatgattatgattcagtgatactatgacttacacggttgacttgggaagcagaaaaatctagtaatttggcagcagcaaaggccctattataaaattgaatgaagctgatatcaaatgtcttcacagtttactttttttaatcttaagcttatttggttgatagtggttttttttttgagtataaagatatggattctcctttttatgtttttggaggcaagactcactgcatcacccaggctggagtacaatggcacaatcacagtttacggctcactgcagccttgacctcctaggctcaggtgatcctcccacctcagcctttcaggtagctaaggcaggcatgcaccaccacactcagctaactttttgtgttttttgcagagatatggttttgctttgttgcccaggatggtctcgaactcctgggctcaagcagtcctcccgccttggcctcccaaagtgctgagattacaagtgtgagccactgcatctggccttacactgcatttctttttcttttttttctttttttttttttttttttttttgagacggagtctcactctgttgcccagcctggagttcagtggcacaatctcggctcactgcaacctctactttccgggttcaagcaattctcctaactcagcctcccgagtagctgggactacaggcacacaccacacccgggaaatttttgtatttttagtaggaacggagtttcaccatgttggccaggctggtctcaaactcctgacctcctcagcctcccaaagtgctgggattacaggcatgagccatcgtgcccggcctacactgcattttaactgggtctacataagtgagagtcagctcctggactctgggtcataatctttgggcaacttcctgttctctctgaaactccatttcctcatctaaagaaacagaccatgtgcccaacctcctaccaagggtctgttgcaggattttacatctgaccatgcctcataaatcatgcagcctgacccaggttgtcagagtacatatttctcttttgttgattctctgtggtatcttatgaagcttatgctttaagaaaaaaattttgtaaacgtttaaatattaagacataaatgagaacttaatctgcagaacttggtcaagaatctcagtaaagttgtatcataactttgtgagtttgcctgatgaaagcgactctccctggagtagccacgtgacttcacagaccagattgacattctctgctgagttcagaaggagtggaggggagcacaagtgctggaggagggtgtggcagggaggcctattccctaacgtgttaggctggctcccctcaccgggagctcgggacccatggagagatttcgaaaccgttgtccccactccctctttgtgccacattcactggcaccctccattctggtttccctagtaacatcactgctgggactctcttccccttccccttctcaaggtctcagggaaatggttaacctttcatgtaaatattcttactctcctctctccaccaagcacaagaaggaagagtacagtctgtctcgagagacagtcagccaggcttcctgtacaaaacagagatgggacccatctcacgctccaggcatggacagcctggttgccttgaaacaaacagctcaagaagaaaagcacccagtaactgcacacagatacttccacttagcatctactgtcttttacatttttcttgcaggaagatccacgtttgaaatttccagtacatatgcggtcaaagacatctttaaaccccaatgaccttggcccccttcctgtgagtacactggagacacatggaatggtctgaatatgtgtgttcctttatccgctcaatgttgatggagaggccctgtggaccttctctatctccaggctttgtggatctcattggtttgtgtttcttaacagaattgggtgtttagctgaattgtagtcatacattatagttcatgtgatatccagtacttatgaaagaggtttctgaattgctagcttctagcagaagatagaccttgtacacaggatgttcaagagacgttctctgcaggtgacaatttaagtggcactcattcattttgtaacttagcaaccactgagtttcagtcatattgctgattaagtcatcagaacaaaaataatgaaaatagtatgggagaagaaggcctaattaatcatagaaagtttctataagtcatttaacctgcagatttttaaagtttttttctcccttgttggtgacggctcagtaggagttgagctatatacttaaaattggattttgaatgaaaccttcacatacatcctcctcaacaactcactctcagtttgaaaaagccgtagggggtgacagggcagtggttggactccaaggctatttttaaatgggaatttttgttgttgttttccaagtgtaagaacctttttgttgcctggtatattagggcaacattggaggctgcgaaggttaaggttcaatcattattcattaggttatacaggtatcccacaccaaatctgtttggttcctgggttcaaatagaaagaaccagagagttagtttagaaaacaaaagaaagcatttttttctgaatctgttggattcctgaggttcagatagcaaatatggatagttcagataatgagggcataccagctttatcttgaaaaaattctccaaatctgtttagttcctgagcttgactagcaaaagttcatggtggataataccaatattagatcatcaactgcctggacaacacaaatacagttaaccatgtctaacatttttatattaaccccctcttcctttcctttagtactcagtatttatttaattaaatggtactcaacttcaaactccactgttccatggaatgctgcggtttccctttgaagcttttgagctagcagcccatttccggttcttcatacagtcagcgcatctgagacctgagcatgtgataaagcgtgttgctagacatggtcaagagttaacttgaaatgttcatatttagttcacgctcaatgcataaatgtaccctagcagaaaagagtacagaggtgttctatagtttttaaatgttgcctttgatttcagaactcctagctaatgtttatattttctctctcccttccttccccggatactagcctggctgggaagaaagaattcacttggatggccgaacgttttatattgatcatagtaagtaggcgctgttatggacacacaggtgttgtgggttagagggaaggagtaattcaaacatcatagtgaaagctttgtcattttactctttatctaggcagccaggtgttatgtggagaaaatgataccagagaatcagtgccttcatacggtactgtgcattcaccccaagtttgcttaaagtagcccatcaaactttcacccaatctgatcttgtgacctattaacgtgtttttttttgttgttgaatttttaaaccacattttgattgttgatcccttgacttttgcacatttaataaatacactctggtaatagaaaataagattagaaatgatgatttagtcaaaaatctgtgaacagtgcttgaagcagcatgtaagttttttagacactgccttcaagggtcctatggcaggatgcccgctctgaccttattttctattcacggcatgtttccttgcacccacaacactaatatatatgtattttctggaaattgtagtatatttgctatagacatttgctccagcacctatgaaaaaatgtgtggtttctcattccaactaaaagttcaactaataaaagtttgctttttagccagtgggattttttttttctcattctttttggaggtgccatgttcttcatggaatcatatttctatgtcccttaaattttctgaaaaagtatattaaaaagaagccttcaaatagatgtaaagtttcaaccatgagtgcaatttggtagtatcttggtctttgctttcatttggttcatgaacagaaagttagggtttccctggcttcacaatttcataactaaacaaaagcacgttgatgagccacctgctgagaagtctggccacaagtaatcctcacacgtataggactggtcactatctcataggcatagtacgtttttcagatgttactaggaggcagagacattgtgaacagaatttcactttcaggccctgggggttgagggcttattataaatggaattaagcaaaagtaccagcagtttacaattcaactgaaagtagaagtcctaggtgtatttagaagagtgagctaccctttgtttaaaccagatatttcaaggtgcagagacttagtttttgtcttttatcaaaaaatgggcagatttgtcaactgtccagaatttcatagtaagcataaagtatttttcctgctttcatgtatgtgtatgccctacttttttccttaatatactatttatttattgttttaagagaatttactaaaatgagagagaggccacacctctttttcccatgaaaaacaaaataatagtttccggcttggtgcagtggctcacacctgtaatcccagcactttgggaggccgaggtgggaggatcagttgaggtcaggagttcgagattagcctggccaacgtgatgaaaccccgtctctactaaaaatacaagtaaaaactagctgggtgtggtggcgtgcacctgcaatcccagctacccggatggctgaggcaggagaatctcttgaacctgggaggtggaggttgcggtgagacaagatcataccactgcactccagcctgggcaacagagggaggctgtctcaaaaaaaaagaaaaaagtttcctttatagtattttttatacatcacaatcacagttttaagacaggtgtgaataatactgatcattttactaaaacaggtctttactggtaacacatcctggtctgtgacgcatggagagcattgcacacaatattgtgacagttttctggctggagatgtgatgcttaggtccccctaaaaagaatgttgtgaatgtgtttcgtataccgcaaggagccaaaaacagaaaaacacaattacaaagggctgtcgtagcatgcgttaaaatagtgaaaatcactgactatttcacttgtggtgtcatagacctagtatgatacatagcagatgcttgatacttggtgacttgaaatcaggttcatggaggatcagtggtgaagctcttaggaagttgttgtgcttgatcagaaagtgaagtgaactcacttgaggagcagtaggtgcccccactccggctttccacccccaccggcctgaggctagcgcaactccggcttactggaaggtggaagaaggtaactacctgctagatgcctgggcttttccttactcctgatcagttctgcagttactccagcactctgcccaaacatttcctcatttcacttgactagagcagcacccccgaggctccattatactctgtacagagcttgatgcaactaagctgtgccaatatatttgtggccattgacttggagattaaaatttattttgcccttgcacaaccatattgaaagattctgttaaagagatatagcataaattaagtgaccaaaaaccatttaagaaaaggatttgtttacgagtgcctccttagctgttcatgctacgtttctaccatcgctccataattcatcaaggtttactcaatggaaagctgtagagcagtacacgtcagtgaaacagttcaagtcatttgcctttccacaaaattcagtctgtgaaaactgcagaatacctatcttttgaagaggctttatcaggctgacacatacattccaaatgggttgaattacctggaagataacatttaggaatgtcctttctgtcagaagaaagcctctttttctaaacaatcttatagcaaaactcagcatctgattttgctgtcataatgcctacaggacaggaaccaggttatgcccacgtatgccacaatgccgtgagtgccgtcatcttgtaaattgcttagagtcatcttgtcttccagcatacctggcacgtgggtccacctggaattacagatgagatgatgagatgcacttgtcttttcaataattaattatttctgctttgtaagtttttcctgagaagttagacattttcaagatgaacaatactcagccatcataaagatttaatttcaagagcaacgaatgatcgagactaacctctttagactcctcgattcctttgtttgctgtctatggaatggctcctggctcttgggcctcaggtgggaggttaaaggaaggtcttaaactgaatctctacatcttgtaggaatgatgtcattcttcttaaagaccagacaggcccagcagctgagtaattggccacaggtatcctatctacaacattagaagagaaaattgaattccagtgattttgtaaagtcctattttagtctttcctgtcaacagtcttactagacctgttcatcgtgactaaactgagtttctcatccttccacttccttcttgtccttttattggaaatatatctcaaagaggaaaagcaagtttaaaagccaaagttctttaaaaaaaaaaaaaaaggataactggctaccccatactttcatacattgaccatacttcactgaagaattctttatcataaccatgtagaaacttgtgttgttccaaaagcttaggctctttttaaagggactttgttttcaccatcagcttcattcagtcattaaaaaaataatgcactaagttcctgttacctgctgggactggtcttaagtggtagagaggggattgcaaagatgaataacacacagtccctgtccttggggaggttaggtccagtgggaagtagcaggggatatttcaggtgttagaggagtgaatcagttgttacacagaaacagaagaggcaaggactgcatctgcttgggaggtggttgggcacagggatggggccctgagagaatgggtcactttttagatggatgacaaacactgagtaggattgaggcagtcaaagccccaggaagagtgatcctaggatgcgtggaggtgtgtgaatatgtgcagatgctgccacgttccgagaaacgcagcattgctccaaacagctagtgtgccgcaggggcgcacaggccaagccaaagggtatctgcccggtgtggggactcgggctcaagccacaggcagtggggagctcccaggggatttcccagaggggagggatggggtcgaaatcgttatttagagagatccttttggcagagctgtagaagatgtcttgaactggggttggggggtgagacaattcccagtgacgcccagagaacagttcagggaaaaaaaatgagccctctagcaagaagcttatcaggaatagagagggttggtccgggaccaccagagtgagtgacacggccaggttcctgattgggacagtgagtggattgtgatactgcacattggaatgtaaggtcacgttaccaattttggtgttgggtgatcagaggtttggttttggtagagttaagctgaagtacttacgggaaacccagatgcagatgtccaggatggagcttgaagttcaggtctggtctgaggacttgcgaatcccaatgagtcaggagttattcatttattttctgctttgatgacaatatccattgatacggttataatccaagttggtgttttaaggcgcaatttttactcagctgtactccgtttcctttatttggttttcttttctcttgttttccatcacaatagcaacttttactttccaaagtcttctgtgaaaggtaagggacccaaaaatgccttttgcttccatcaagattctctcaaacagttgagatttttcatgtaaattttctgccagcatgaaaacaaaagattctagaatcatcacttgctttggtagcttttttttttttttttagcttttcctctgcttctattaaaataataataagactttttaaaattcctaatatggtcattaaaacagaattcactaattattcttatcagctcacttacagtgtatacagaaaataaacagaatcaatattcttcccctgcactcccccgccccggtccccaagacaggtgcagatcgggctggagcatagtggcacagtcatagctcactgcagccttcccctgggctcaagcgatcttcccacctcagcctcccaagtagctaggactacaggcgtacaccaccacacctggctaatttttttttttaaccttttgtagagatgggtcctcactatgtttcccaggttgggcccgaactcctggcttcaagtgatcctcctgcctcagccccccaaagtattgggattacaggcatgagccactgctcctggccaccctttacttttcaaataattttcttcttctttttttttttttttgttttttactaacctagatatccaaaaactctggagaacctactctccctagtgtgccccaccccccgtacctttcatgtgagtccattgcactctctctccctccactccaccctagtacacacacaggaatccgtaaggtctttttaattatgcacagttacaaacaccaagtgatctaaggcagctgttgtgtattatcacctacacaagtaatggtatagatagcattgaacagtcttgtgcacattaactcacttggaagaaagcaaaccaccttttgccagtgtgaccaaacagaaaccatgctctgtaatcttcctctgctgtatctaattctcttttttggactgcctaagctgctttgatttttgtgtgcatatgagtgacgcatgtaatagattattcatttgttcaatgcaggattcttaattgaaagcagtttttcaacaccataagtaaagagaaaagtatttgaaatgagaaaaaaaatcgtgtgtgtgtttgttagccttttgggaaactaagactttttaaatgctttttttcaactaaatgaaaatcaagatcaagttataaatccttaaaagaacaggggtttataattcccattttagcaagttatttgcccaaaacagtatgaatagatggtttatctttagttgtgcatttttgattgagtgtttttaactaaacagtatgtgaggggggttgccatatagactgtagttacctgctaactctgaaatactaatttgtctttagaacacagggtgggggactggacagcttttgaagaatccagaaatacttccttccaaaactttctttacatagaatagatttgaactaatgttctgattttttttttttttttttaacattttcagatagcaaaattactcagtgggaagacccaagactgcagaacccagctattactggtccggtcagtattttcaaattctgcctcttcagtataagattttggttacgtgtttacgtgtttcttgtgtattactgataacggtgatgtcaagggacaacggtgattgagtagttgactagaaacagagcactttgctgccatctccttactgaagtttccaggtgaggtacagctgcatgtgccttttcagatggatttcattataaaggaaaacagcttgataacgacagcagaattaaacatttttcttctcctttataaaagggtggactataagtatagtcaaaattccctcccttcctttttatatgaagcaaaaccaagccctatcttcataaagtgttatcatttctttaggccaggagtagtaaatgtatgccaaaagtttaaatcttttacaactttcaaaaatctgtagaagtttgggtgagattatagctaagttcaggagctggcagagttaagagtttggaactctgccgacccacagtgttctcatggtcccctcatatatgccgggtggaaaaagaatcaattctgatgcaggcgtgatgacctcttaaaacaccgtagcacactgtgttcataaggcagtgtgagaaccccactccctgaagcatcaggagacatttggtatctcacacttgttaacaagtctgtagaatgggaccacactcatcagcagagagcacaccaccagcttttgtcagtggctatacgagtgcagggaggtaatttggaattacaggagttgtactgcgatcagcagaagattgcaaatttgggattcttagaccagaggagcaaaaggaactgaagtttgttatatcacatgctgagctaagaggccagtaggtagatttaaaattccaatttcatggctttaatctgattccattcaaattatgttttgaattagtaagatacatccatttgaaaacttaaaagatactacattgcaaatgtgccccggtaactgactcctgttagttacttgttggatttgtttagtcatttgggtactatttcctaggcaaagtgcccagtagtacctgggtaaaaacagtcaacttttttctctatgcggcttaaacactgccttactctgatgcttccttttgatatatatatattcgcagtcacagttgtttattggaatattattttctcttctacatcattagaacaaggtaaattcaagaaactattctttttaagtgacagaagacttttgacataactttgaagccttttcaaaatatttttatcacttatcaattaccggccgtcactcaaacactgctagtccttattaaatcttactgaaaattgctcagcatcataaagccaagtagatttgcccccaagaaagagctctctgctttctgttaataaaagacccttgtttattccttcatttttattattattattcattatattgctttcaatagaggagaatctgtatttaggtagttatcaacttagtgtagacatttggcctttgttgtcattcagttctaaatatttggcaattttcatttgatttttcttcttgacttatacattatttaaaatatgctttttagtttctaagagtatgggagggttgtgcctattcatttttttttattgatttctaattttattgcattatggtcagataatattgtctagagaatatttattattggatgtttatttaaaacttcattggaagcttgatcactttttgtaaatgttctttttctttttaaaaagaatgtgtattgtgtctttggttgaatatagagttctttttgtgtccattaggtcaagcctgtggtgttcaaattatctacattctcattgctattttgctattggatctaatcaagatagttctgtcagtctctgtggtgactgttgccctgtcgatgttgtctgaggtgtatataactacattctctttctctgttcgtatttgctgtttagcatatcttttctctctcgcgatattttatcttctttcttgccttttgttcaattgatagagtactctctattttattttctctatgtgcttgcaggctgtactttctatttgtattcttatagctctcacattaaactgtgtaacccacatacttgacttagggttaaaatgaatcactgtctactctccttaacatcaggacctcagggaacttgaatgccattcttcccactcccatttgccacgtcattgttgggtggaattttaaaactaccttgctttaaactttcctaatagtcaaatagtcattaatatgttttatcttttttctccctactgtccatactgatttaattttaccaaatgtagacaaatttctttgcccaccattggtttgcccaccattggttcttccatccattttttttctggggtgagttttgatcttagtgaagcctagtccagtttatggttcttactgtgtggtctgctgaccagagtatcagtatcgcctgagagcttgttggaaatgcaagttttcaggtcctaccccagacctactgaatcagcacctctgggggtgggacaccagtctttgttttaactgctctcccagggattccagtgccccctcaggtttgagcacttctgcccagcagcactgccagtgacagtctgagagtggtaatatctcttagtctttgactcaaaaggtctcatttgacctctctgaagttcagctgatgatagaaatcttggttaaattattttccctcagtgctttggagattctcctgcatgtcttctggcctccataaatgatgataacaggtttgctgtcaatctcattgtcgtttctgtgtaggtaaatggctctgttctctagtgattttgaatttttatttgtgtcttagatgttttacaatttcgctatcatcatgtacctatgtgtggagttctcttcatttatcttgctttggactcaggcttcttcagtatggggattgtgtctttcctctgttctggaaaattctcagctattatttcttaacatactgcatttttccgattctctctaagtatctgtttctgtaactcctattggacatttacttctctttcacattgtctgctttatctcttaacttttgtgttttctgtctctcactgctgtattgtgagttattacttagctcttccagttaattctttaagctttttttgtaatctctttatcagttcattgtgtttattatttcagtgactaaatttaattgctcaaagttttatttggtcgtttaaaatttgcttttgtcttcatagttattttgtcctgttctctttatctctttatttatttttgatgctttcatctgcttattatttttaagatatttatttcttagcctctttgagattattctattatctctggtcctaggatcattaaatctcccactacgtctgtagactctccttaagagtgtagattattgcctagttcagtttataatttgtgtgtgtgtgtgtgtgtgtgtgtgtgtgtacatgcatgcaagagggagagagagagcatgagcaagctcatttttttttcctatgaggcttttgtaagtcctgacctgtatttactgttaacttcttagcttgggttcatgcacccccagtcagtataactgtggacctcatacccactttggcacaggcttggagtatggatttattacaggtctgtttctttttgtttttctcccatttatggtcctggacagaaggtaagcttccttgcaacttccctggtccggtgggtagagttttcttgtcccctttccagatgttaggttttaaacaatgactgttctttctccatcatgtagaccaaaggccaagttctgtgtccccatgggagattaaaacccaagcccctatgtctaggtccagtgcccactgatttctctaattgtgagtctttctgcttacctagtacctagagtttctcttcccaagttttaaaaatatcagttctaagtaggcctagcgtttctacatatttttagggagaggggaccctttctgtggcagctcagtgttcagcattcctgtaagttagcatgctctgtgtatagcagatatcactagtaatagcatttagtaagtgatgttcacacatgctgctgtcatgaacactatctcatgttgtgtaacactttcatttttccaagaactttataatcagccgacttgaaactcacagtcgtcccctcagaaaggcagggcaaatgttgttatttccaatttgtcagaagctcagaaagcttattctgttgctgacagtccttgcaagggtcagaatcaggaccggagccccagatgcgctggtgtcactgatgtccccgtgccgggcatgagcccttctgtgcaaggagctccaatgtctcccggccagtgatgatgtgaaaacatttagaaccgacctacacaataaggcagattttcattctgtacccaaaacaggaacacagatttaatgcagagcaaaagggctttaatcaacagatatgttcatttttcacgtagacctattttacaagctaacttgtaagccagaaaatgacattcgagattttcaagtgagaacaaatgatttggtccaataattaaaaaaaaaaaaatgtacgtgtaatctcttcagaagctgcactggaaggatgtttgtcctggtacgttgttggaaagttttcaggaagagaatgtaaaaacttgctcttcttattctatcatgagaactacctctggagagaaaagataatgaatgcaagaggaagacagtgatttttcagctttcacgcatcattccccagaaggagaaaggcttgaaatatctcaacatttccgcccacacctcctcagcccctgccaggagacagagcaggcaagggaacgggcatcataggctgggggccacatgttgaaatctgctccttggggaatttgtcatttaagacagtcgaacttgcatacacagtgatgactgtttaagctttttttccaacattttcaggagtagggggaactgtgatgtaacagtgatgtttggagaaccctcacgaatacaggaacaaacaaaaaatagttgctgggaatcacagactttcagaaatagaagagctgagtcacgtttgttcatccagtcaaattcttccacttaacagtgaagaaactccttagtagagaggatgtgatgcagagccagctgatgagagtctacccgagagtgcaggccttctaacgctggcaggagagagtggaaggaagtcacttttttagttcctatggggtcatttttttcctcatggcactaccacggtttattcattgatttcctattgcctgttgctctcaacagcttgtgagctccaggagggaactgggaaagctacctttggtttatgtactgttatttttctggcacctaaaatgccttgcacagagtaggtgctcaagaagtatcggttgaatggacgagaatgaacaccagaaagtgagaaataaactagagaggaaatcttagtttgtgacgggatgtactgttggtagagcagaaaacatgatctctttattggcttatgtcagaacattgtattctttttcttattttggtggttgtccaggtattaacaaatttataccttgcctagactcagaattgctatgtggtttcatcagactaaacactgcctaaaataaataggatgtgaggtcaaatctgttgttcagtggaaaatcaaagaagactggcttaaaaactaagaaaagaccataggcagtgtaaattgtctatagattgctcctaagctatataaagatgatgtattttttgctctggaattacagagcaaaacactgacgtgctcattttctggaagagtggttgtttgttttgtttcgggttttttatataaacatgtcttaaaactataaaagagggaagttcatgttcttcatttcttctgtttctggtcaagataagtatggtgaaagtgtaaggtttggagtctggagatactagactaaggatcagtatattttattaacttaggccgaaatgataacagcagaagtattctctgctgaaattcatactagtggctaaaaagcaatgagttgttttcgaggctgtcatagttaatgtctgtgtatatgcaaaacccaatcctttttgctcataaaaatatcgtaagctttttcagtagccaaaactcacggccctgactctaagatattgtaagctactttacaaggtagcaaataaagattatttaaatgactacattaacatgttactcctttgcctgaagagatatcacaacttttgaatgactatagttaactcaattcctgagccctcaactggggtactatcactctatttcatagataaagcatttgagatttaaagaaatccttgcccaagaccactaaatattaagggacaaaccttctgacttctaaattgaggattttttgagaatgccatctgcctgattttatattccttgataggagtgtcagttcacttccctttctgctcgttgtatttccccacaaaaattcgttaaaccgtaattagacttttcaaaatgccaacgtaggtcattctgggttgtatgttagaagtatttagccaataactcttacctaaaccacagcaaaaaagaaaaacagaaacaccctgactcatattctgacaaaagtcacttggaaagcatgtttagtttgtgtagcgtaccccaaatggtttctcaaatagcagtatagctgttttcaggggtgaaagctgcattcaaagctgattagccctcatcagttctaggactttcccctgtgctgtgtagacattatctaatttttccatggataatctgagaagtgagaatttacggtttatgactcatgaaattctcttgaatgttgcctcagtatacataaaatttcaggtgtattgtttgcattatctatatttataaatttatcttccaggctgtcccttactccagagaatttaagcagaaatatgactacttcaggaagaaattaaagaaacctgtgagtaatcatgccttccaaaaatgctttgtgttagtcatttgtaagttaccacagtcacttcagtgagaacaacttggttgttacttttatatgaatgctaaaaatattaccttccttttctctctgcactttgatcctcctggttctaaaatctgaattttctttttaacagaaatggtaagtgaaaattaactcagtgttacatatcaaatcacctgaagggtaatttcctatgtaatacataaaaagacaggaatgatttttatgggatataaaataattcagaaccctagtcacaaaatctgtggaagcctattcaattatgcgcagttatcatatttaattaacctggatcccggtaagaaaaatgttcaagtccttccttgtaaatgcaaaagcttagtttcaactacagtatttataatatagtgttatatttaattataataattggcagtattacgtccaaatgtaattttctacttataattatccctggcagtttaaaacctagaaactctaaaaatttttatgggcaacattttaaattataacattgcattttctaatgacacaccatccccaaattgttcaacattgcatcctctgttttactttccgattcctccttagcagttttctcttcaattttctccatcactatggttacacctctagggaggggagagaagcggctgcagctcagaccccctctggtggcctgggctccattgccatctctccctgcctcctcccctcagctggttcctcctgcccgggaacatctctggatggggcacaatgtctgctctcatttaggggttttggtctctgttccaaagtcctgattcaagagcttgcgggtaactgctattgaaatgattctttgccttcaaaactcaaatgatcactgaaaattccacagttttctttgtgagtcatgtcccctggtaatgatgatgactgtggtcatcacccagtctcactctatggctctcacatagcagtgtctgctacccggaaaccatctctgcacattcattcttacagccagaccaagcctggaccgcctctttgcaggaggcagttgccacaaccttggtcaagtgttttgttattttcatgtcagcgaaccatgctttgggctgctttccccacacttggcataatcaggatcccactccccatggcaacaaaatggacttgaggcagcgtggattatatctaccacctggcggccctgccacagttgagcatggataggcccaacctggccaggtggggattattacaggttttgactacctgggctaaagtctcttgagtgaagcaacagatacagtcgactgcctttcggttgccctatgctggagaagtgcccacagttgtttattaaagggggcaaggaggatgttctccttcgcagcagagttctaactttcctgagcatcgctagtggatgccgtgtgtcccatcgtgacacagcaaggctttctgtctgcaagggaaaacccctaaaggagagaatctcagttggaaccatttgtcactgcctgtttctttgaggactgccaacttttctaccatttgctgttgtttttattagaaaacaaagtgtgtatttactgtatgattatgtgttttcttttgtcttttgtgtaggctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacgtaagtatatggccacacccagtgtgtgtcccccactgagacagttgtatgaatttaaacagaatgaaaggataagcagctcatgagttcgagatgcattaactctgctgagtttgttctctcctcaccccacagccccttgcaagtctagaacaggttctgactctacgtggtgaaatagtgtactctgcgaacatctaacattgattttttttcttgtctattgggttcatggagttgaaattgaaaacgtggataattatgataaggaacaggaggcgattttcatttgcagtgttcaagaggaggacttgtcagtgttagaattcaaaaacaaaggaggtgttttttgaacaagagtcgtttctgaggtctcctgtgtaaaggcaaattgtaaggactttgaggacctaggaccatgccctgctcctgtaaggaaagaaccaggtttccccttgcattaggatttcatcattgccaggcttggaaactgaggcagactaaattgctttctgagcaattaaacatcagctacttccaggccagccattcctccttcagactctgaatcctccaagggttgggcagtacccaagcccccagtagaatagtcattgaccttgctgaagggcatttgtcatcactccagccctgggctctaaggggacgtgggttcccccacatctcccacctggtaccagtctcctgggggctcatttggaaggccgcctcagtacattcttcctggttgctctagccaagaattgcccaattgatttgttgttactgagtcccatgagaaagcccactatgtacaatgtatccaactattgaaggtggagggcagggtggagagagacaagatctttacctcttctccacattttcaaagatacccccaccccggaggaatcagtctgtgatcccagtttgaaaactactgatccacatgattgtgaaccaagcaagatcatttggacaaatctaaaaatttgcaggtttgcatcattgagagagccatatcctgttcctaatcccctcgaggccttttcagagcagagtagtggtcagaagacagaccaagggttgggcccatagcaaacatcttatggccaagccaaaagcctctggaaggaattaaacttccttcatcacagagacgacctaaagtactcacatctttgggaatccctgcacagcccctttattatgtggcgtttctcatctccccaagatatttggctgaaccatatgaaatagacacttcgtaggtcaaaaacagtcaaacatcagcgtctcttatggtttaattgactttagagggcacatgcgtcagcctccatggtgggtcacggttgagcatgtggtaagtcaggtcctctagcccacacattttcccactaggtacagaatgctcgcagggacactgtaaaagttgagtgacaggtggcaaacaaatatgcaaaatcttgcatttgaaagtgtgattgggcttttcttctctttctcctcaaaagggacaactacacccttcagatcaaccctaattcaggcctctgtaatgaggatcatttgtcctacttcacttttattggaagagttgctggtctggccgtatttcatgggaagctcttagatggtaagtcttgaagtaataaaaataggtcagtgctgcttgcggtttgctagtaggtgtcttatctttcgcatcatgggtttttaagcaaaagcttcactggtttactcataaagtatttttatgttttgcccataatggatgtccttttctctggtagcttattttgggcactgaagacatagtgtctacacacagttaaaaacaaaagtttttttttaacttcagtgtaaaacacagaacattatgtatttatacatttgtatgttttctggaataactttagtgaaagcaagatttgataacagatggaaaaactaattgaaaagttaattaaaatgatctcatctatctaatatcctgtgattattgcgtttgtccccaaattattttttaatttgaagggactaaataatttcaaaataatgttactgcttagctaagacattaaccctgaaacaaataacacaaaataatttcattcttatttaaagcatatagatcatggaagaatttcaaggtaccttgaaagttagaagtcaaaccctctacctggcggtatctataggcttagagacttctaaaagatttggccaattcaaaatagtgtccttaagaaaacactacagaaatcatcagtgagaaaaaagcatcagaaatactttttaagagcctggaggtagagtggttgaggatgggaagagggagagtaggatttgagattgacattgcagtaacttataagaaagatatttccaagggaactgtgttcattgcatttatagagccttagctattctaataaataatttgtataccctctgtacttttcccatgaatatgtttttactgttaattggtagctgatattgatgctctgattttgttgctttatttggtagcagggcgacagtaaacactttttttcctctagaaggaaactaaaccacacactctttccatgtatgtatagtgtgaatatattttaatttccagctttggagtcatgccatagattgtttcagcctgctccaattcactccaactttctcagaaggtcagtttagtcaccgtatgcaagtgagaagatgctgttagttgctacagtgtctttaataccctctctaacagggggtaatagagaagcataagatatgatcctgaccttagtcagcttatattttagcggggggaaaagagatgagaacaaaaccagaagaatcacagtgatagtgtctgagcagaagggagtgcattgagtcctcagaggagaaagtgacttggctggaggctcagggaatctttcccaggatgagctgcacctcggaggtggggtagcatttgagcgttcaaaggctggggtcatttcagatgagtgaggaccagcccagagtcaaggccttgtggaagaagatgggcggaagcaggaaccactcagggtctcaaggagctggctaaagtgaccccgacaagagtgatcacgatataaacctggaattgcacatttaagagtccaagtgcacatagatgggcctagtcagcagtgtcttcaaatttcccacagatgggaatgtccctgtcggcagggctcattctgcacatctgtccgatgggaatgtccctgtcggcagggctcattctgcacatctgtccgctgctccccctggtcggcttcatgcttttacttccccagacactggagtctattggagcctgcatagggccacgctgagccggggagaattggcctgtactgcagagggtgggtgcctggtggggggatgaaggagcagaagcaggagcctggggtgccaggtctggtggggtcaggaccccctgtgaggaggtgcgtgatgcctttggatcaggtgaaatggaggcacctgcaggacgcctgaatggtgggaggccgacctcgctgctgttgcctccctgaggcctctgccgtccctccgcggggggttgcacaggccctctccggggccttccccatttccagcctgtccccttcctgtgctttctccccatcgctgcccaagcaatgataacagaagacagatggaatttcacctggcctttcactgactccccactgccctcagagagcaccccaactctccagcaccccatgtgggctctgcactgccggccccttctctggcctgtctacccgccctggcaaacggctggcaagtccccagccttgttcctctgcttagtgtgcccttttctcccccaattcctacgcctgatttgcactgcggactctgcttgaccttgaagctcatctgtcacctcccagaggcctttctggccccactctgctgcgccatctctcttagcacagctgccctcctcatgccactctccccctgatctcgcctcctcgcttgtgcaatactgtagaaggcccttggttactccttcatggtttctcatttacattgaaagcaggttaatcttttctttcatgctctgatacatagcagtgtcaaagacctgaaactaaacccagtgtttaccgtgttttaggtttcttcattagaccattttacaagatgatgttgggaaagcagataaccctgaatgacatggaatctgtggtaagtaaatgcacgtcacacactggccatcaccgggcactcgtgtcttatgagaaggtattggagagccatatttgtagtctcagccacttttatgggtgttgcattccatgtgaacaacaggagacatttaattgcttgaaaagttcattgatctaattcttgtaataaatcattgttcacatttcctggaaccatggtacctagggtggatacatccaaatcccagttgcctctgcttacccggcataggacttgtgtgaacttactaaatccaagagacagctgaagatctatggtagcctgtgttttctataaaaactgctgctgctgaatgtttagagcttcatccgctctccctctgtttcctgacatcataaaaatggcagaggcaaaggtcaaaacaataggaagagtatcaggcagacgtcctagacatggatgagtgacgccacagctcaggcaagtgaacacttgagacgttctccaagtagtgatttgggttttcatgttgcatttaacattaattttacctagacagaccattttagagttggaaggattttattttttttttattttttttattttttgagatgatgtcttgctctttcacccaggctagagtgcagtggcgcgatcttggctcacttccgccttgcggattctagcaattcccctgcctcagcctctcagatagctgggattacaggcacacgccaccatgcctggctaattttttttttttttttttttttttgtattttagtagaaacgaggtttcgctatgttggccaggatggtctcgatctcctgacctcatgatcctcttgcctcatcctcccaaagtgctgggattacaggtgtgagccaccgcacccagcacggatttgctataaaacaaaaattgatgttctttttataatatacttctgcttctgtttttcttttaatgtacctataccccacatctacctccagagtgtatgggtgtttgcccacacgccagcacacacacagggccactgggaagcgcgtttgtccatttggggtatttgcagctcattcgtgtttgctcacttgagttaaaaagcccctgcatgatgtcacctgcctgtgataccaactgtattttgggcacggttgggtagatgagctgtgtcctcggccctgaccagcgctgactcagtgtttctgttgctcctcattgtatgaaaggagaggaagaggccagcagtgcccttgaacccactgctggtttgccagtcatggtgggttgggatgcacacctgtgattccggtctccactttgaccctgtgtttgtgctttctgctttcatcactgtatgatgaagcagctaggagcacaggaatttctcttaacgtctaactttataccacatgctgacagttagtgcctaaagtacagtgcgtgtattatttttaccctcttcacagcccgtcgtgtccccagcaggagagaccgtggagggaccagggacaggcgcttaggcctgtgactgatcctctggattctgaaaccagagcagccaccctttttgctgacattaacctggagagtgatcaaagcacaagaaaggggataacttaaaactattggtatttacttagttggaacattattttacttatttatttttttggcgatagaatcttattctgtttgccaggctggagtacagtggcacaaccatagttcactgcagcctctatcccctggcacaagtgatcctcctgtctcagcctcccaagtagctaggaccacaggcacacaccaccatgcctagctaatttctttttttttttttgtagtcatggcgtcttgctatgctgcttaggctggtcttgaacagcctcaagccctcctcctgcctcagcctcccaaagccgaggattacaggattataggcatgagccagcaagcccagtctagaacattctttatccttcctgttttcctcagatactttcactctttttttaaactttatttattttttatagagacagggtctcactatgttgcccaggctggtctctaactcctgggctcaagtgatcctcccaccttggcctcccaaagtgctggaattacagcgatgatattttcactctaaaaagatagaaagtatattgaccagcctggccaacatggcgaaaccccgtctctactaaaaatacaaaagtcagccaggtgtgatgtcacacgcccgtaatcctagctactcaggaggctgaggcgggagagtcactagaacccgggaggcaaagattgcagtgagccaaggtcatgccattgcactccagcctgggtgacaagagcgaaactccatctcaaaaacaaaaaaaaaaaaaaaaagagagagagaaaagaaagtatacttatgtctgtatgttaaattttgtagaccagtatggcagctccatccaccatccagagtaggtgccttccactgtgcatcactaaggacaggatttaactgcaagctcattacaactaaagcagtttgtttgtttatagtttaaagaggaaggtttggtttaggtggagcaggaaacattgcattagtaacatatttcttgagcattctgcacagaattacattagaatgtaattaaagaagtcaaatgagtttgtattttggggaaaaaatgctgaattttcactcctgtgtcattctgtaggatagtgaatattacaactctttgaaatggatcctggagaatgaccctactgagctggacctcatgttctgcatagacgaagaaaactttggacaggtacatgtgggtaaccctgggaactcttctttagctttcaaggggcatttttcttcttgacgggctgtaacacaactttgctggaccatcagaaacaaccagcaggagctgcaggtgttaattttcatctcattgtagcagcacctttttgtcatgccaagacgccacactccaattacaaccatttctctcttccagcaaagttacagaaattccaagaggccttggggcatgaaccagcacctcttctaatccaaagcaaatgtgaatgtttcatttgtgcctcggttttagaatttaggtacttgaaaaaatagtgagattgttgacttagcgatgtaaatacatgaccaggagtgggtgatgttggaagggccattggtttttctctctgttttatggttctgttgtcatggcgacatttgtcttgtgaggctcagcatggcaggctccggtttttctaccattccctctgctttgcccccacgctgttattcaggctggactaatcctcaaagtcagtttcagactggtcagcagggatttggatgggccactgaaagtggatttcaaaggtaaagttgcctgcttggaataattctcatcatgctaaggatctatgcgatgtatttgtctcaattctgaagcctttgcttcatctgccactcagtaataatgatgcagcttgggaggtgttctcaatgtattctctgaccctggtacctgtccttccgcaaaagcccagaggtatttcccatccttcagactaaatctccacggctatgaaattaattagaccatagaactcattgtaatggttgccctctgaaaaatgaaatagacgtgttctttattgctagatgtcaaagattgctagagaatctttgagaaaagaagctgatgccactagaggatttaatgacaactgagggtaaaaattgttggataagaaaataatcacattaatatcagaatcacttgttattcccatgtcctgtgtttgagccaggagagttcagtctctagtgtgagtcctggtgtgggtggaacctggggagtccatagggcccttcagtggctaggcaaggctcagagaaggacttcaggcaccagaggcaagagaccttgtggcttaatgtaaatgatcaagtcctttaaaaatctccttttgtgccccctccccactgcccccctttcccggcctcctccccaccccactgtgttgtgctttatgcctgagaatccttacagacaggcggacactgttctcctgcacggggaccttgtgatggggcagggaggggtcttttcagctgctgcccctctggtctccagcagcctccccaggaccgagagtactggtgtctccagccctgtctacctccttcctctgctgcctttgacacaaatagctcactccccccagagacacctgcaaggcacagtgcctttcactccgggctctcacccctccccactggtctccagtcccctggaagtgcatgctcctctgctggccctgtggatgcaggtgtcaccctgccagctgtcctgcacccgctgcttttttgtctcacccttgctatctggatttcctcatcctacctgtggctccggctgcacccatctccgctcaccaccagcctcttatctcaggccggcgtctgccttccagcctccagaattcccaggtctaacggcctcctcaactgcttctcctgagaggctcacgggtcccagtgctaaagccactttttcctccccagcccccacatcggttatggtctcatcagtgccaccactgtcccctcccacctgacacttgggcatcccctcagaatctccctctctcctcatgccatctcccctcctcatgccatctccccgtgcccaggcttgctgtgccccgtgactccccgtccttggcacgtccctgtctgtgcactcccctagccctgctccgatcacttcttgactgctgttctcacagcctcctgtcggccgccccctttcctgctttgctgttctccagtgccctgttctctagtgcctaccctgttctccagtgccctgttctccagtgcccgcctagctgtgagccccttatggactatttctgaaatgcagccctgaaaatgtccttcttctacttggccatcttgtgaccttcagcataaatgaaaactgttagcatggcatatgacccctcataatccagacctacccccctctggcattaccctctgacacctccttcctgtcaacctgtcaacaaacagtgtgccttcaagggtttccccagagatgcttcagtgccacattgctggctcgacactgcccactctgtaccgatgcattctcattctctctcccatcacctctttccctgccatcctcgaggtattgactgacgtgctgtgtgctgtatgccccctgaggtcttctgggtctctccagggctcagcatgtagttgatgcctgccagtgctcattgagcaaacgaatgaggagtgctaacctggcttattaaaaatatgaataaggtggtaagtaatagaaatcctaagtgtgagaaaacaaaatcagtcaagtcttggtggctttttttaaaaagccggacctattgaagggtcttaaactgccaattttcaaacgattagacatggttagtcaccctggaagtatcttcctttcatttttaacatatgaaggaaggagtggaagtaaagaaccaaggaagactgaaagtcagtcttgtgtgtcacatgcattaaaaaagtcttttcgtagattcattcatagaaaaccatatgaccaaattaacccaattcagaaaactattaagccacaatcagaagcaaatgatctgggaaaccttcctaattacatttaaatgtaattgtttccacctcataaatgaaagtcaaacacagggtgttggagtttttaagcaggcctctaataaatcttctgatgatcgttcaggccagattttcattaaaaacagttctcagacctcctcgtttaatgttgtgaattcttctgacttttgggtaggatcaagtagagtaatgcattaatcaaaacagacccatgagatgagtaagataagaattaggtgcccagtttgttgaattactgccttgcctttttgagagagctatcctgattaacagggaactcacgtatttattttgcactctgggaaggaatttagagcaagttaaatagctgcatatggatctcatctcccatatccagtccatcagcgagttgttcacacacctcccaaacctttctcatttctgtgcattctgccgatctcttctctcactcacacccaagtccaagctcccatcttccccactagacccccgacccgtctctacatccaatatggcttcttataatggcagagtggtctttttggaatataaaaatgagcatgtcattcctctgccagaaaccttcaggggtctcccctgcccttgagataaagaccccaaccttactttgtcccacaaggtcctgcgtggcctggctctccctgctcctcctccagactcactgtgaatcacaggccccactgggcttgtccaacacaagccccttcctgcctcaggccctctgctttctcttccatctgttgggagcatactccctcccagtctcagttcaggtgtcccttcatcagggaagccagcccttgacccagaccctatgcatcttccatgcctgccctgtggcatgtaccacagacgactttgcatttgtctgagtatgtttgtgtttagtgcctgtctctcctgctcagcttcctcccgatgatggcagccgcatgcctgccctgtgctcactcactgtcgtgtacttcccacaagcactttgcctggtgcatggtgggtacttgtagttgttgatgtggaaatctaggtgttagcatgatgcccggacaagcctctcacctctttggccctctgcaaaactgttgactctgacccagaagaaagcccattcctccccaatgccaaacagtcaagtcacttccacagtggggcaaagcaagtgcagttgtcatggggtgaaacagacatgcagtcagccttccacggtgtttcaaccttctttttttttaatttcacagttagaaatatcagaccatgaagtttggtagctgttccttcatgtaatgaagctgcagctgttgccattattttgcaatttgttttctttctgattggtaagaatctggggctgctggtttctagggcctaatatgaaagtttccactgaagaaatgtcagtgtttgaactatgatcctgtgccaagcaggaagaataagccgtacccccattcactgctgtgcacacatacctgcccccaccgggaggaggggcccttttagggctgtaacaagtgaaaattctttattgcaagttcagcagagcaacgtctaatttggttatgggtttgtttgtttgtttgttttttgagacagagtctcgctctgtcacccaggctggagtgcagtggcagaatatcggctcactgcaacctctgcctcctactttcaagcaattctcaatgcctcagcctgctgagtagctgagactacaggtgcaagccaccatgcccagctaatttttgtatttttagtggagacaggacttcatcatattgcccaggctggtctcgaactcctggcctcaagtgatctgcccacctcagcctcccgaagtgctgggcttacaggtgtgagccaccgtgcttggcctgatttgattgttttgaagagctgccagaagtttcaagaggctgtgcagtagttggagggagaccatgggcagcagccatataggaaggttctttaggatcgggtcgagccagtctccataggaatgattacccaaagggaacaaggaggacatcatctcagacattgaagtggttagtggccctcccagaagccacagcacattccacaggtgtactgtggatctctctgttaagatttcatggagagaggtgggccactggcagaaagaatctaaaaaggctctttagggggtgataatgaattaaaaggtagagaaacacttgggggaaggcagtgagtgaaaaggcagaaatggtcaatgagggagagggcagagcatgtcctggcggaagccaggagccagacgaagagcccggagctggaggtggctcccaggagcgctgaggccccggccccttcaggccagtcagggacacagaggggtggcacctggggagacgggaagtgggaaggctggcatggacttgctgcctccctgctgtcggtagttgcgctgctggagaggctggggaggatgaggaaggcctggttggtggagtgatgggggtgcagggtgctgactgcccccgaggccgtgccctgcaggatttgtgagcacccatcagtgggcgtttgcccagcagtgctccgtttccaagaatggagtctgacaagcagataagccttgctccacccaagtccagcatattccatatgagccaagctgtttctcagcagcttcccaggcagcgcaaagcatttatggagggtcagggagatgtgtgggaatcgggttgttaactggccaaggcagggagtcccccagaagttcctctgatctctgtgaggaagagagccaagtttatgctggggccagtagacttaaagaggaatttttctactgcagtgttctcccacgagctcttgctgagcgcctgctgtggctgggcattgtgttccttgtgtggggtcctccaggagcccaggcacaggagggcgatggcccagaaagccctgggagggcggggtgtgcgtttcgtggctgaaggtggcaagaatccaccccacagagcaggctctgcagactgctcaccagggagcagggcccctgggtgccagcaccagctccatctgcagttcgctgtttctccttggtcactttgctggatttctgcctccgggatgctgtccaccctcatgacgggggccagggcgctgccgcagcacctataggagatgcgcagggcagagagcagcccctgtgtgacgctgccccgatttctcccacttagggttgacactgtcacggtaccacccggtgttcagtgctgaggacatacagtccctcccctcctgtggtggcaacagctgatgttaagaagggacaaatttgggcgaaaggagaacacaggctgtagttagagtgatggtgagacaggaatgaaggcccgcagggaatctgtgtgcctggaggcgagcattagagtcagatagccgcctctccaacccccatggagcctcgtgcccaaccagcaaggactctggggcatcggccagtggcagtaagccagtgtggtgggctggacaatatgcctgcactgcaggagacggtgtcacgatcagcccccgacagccggtctcaaaagtggaatttgaagagtggtcattgcttctcatgaaagtgtcgccgtaactcatgaaagtgtcacctgtaacgaatcctagagggatgcatttctgtaccacggagggctcgagccgagacaccgagatctgggggcgtgacccctaacctagtagcttagggagcagaattacaaacccctcatcctgtgcccatccttgggccctctcctcactcccccacagagaaacccagggtcagaatcaaccatgagacttgacggctgcccacaggatgatttactgctgtgacctcatttagcatgggttagtgtgggcccattttttcttttctttcaaaaattatttatttatctaattataaaatattaacctccataataaaaataaagccagtataataaaaatatgtaaaataaataaatgtcacttattcccagtatctcaagaaaactcaggattaaaatttttatttacattttccagaattattctctgtaagcacaaaacagatttttttttttacagaagtggaattgtactctgcatactgttttcttttttattttatttatttatttatttatttatttatttatttattttgagatggagtctcgctctgttgcccaggctggagtgcaatggcatgatctcggctcagtgcaacctcagccccctgggttcaagcgattctcctgcccaagcctcctgagtagctgggattacaggcgtgagccaccactcccagctaatttttgtatttttagtagagatgggttttaccatgttggccaggctggtctcgaactcctgacctcgtgatctgcccacctcggcctcccaaattttttttattatactttaagttctgggatacatgtgcagaatgtgcaggtttgttacataggtatacacatgccatcgtggtttgctgcacccatcaacccatcatctacattaggtatttttcttaatgctatccctcccctagccctccaccccatgacaggccccggtgtgtgatgttcccctccctgtgtccatgagttctcatcgtttaactcccacttatgagtgaagctggaaaccatcattttcagcaaactaacacagaaacagaaaacagttttcaaattaatatatcacacataccttttcatgtcactgaatatcagtctataacattctaatgactgtgctgaattttactttgtagacgtgccatatatttttaaccatgccctgttggtagacgtttaggctattttagtattttctatataaacatataattctgtataaccctggaatactcacatatatacataatatatatagcatacaaatgtataatctggaatatacattttgtgtacttgactaatttttttccattgctataaactccaaaagagaaattactgtgagaagggatatataattttaaaattgtgatgcctattgccaaattataatatcttcagaaaaagattgaaccaattagactcccacccacagtgaaggggagaaccagttctctgcactcagcaacactggcttattttctctctcatccctgggcgctgcttttggaggaagcagagtgttttcctccttagccctcccctccccactttgtctgtggccgacccgctcattcagtagctctctggaaattacaactttcaggcaacaccaaagaaggctctgtgatcaaagagagagggctggatgaatgctggaaggagtgtattacagggattgtggatgcttatctacttgacctccttccccttccctgtgcgaggggaaagggtgggaggaaagattcggcttgagtgtagccgagctgtaccgaaagccttctcgataccaatgcctgggctgggatttattaacttgtcattgacgtggatctgataagaaaactcttaccgaaggaacaacaaggcagtctggggaggaataaatggggaaaagaagaaaagccaacctgggtttattaacacatagaacaaaaggtatggatgagcactgtccacgcacctctgagaaccagccacgaactggcagagtttcactgtagaaacatccccaccccacccccaagtggaagctgtttggcatcctccatccccaacacggcctgtacggggcaggagcagatgacccctgggagaaaatgggatgcactgggaagatggcagctagaaagggaaatggagaaatgaaggatgtcagcaaaaccggaaacgcagcaaagggattaaaattcacattgggcacagtgaagagttcagttgactttgcagaaatggaaagcatagtggaaaaatgagattccttcctgaataaagagaaaaagagaatgtcgagggaccggatggatgatgaatgtgttttacgttgaaaagccatctcagtgtgaatgggaacaagagccagtgcgtggagaagccgcatgagagacccaggtgtgagtgctcgggtcagaggggctcaggatctcctgggtgcaggtggcaggaccaaggaacccctggaggtggcctctgccagggcatagagttcatcacaacatggaacctccatccctcctgggtaccaccctaacacttactttggaaaaccctctcccctggcttggtttactctataatgcaaccagcaggcagaggaaaaaagatttagagggaaccccaccaaaatagtggcatggctttctttcagtaggagaggtgtaggtgctatatttttccaccttttttccccaaaatttctacaatgagaaaaaaacattttattttaaaatagtaggcctctctcccttccctcacgctggttactgtgctatgtggccttatggtaagctaggtctgggtcagggcataggagaaagggctgccctgattgcagctgcttttttcatgatcatgggtggtgctaagggaggaagcttcagggcagatgagtgaggagaaggcgtccaatgcaaataaatgccttcaaaaaccagataaatctgtctcttcagcctggatccttaagcttctttcttgtctttaagtttctgtttgcatttttgaaagaaataccatgcatcctttttttatgactagaaaatataattttcagaaacagaatgcattagaatttggatttctggagtcttgatgggtgtaccctgttcttttctctgttaaacataattagtttcctgataaatgaatagcagaggaaaaaatgttgctcttcattattgctaatatatgaagaataattaaaagtcataaataaccataaaacaatttgcctttaatgatagcatctttaactgtgtaacatcctagcaaaaaagcctcattggacattctcttgtgcacagagcccatttgccgggaaatatttctgaatacatgttgtcttccctttatagtaagacattgtcactcaataataatatgcaagataacttcgtgatagtaattgttgttttgtctttgtaattacagacatatcaagtggatttgaagcccaatgggtcagaaataatggtcacaaatgaaaacaaaagggaatatatcgagtatgtatacacatatttactgccttttctttgaataattgaaatgcatgtttggtcactgtcccttgctgaaacagctcatgcatttattatgggcatgagtttattggtcagttttcaacaaggtaagttcttttctggcttgggctaatgggaagttgaatatgggatggaaagtctgtcttgtatcatgactccattgagctgccctataaaggagcagaggagggagaaaaggtctatattattgagtaatacggcaaatactcttttggaatatgtggatatcaactaagatttagaattgcaacggagaaccacggtgtcagtgaccagcagggcaaaattaggaaccaacaaagaaaaaatattttgtctctctcccaacttcttttccataatagaaatgtatagaaatagttaaaatggataggaagaattcagcatgtgcaaccctaaggaattctttctggtatttgtaatattcttaactcaacttcagcctcagtggaacaaaatgttgagtctttttggaagtctagctgttaagtgattctaagacatttgtaccaggcagaagtttttctctgttgggcacgttacgttttcttttttagtgtatatgtgtgcttcaaatgcataactctgggatcttccaaggagatcccctggtgcaggggtgaggtgagcactgcagtgtgtggttcctggcacctggtttctgctctcttgtgggtcgccacagtggccctgagttcctcgtttgcattcagtgcttcgtttcaacaggctaggctccctcctttcttctcatttggaattggtcagaggtcttgaaatcagacagctcttgcattgactgtgggtcttagcacctttcagtgtgatctgcaaagaaaaacctactgtccttcctaattaggaagaaacggagcgtgttagagtctctactcagagatcctggcaaggagtggaactattacatggcggactcttttcactctcaagttgaaagcttgttatgctgaatacatagactagaaataaggtacctcggttcttacctggccacattaattggcactctcgttctgaatatgaacaatcttagctgatgacatcctggcagacaccattgtcttccttcagaaagtacagactgtgagtggggtttaaaaaatattctagctcagggtgacccaggtctcaagtctgaggactctgacatgcgtggtatgtgtcttgcatcattctttgcagttaattcttttagggttcaaacgtggtgggagtcagggccaggcagggttccagctagctttacttttaaaagtataattgaaatcaccaactgctccctgatacctctgctcccctggtgccagaagaatgctgttgagctgttcattgtagcccaggaggtgccacctgagaggccgcacagtggttgtgctgcaggctggtcctcagggccattaccatggttacagtgtaccaccccgaaggtgagaacccttcagaatgatggaatgcctgagatgacatccagagtacatttgccttgcgatcagttttgctgtgcattaaacactgtcgcagggaacttggttttggtgaacttcagttgcataaggaggtcttaccattcatcctctaaattttggcattaaattatgagtgcaaatcattttgctgataaagcttatatgttattgttgaatattagcacattggatactagaatgaaatattagataactaaatgcattgtcctgagtacactttacaaatatgtatacatatatgtgggtgtgtatacgggtgtgtgcatcttatacatgtatacctgtgtgttcataccccatgaacttttcccttttctccatgttgtcacattttaaagcagttaaagcatttaaagcattttaaagcagttaagcatttaaagcagtttaggtcagccaaaccttgatgtgggggaagttaattactcttttgaaatctccagtgagattttaggtttttcacgggtgttccagggagtcttgaaagaagccatgaataaactaaacagtgggggcacagaggagaccctcccctctgctctgctgtcgtggatggaggagaagcactccctgttgcggagaaagcagtgagcactagtgatgatggaggtggttcaatattgtcctcttttctcctgcagcttagtcatccagtggagatttgtgaacagggtccagaagcagatgaacgccttcttggaggtaagccatgctggccagggttctctgccatgtgcctctggtcccgggtcgatgggggatcgcgcttctcctttagctagtgtggtggagctgatcagagacagaggcgaggctggggaccctgcaggaagaccgggatcgcacagctcacaccgctggccagtctccagcacaagagtgttcagccaccgtgccgccacctccttttccttttcacgggttctcctgcttgacttctcttatttttacatttgtcctgtttttaaagaggacgttttacagacttgatctagaaccattgtcttggagtttcaggcggtacccaagagaaggctgccttttgctgcagtgacaggttccaagaagcccgagggctcagagctgaatgatgaagcgcagtccccaaagtgcctggccacccctccctccctggatcactgctgcctgggcttgattgattgattgattgattgattgattgattttgagagagattctcactgtcacccaggctggagtacagtggtgcgatctcggctcactgcagcctctgcctcccgggttcaagcaattctcctgcctcagcctcccaagtagctgggactacaggcacgcgccaccacacccagctaatttttgtatttttagtagagacggggtttcaccatgttggccaggatggtcttgatctcctgacctcatgatccacccgccccggcctcccaaagtgctgggattacaggcatgagccgccgcgcccagccatggacatttttttttaatcccctgccctttttctttgtggcataattcattgcaggttctcttctatacagatacatggaaaacacattttcttaagctgagtttttatttattttatacccagccacctcatgacagcttttaccctgttacagagcaaagtgggcactgcccagatcgaacttttaatatcagggatgctcccaggcccctagcagtgggtgagggattcatcattttaaacagtattccacttggaccaaaagcaaaattctttcttaaatatgatatagtattagttacctcaaagatcacacaggaaacagcctcgtgtccacgtgactgattttctctgcactgaggcagagctctcgtggggtgatgctttcccttcactcaggtacctacacaggatgcgtgaaagcccgggcagcacactcagaaacagctcggggccttagggacttaaaatgggaggacagaatggcaggtttcactctttatttagataagaaatcccaggaagcagattcctgggccccaccgcaggcggtccacaccttcagaaacgcggcgttcgcagcgcgcagggcccagcagagctgagctcatcctagatcacgccacagttagtggtacctgggaactggagcatggttttctgtacgcacagtgtctgcacccatcacctgcctccctctgtgtttcttctccctgcctccagcacgttatttttaagcgtgtctcttacacatctgtagcctgtgcatcttaggacgcactgactcacagcagtgttataaatggcttttggaatccaccctgcctacagtatcccatattttcatcaggtttcagaaattactccactacttgaatcttctagattctatgctttttcgaaactgttatattgtgactgtgctgtttttactggaactatagttacttgatactgtcactgacataggaatcatcaggagagattatatttttttctggctaaccagaaaagtttgtttttcctgtgaaattttttttttttgaaggcaataggtgtttaagatgttttttttttttctttcagggattcacagaactacttcctattgatttgattaaaatttttgatgaaaatgagctggaggtttgtattataaacattattttatgtaaagtggattttttaaagtatctctcattactttacaagtaatattttaatattcttggatccataatgtcctagagagtaactttagatgaattatattacatgtctcttatagggcttatttgccaatgtggttctattacatgtctcttatggggcttgtttgccaatgtaggagtaaggaacagcaaaaataatcagctgtagttgccttgtccatcagagggcagttctggaacatgcctagtaagtagaggccactgagcagagctgggatcatggccctatgcaccaggcgaggcaaattgccacaacaagtagaaacaaggtttgcagtagccctctggacagcagaaacctgcttacactaccagactggccaagtatcctctgctgcatccccatccacccccaccgtggcagccaaatgggggtcttggccacaaggtggccgtaagcatctacttgggcctgtagaacgacgtctcattgaacttgaagtggtttatgttgagtgaggaaaaatgtcagaaggcaacagggttgtagcaaaaggtagtttagtgattgtctagttcagcttcttggtgttgacatagttgagtaacagccattttaaaaacaaaggaatatctcgccctttgttgtgtcgagagaaacaggggaaagactgtctgttgagaagccgttggtgggactcggccatttgccccgtccatagccagtgagtgtttcccatacactggccttctatacttttatctcaaaacagaaagttcaagatcgatgatgtctatagaaatcatatacctcaagcctttcctctgataggtgggaaactgaggcagagagcagctgggtttacccagcaaggttttggcacggtcaggcctaggaactttatttccaggcttctggcttagttctgtttccatttcattctggtacctttgcataaatatcttccatttgtccacaggaaacctcccctgatggagagttgcataataggaacggcaaaccttagactaaatgctaagtcgaagacccagttgtgccttagcgtatgtctgccattcttaggcctctgggcagatgaggtgatttgtcatcactggaattgatcaacctggaggagagtcataagcactaattcttttgtgggtagggagatgcttttagatattcagaggaataacctctcaggaggagaagcagccagggtaatgcacagatgctgctggagcgtgagagaatccacaggtcccatgcgtaagactagaggggtagggaggacgactgtggcatttttatatccgatacgacatgccgtggcttgcgcaaggttcgaattgagctgccctcgtgactgctgcctctgtgatctgctatggcaggtggagcctggaacaggtgtctgccttcctctgttgttatgatttgctagcttaccttcgcggcacagtgaccacatgctggcactgagggtggtcatttctcagtgtgtgatctcgcacctttcacatcctgcttttacatctggtaaattttcttcctagttgctcatgtgcggcctcggtgatgtggatgtgaatgactggagacagcattctatttacaagaacggctactgcccaaaccaccccgtcattcagtggttctggaaggtaactccggggcccagccccagccggtgtcctccacctgaggcaggattgagggctgtgtggcgccctccattgtggtctgacttcaggactgatgctggctttggggtctgttagagcttggactgctgggggagggaagaggcaaactgagccagcagccccgtggtggctcctctgtaactgtagcagacagctagacttgaatgaagttggtccctacaaaagcacactggccttccttgaccacctcgtgaaagtgacttttatttgagggactggtcctccaatgcgtaaatagaaatggggggcttctcagggcagaaacaggcttcagggatggctttgcaagatgccactttctgttaagaagtcgttttctcaatttttaaaatatattccaacaaatattttgatctgtgtatctttcaaccaaagataacacaacatttgatttattatcttagggccactgtttgtatcctgttcagcttaggagggaagtaaaataggaatgggttagtccaaaggagatatagtaaacaaacttaaaaaaaaaaaaacagttatcccccaaaaaggctgattatttacttcaccccaaaaaggaatcatttaactttttcaaatttatttttatttatttatttatttattttaaagatggggtctcactgtgttgcccaggctggattcaaattcttgggcttaagcaatcctcctacctcggcccctcaaagtgctgggattacaggcatgagccaccatgcccggcctttttcacatttctaaatgccttttattactatttcaaatgccagcaggaatacagagataaaaaagcaaaatcttcaaagcctagtccaggtctcacttggcttgttttagatgtttgcctacagcagtccaagtggcattaccagtaaagccacctggattcccataataatgcagatatgtaagatacacagattattttagaaccccatctttgcctttgttgttttgtttttgttttgttttgttttgtttcattttataaaatgatgtcttctagggaacgtgtctacaatctgaaatcccatctcataacctggcttccccttgtgatgacaaatcttaaatactgtatatttataattggcttagggggaactggtaagcctcgaacacagaaacatgagcttggtgccatcagagagtttgctttttatatcatttattttggcctattggcagaagaagaaagagacggacattgtgatcactcagtctcttagattagagaggccagctagtattgtggccataaaaaacctactcagttttcagagtcaactgtctaggtcttacattgcaatataaataagattaagttctgcctgcctgcatgacagcagcatgtgccatgggtcacgtggggggtataatgaccttctgcctctgttcataggctgtgctactcatggacgccgaaaagcgtatccggttactgcagtttgtcacagggacatcgcgagtacctatgaatggatttgccgaactttatggt

SEQ ID NO: 2 - DNA, 5157 bp, Homo Sapiens
caagcgcgcaattaaccctcactaaagggaacaccaacacgtcgccaggactgcgccgttcgctgcgctcataggcggcgatttcatcaagggtggcaaggatcgcctggtcgacggtcaggtcgtcctcgacgcggttgccctcctcgtcctgttccagggtgagtgggcgataccaggtgtccaccgggaaggtacggcccgacacctcgacaatcggcgcatcgtcgaagtgcttggaaaagcgctccaggtcgatggtggccgaggtgatgatgactttcaggtcggggcgacgcggcaacagggtcttgaggtagccgagcaggaagtcgatgttcaggctgcgttcgtgggcttcgtcgacgacaggctcgcgttatggctccgctttctgcggctctcctaccctggcatggtgtgtgtgtgtgcctgtgtgctacggagagtcccgtattctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtgtggttttgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgccttcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaagcaccctgtgcctcgggggtggttgttcttcaagcaagttctgcttgcacttttgcatttgcctaacagacttttgcagaggcgatggcagagagcagctgcaggcatggtccctggagccgagccttcaccacgcactcgtccaagttcgggatgcgggaacctggtcccagcttgagttcctgcctttcccaccacaaattatcaactggttgatgtgtacactaattacatttcaggaggacttaatgctatttatgttgtcctctgcaggcaaagcccttaataaatattttacatcctttctaatgacaatgaatggaattaatcactcaacaggtatagtattacgactcatgtttactttttaaaatgatttagaccgattttcagattttatttcgttatgattaaagatgtctcatgtacttggaaaagtgagcatttttttttttttttgtatttcactttcataccaggcttaatgtcaatgacatttttatttttgaagtactctgacacctccaccctctactttattagaattggaaggcaaatttttgtccaaaaacctacagacaagtactttgagagaatttccaatataatattagacataatgataattttttccatactcagaatgaaaaactggatattacgtttttgttttggggtttttttgtacaaatttagctaatagctacaggctgagagaattgtaacatagcatgacaaattttgtgttgacttgaaaggaatcacaccattattccttagaagtaattacatgtgttctaacacatttgagacagggttggactcccatttctcatccgagaaattacttaacccttcctgggcgctgtacagtcatcttttattctatttcctctttgctgtttgtagtagagacattttgaatgaaacttggcactgcttgattcaaaactgtggaaaccagatctgtttagtctcctgtttgtatgcgtttgctaatggtagctaaataaccagtttttgttgtaaatgcaccaattctgaaggcactttatgtactacatggaggtcatatctggttttgtttttatttttttatcatgaacattaaatgtgatgatgatttcttttccctgcacacatctttccggtgcaatatctatcaattgtgaatctggctgctggtgtataaaaacctggatgtaaagctgagcctacagacctgtcctcaccaactgttttgtgatttctactcaactacaaagatttatttaatgtactcttaatctaactgagttttgttaccaatgacctgttgcatgcttcaataccgtgtactgcctgagttgtgcctcttgtgtgctagattaaaagtgagacagagacttgacttgatcctctgagcctcaagctattgagctggtagtggcagaggactgagggtacctgcacagtttgattcttttcccacgttgtaagtctccattgcagaattgtcgtgcgtttgagaaaacacctgaggcagtgtgggagttgaacgaccctgctgtcctttttaacctgtgttgtcctagacctgtcggggcagtcaggggacactagagatttgatctcatgcgagtcatcaataggacaaaaaagttgtggtttggggaggtctgtttgttacataaaaaggacctttcggtgtaagaaattgccgtttttaccctgccctggctggcatgtgagaagccatggaaggttgtggttgtaaatgagttgtctaaaggggtgcagaggcctgaggtttctaaaagaaggtagatttctacagagctgagtgttggttcctttttcttattggttgaaaattacctggtagtgatcagaaaacttagatgctatgtaactc

SEQ ID NO: 3 - protein, 975 aa, Homo Sapiens
MFRLRSWASSTTGSRYGSAFCGSPTLAWCVCVPVCYGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGVVLPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 4 - DNA, 24 bp, Artificial
  Description BP24 forward primer
aagagatcattattctctgctttt

SEQ ID NO: 5 - DNA, 24 bp, Artificial
  Description BP24 reverse primer
ctgttacattttcatgagagattc

SEQ ID NO: 6 - DNA, 24 bp, Artificial
  Description BP42 forward primer
ctgcttctaaggcatataataaca

SEQ ID NO: 7 - DNA, 24 bp, Artificial
  Description BP42 reverse primer
aaacgttgttctaacagattatga

SEQ ID NO: 8 - DNA, 30 bp, Artificial
  Description BP24 extension primer
cagtttacagaaaattgttcatatttcata

SEQ ID NO: 9 - DNA, 22 bp, Artificial
  Description BP24 extension primer
gagtaagagacgggcagctcgt

SEQ ID NO: 10 - DNA, 26 bp, Artificial
  Description BP48 forward primer
tgctttttgaacttcagtgtaaggaa

SEQ ID NO: 11 - DNA, 28 bp, Artificial
  Description BP48 reverse primer
tctactcagtccagtattgggatacaga

SEQ ID NO: 12 - DNA, 24 bp, Artificial
  Description BP59 forward primer
acagtctcatgtttgatgcttcgt

SEQ ID NO: 13 - DNA, 22 bp, Artificial
  Description BP59 reverse primer
acgtgcttcatttcagccttct

SEQ ID NO: 14 - DNA, 20 bp, Artificial
  Description BP64 forward primer
ccacctgaggcaggattgag

SEQ ID NO: 15 - DNA, 19 bp, Artificial
  Description BP64 reverse primer
ccccaaagccagcatcagt

SEQ ID NO: 16 - DNA, 19 bp, Artificial
  Description BP65 forward primer
gccctcctcatgccactct

SEQ ID NO: 17 - DNA, 23 bp, Artificial
  Description BP65 reverse primer
ggagtaaccaagggccttctaca

SEQ ID NO: 18 - DNA, 25 bp, Artificial
  Description BP69 forward primer
gctgttggaaatctattgctgtttt

SEQ ID NO: 19 - DNA, 31 bp, Artificial
  Description BP69 reverse primer
acccagataattattttctctagacaaagtt

SEQ ID NO: 20 - DNA, 17 bp, Artificial
  Description BP48 Vic Tag primer
cttatgcttgaattgat

SEQ ID NO: 21 - DNA, 16 bp, Artificial
  Description BP48 6FAM Tag primer
cttatgctcgaattga

SEQ ID NO: 22 - DNA, 16 bp, Artificial
  Description BP59 Vic Tag primer
agtcagtgtattttca

SEQ ID NO: 23 - DNA, 16 bp, Artificial
  Description BP59 6FAM Tag primer
tgtagtcagcgtattt

SEQ ID NO: 24 - DNA, 16 bp, Artificial
  Description BP64 Vic Tag primer
attgtggtctgacttc

SEQ ID NO: 25 - DNA, 16 bp, Artificial
  Description BP64 6FAM Tag primer
tccattgtgatctgac

SEQ ID NO: 26 - DNA, 15 bp, Artificial
  Description BP65 Vic Tag primer
tcgcttgtgcaatac

SEQ ID NO: 27 - DNA, 14 bp, Artificial
  Description BP65 6FAM Tag primer
tcgctcgtgcaata

SEQ ID NO: 28 - DNA, 21 bp, Artificial
  Description BP69 Vic Tag primer
tgtaagtgttttctatataaa

SEQ ID NO: 29 - DNA, 19 bp, Artificial
  Description BP69 6FAM Tag primer
agtgttttctgtataaata